(19) United States
(12) Patent Application Publication
(10) Pub. No.: US 2025/0115628 A1
(43) Pub. Date: Apr. 10, 2025

(54) FLUORINATED FLUOROPHORES

(71) Applicant: HOWARD HUGHES MEDICAL INSTITUTE, Chevy Chase, MD (US)

(72) Inventors: Luke D. Lavis, Ashburn, VA (US); Jonathan B. Grimm, Ashburn, VA (US)

(21) Appl. No.: 18/893,747

(22) Filed: Sep. 23, 2024

Related U.S. Application Data
(60) Provisional application No. 63/584,647, filed on Sep. 22, 2023.

(51) Int. Cl. C07F 7/08; C07D 487/04; C07D 487/22; C07D 498/14; C07D 498/22; C09K 11/06

(52) U.S. Cl. CPC C07F 7/0816; C07D 487/04; C07D 487/22; C07D 498/14; C07D 498/22; C09K 11/06

(57) ABSTRACT

Fluorescent compounds are provided, including polycyclic chemical fluorophores, as well as method for making and using such compounds.

### RELATED APPLICATIONS

This application claims priority from U.S. Provisional Application Ser. No. 63/584,647 filed Sep. 22, 2023, the entire disclosure of which is incorporated herein by this reference.

### TECHNICAL FIELD

The presently-disclosed subject matter relates to fluorescent compounds. In particular, the presently-disclosed subject matter relates to polycyclic chemical fluorophores as well as method for making and using the same.

### INTRODUCTION

Fluorescence microscopy depends on the labeling of cellular components with fluorophores. Beginning with the development of fluorescein isocyanate (FIC) by Coons in the 1940s,¹ followed by the shelf-stable analog fluorescein isothiocyanate (FITC) by Metcalf in the 1950s,² small-molecule fluorophores have become ubiquitous labels for antibodies and other affinity reagents used in cellular imaging.³

Chemical dyes are also employed in self-labeling tag systems, such as the SNAP-tag®^{4,5} and HaloTag®,^{6,7} and in fluorogenic ligands that bind endogenous biomolecular targets.^{8,9} These strategies have expanded the utility of small-molecule fluorophores to label proteins inside living cells and animals, complementing genetically encoded labels such as green fluorescent protein (GFP).¹⁰

Of the extant classes of fluorophores, the rhodamine dyes remain in wide use as biomolecule labels, self-labeling tags ligands, and fluorogenic stains due to their excellent photophysical properties, tunable structures, and bioavailability.¹¹⁻¹³ Elucidation of structure-activity relationships in rhodamines combined with new methods to synthesize functional dye derivatives leads to the creation of optimized reagents.

Accordingly, there remains a need in the art for unique functionalized rhodamine dyes and unique synthetic methods for preparing such dyes.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes polycyclic chemical fluorophores as well as method for making and using the same. In some embodiments the compound has the following formula:

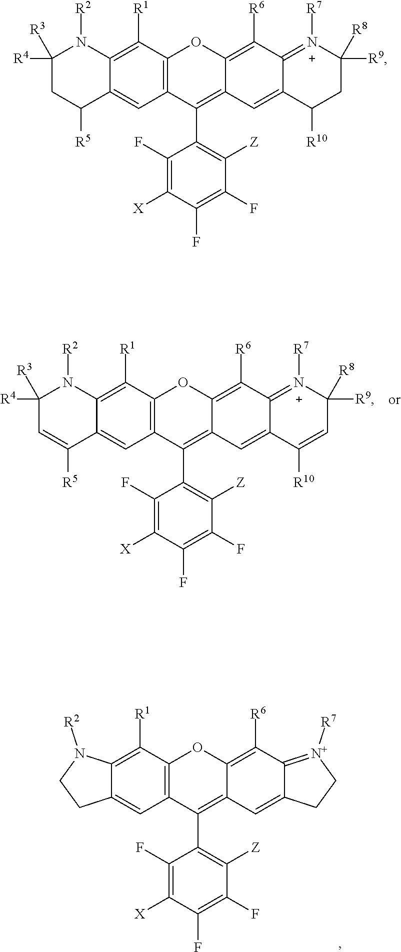

and salts thereof.

In the compound, Q is selected from the group consisting of C(alkyl)₂, NH, N(alkyl), O, S, SO₂, Si(alkyl)₂, P(O)(aryl), P(O)(alkyl), PO ₂H, PO ₂(alkyl), Se, and replaced with two H atoms.

R1 and R6 are independently selected from the group consisting of H, D, halogen, CN, OH, O(alkyl), O(aryl), SH, S(alkyl), S(aryl), N₃, NH₂, NH(alkyl), N(alkyl)₂, NH(aryl), N(aryl)₂, NO₂, CHO, C(O)(alkyl), C(O)(aryl), COOH, COO(alkyl), COO(aryl), C(O)NH(alkyl), C(O)NH(aryl), PO ₃H₂, SO₃H, alkyl and substituted alkyl, aryl and substituted aryl, alkenyl and substituted alkenyl, alkynyl or substituted alkynyl, or wherein the R1 substituent and R2 substituent and/or the R6 substituent and R7 substituent, taken together with the carbon atoms to which they are bonded, independently form a substituted or unsubstituted ring containing 3, 4, 5, 6, 7, 8, or 9 atoms.

R2 and R7 are independently selected from the group consisting of H, alkyl, deuterated alkyl, and aryl so long as when Q=C(CH₃)₂, R1 and R2 form a 6-membered ring, and R1, R9, and R10 are methyl, then R7 cannot be methyl.

Each R3, R4, R5, R8, R9, R10 is independently selected from the group consisting of H and alkyl, so long as when R1 and R6 are H, and R3, R4, R5, R8, R9, and R10 are methyl, then Q cannot be O.

Z is selected from the group consisting of C(O), COOH, COO⁻, SO₃⁻, PO ₃²⁻, or CR₃ where each R is independently selected from the group consisting of H, OH, alkyl, and substituted alkyl.

X is selected from the group consisting of H, alkyl, substituted alkyl, aryl, substituted aryl, alkenyl, substituted alkenyl, alkynyl, substituted alkynyl, CN, COOH, COO⁻, COO(alkyl), COO(aryl), C(O)NH(alkyl), C(O)N(alkyl)₂, C(O)NH(aryl), C(O)N(aryl)₂, PO ₃H₂, and SO₃H.

The presently-disclosed subject matter further includes a method for detecting a target substance as disclosed herein. In some embodiments, the method includes contacting a sample with a compound as disclosed herein.

In some embodiments of the detection method, the target substance is selected from a protein, a carbohydrate, a polysaccharide, a glycoprotein, a hormone, a receptor, an antigen, an antibody, a virus, a substrate, a metabolite, an inhibitor, a drug, a nutrient, a growth factor, a liprotein, and a combination thereof.

In some embodiments of the method, the detecting step is performed with a microscope. In some embodiments, the contact step and the detecting step are performed in a live cell.

Some embodiments of the method include contacting the sample with a first compound as disclosed herein and a second compound as disclosed herein, wherein the first compound is selective for a first target substance and capable of emitting a first emission light, the second compound is selective for a second target substance and capable of emitting a second emission light, and the detecting step includes detecting the first emission light that indicates the presence of the first target substance and the second emission light that indicates the presence of the second target substance.

The presently-disclosed subject matter further includes a method of making a compound, including an intermediate compound, as disclosed herein.

In some embodiments, the method of making a compound comprises preparation of diarylether and diarylsilane intermediates, as disclosed herein.

In some embodiments, the method of making a compound comprises rhodamine synthesis via lactol (phthalaldehydic acid) condensation, as disclosed herein.

In some embodiments, the method of making a compound comprises preparation of 2,2-diarylpropane intermediates, as disclosed herein.

In some embodiments, the method of making a compound comprises preparation of anthrone intermediates, as disclosed herein.

In some embodiments, the method of making a compound comprises xanthene synthesis via lithiation of tetrafluorobenzoic acid, as disclosed herein.

In some embodiments, the method of making a compound comprises synthesis of carborhodamines via AlCl₃ Condensation, as disclosed herein.

In some embodiments, the method of making a compound comprises rhodamine 110 and N-aryl rhodamine synthesis via cross-coupling, as disclosed herein.

In some embodiments, the method of making a compound comprises MAC substitution of 4,5,6,7-tetrafluoroxanthenes, as disclosed herein.

In some embodiments, the method of making a compound comprises conversion of MAC xanthenes to HaloTag® ligands, as disclosed herein.

In some embodiments, the method of making a compound comprises synthesis of HaloTag® and SNAP-tag® Ligands, as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A: The lactone-zwitterion equilibrium of JF₆₄₆ (1) and JF₆₆₉ (2) with scale showing the different categories of dyes based on K_{L-Z}.

FIG. 1B: Synthesis of functional fluorinated rhodamines using masked acyl cyanide (MAC) reagent 3.

FIG. 2A-2D: Spectral properties of fluorinated fluorescein dyes. (FIG. 2A) Normalized fluorescence excitation and emission spectra of carbofluorescein 51. (FIG. 2B) Normalized fluorescence excitation and emission spectra of carbofluorescein 53. (FIG. 2C) pH titrations (i.e., absorbance vs. pH) for fluorinated carbofluoresceins 51 and 53. (FIG. 2D) pH titrations (i.e., absorbance vs. pH) for fluorinated fluoresceins 60, 76, and 77.

FIG. 3A-3P. Normalized fluorescence excitation and emission spectra of rhodamines. (FIG. 3A) 70. (FIG. 3B) 71. (FIG. 3C) 72. (FIG. 3D) 44. (FIG. 3E) 40. (FIG. 3F) 25. (FIG. 3G) 8. (FIG. 3H) 14. (FIG. 31) 94. (FIG. 3J) 47. (FIG. 3K) 29. (FIG. 3L) 16. (FIG. 3M) 97. (FIG. 3N) 57. (FIG. 30) 98. (FIG. 3P) 31.

FIG. 4A-4D: Absolute absorption spectra of N-arylrhodamines. (FIG. 4A) 64. (FIG. 4B) 65. (FIG. 4C) 66. (FIG. 4D) 92.

FIG. 5A-5B: Trends in spectral and chemical properties of rhodamine dyes. (FIG. 5A) Plot of Φ_{f} vs. λ_{abs} for nonfluorinated rhodamines (white points) and fluorinated rhodamines (magenta points). (FIG. 5B) Plot of ε vs. K_{L-Z} for rhodamine dyes.

FIG. 6: The effect of auxochrome type and fluorination on K_{L-Z}. Plot of K_{L-Z} vs. rhodamine auxochrome structure; arrows show shift in K_{L-Z} upon fluorination (open circles: Y=H; closed circles: Y=F); colors indicate Si-rhodamines (X═Si(CH₃)₂; magenta), carborhodamines (X═C(CH₃)₂; yellow), or rhodamines (X═O; blue).

FIG. 7A-7L: Spectral properties of rhodamines. Normalized fluorescence excitation and emission spectra of rhodamines: (FIG. 7A) 99. (FIG. 7B) 18. (FIG. 7C) 101. (FIG. 7D) 20. (FIG. 7E) 103. (FIG. 7F) 11. (FIG. 7G) 100. (FIG. 7H) 34. (FIG. 7I) 102. (FIG. 7J) 36. (FIG. 7L) 38. (FIG. 7K) Normalized absorption spectrum of 104.

FIG. 8: Plot of K_{L-Z} vs. auxochrome structure for rhodamines 11, 18, 20, 34, 36, 38, and 99-104. Arrow colors indicate Si-rhodamines (X═Si(CH₃)₂; magenta) or rhodamines (X═O; blue); white points indicate Y=H and colored points indicate Y=F.

FIG. 9A-9O: Labeling rates of nonfluorinated and fluorinated HaloTag® ligands. (FIG. 9A) Chemical structures of 84_{HTL} and 85_{HTL}. (FIG. 9B) % of 84_{HTL} bound vs. time using equimolar ligand and HaloTag® protein. (FIG. 9C) % of 85_{HTL} bound vs. time using equimolar ligand and HaloTag® protein. (FIG. 9D) Chemical structures of 81_{HTL} and 70_{HTL}. (FIG. 9E) % of 81_{HTL} bound vs. time using equimolar ligand and HaloTag® protein. (FIG. 9F) % of 70_{HTL} bound vs. time using equimolar ligand and HaloTag® protein. (FIG. 9G) Chemical structures of 87_{HTL} and 8_{HTL}. (FIG. 911) % of 87_{HTL} bound vs. time using equimolar ligand and HaloTag® protein. (FIG. 91) % of 8_{HTL} bound vs. time using equimolar ligand and HaloTag® protein. (FIG. 9J) Chemical structures of 86_{HTL} and 44_{HTL}. (FIG. 9K) % of 86_{HTL} bound vs. time using equimolar ligand and HaloTag® protein. (FIG. 9L) % of 44_{HTL} bound vs. time using equimolar ligand and HaloTag® protein. (FIG. 9M) Chemical structures of 88_{HTL} and 40_{HTL}. (FIG. 9N) % of 88_{HTL} bound vs. time using equimolar ligand and HaloTag® protein. (FIG. 9O) % of 40_{HTL} bound vs. time using equimolar ligand and HaloTag® protein. % bound for all ligands was measured in a stopped-flow instrument using fluorescence polarization.

FIG. 10A-10D: Stability of HaloTag® conjugates of 84_{HTL}, 85_{HTL}, and 18_{HTL}. (FIG. 10A) Chemical structures of 84_{HTL}, 85_{HTL}, and chase ligand JF₆₄₆-HaloTag® ligand (1_{HTL}). (FIG. 10B) In-gel fluorescence of HaloTag® protein labeled with nonfluorinated dye 84_{HTL} or fluorinated dye 85_{HTL} followed by incubation with 1_{HTL} or DMSO; n=3; error bars represent SD. (FIG. 10C) Chemical structure of 18_{HTL}. (FIG. 10D) In-gel fluorescence of HaloTag® protein labeled with fluorinated dye 18_{HTL} followed by incubation with 1_{HTL} or DMSO; n=2; error bars represent SD.

FIG. 11A-11D: Synthesis of fluorinated rhodamine 110 HaloTag® ligands and cellular imaging. (FIG. 11A) Synthesis of HaloTag® ligands 70_{HTL}-72_{HTL} from 67-69. (FIG. 11B-11D) Fluorescence images of live U2OS cells expressing histone H2B-HaloTag® fusion proteins and labeled with ligands 70_{HTL} (FIG. 11B), 71_{HTL} (FIG. 11C), and 72_{HTL} (FIG. 11D); scale bars for all images: 10 m.

FIG. 12A-12G: Performance of fluorinated carborhodamines. (FIG. 12A) Chemical structures of JF₆₃₂-HaloTag® ligand (44_{HTL}) and JFX₆₃₇-HaloTag® ligand (40_{HTL}). (FIG. 12B) Chemical structures of JF₆₄₆-HaloTag® ligand (1_{HTL}) and JFX₆₅₀-HaloTag® ligand (89_{HTL}). (FIG. 12C) Nuclear fluorescence vs. time upon addition of ligands 40_{HTL}, 44_{HTL}, 1_{HTL}, and 89_{HTL} (200 nM) to live cells expressing HaloTag®-histone H2B; error bars indicate SEM; n=100 nuclei from three fields of view. (FIG. 12D) Plot of fluorescence from fixed cells expressing HaloTag®-histone H2B labeled with 40_{HTL}, 44_{HTL}, 1_{HTL}, and 89_{HTL} (200 nM, 30 min, 3× wash) over 30 bleach cycles; error bars indicate SEM; n=3 independent cellular samples. (FIG. 12E) Airyscan image of U2OS cells expressing TOMM20-HaloTag® fusion proteins labeled with ligand 40_{HTL} and counterstained with Hoechst 33342; scale bar: 10 m. (FIG. 12F) Violin plot of intensity (photons/s) of ligands 40_{HTL}, 44_{HTL}, 1_{HTL}, and 89_{HTL} in SPT experiments. (FIG. 12G) Violin plot of track length (frames) of ligands 40_{HTL}, 44_{HTL}, 1_{HTL}, and 89_{HTL} in SPT experiments; only tracks >5 frames are plotted; imaging was performed at 50 Hz (20 ms/frame).

FIG. 13A-13F Performance of JF₆₅₇. FIG. 13A: Chemical structures of ATTO 647N (105), ATTO 647N-HaloTag® ligand (105_{HTL}), and JF₆₅₇-HaloTag® ligand (59_{HTL}). FIG. 13B-13C: Fluorescence images of live U2OS cells expressing histone H2B-HaloTag® fusion proteins and labeled with Hoechst 33342 to stain the nucleus, MitoTracker Green to stain mitochondria, and either 105_{HTL} (FIG. 13B) or 59_{HTL} (FIG. 13C); scale bars: 10 m. FIG. 13D: Chemical structure of JFX₆₅₀-HaloTag® ligand (89_{HTL}). FIG. 13E: Violin plot of intensity (photons/s) of ligands 89_{HTL} and 59_{HTL} in SPT experiments; **** indicates P<0.0001 by unpaired t-test. FIG. 13F: Plot of localizations per region of interest (ROI) vs. frame for ligands 89_{HTL} and 59_{HTL} in SPT experiments. SPT imaging was performed at 100 Hz (10 ms/frame).

FIG. 14A-14D: Spectral properties of rhodamines and fluorinated Malachite Green. (FIG. 14A-14C) Normalized fluorescence excitation and emission spectra of rhodamines. (a) S57. (b) 59. (c) 108. (FIG. 14D) Absolute absorption spectrum of 49. Numbers below each structure indicate: λ_{abs} (nm)·λₑₘ (nm)·ε(M⁻¹cm⁻¹)·O_{f}.

FIG. 15A-15D: Evaluation of NIR-excited fluorophores. FIG. 15A: Chemical structures of Si-rhodamine-based HaloTag® ligands 31_{HTL}, 36_{HTL}, 38_{HTL} and 108_{HTL} and phosphine oxide-rhodamine-based 106_{HTL} and 107_{HTL}. FIG. 15B: Synthesis of 104_{HTL}. FIG. 15C: Nuclear fluorescence vs. time upon addition of ligands 31_{HTL}, 36_{HTL}, 38_{HTL}, 104_{HTL}, 106_{HTL}, 107_{HTL}, and 108_{HTL} (200 nM) to live cells expressing HaloTag®-histone H2B; error bars indicate SEM; n=100 nuclei from three fields of view. FIG. 15D: Four-color image of U2OS cells expressing the following fusion proteins: histone H2B-HaloTag®; TOMM20-SNAP-tag®; GFP-SKL (cyan, peroxisomes) and labeled with 31_{HTL} (green, nucleus), JFX₆₅₀-SNAP-tag® ligand (89_{STL}; magenta, mitochondria), and JF₅₄₉-Hoechst (84_{HST}; red, nucleus); panels show individual channels; scale bar: 10 m.

FIG. 16A-16H: Performance of NIR-excited HaloTag® ligands. (FIG. 16A-16H) Chemical structures and fluorescence images of live U2OS cells expressing histone H2B-HaloTag® fusion proteins and labeled with ligands 31_{HTL} (FIG. 16A), 36_{HTL} (FIG. 16B), 38_{HTL} (FIG. 16C), 104_{HTL} (FIG. 16D), 106_{HTL} (FIG. 16E), 107_{HTL} (FIG. 16F), and 108_{HTL} (FIG. 16G) and counterstained with Hoescht 33342; scale bars for all images: 10 m. (FIG. 16H) Plot of fluorescence from fixed cells expressing HaloTag®-histone H2B labeled with 31_{HTL}, 106_{HTL}, and 107_{HTL} (200 nM, 30 min, 3× wash) over 30 bleach cycles; error bars indicate SEM; n=3 fields of view.

FIG. 17A-17E: Semi-synthetic sensor for cAMP. FIG. 17A: Structures of N,N′-diaryl-carborhodamine ligand 91_{HTL} and the fluorinated analog 65_{HTL}. FIG. 17B: Absorbance spectra of 65_{HTL} (5 M) in the presence (solid line) or absence (dotted line) of HaloTag® protein (10 M) and absorbance vs. time of 91_{HTL} and 65_{HTL} (5 M) upon addition to HaloTag® protein (10 M) FIG. 17C: Schematic of the semisynthetic cAMP indicator (ScAMPI). FIG. 17D: Fluorescence lifetime image of live U2OS cells expressing ScAMPI and labeled with 88_{STL}/65_{HTL}±forskolin; scale bar: 50 μm. FIG. 17E: Quantification of the performance of ScAMPI in cells incorporating the 88_{STL}/65_{HTL} pair±forskolin; error bars indicate SD; n=20 cells.

FIG. 18: Cellular labeling of N-arylrhodamine-based HaloTag® ligands 65_{HTL} and 91_{HTL}. Plot of nuclear fluorescence from JF₅₅₂-HaloTag® ligand (200 nM; chase) vs. incubation time with 200 nM of JQ₆₄₅-HaloTag® ligand (65_{HTL}) or 91_{HTL}; n=100 nuclear signals per compound.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Fluorescent dyes are important tools for biological research. A key challenge in the field of fluorescent dyes is the creation of derivatives with reactive groups for conjugation. It was previously discovered that masked acyl cyanide (MAC) reagents could efficiently react with rhodamine dyes that bear a fluorinated pendant phenyl ring, providing a facile strategy for regioselective installation of an amine-reactive moiety. As disclosed herein, the scope of this late-stage functionalization chemistry is expanded to more fluorophores. Using both established and unique chemistry, an array of fluorinated triarylmethane dyes are prepared, including variants of rhodamines, fluoresceins, and Malachite Green. The generality of the MAC chemistry is also demonstrated, which provides a variety of self-labeling protein tag (e.g., HaloTag®) ligands for use in imaging and sensing applications. This efficient umpolung synthetic method will enable the facile construction of new molecular tools for biological studies.

The presently-disclosed subject matter includes polycyclic chemical fluorophores as well as method for making and using the same. In some embodiments the compound has the following formula:

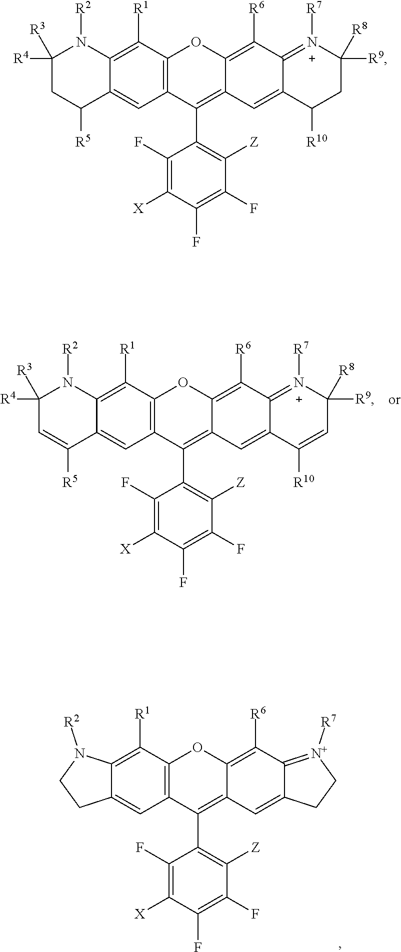

and salts thereof.

In the compound, Q is selected from the group consisting of C(alkyl)₂, NH, N(alkyl), O, S, SO₂, Si(alkyl)₂, P(O)(aryl), P(O)(alkyl), PO ₂H, PO ₂(alkyl), Se, and replaced with two H atoms.

R¹ and R⁶ are independently selected from the group consisting of H, D, halogen, CN, OH, O(alkyl), O(aryl), SH, S(alkyl), S(aryl), N₃, NH₂, NH(alkyl), N(alkyl)₂, NH(aryl), N(aryl)₂, NO₂, CHO, C(O)(alkyl), C(O)(aryl), COOH, COO(alkyl), COO(aryl), C(O)NH(alkyl), C(O)NH(aryl), PO ₃H₂, SO₃H, alkyl and substituted alkyl, aryl and substituted aryl, alkenyl and substituted alkenyl, alkynyl or substituted alkynyl, or wherein the R¹ substituent and R² substituent and/or the R⁶ substituent and R⁷ substituent, taken together with the carbon atoms to which they are bonded, independently form a substituted or unsubstituted ring containing 3, 4, 5, 6, 7, 8, or 9 atoms.

R² and R⁷ are independently selected from the group consisting of H, alkyl, deuterated alkyl, and aryl so long as when Q=C(CH₃)₂, R¹ and R² form a 6-membered ring, and R¹, R⁹, and R¹⁰ are methyl, then R⁷ cannot be methyl.

Each R³, R⁴, R⁵, R¹, R⁹, R¹⁰ is independently selected from the group consisting of H and alkyl, so long as when R¹ and R⁶ are H, and R³, R⁴, R⁵, R⁸, R⁹, and R¹⁰ are methyl, then Q cannot be O.

Z is selected from the group consisting of C(O), COOH, COO⁻, SO₃⁻, PO₃²⁻, or CR₃ where each R is independently selected from the group consisting of H, OH, alkyl, and substituted alkyl.

X is selected from the group consisting of H, alkyl, substituted alkyl, aryl, substituted aryl, alkenyl, substituted alkenyl, alkynyl, substituted alkynyl, CN, COOH, COO⁻, COO(alkyl), COO(aryl), C(O)NH(alkyl), C(O)N(alkyl)₂, C(O)NH(aryl), C(O)N(aryl)₂, PO ₃H₂, and SO₃H.

In some embodiments of the compound, X is selected from the group consisting of alkyl, substituted alkyl, aryl, substituted aryl, alkenyl, substituted alkenyl, alkynyl, substituted alkynyl, CN, COOH, COO(alkyl), COO(aryl), C(O)NH(alkyl), C(O)N(alkyl)₂, C(O)NH(aryl), C(O)N(aryl)₂.

In some embodiments of the compound, X is selected from the group consisting of

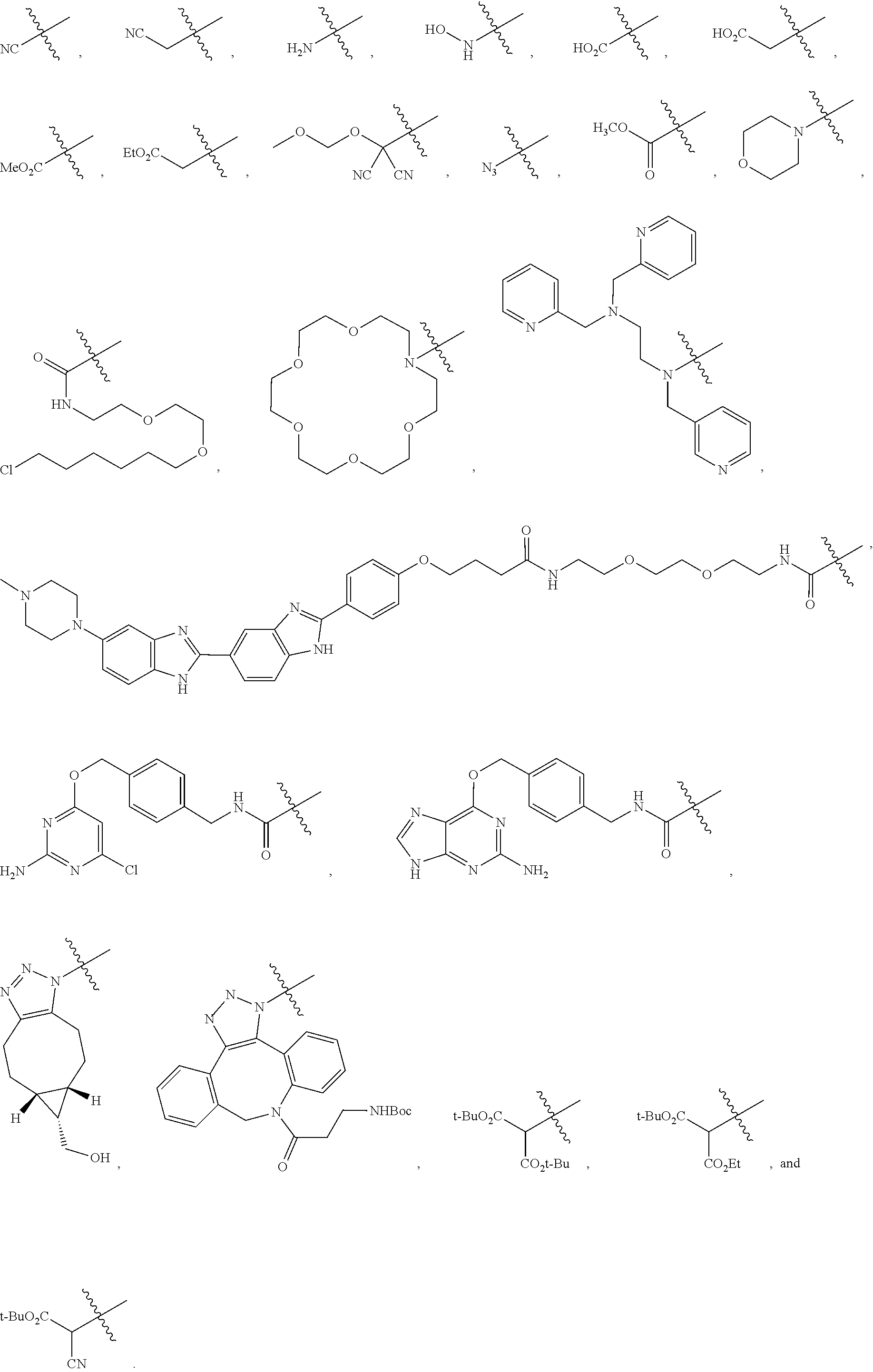

In some embodiments of the compound, Q is selected from selected from the group consisting of

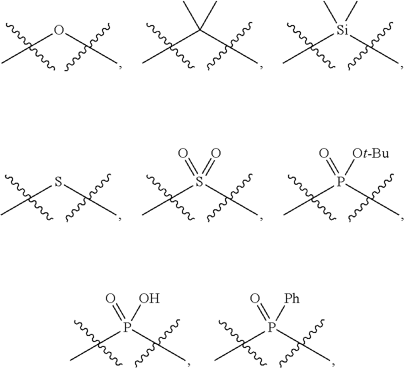

and replaced with two H atoms. In some embodiments of the compound, Q is selected from selected from the group consisting of

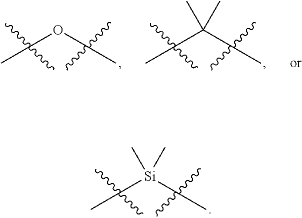

In some embodiments of the compound, R² and R⁷ are independently selected from the group consisting of H and alkyl.

In some embodiments of the compound, the R¹ substituent and R² substituent and/or the R⁶ substituent and R⁷ substituent, taken together with the carbon atoms to which they are bonded, independently form a substituted or unsubstituted ring containing 3, 4, 5, 6, 7, 8, or 9 atoms.

In some embodiments of the compound, the R¹ substituent and R² substituent and/or the R⁶ substituent and R⁷ substituent, taken together with the carbon atoms to which they are bonded, independently form a substituted or unsubstituted ring containing 6 atoms.

In some embodiments of the compound, each R³, R⁴, R⁵, R¹, R⁹, and R¹⁰ is independently selected from the group consisting of H and methyl.

In some embodiments the compound has the following formula:

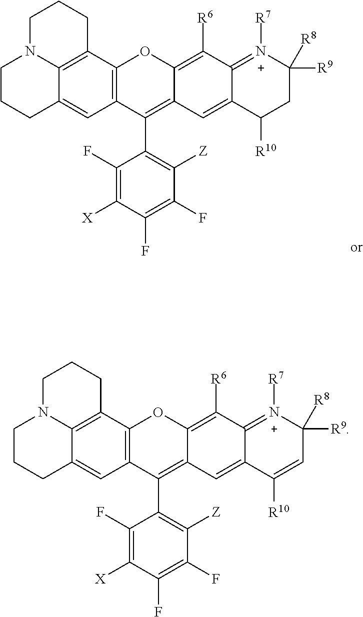

In some embodiments of the compound, Q is selected from the group consisting of C(alkyl)₂, NH, N(alkyl), O, S, SO₂, Si(alkyl)₂, P(O)(aryl), P(O)(alkyl), PO ₂H, PO ₂(alkyl), Se, and replaced with two H atoms.

In some embodiments of the compound, R⁶ is selected from the group consisting of H, D, halogen, CN, OH, O(alkyl), O(aryl), SH, S(alkyl), S(aryl), N₃, NH₂, NH(alkyl), N(alkyl)₂, NH(aryl), N(aryl)₂, NO₂, CHO, C(O)(alkyl), C(O)(aryl), COOH, COO(alkyl), COO(aryl), C(O)NH(alkyl), C(O)NH(aryl), PO ₃H₂, SO₃H, alkyl and substituted alkyl, aryl and substituted aryl, alkenyl and substituted alkenyl, alkynyl or substituted alkynyl, or where the R⁶ substituent and R⁷ substituent, taken together with the carbon atoms to which they are bonded, independently form a substituted or unsubstituted ring containing 3, 4, 5, 6, 7, 8, or 9 atoms.

In some embodiments of the compound, R⁷ is selected from the group consisting of H, alkyl, deuterated alkyl, and aryl, so long as when Q=C(CH₃)₂ and R⁸, R⁹, and R¹⁰ are methyl, then R⁷ cannot be methyl.

In some embodiments of the compound, each R¹, R⁹, and R¹⁰ is independently selected from the group consisting of H and alkyl.

In some embodiments of the compound, Z is selected from the group consisting of C(O), COOH, COO⁻, SO₃⁻, PO ₃²⁻, or CR₃ where each R is independently selected from the group consisting of H, OH, alkyl, and substituted alkyl.

In some embodiments of the compound, X is selected from the group consisting of H, alkyl, substituted alkyl, aryl, substituted aryl, alkenyl, substituted alkenyl, alkynyl, substituted alkynyl, CN, COOH, COO⁻, COO(alkyl), COO(aryl), C(O)NH(alkyl), C(O)N(alkyl)₂, C(O)NH(aryl), C(O)N(aryl)₂, PO ₃H₂, and SO₃H.

In some embodiments the compound has a structure selected from the following:

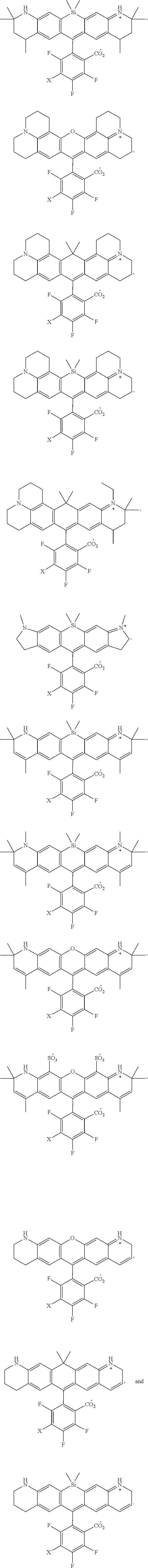

In some embodiments of the compound, X is selected from the group consisting of alkyl, substituted alkyl, aryl, substituted aryl, alkenyl, substituted alkenyl, alkynyl, substituted alkynyl, CN, COOH, COO(alkyl), COO(aryl), C(O)NH(alkyl), C(O)N(alkyl)₂, C(O)NH(aryl), C(O)N(aryl)₂.

In some embodiments of the compound, X is selected from the group consisting of

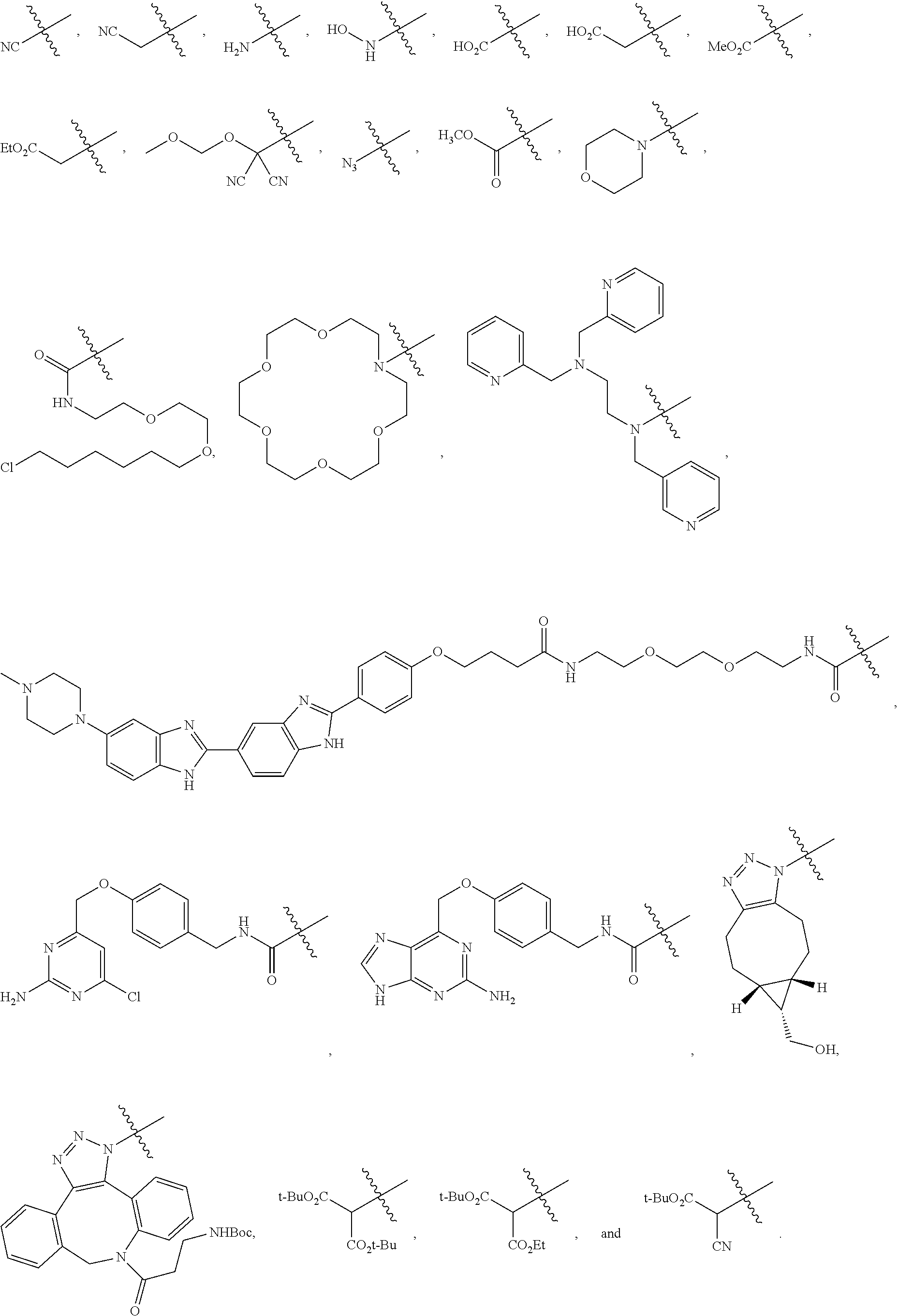

The presently-disclosed subject matter further includes a method for detecting a target substance as disclosed herein. In some embodiments, the method includes contacting a sample with a compound as disclosed herein.

In some embodiments of the detection method, the target substance is selected from a protein, a carbohydrate, a polysaccharide, a glycoprotein, a hormone, a receptor, an antigen, an antibody, a virus, a substrate, a metabolite, an inhibitor, a drug, a nutrient, a growth factor, a liprotein, and a combination thereof.

In some embodiments of the method, the detecting step is performed with a microscope. In some embodiments, the contact step and the detecting step are performed in a live cell.

Some embodiments of the method include contacting the sample with a first compound as disclosed herein and a second compound as disclosed herein, wherein the first compound is selective for a first target substance and capable of emitting a first emission light, the second compound is selective for a second target substance and capable of emitting a second emission light, and the detecting step includes detecting the first emission light that indicates the presence of the first target substance and the second emission light that indicates the presence of the second target substance.

The presently-disclosed subject matter further includes a method of making a compound, including an intermediate compound, as disclosed herein.

In some embodiments, the method of making a compound comprises preparation of diarylether and diarylsilane intermediates, as disclosed herein.

In some embodiments, the method of making a compound comprises rhodamine synthesis via lactol (phthalaldehydic acid) condensation, as disclosed herein.

In some embodiments, the method of making a compound comprises preparation of 2,2⁻diarylpropane intermediates, as disclosed herein.

In some embodiments, the method of making a compound comprises preparation of anthrone intermediates, as disclosed herein.

In some embodiments, the method of making a compound comprises xanthene synthesis via lithiation of tetrafluorobenzoic acid, as disclosed herein.

In some embodiments, the method of making a compound comprises synthesis of carborhodamines via AlCl₃ Condensation, as disclosed herein.

In some embodiments, the method of making a compound comprises rhodamine 110 and N-aryl rhodamine synthesis via cross-coupling, as disclosed herein.

In some embodiments, the method of making a compound comprises MAC substitution of 4,5,6,7⁻tetrafluoroxanthenes, as disclosed herein.

In some embodiments, the method of making a compound comprises conversion of MAC xanthenes to HaloTag® ligands, as disclosed herein.

In some embodiments, the method of making a compound comprises synthesis of HaloTag® and SNAP-tag® Ligands, as disclosed herein.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “absorption wavelength” as used herein refers to the wavelength of light capable of being absorbed by a compound in order to excite the compound to emit a light. The light emitted from a compound that has been excited with an absorption light will have an “emission wavelength.”

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used herein, the term “derivative” refers to a compound having a structure derived from the structure of a parent compound (e.g., a compounds disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

The term “detect” is used herein to refer to the act of viewing, imagining, indicating the presence of, measuring, and the like a target substance based on the light emitted from the present compounds. More specifically, in some instances the present compounds can be bound to a target substance, and, upon being exposed to an absorption light, will emit an emission light. The presence of an emission light can indicate the presence of a target substance, whereas the quantification of the light intensity can be used to measure the concentration of a target substance.

As used herein, the term “protein” means any polymer comprising any of the 20 protein amino acids, regardless of its size. Although “polypeptide” is often used in reference to relatively large proteins, and “peptide” is often used in reference to small proteins, usage of these terms in the art overlaps and varies. The term “protein” as used herein refers to peptides, polypeptides and proteins, unless otherwise noted.

With regard to the term “salt,” as used herein, as will be appreciated by one of ordinary skill in the art, a compound disclosed herein can be provided as an inner salt, where the multi-ring system is positively charged and the substituted pendant phenyl ring has a negative charge. Alternatively, a compound disclosed herein can be provided where the multi-ring system is positively charged, the pendant phenyl ring is not charged, and a counter ion is provided making the compound a salt. Accordingly, unless otherwise apparent, structures as provided herein are inclusive of inner salts and salts of the compound/counter ion. Placing either the inner salt or compound/counter ion salt in water will cause any ions to dissociate yielding the same inner salt species in solution.

The term “selectively bind” is used herein to refer to the property of an atom, moiety, and/or molecule preferentially being drawn to or binding a particular compound. In some instances the atom, moiety, and/or molecule selectively binds to a particular site on a compound, such as an active site on a protein molecule.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds.

Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless stated otherwise, all chemical groups described herein include both unsubstituted and substituted varieties.

Where substituent groups are specified by their conventional chemical formula written from left to right, they equally encompass the chemically identical substituents that would result from writing the structure from right to left. For instance, —CH₂O— also encompasses recite —OCH₂—.

It should be understood that the bond types and locations in the chemical structures provided herein may adapt depending on the substituents in the compound, even if not specifically recited. For instance, —X— where X can be either C or N can refer to, respectively, —CH₂— or —NH—, where the lone pair of electrons on N is not illustrated. Thus, even if not specifically illustrated, the chemical compounds described herein include any hydrogen atoms, lone pair of electrons, and the like necessary for completing a chemical structure.

The term “target substance” refers to a substance that is selectively bound directly by the presently-disclosed compounds and/or indirectly by a molecule that is bound to the present compound. A target substances can include, but is not limited to, a protein, carbohydrates, polysaccharide, glycoprotein, hormone, receptor, antigen, antibody, virus, substrate, metabolite, inhibitor, drug, nutrient, growth factor, and the like. In some embodiments the target substance refers to an entire molecule, and in other embodiments the target substances refers to a site on a molecule, such as a binding site on a particular protein.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also refer to both substituted or unsubstituted alkyls. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term. The term “alkyl” is inclusive of “cycloalkyl.”

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

In this regard, the term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3⁻oxadiazole, 1,2,5-oxadiazole and 1,3,4⁻oxadiazole, thiadiazole, including, 1,2,3⁻thiadiazole, 1,2,5⁻thiadiazole, and 1,3,4⁻thiadiazole, triazole, including, 1,2,3⁻triazole, 1,3,4⁻triazole, tetrazole, including 1,2,3,4-tetrazole and 1,2,4,5⁻tetrazole, pyridine, pyridazine, pyrimidine, pyrazine, triazine, including 1,2,4⁻triazine and 1,3,5⁻triazine, tetrazine, including 1,2,4,5⁻tetrazine, pyrrolidine, piperidine, piperazine, morpholine, azetidine, tetrahydropyran, tetrahydrofuran, dioxane, and the like.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹⁻OA² or —OA¹-(OA²)ₐ—OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. The term is include of linear and ring-forming (i.e., cycloakenyl) groups. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, haide, hydroxy, ketone, azide, nitro, silyl, sulfo-oxo, or thiol, as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “ring” as used herein refers to a substituted or unsubstituted cycloalkyl, substituted or unsubstituted heterocycloalkyl, substituted or unsubstituted aryl, or substituted or unsubstituted heteroaryl. A ring includes fused ring moieties, referred to as a fused ring system wherein a ring may be fused to one or more rings selected from a substituted or unsubstituted cycloalkyl, substituted or unsubstituted heterocycloalkyl, substituted or unsubstituted aryl, and substituted or unsubstituted heteroaryl in any combination. The number of atoms in a ring is typically defined by the number of members in the ring. For example, a “5- to 8-membered ring” means there are 5 to 8 atoms in the encircling arrangement. A ring can optionally include a heteroatom. The term “ring” further includes a ring system comprising more than one “ring”, wherein each “ring” is independently defined as above.

Some of the unsaturated structures described herein, such as ring structures including cycloalkyl and aryl, are illustrated with dashed bonds to signify the potential existence of a resonance structure. Structures having dashed bonds are intended to reflect every possible configuration of the structure, but does not necessarily imply that all possible structures are in existence. It should be understood that the types of bonds (e.g., single bond, double bond) in such structures will vary depending on the atoms in the structure as well as whether the structures are substituted with one or more additional atoms or moieties.

The term “aldehyde” as used herein is represented by a formula C(O)H. Throughout this specification “C(O)” is a short hand notation for a carbonyl group, i.e., C═O.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein. In specific embodiments amine refers to any of NH₂, NH(alkyl), NH(aryl), N(alkyl)₂, and N(aryl)₂.

The term “carboxylic acid” as used herein is represented by a formula C(O)OH.

The term “halide” or “halogen” refers to at least one of the halogens selected from fluorine, chlorine, bromine, and iodine.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Lactone-Zwitterion Equilibrium of Dyes

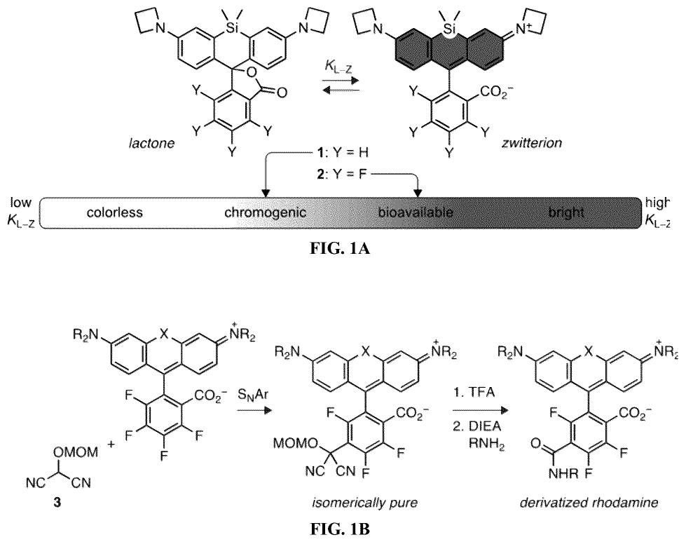

Rhodamine dyes bearing an ortho-carboxyl substituent on the pendant phenyl ring exist in equilibrium between a lipophilic lactone (L) and fluorescent zwitterion (Z). The lactone-zwitterion equilibrium constant (K_{L-Z}) can be used to rationalize the performance of rhodamine dyes in biological systems (FIG. 1).¹⁴⁻¹⁶

Four different classes of dyes were categorized based on K_{L-Z}. First, rhodamines with very low K_{L-Z} values are colorless and not useful for biological imaging. Second, rhodamines with slightly higher K_{L-Z} values also preferentially lactonize in aqueous solution but have some propensity to adopt the zwitterionic form. Such dyes can be used to fashion chromogenic compounds; the change in environment that accompanies dye ligand binding shifts the equilibrium to the zwitterionic form.^{15, 16} Third, dyes with modestly higher K_{L-Z} values mostly adopt the zwitterionic form in aqueous solution but can lactonize in hydrophobic environments; ligands based on these fluorophores often show improved membrane permeability making them more bioavailable.¹⁶ Finally, fluorophores with large K_{L-Z} values exhibit high absorptivity, making them bright, environmentally insensitive labels.¹⁴ Understanding the K_{L}Z trends of a particular dye scaffold and tuning this property through structural modifications can allow optimization of fluorescent labels for biological imaging experiments.

Fluorination of the pendant phenyl ring increases the K_{L-Z} of azetidine-containing rhodamine dyes. For example, Janelia Fluor 646 (JF₆₄₆, 1; FIG. 1A) and similar Si-bridged rhodamine dyes preferentially adopt the nonfluorescent lactone form in aqueous solution, allowing the creation of chromogenic (and therefore fluorogenic) molecules.₁₅₋₁₈ Incorporation of fluorine atoms on the pendant phenyl ring results in JF₆₆₉ (2; FIG. 1A), which shows a higher K_{L-Z} and can be used to make bioavailable fluorescent labels.^{14, 16} In addition to modulating K_{L-Z}, fluorination of the pendant phenyl ring increases the absorption maximum (L_{ab}s) and emission maximum (em) of xanthene dyes and their analogs.^{14, 19-21}

### Example 2: Synthesis of Functional Fluorinated Rhodamines

Fluorinated phenyl rings also enable facile derivatization of rhodamine dyes using Yamamoto's masked acyl cyanide (MAC) reagent concept.²² In this umpolung approach a 2-hydroxymalononitrile MAC reagent is used with a methoxymethyl ether (MOM) protecting group (3; FIG. 1B). This reacts with a rhodamine dye bearing a perfluorinated pendant phenyl ring in a regioselective nucleophilic aromatic substitution (S_{N}Ar) reaction.¹⁶ Removal of the MOM group using acid, followed by neutralization with a tertiary amine base generates a putative acyl cyanide that is primed for subsequent chemistry to generate an acid, ester, or amide at the 6⁻position of the rhodamine; this regiochemistry is optimal for self-labeling tag ligands.²³, 24 The late-stage derivatization chemistry using an acyl anion equivalent circumvents the isomeric mixtures that result from classic synthetic strategies^{2, 13,25-28} and the protecting group challenges that come with modern methods,²⁹⁻³⁴ allowing the transformation of an unsubstituted dye to a functional derivative in just two synthetic steps.

In previous work, the structure-activity relationships governing K_{L-Z}, the effect of fluorination, and the generality of the MAC chemistry were investigated for a limited set of rhodamine dyes containing four-membered azetidine substituents.¹⁶ As disclosed herein, thinking outside the azetidine “box” and dyes containing different auxochromes are explored. Different synthetic methods were investigated to prepare rhodamine and related dyes, expanding several known routes and developing a new, succinct strategy for preparing fluorinated dyes using Li/H exchange. This suite of methods was used to synthesize a comprehensive collection of fluorinated and nonfluorinated rhodamine dyes with different nitrogen substituents. Evaluation of the properties of these known and unique molecules reveal that the range of accessible K_{L-Z} values within a given rhodamine scaffold is strongly dependent on the identity of the auxochrome group. Despite these large differences in lactone-zwitterion equilibrium, fluorination universally increases K_{L-Z} regardless of dye structure. For rhodamines bearing electron-donating julolidine substituents, which exhibit high K_{L-Z} values, incorporation of fluorine substituents results in a bathochromic shift and increased absorptivity, yielding bright, red-shifted fluorophores useful for multicolor imaging and live-cell single-particle tracking (SPT). For dyes containing electron-withdrawing aniline substituents, fluorination improves chromogenicity, giving a live-cell compatible Forster resonance energy transfer (FRET) quencher dye that is useful for fluorescence lifetime imaging microscopy (FLIM). Together, this work expands the synthetic methods available for rhodamine synthesis, demonstrates the practicability of K_{L-Z} tuning beyond azetidine-containing dyes, and yields new probes with improved properties for advanced fluorescence imaging experiments such as SPT and FLIM.

### Example 3: Synthesis of Fluorinated Rhodamines

Different methods were considered for constructing rhodamine dyes, focusing on the synthesis of the less-explored fluorinated derivatives. In prior work¹⁶ fluorinated rhodamines were made using a synthetic approach^{14, 35-37} where a di(bromoarene) undergoes metal/Br exchange and addition to tetrafluorophthalic anhydride (4) to yield fluorinated rhodamine dyes (Scheme 1A: metal/Br exchange). This complements the standard method of fluorinated rhodamine synthesis involving protic or Lewis acid-catalyzed condensation of 3⁻aminophenols and 4 (Scheme 1B: acid-catalyzed condensation).^{38, 39}

In addition to these established methods to construct dyes with fluorinated phenyl systems, consideration was given to repurposing other strategies for fluorinated rhodamine synthesis. These include the Pd-catalyzed cross-coupling using fluorescein ditriflates^{40, 41} (Scheme 1C: Pd-catalyzed cross-coupling) and the oxidative condensation of phthalaldehydic acids with 3⁻aminophenols or diphenyl ethers⁴² (Scheme 1D: lactol condensation and oxidation). Neither method has been used to make fluorinated rhodamines, but it was surmised that use of fluorinated fluorescein ditriflates or tetrafluorophthalaldehydic acid (5), respectively, could allow synthesis of the desired compounds. A unique strategy was also considered to prepare fluorinated dyes by lithiation of 2,3,4,5⁻tetrafluorobenzoic acid⁴³ (6) and addition to substituted xanthone derivatives and their congeners (Scheme 1E: Li/H exchange). The scope of each synthetic method was set out to be explored, evaluating of the utility of these strategies to prepare standard oxygen-containing rhodamines, Si-rhodamines, and carborhodamines along with related dyes based on fluoresceins and Malachite Green.

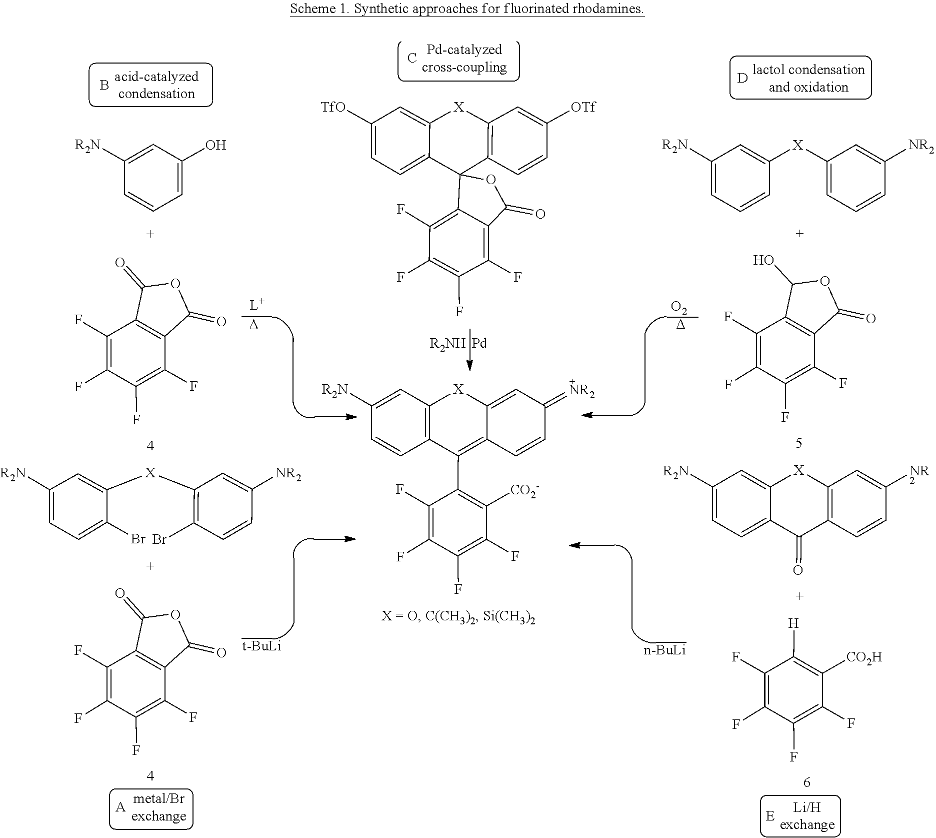

The reaction of aminophenols with partially reduced phthalic anhydrides (phthalaldehydic acids) in the presence of O₂ was considered first (Scheme 1D). This recently described strategy⁴² is an efficient and versatile method for the regioselective synthesis of rhodamine dyes including carboxy derivatives but has not been applied to fluorinated dyes. The shorthand term “lactol condensation” is used to refer to this approach, as phthalaldehydic acids mostly adopt the closed, 3⁻hydroxyphthalide (lactol) form in solution.⁴⁴ This strategy was compared with previously described methods of rhodamine synthesis. Metal/bromide exchange of the dibromide 7 (Scheme 2) and addition to tetrafluorophthalic anhydride (4) afforded the unique deuterated dye 8 in 34% yield (Scheme 3); this fluorophore was named “JFX₅₇₆” due to the deuterium substitution⁴⁵ and its absorption maximum in aqueous solution. Reaction of diphenyl ether 9 (Scheme 2a) with tetrafluorolactol 5 in 2,2,2⁻trifluoroethanol (TFE) allowed convenient access to 8 in higher yield (85%), demonstrating the utility of this lactol chemistry in making fluorinated rhodamines.

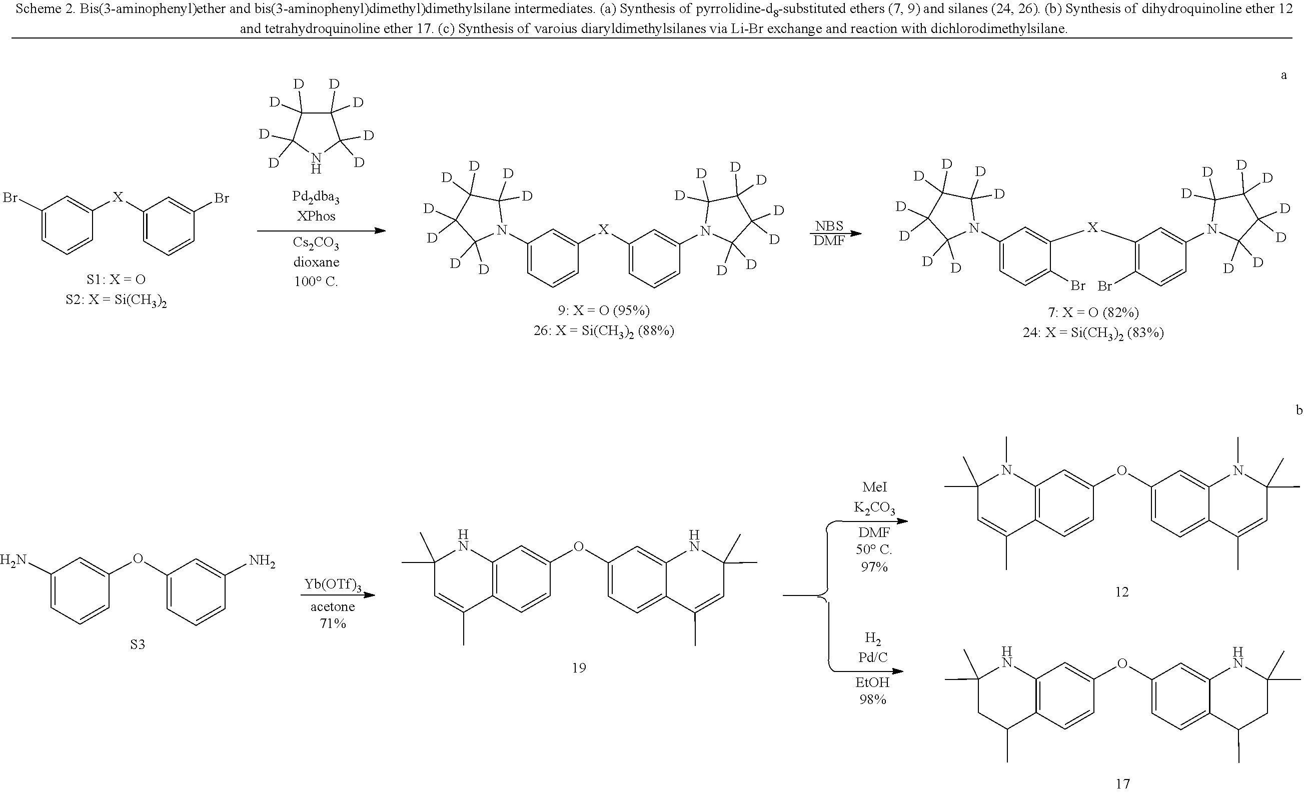

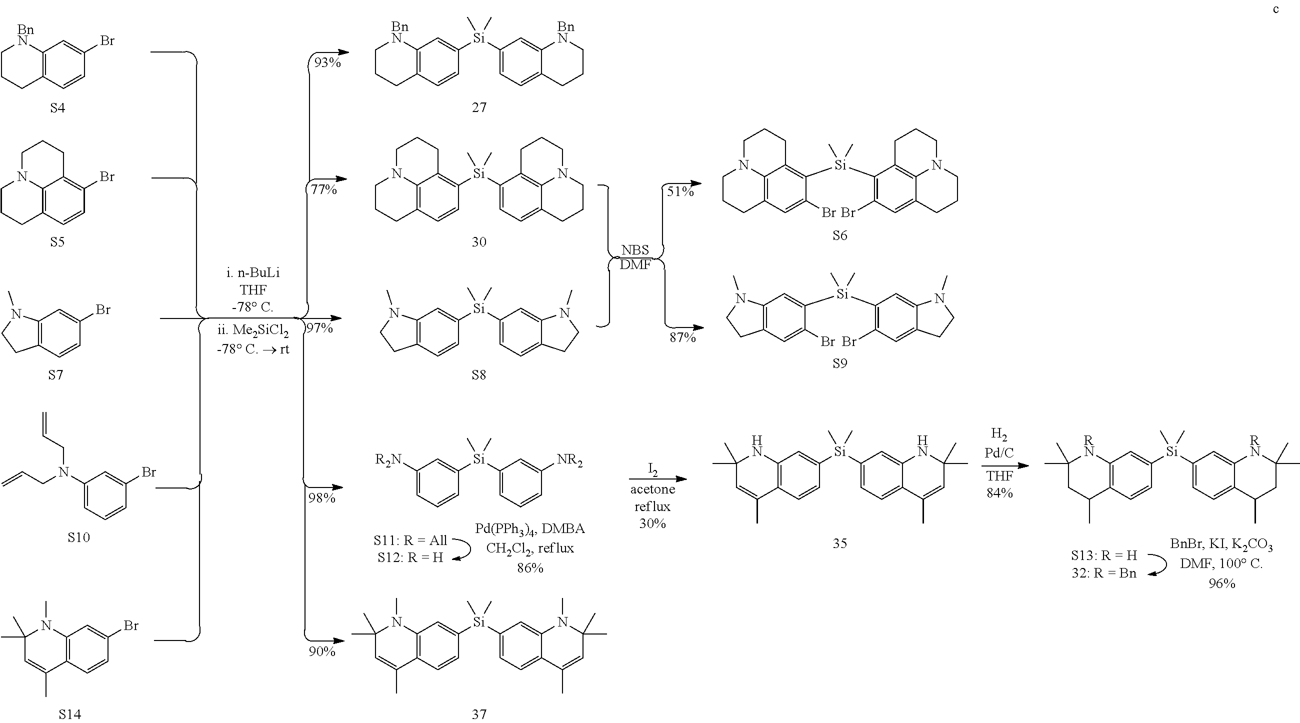

This lactol condensation strategy was then compared with the classic acid-catalyzed reaction method, which remains the most common protocol for rhodamine synthesis but shows variable results when preparing fluorinated rhodamines.^{38, 39} Reaction of dihydro-quinoline 10 with 4 in refluxing propionic acid gave rhodamine 11 in low yield (8%; Scheme 3); other condensation reaction conditions were unsuccessful (Table 1). This is consistent with previous reports of the synthesis of 11,38′39 where the dye was prepared in low yield using standard acid-mediated chemistry. The lactol strategy proved superior, with diphenyl ether 12 (Scheme 2b) reacting with 5 to give 11 in 66% yield (Scheme 3).

TABLE 1
Comparison and optimization of reaction conditions-acid-mediated condensation of
phthalic anhydrides versus oxidative condensation of phthalaldehydic acids (“lactols”)—for the
synthesis of dihydroquinoline rhodamines 11 and 103.

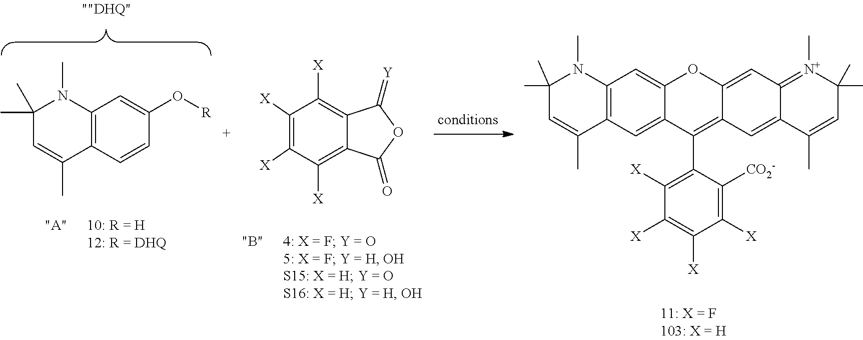

| R | X | Y | solvent/reagents | temperature | time | equiv A:B | yield |
|---|---|---|---|---|---|---|---|
| H | F | O | EtCO₂H, p-TsOH | reflux | 24 h | 2.1:1 | 8-10% |
| H | F | O | EtCO₂H, p-TsOH | reflux | 72 h | 2.1:1 | 0%ª |
| H | F | O | ODCB | reflux | 18 h | 2.1:1 | 0% |
| H | F | O | TFE | 80° C. | 24 h | 2.1:1 | 0% |
| H | F | H, OH | TFE, O₂ | 80° C. | 24 h | 2.1:1 | 24% |
| DHQ | F | H, OH | TFE, O₂ | 80° C. | 24 h | 1:1 | 66% |
| DHQ | F | H, OH | TFE, O₂ | 80° C. | 24 h | 1:1.5 | 59% |
| DHQ | F | O | TFE | 80° C. | 24 h | 1:1 | 9% |
| H | H | O | EtCO₂H, p-TsOH | reflux | 24 h | 2.1:1 | 22% |
| H | H | O | EtCO₂H, p-TsOH | reflux | 72 h | 2.1:1 | 58% |
| H | H | O | ODCB | reflux | 18 h | 2.1:1 | 37% |
| H | H | O | ODCB | reflux | 72 h | 2.1:1 | 84% |
| DHQ | H | H, OH | TFE, O₂ | 80° C. | 24 h | 1:1 | 69% |

^{a}11 slowly decomposes with extended reaction time under these conditions to afford a mixture of several other products, most notably the analog resulting from protodecarboxylation of the ortho-carboxyl group.

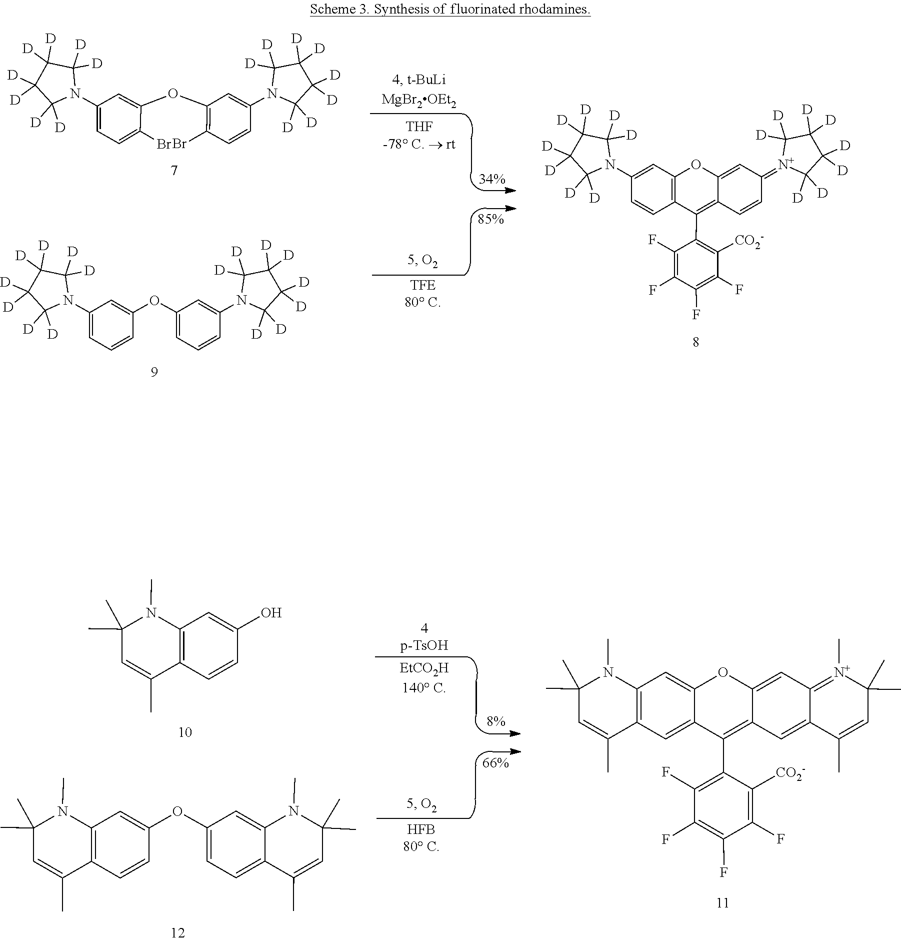

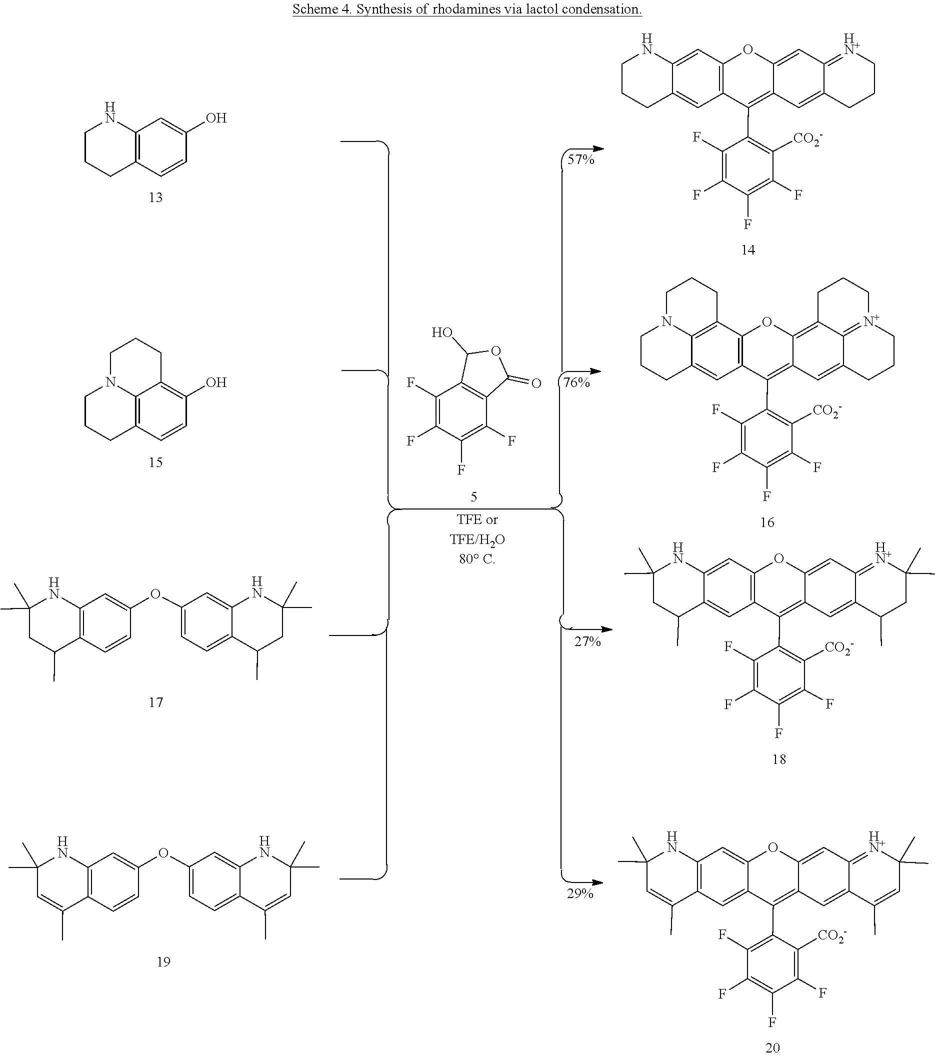

The generality of the lactol synthesis was then explored for other fluorinated rhodamine dyes (Scheme 4). In addition to diphenyl ethers such as 12, 3⁻aminophenol derivatives were also accommodated by this chemistry. Reaction of 7-hydroxytetrahydroquinoline (13) with 5 afforded the 4,5,6,7⁻tetrafluoro-Q-rhodamine (FRh_{Q}, 14). 8-Hydroxyjulolidine (15) reacted with 5 to give the fluorinated derivative of rhodamine 101 (FRh₁₀₁, 16).^{46, 47} Diphenyl ether 17 (Scheme 2b) produced 18, a fluorinated analog of ATTO 550 that was called “Janelia Fluor” 563 (JF₅₆₃). Finally, reaction of 5 and ether 19 (Scheme 2b) gave 20, the des-methyl analog of compound 11.³⁸ Reactions using the secondary amine reactants 13, 17, and 19 afforded lower yields (27-57%) than those using the fully substituted anilines 9, 12, and 15 (66-85%; Scheme 3, Scheme 4), likely due to side reactions involving the secondary amine and the aldehyde tautomer of 5. Several nonfluorinated analogs of these dyes were also prepared using the lactol condensation (Scheme 5).

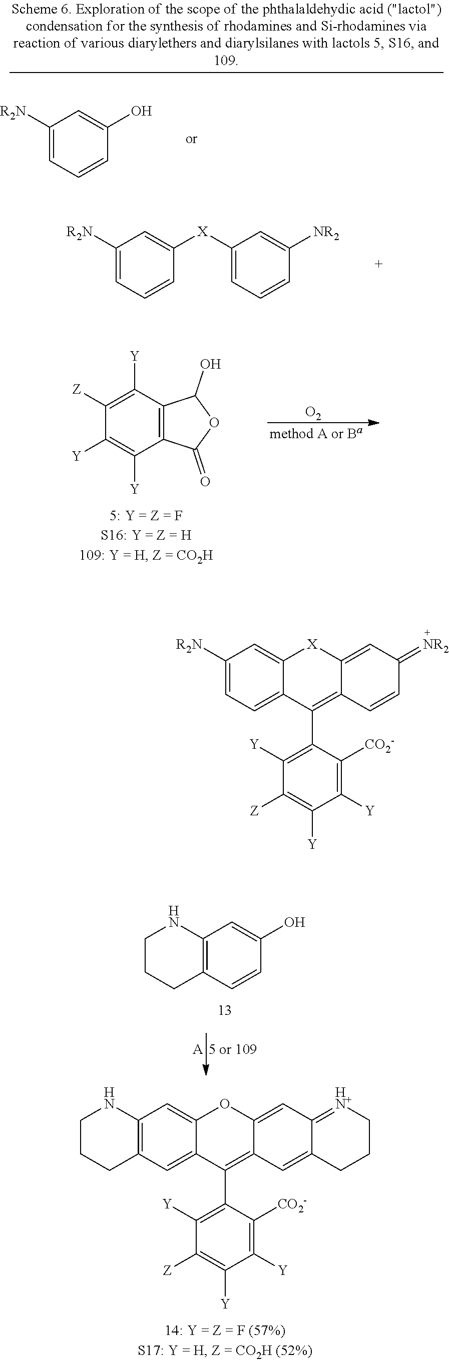

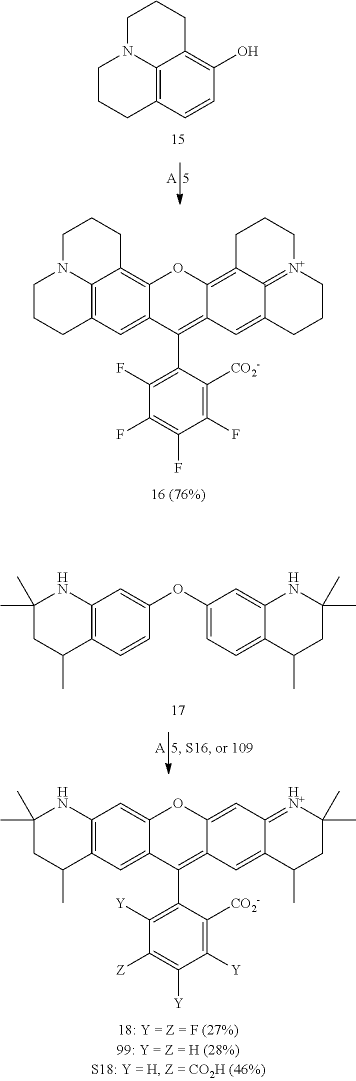

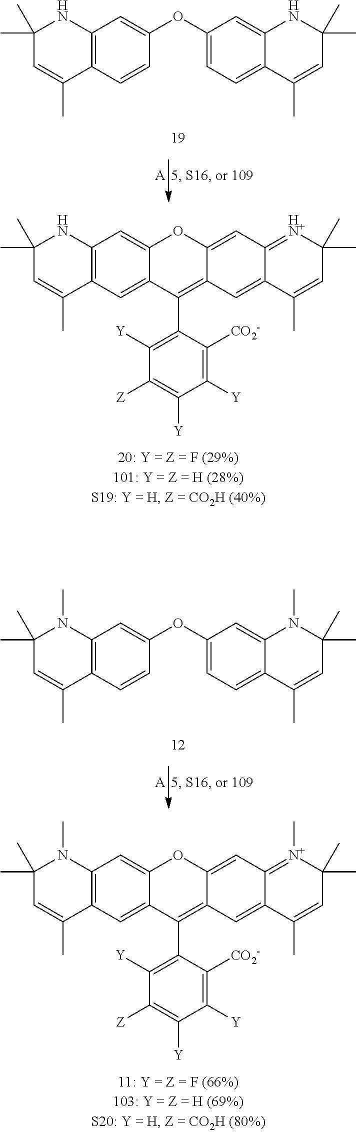

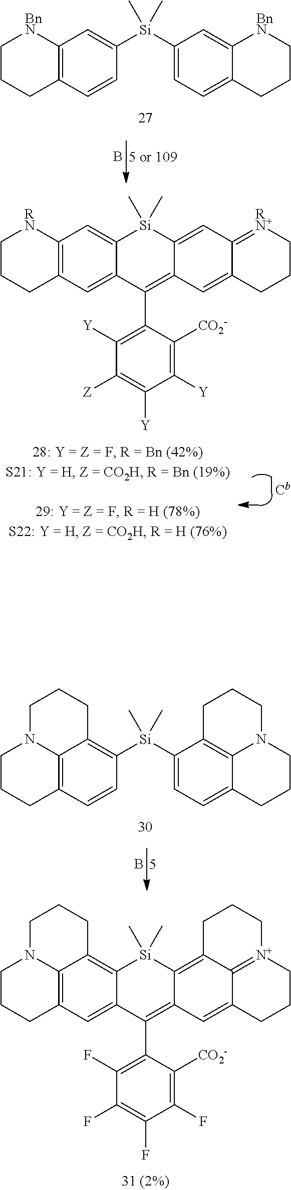

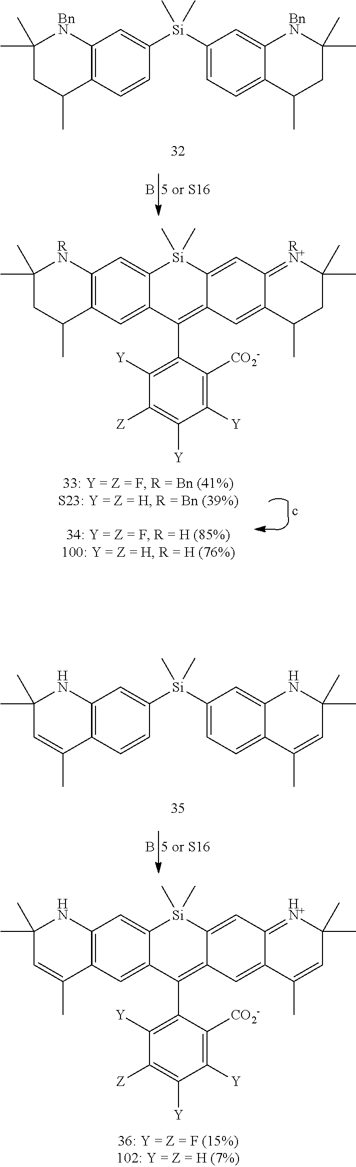

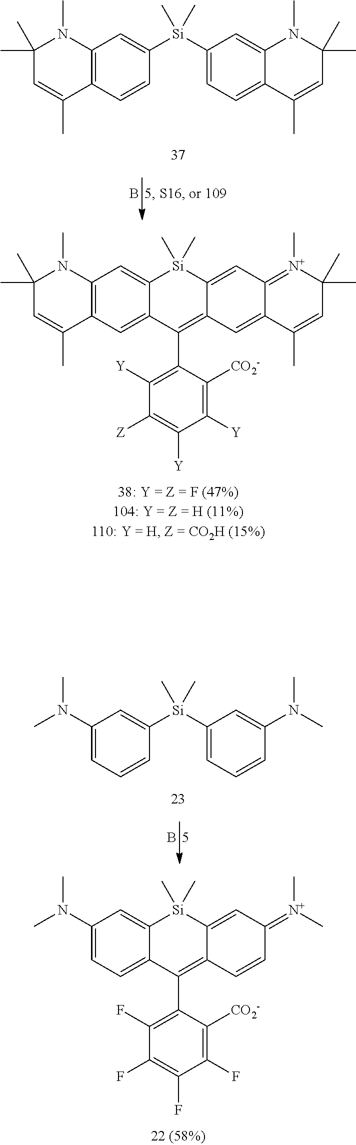

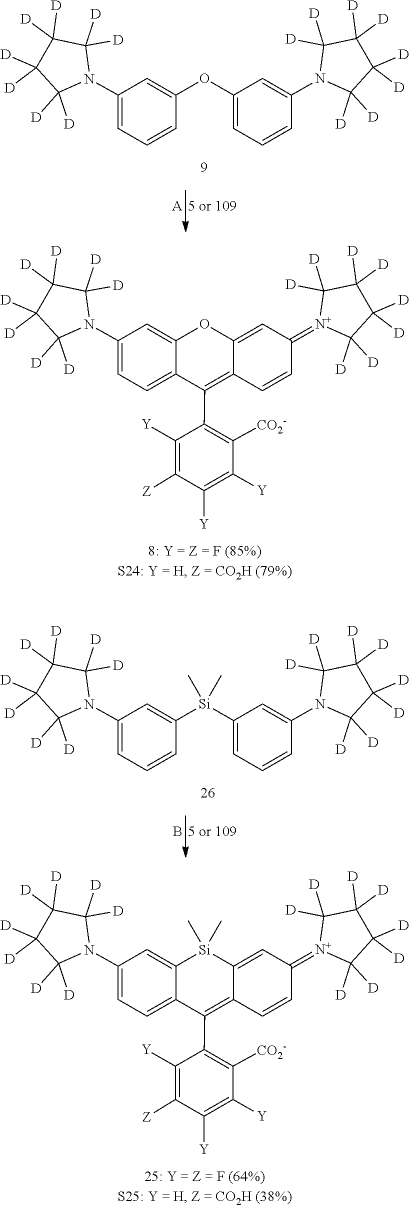

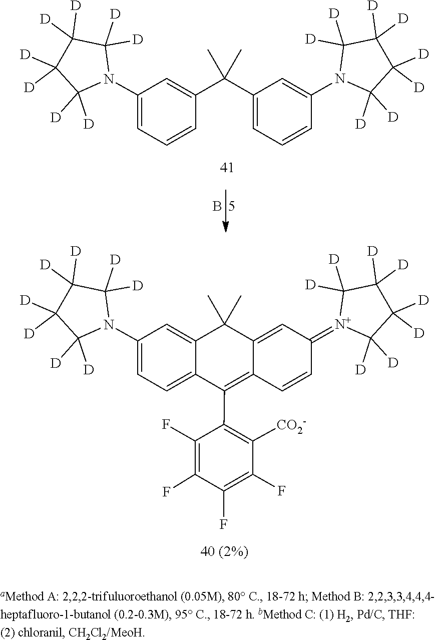

### Example 4: Synthesis of Fluorinated Si-Rhodamines

Substitution of the xanthene oxygen in rhodamines for a silicon moiety causes a substantial bathchromic shift of ˜100 nm, making Si-rhodamines useful fluorophores that are excited by long-wavelength light.^{21, 29, 31-34, 48-50} This substitution also decreases the K_{L-Z} compared to standard oxygen-containing rhodamine dyes. As mentioned above, some Si-rhodamines such as JF₆₄₆ (1; FIG. 1A) preferentially adopt the nonfluorescent lactone form in aqueous solution and can be used to create chromogenic/fluorogenic labels.^{15, 17, 18} Installation of fluorine atoms on the pendant phenyl ring shifts the lactone-zwitterion equilibrium towards the zwitterionic form, yielding JF₆₆₉ (2; FIG. 1A), a dye that shows higher e values and improved bioavailability compared to the nonfluorinated congener 1.^{14, 16}

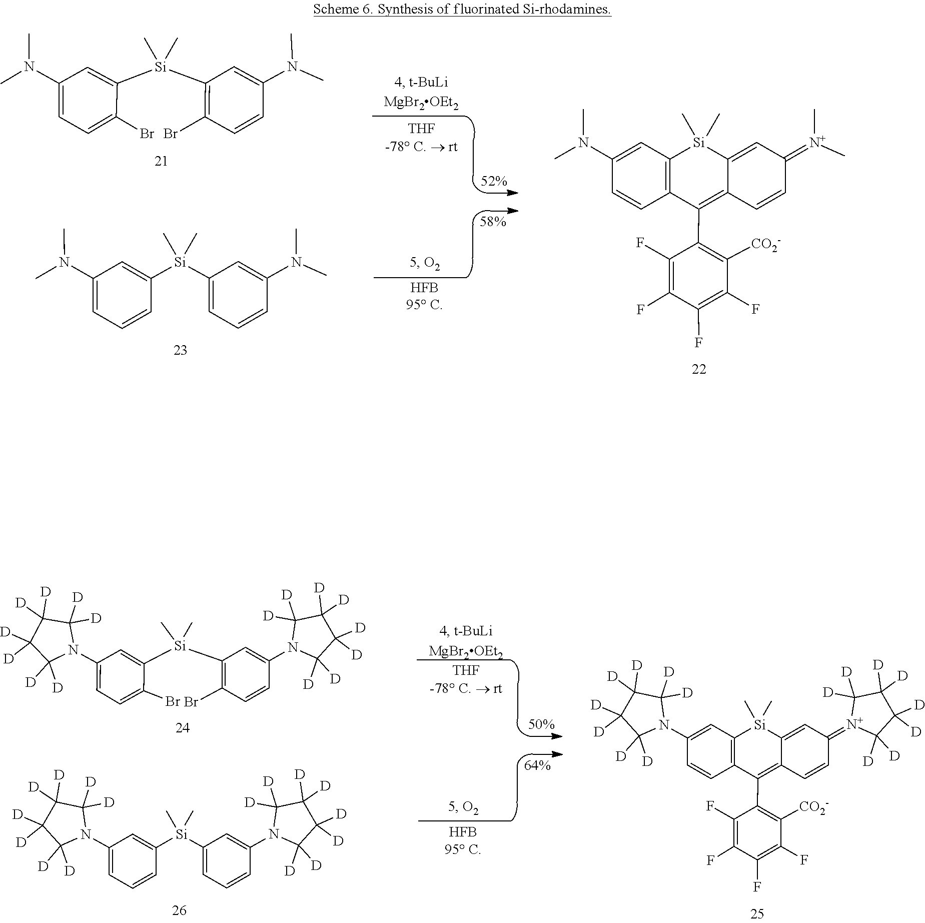

Given the utility of JF₆₆₉, the condensation of fluorinated lactol 5 was investigated to synthesize fluorinated Si-rhodamine dyes, again comparing this chemistry to the bis(arylmetal) addition strategy (Scheme 6). This lactol condensation approach has not been used to make any Si-rhodamines, fluorinated or otherwise. As described previously, metal/bromide-exchange of dibromide 21 and addition to anhydride 4 gives the fluorinated derivative of Si-tetramethylrhodamine²⁹ (SiRF₆₆₇; 22) in moderate yield (52%).

Application of the published protocol⁴² for the lactol condensation using 2,2,2-trifluoroethanol (TFE) at 80° C. and a concentration of 0.05 M (Scheme 3, Scheme 4) gave poor yields of the silicon-containing variants. To remedy this problem, different reaction conditions were explored using diarylsilane 23 as a model reactant (Table 2). Replacing TFE with the higher-boiling solvent 2,2,3,3,4,4,4⁻heptafluoro-1⁻butanol (HFB) allowed reaction at elevated temperature (95° C.).

This solvent change, combined with performing the condensation at higher concentration (0.2-0.3 M), afforded Si-rhodamine 22 in a similar yield (58%) to the more operationally complex reaction involving the bis(arylmetal) species (Scheme 6). Reaction of the pyrrolidine-d₈⁻containing dibromide 24 (Scheme 2a) with 4 produced fluorophore 25 (JFX₆₇₃) in 50% yield; synthesis of the same dye via the straightforward condensation of silane 26 (Scheme 2a) and lactol 5 gave a higher yield (64%).

TABLE 2
Optimization of oxidative phthalaldehydic acid (“lactol”) condensation of 5 with
diaryldimethylsilane 23 to access fluorinated SiR dye 22 (SiRF₆₆₇).

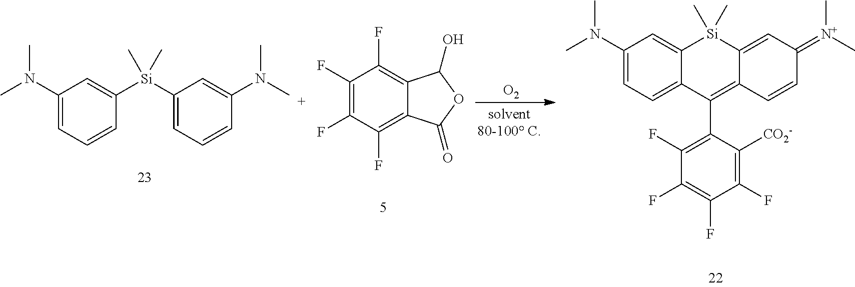

| equiv 23:5 | solvent^{a} | temperature | concentration | time | yield |
|---|---|---|---|---|---|
| 1:1 | TFE | 80° C. | 0.03 M | 72 h | 16% |
| 1:1 | TFE | 80° C. | 0.03 M | 168 h | 18% |
| 1:1 | TFP | 100° C. | 0.03 M | 72 h | 11% |
| 1:1 | HFB | 95° C | 0.03 M | 72 h | 18% |
| 1:1 | TFE | 80° C. | 0.3 M | 72 h | 25% |
| 1:1 | HFB | 95° C | 0.3 M | 72 h | 36% |
| 1:1.5 | HFB | 95° C. | 0.3 M | 48 h | 44% |
| 1:2 | HFB | 95° C | 0.3 M | 48 h | 47% |
| 1:2 | HFB | 95° C. | 0.3 M | 72 h | 52% |
| 1:3 | HFB | 95° C. | 0.3 M | 72 h | 58% |

^{a}TFE = 2,2,2-trifluoroethanol;
TFP = 2,2,3,3-tetrafluoro-1-propanol;
HFB = 2,2,3,3,4,4,4-heptafluoro-1-butanol.

The investigation of the lactol condensation chemistry was expanded to synthesize other fluorinated Si-rhodamines (Scheme 7). As with oxygen-containing rhodamines (Scheme 4), the use of secondary amine reactants gave lower yields of fluorophore products. This problem prompted protection of the aniline nitrogens in some cases to mitigate side reactions driven by the higher reaction temperature.

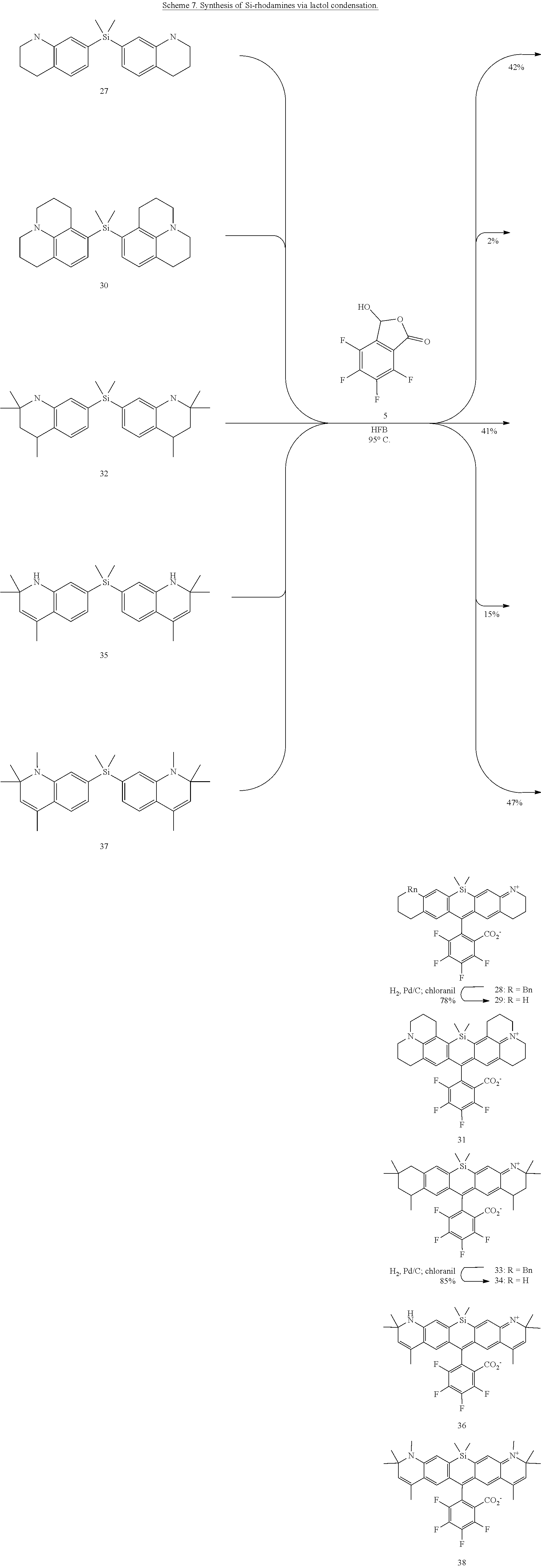

N-Benzyl-protected silane 27 (Scheme 2c) gave Si-rhodamine 28 in 42% yield; hydrogenation of 28 yielded the fluorinated Si-Q-rhodamine (FSiRh_{Q}, 29). Compound 30 (Scheme 2c) combined with 5 to furnish dye 31, which was called “JF₆₉₈”, albeit in very low yield (2%) due to competing protodesilylation of 30. These conditions were also used to prepare the silicon analogs of rhodamines 11, 18, and 20 (Scheme 3, Scheme 4). Condensation of 32 (Scheme 2c) with 5 afforded dibenzyl dye 33 in 41% yield, which was deprotected to give Si-rhodamine 34. Direct reaction of 35 (Scheme 2c) provided a 15% yield of dye 36; here, the dihydroquinoline moieties precluded use of N-benzyl protecting groups, which resulted in the relatively low yield. Reaction of silane 37 (Scheme 2c) and phthalaldehydic acid 5 formed Si-rhodamine 38 (47% yield). Attempts to synthesize dihydroquinoline dyes such as 38 via the dibromide/bis(arylmetal) route failed. The yields for some of these reactions were modest and, in a few cases-most notably JF₆₉₈ (31) the dibromide strategy gave better results (Scheme 8). Nonetheless, these optimized, simple conditions using HFB and elevated temperature were general for the synthesis of fluorinated dyes and also useful for expediently accessing various nonfluorinated Si-rhodamines (Scheme 5).

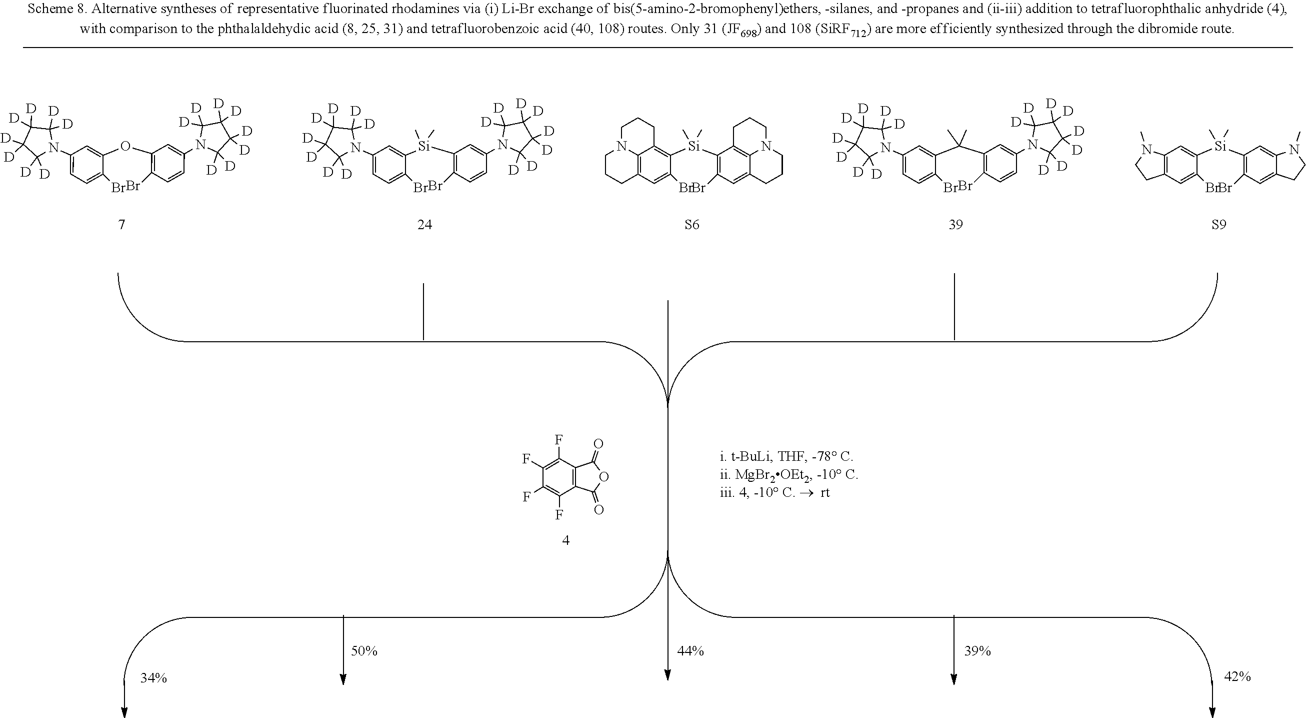

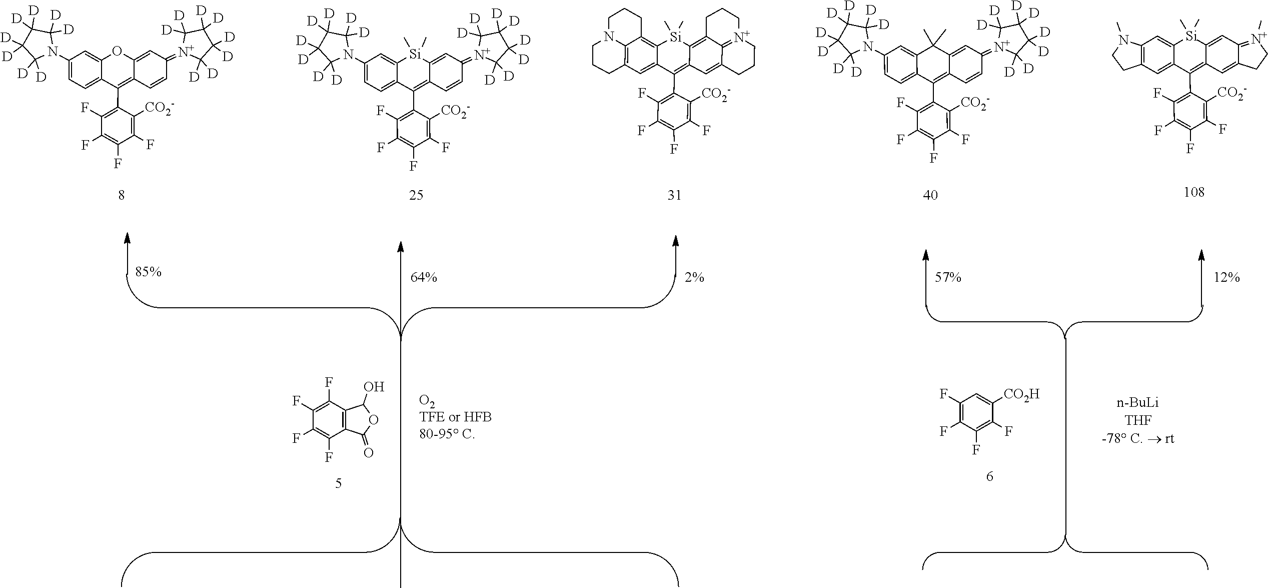

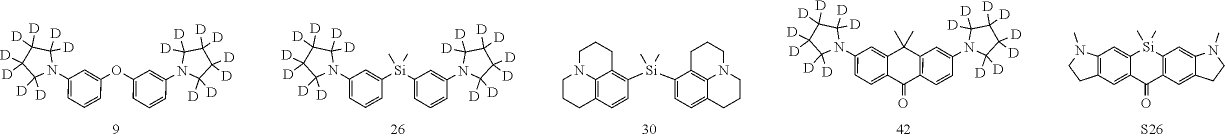

### Example 5: Synthesis of Fluorinated Carborhodamines

The synthesis of a unique class of fluorophore were then explored: carbon-containing analogs of rhodamines and fluoresceins bearing a fluorinated pendant phenyl ring. Relative to the standard oxygen-bridged rhodamine dyes, the gem-dimethyl carbon substituent elicits a ˜60 nm bathochromic shift in absorption and emission wavelengths and a shift towards lower K_{L-Z} values.^{41, 51, 52} As with other rhodamines, fluorination of carborhodamines should elicit a further bathochromic shift in λ_{abs} and λₑₘ and increase K_{L-Z}, leading to higher absorptivity. An initial attempt was made to access fluorinated carborhodamines using dibromide starting materials (Scheme 1A) or using the lactol condensation chemistry (Scheme 1D, Scheme 9)

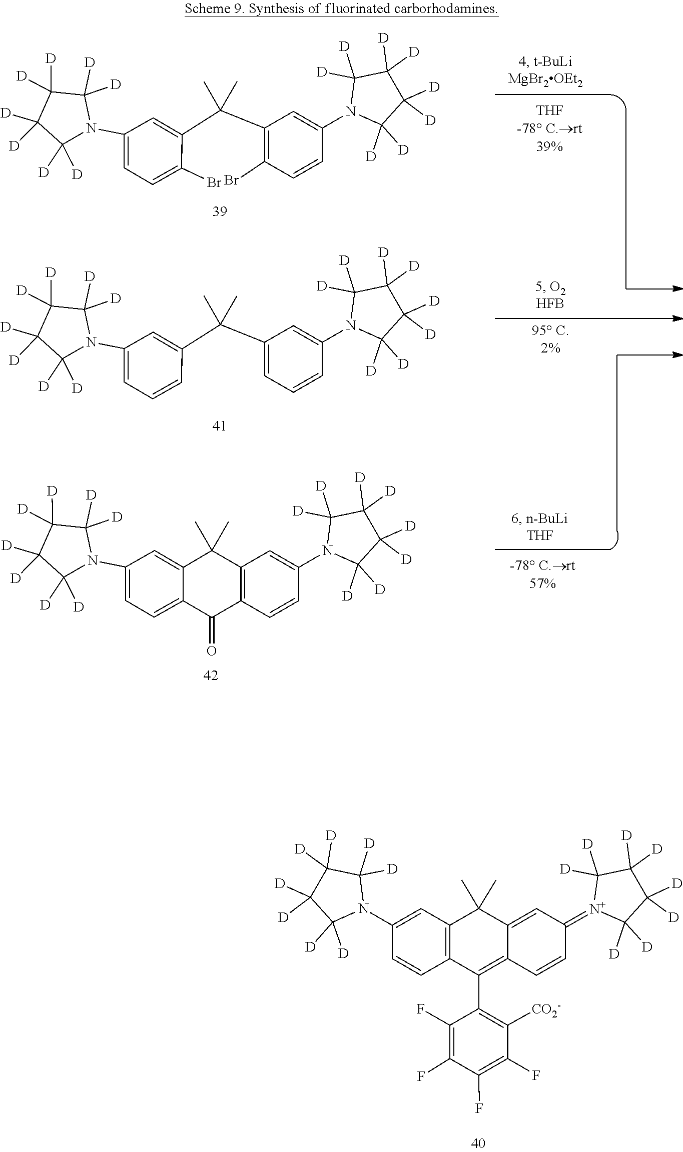

Li/Br-exchange of deuterated dibromide 39 (Scheme 10a) and addition to tetrafluorophthalic anhydride 4 gave carborhodamine 40 (JFX₆₃₇) in modest yield (39%). This route was complicated by the longer, lower-yielding, and relatively complex synthesis of 39 compared to those of the oxygen and silicon dibromides (e.g., 7 and 24; Scheme 3, Scheme 6). Use of the lactol condensation conditions to react 41 (Scheme 10a) with 5 was unsuccessful, yielding only a small amount (<2%) of dye 40 even after extended reaction time (>72 h).

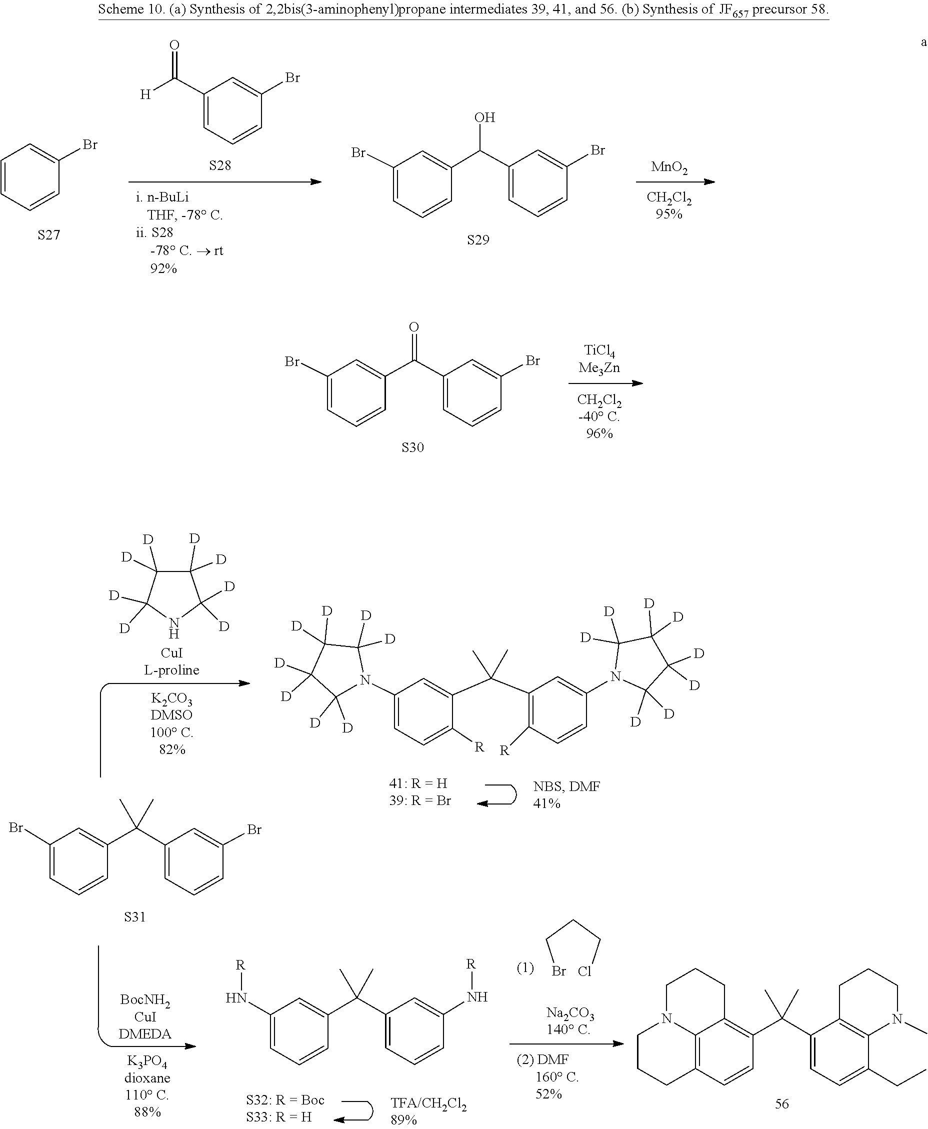

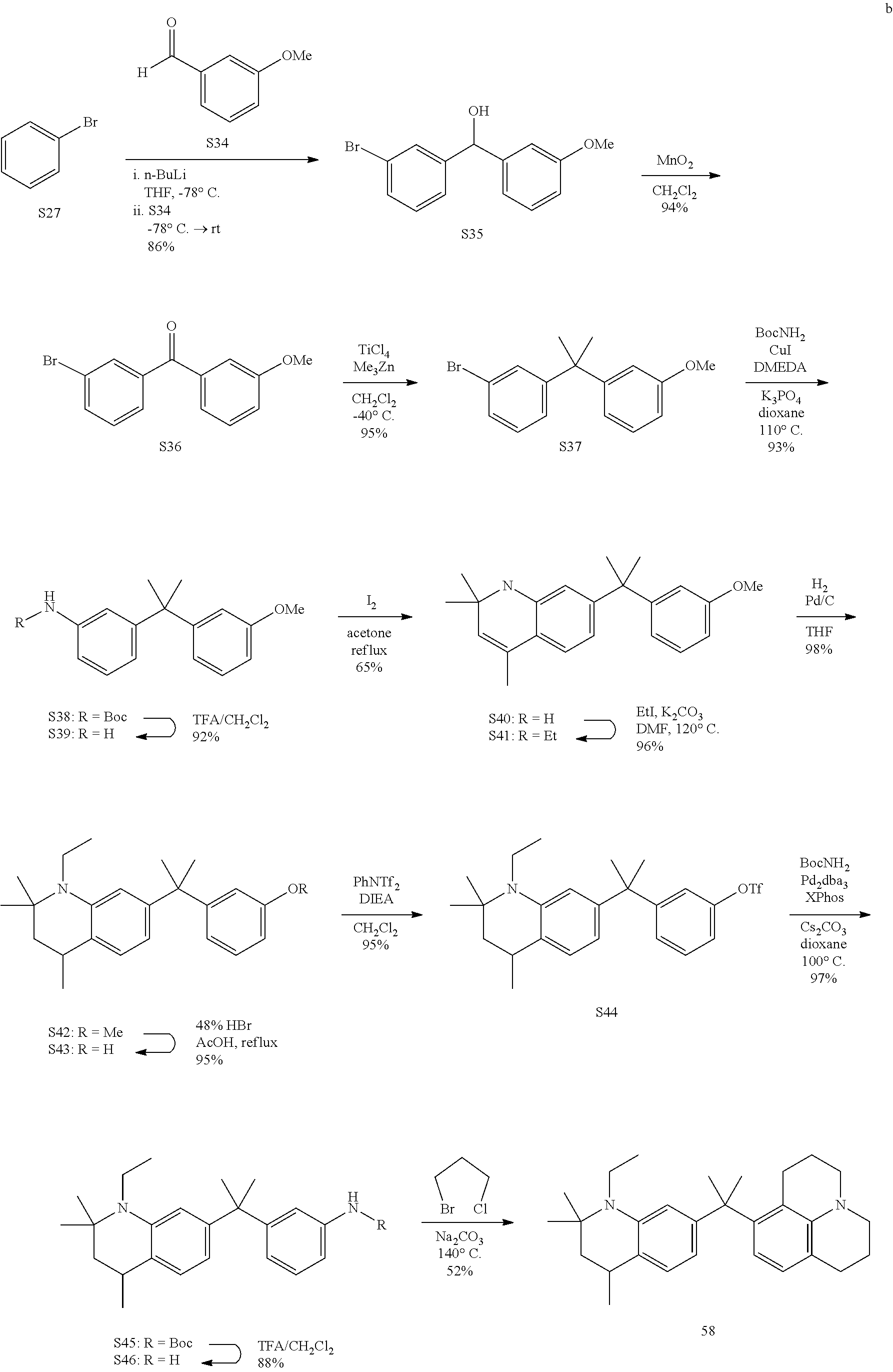

These issues with existing synthetic methods prompted consideration of a unique method to prepare carborhodamine dyes. In previous work, carbofluoresceins were synthesized by reacting aryl Grignards with dihydroxyanthrones; these could be converted to carborhodamines using Pd-catalyzed cross-coupling.⁴¹ The use of a Grignard was necessary to accommodate t-butyl-protected carboxyl groups, but this arylmagnesium species was not reactive enough to add to more electron-rich diaminoanthrones and directly generate rhodamine dyes.³² Aryllithium reagents are more commonly used for addition to diaminoanthrones due to their increased nucleophilicity, which limits functional group compatibility. Aside from one recent example,⁵³ all existing syntheses of carboxy-substituted carbo- and Si-rhodamines involve lithium/bromide exchange on a rigorously protected phthalic acid equivalent and addition of the resulting aryllithium species to a diaminoanthrone.²⁹⁻³⁴ It was surmised that later-stage incorporation of the 6⁻carboxyl group using the MAC chemistry approach would minimize protecting group issues and simplify the use of the more reactive aryllithium reagents; the bespoke fluorinated phenyl ring could also facilitate the exchange, transmetalation, or deprotonation needed to form the desired organometallic species. Nonetheless, previous attempts to generate tetrafluoroaryllithium reagents containing protected 2⁻carboxyl groups via metal/halide exchange have proven unsuccessful.²¹ Direct metalation (i.e, deprotonation or Li/H-exchange) of unprotected 2,3,4,5⁻tetrafluorobenzoic acid (6) with 2 equivalents of n-butyllithium⁴³ (SCHEME 1E) generated a dianionic aryllithium species that underwent facile addition to anthrone 42 (Scheme 11a), affording deuterated rhodamine JFX₆₃₇ (40) in higher yield (57%) compared to the other two synthetic methods (Scheme 9).

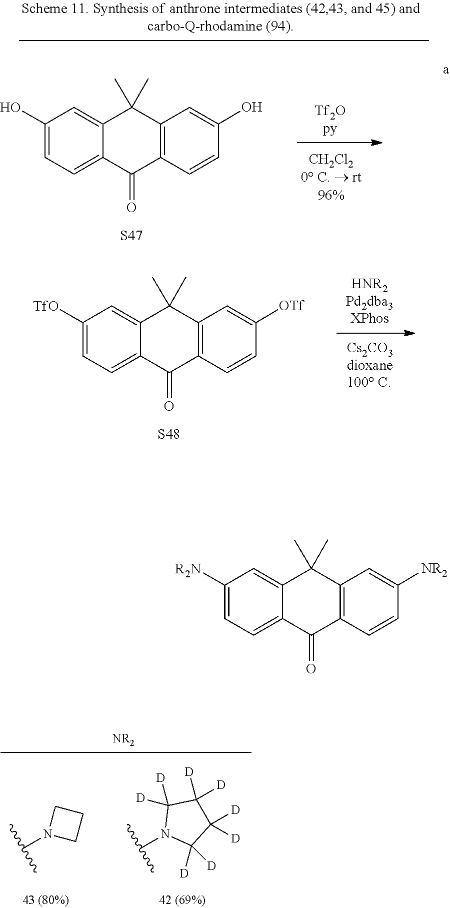

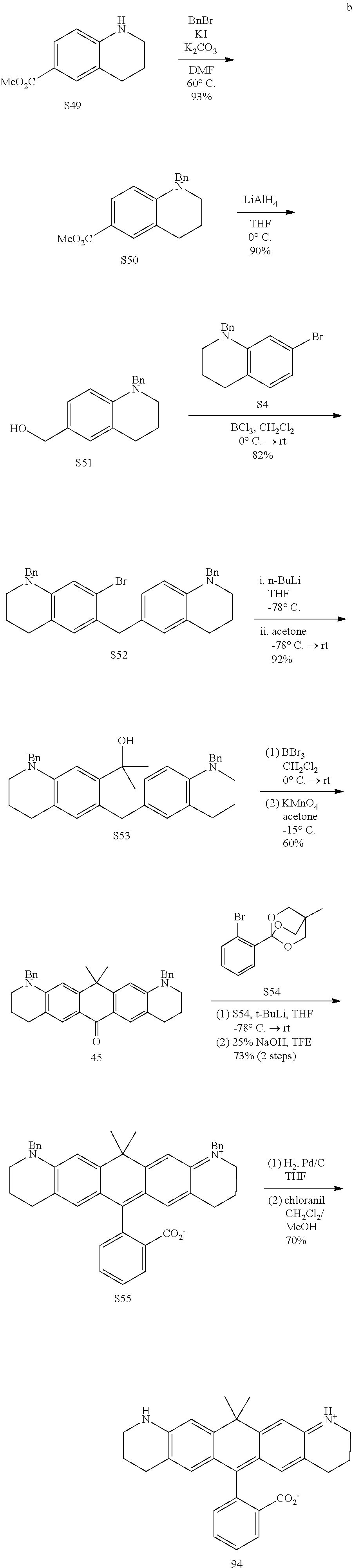

This new Li/H exchange approach was then applied to prepare other fluorinated dyes. Use of anthrone 43 (Scheme 11a) gave the unique azetidinyl dye 44 (JF₆₃₂; Scheme 12) in reasonable yield (53%). The dibenzyl anthrone 45 (Scheme 11b) afforded compound 46, which could be deprotected to yield 4,5,6,7⁻tetrafluoro-carbo-Q-rhodamine (FCRh_{Q}, 47). Similarly, reaction of Michler's ketone (48) with the dianion of 6 furnished the fluorinated analog of Malachite Green lactone (FMGL; 49) in excellent yield (80%).

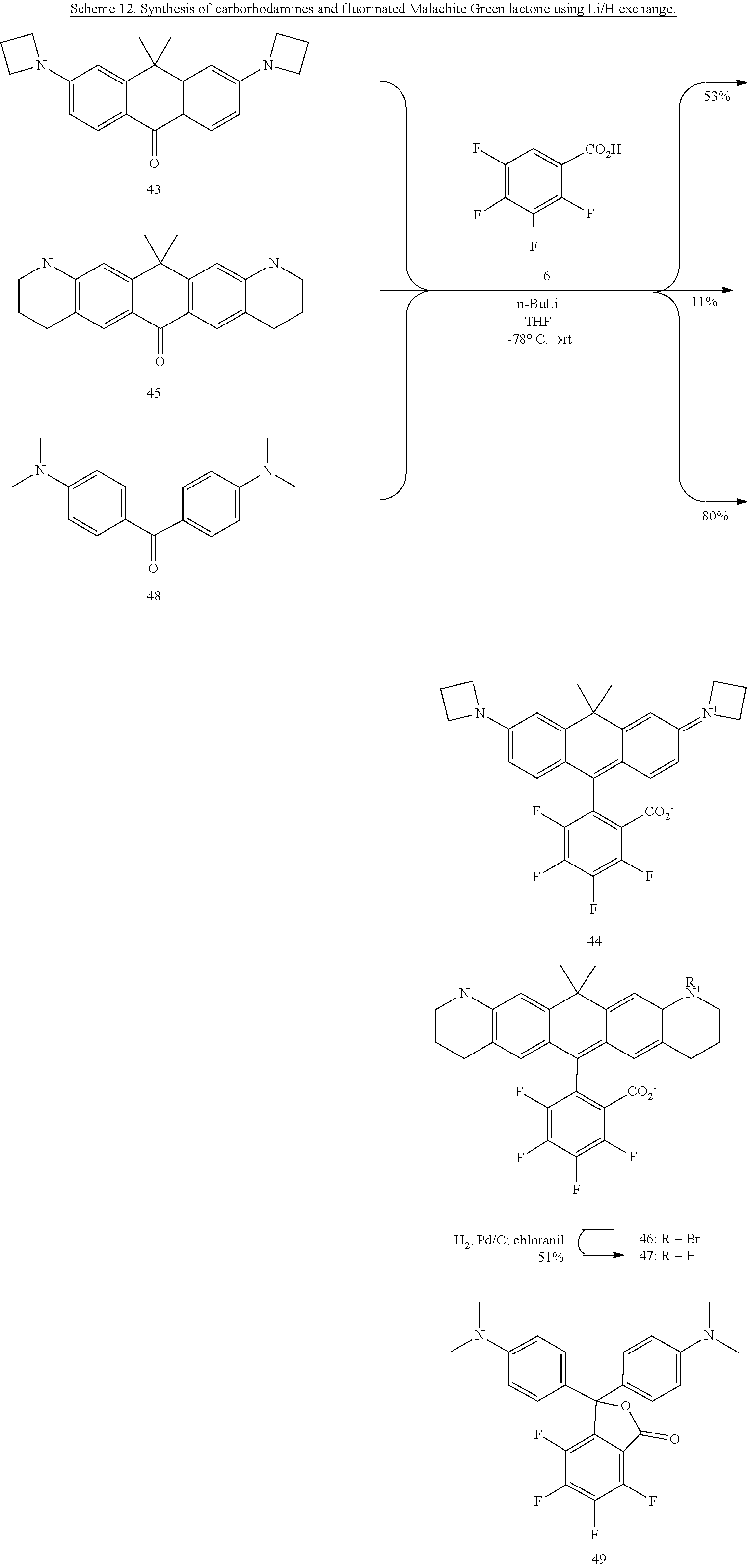

### Example 6: Synthesis of Gluorescein Analogs

This chemistry could also be used to synthesize fluorescein analogs though a two-step, high-yielding protocol (77-94%; Scheme 13). Addition of lithiated 6 to di(silyloxy)anthrone 50 yielded the fluorinated carbofluorescein 51 after TBS deprotection with TBAF. Use of anthrone 52 in this sequence gave highly fluorinated 53, an analog of Virginia Orange.⁵⁴ This method could be extended to Si-anthrone 54 to provide the known, 14 pH-sensitive, fluorinated Si-fluorescein 55 in high yield. These examples demonstrate the broad scope of 2,3,4,5⁻tetrafluorobenzoic acid (6) lithiation/addition in the synthesis of both rhodamine and fluorescein dyes (Scheme 14).

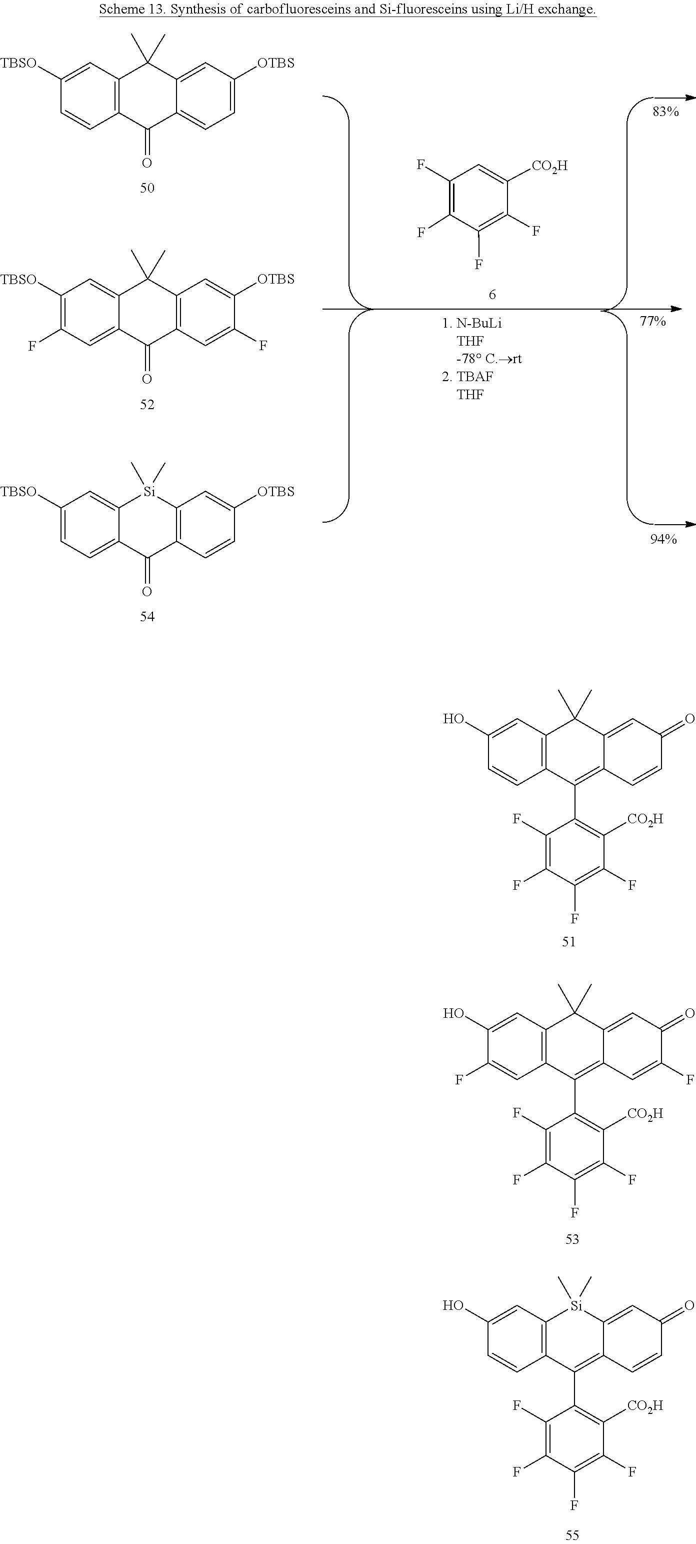

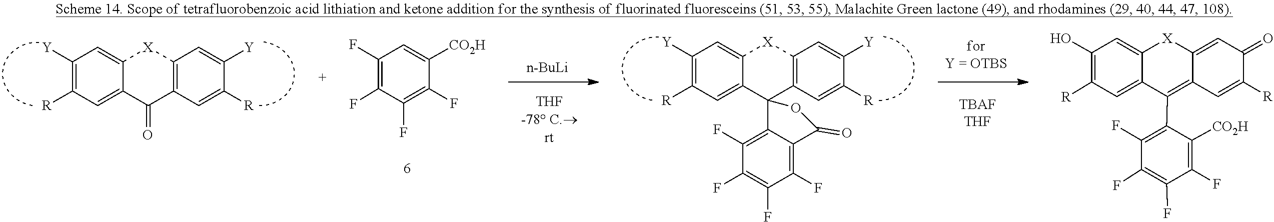

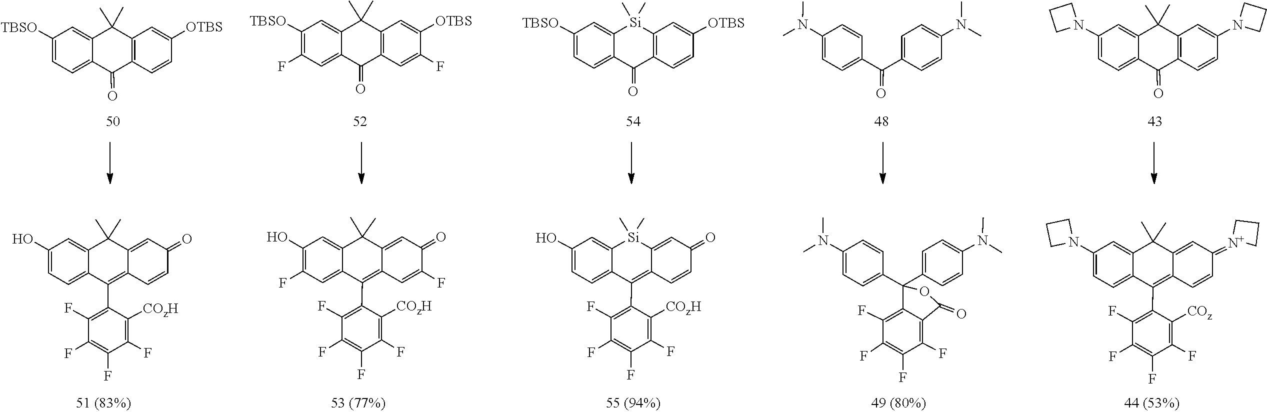

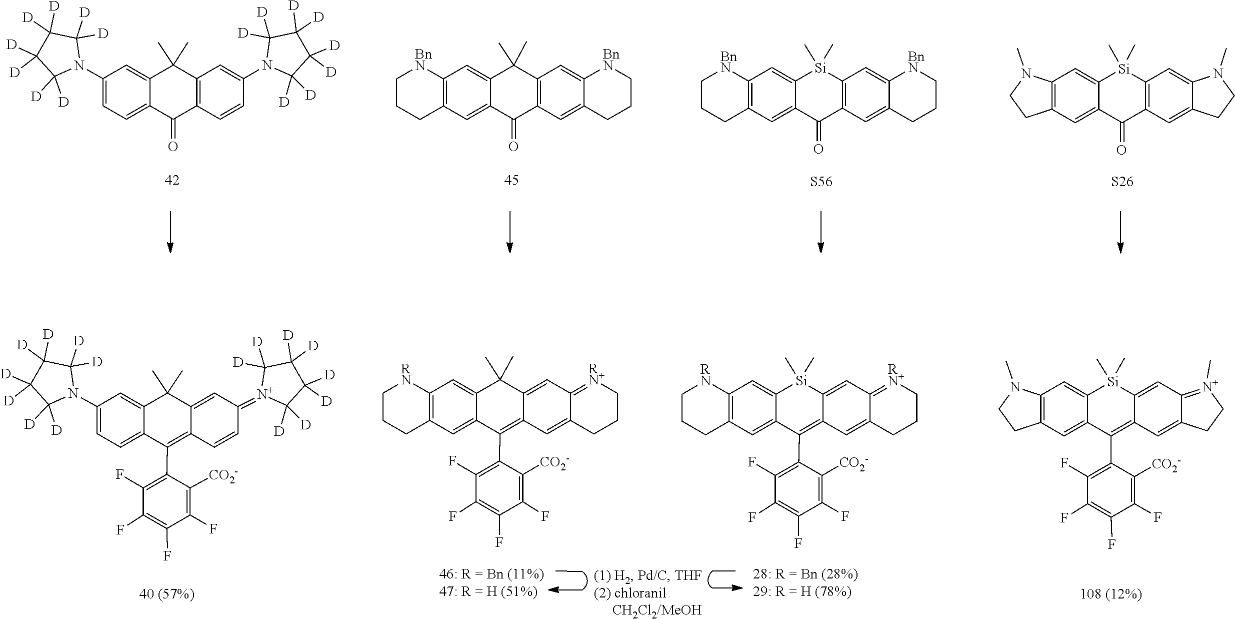

### Example 7: Lewis Acid-Catalyzed Condensation for Carborhodamine Synthesis

The collection of fluorinated carborhodamines was further expanded. Although the dibromide and site-selective lithiation routes (Scheme 1A,E) give good yields of carborhodamine dyes with compact substituents such as pyrrolidine and azetidine (Scheme 9, Scheme 12), this chemistry proved ineffective for dyes with highly substituted, fused-ring auxochromes such as those derived from reduced quinolines or julolidine.^{34, 55} Inspired by a brief report in the patent literature,⁵⁶ a different approach was explored, returning to classic acid-catalyzed chemistry but using the Lewis acid AlCl₃ (Scheme 15).

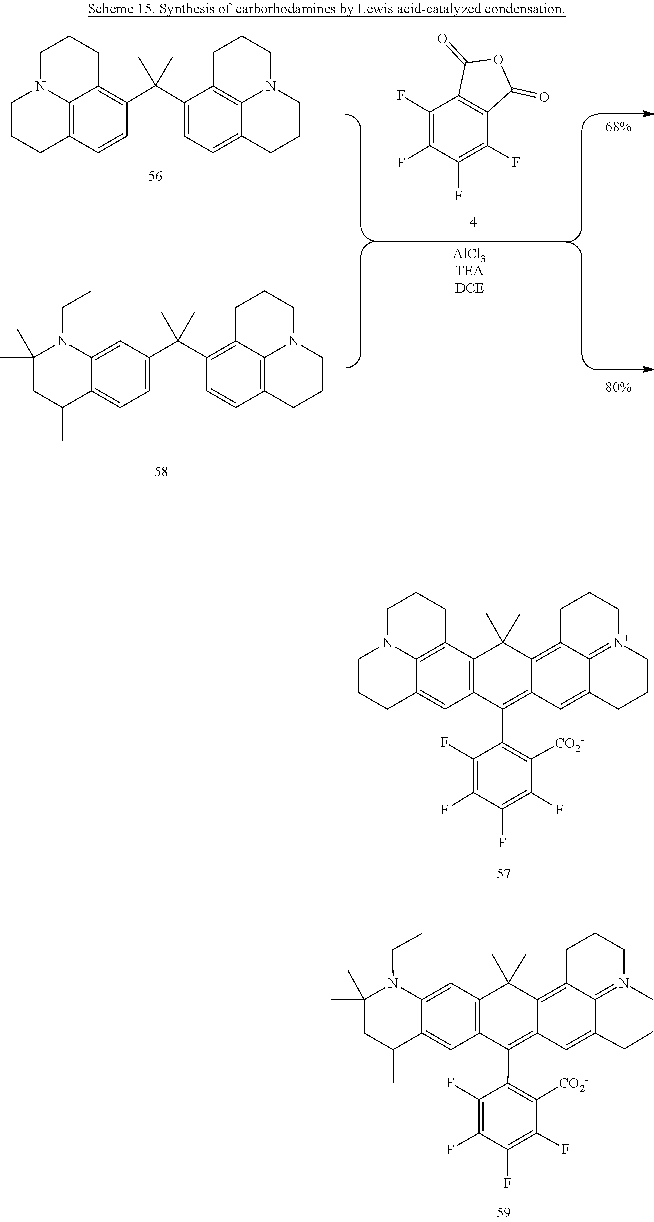

Reaction of bis-julolidine 56 (Scheme 10a) with tetrafluorophthalic anhydride (4) in the presence of AlCl₃ afforded a 68% yield of the fluorinated carbon-containing analog of rhodamine 101 (JF₆₆₀, 57). Likewise, AlCl₃⁻mediated condensation of 58 (Scheme 10b) and 4 gave 59, a fluorinated analog of ATTO 647N, in excellent yield (80%). High-yielding syntheses of the des-fluoro analogs of rhodamines 57 and 59 was also achieved using this chemistry (Scheme 16). In contrast, all attempts to synthesize 57 or 59 through addition of lithiated 6 to anthrones containing a julolidine motif⁵⁷ or via dibromination of 56 or 58 failed to provide appreciable amounts of the desired carborhodamine products.

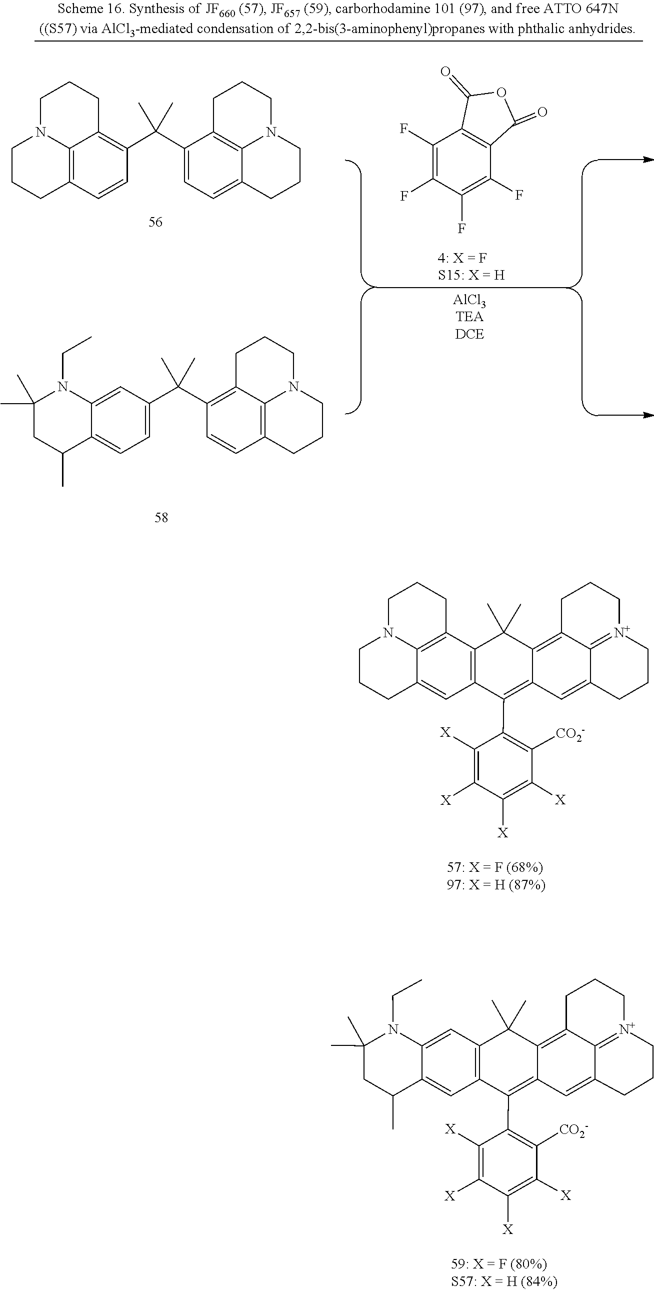

As will be appreciated upon study of this document, in Scheme 16 and in other Schemes set forth herein, X is defined as F, H, or a combination thereof. As is apparent from consideration to the disclosure as a whole, the compounds and methods of making such compounds are inclusive of those in which each X is independently selected from F and H.

### Example 8: Synthesis of Rhodamines from Fluoresceins Using Pd-Catalyzed Cross-Coupling

In a prior report cross-coupling of fluorescein ditriflates with amines was demonstrated as an efficient method to synthesize rhodamine dyes.⁴⁰ Nevertheless, use of the known¹⁹ 4,5,6,7⁻tetrafluorofluorescein (60) or analogs such as 51, 53, and 55 (Scheme 13) requires special considerations due to the electrophilicity of the perfluorinated arene. Cross-coupling reactions using primary or secondary aliphatic amine partners were unsuccessful due to facile substitution reactions by these nucleophilic species on the tetrafluoroaryl moiety^{16, 58, 59} under the requisite reaction conditions (Cs₂CO₃, 80-100° C.). Less nucleophilic aromatic amines or N-acyl derivatives are compatible with these conditions, however, allowing the synthesis of unique dyes (Scheme 17).

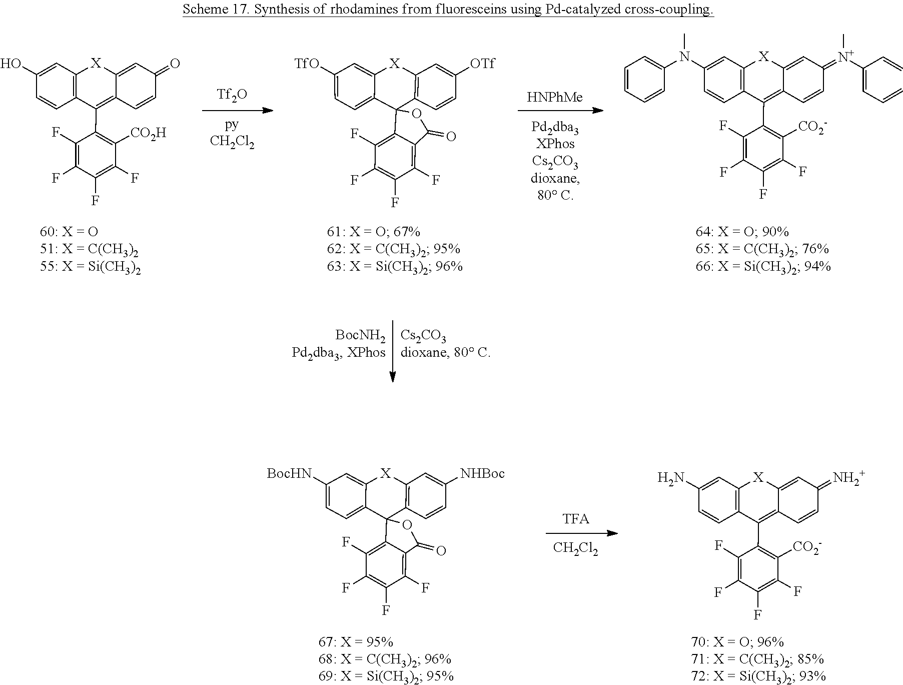

Reaction of 60, the carbon-containing analog 51, and Si-fluorescein 55 with triflic anhydride yielded ditriflates 61-63. Cross-coupling with N-methylaniline provided the N-arylrhodamines 64-66. Likewise, diacylrhodamines 67-69 were accessed in nearly quantitative yield through cross-coupling of the fluorescein ditriflates with t-butyl carbamate. Deprotection of the t-Boc groups with TFA gave rhodamine 110 derivatives 70 (FRh₁₁₀), 71 (FCRh₁₁₀), and 72 (FSiRh₁₁₀). Fluorinated rhodamine 110 variants are largely unknown, with only a brief report in the patent literature³⁸ detailing the synthesis of 4,5,6,7⁻tetrafluoro-Rh₁₁₀ (70) in low yield (˜5%) through an acid-catalyzed synthesis. These results demonstrate that fluorinated fluorescein ditriflates can be used in cross-coupling reactions, albeit with a reduced scope limited to less nucleophilic nitrogen reactants.

### Example 9: Investigation of Spectral Properties of Fluorescein Variants

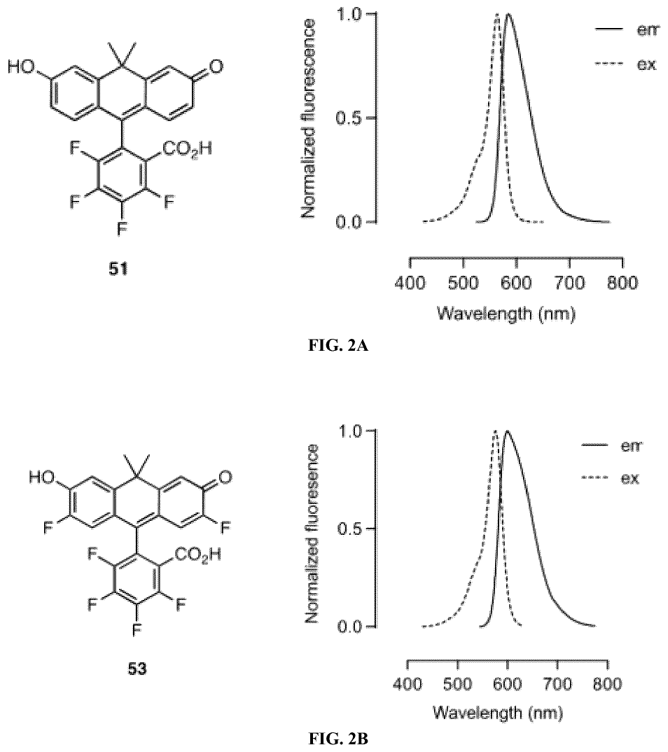

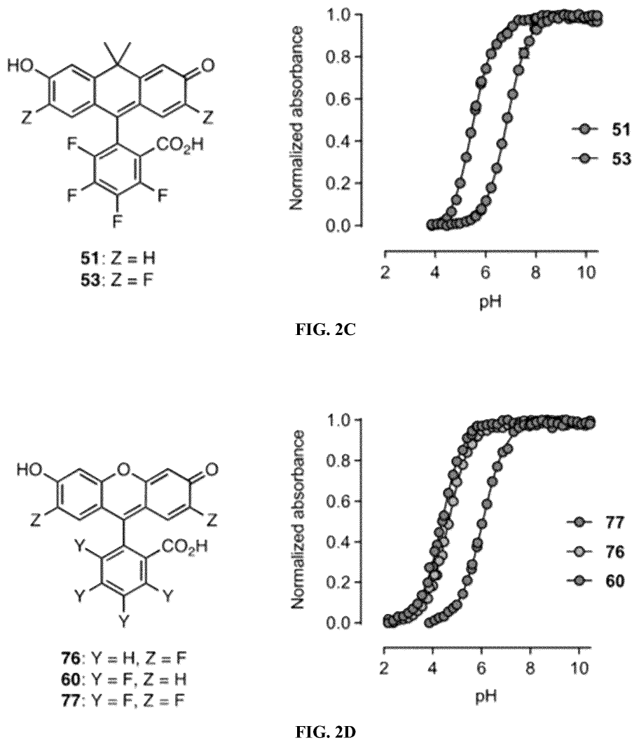

Combined with previous investigations,^{14, 16-18, 32, 40, 41, 45} the work described herein (Schemes 1, 3, 4, 6, 8, 9, 12, 13, 15, and 17 and Schemes 5, 14, and 16) represents a comprehensive collection of fluoresceins and rhodamines containing 0, C, or Si bridging substituents, fluorinated or nonfluorinated pendant phenyl rings, and different auxochrome groups. The investigation of their spectral properties was initiated by examining the fluorescein variants, measuring λ_{abs}, λₑₘ, extinction coefficient (ε), and fluorescence quantum yield (Φ_{f}) of each dye (Table 3 and FIG. 2A-2D). Fluorescein and many of its derivatives exhibit pH-sensitive spectral properties, so the pKₐ values were also determined and the cooperativity of the pH-mediated spectral changes was investigated by determining the Hill coefficients (η_{H}) calculated from the pH titration data.

Consistent with previous work describing 4,5,6,7⁻tetrafluorofluorescein (60)¹⁹ and 4,5,6,7⁻tetrafluoro-Si-fluorescein (55),¹⁴ the tetrafluorophenyl ring system in dyes 51 and 53 elicited a >15 nm bathochromic shift in λ_{abs} and λₑₘ and a lower pKₐ relative to the parent carbofluorescein (74) and Virginia Orange (78) dyes. Fluorophores 51 and 53 showed a noncooperative pH-driven transition with η_{H}≈1. This is in contrast to the behavior of carbofluoresceins 74 and 78, which do not contain the perfluorinated ring system and show cooperative transitions (η_{H}>1) from a colored to colorless species as pH decreases.^{41, 54} The Si-fluoresceins 55 and 80 maintain a cooperative pH titration even with the fluorinated ring system installed. These data reveal a subtle interplay between the nucleophilicity of the ortho-carboxyl group and the propensity of the dye to lactonize that controls the cooperative quinoid→lactone transition upon protonation. This understanding will help guide the future development of pH sensors based on red-shifted fluorescein analogs.^{41, 54, 60}

TABLE 3
Spectral properties of fluorescein derivatives.

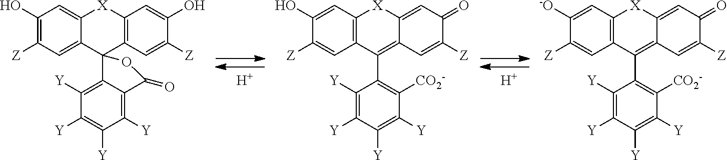

| compound | X | Y | Z | λ_{abs} (nm) | ε (M⁻¹cm⁻¹) | λₑₘ (nm) | Φf | pKₐ | η_{H} |
|---|---|---|---|---|---|---|---|---|---|
| 73 | 0 | H | H | 490 | 90,000 | 514 | 0.92 | 6.25 | 0.97 |
| 60 | O | F | H | 508 | 85,600 | 527 | 0.85 | 6.04 | 0.92 |
| 74 | C(CH₃)₂ | H | H | 544 | 108,000 | 567 | 0.62 | 7.54 | 1.65 |
| 51 | C(CH₃)₂ | F | H | 564 | 96,700 | 585 | 0.54 | 6.86 | 1.02 |
| 75 | Si(CH₃)₂ | H | H | 579 | 93,000 | 599 | 0.53 | 8.27 | 1.69 |
| 55 | Si(CH₃)₂ | F | H | 598 | 84,100 | 614 | 0.50 | 7.08 | 1.23 |
| 76 | O | H | F | 490 | 82,400 | 514 | 0.97 | 4.65 | 0.98 |
| 77 | O | F | F | 508 | 81,200 | 527 | 0.18 | 4.37 | 1.00 |
| 78 | C(CH₃)₂ | H | F | 555 | 90,900 | 581 | 0.40 | 6.75 | 1.46 |
| 53 | C(CH₃)₂ | F | F | 576 | 91,100 | 599 | 0.29 | 5.47 | 1.00 |
| 79 | Si(CH₃)₂ | H | F | 596 | 60,900 | 618 | 0.42 | 7.38 | 1.30 |
| 80 | Si(CH₃)₂ | F | F | 617 | 67,900 | 635 | 0.32 | 6.08 | 1.50 |

All properties were measured in 0.1 N NaOH

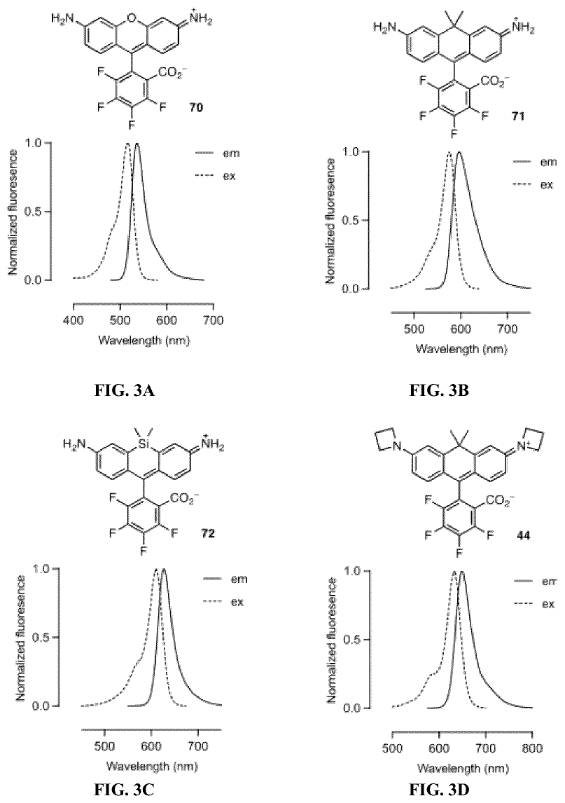

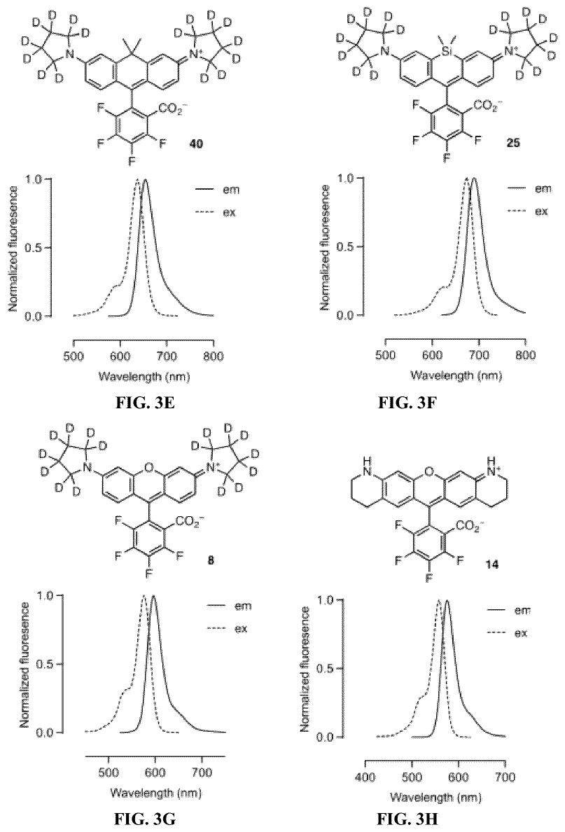

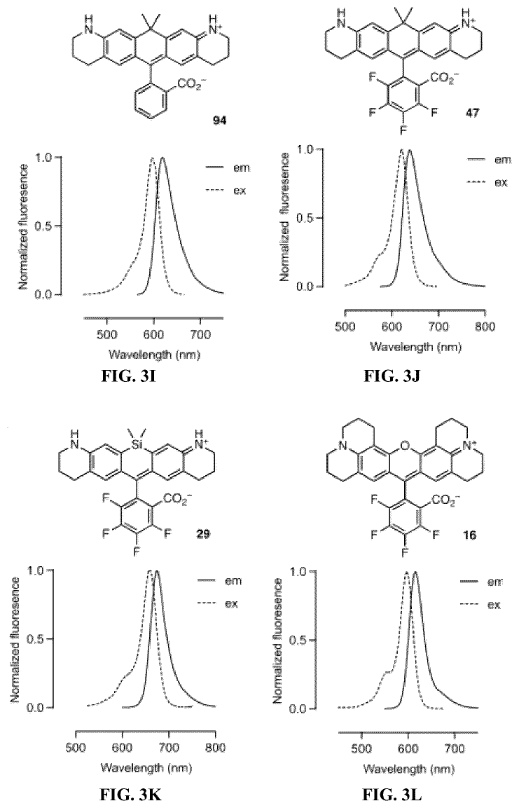

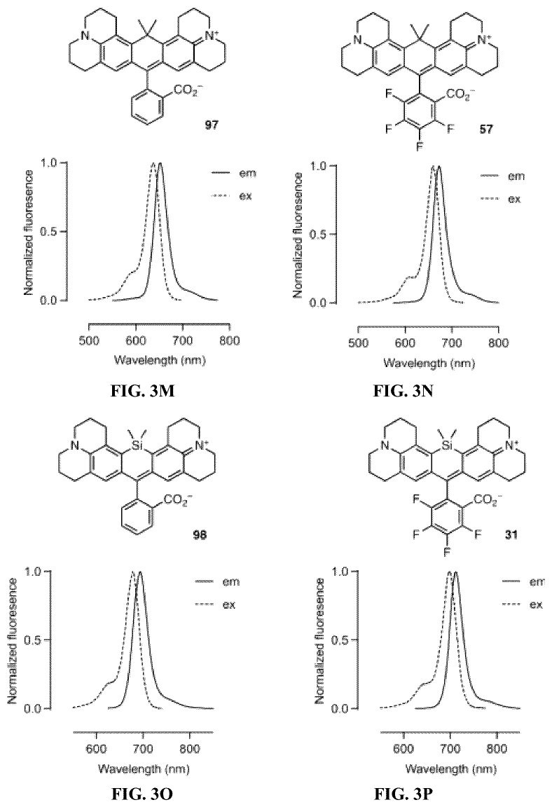

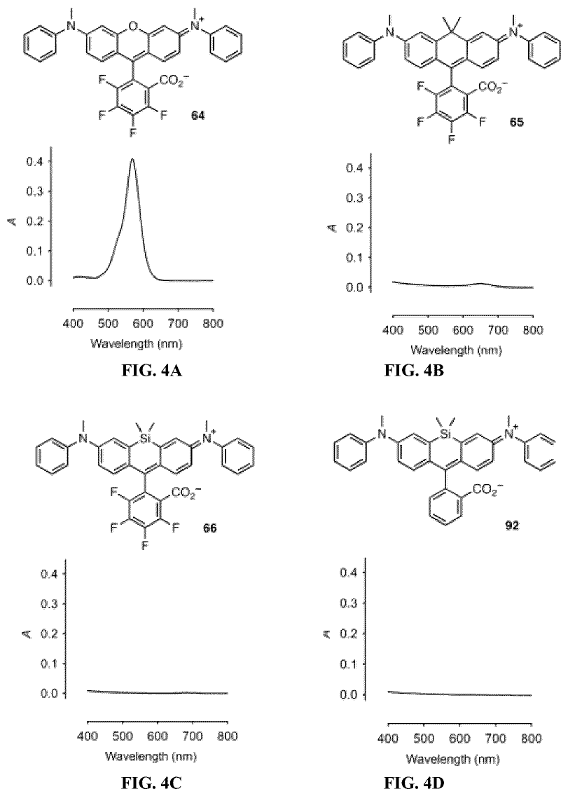

The rhodamines (Table 4, FIG. 3A-3P, and FIG. 4A-4D) were then investigated by measuring the standard spectral properties (λ_{abs}, ε, λₑₘ, and Φ_{f}) along with K_{L-Z}.¹⁶ Consistent with previous data, fluorination of the pendant phenyl ring increases λ_{abs} and λₑₘ by 15-25 nm; the fluorinated dyes also exhibit a ˜5 nm smaller Stokes shift compared to their des-fluoro analogs.

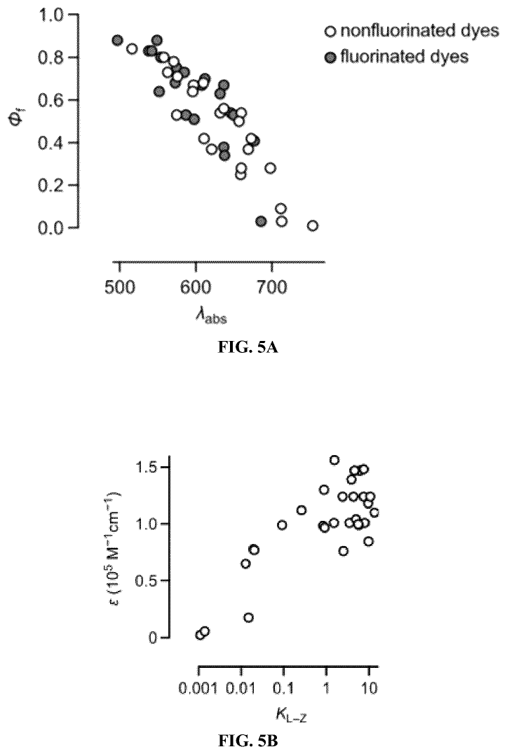

This modification also modestly increases e and decreases O_{f}. The lower quantum yields could be caused by the energy gap law⁶¹ or photoinduced electron transfer (PeT) from the xanthene system to the electron-deficient pendant ring;⁶² plotting Φ_{f} vs. λ_{abs} showed no substantial difference in trends between fluorinated and nonfluorinated dyes (FIG. 5A) thereby supporting the energy gap law hypothesis. Carborhodamine 86 (JF₆₀₈) shows λ_{abs}/λₑₘ=608 nm/631 nm with ε=99,000 M⁻¹ cm⁻¹, and Φ_{f}=0.67. The fluorinated analog JF₆₃₂ (44) exhibits red-shifted spectra (λ_{abs}/λₑₘ=632 nm/649 nm), a higher absorptivity (ε=139,000 M⁻¹ cm⁻¹), and a lower O_{f}=0.54. These trends are mirrored in the analogous deuterated JFX dyes 88 and 40. Likewise, the previously reported Si-Q-rhodamine³² (95) exhibits λ_{abs}/λₑₘ=637 nm/654 nm with ε=77,000 M⁻¹ cm⁻¹ and Φ_{f}=0.38. The fluorinated analog 29 shows a bathochromic shift with λ_{abs}/λₑₘ=659 nm/675 nm, higher ε=124,000 M⁻¹ cm⁻¹, and lower O_{f}=0.25. These trends in λ_{abs} and ε are also present in the nonfluorescent N-arylrhodamines. Rhodamine 90 displays λ_{abs}=549 nm with modest absorptivity (ε=16,000 M⁻¹ cm⁻¹); incorporation of fluorine atoms to give 64 increases the absorption maximum and extinction coefficient (λ_{abs}=570 nm; ε=81,700 M⁻¹ cm⁻¹).

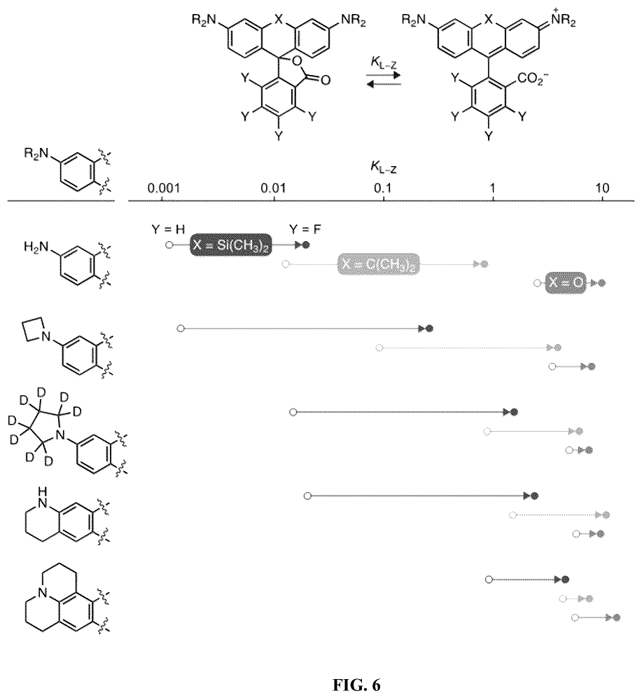

The lactone-zwitterion equilibrium was then examined across the different dye series (Table 4); K_{L-Z} was measured in 1:1 (v/v) dioxane:water mixtures. These conditions provide the broad range of values necessary for identifying structure-activity relationships.¹⁸ Plotting auxochrome structure vs. K_{L-Z} (FIG. 6) showed two general trends. First, fluorination universally increases K_{L-Z}; this shift ranged from two orders of magnitude (e.g., 1→2) to two-fold (e.g., 97→57) depending on dye structure. Second, K_{L-Z} is strongly dependent on both the bridging substituent (Si(CH₃)₂; C(CH₃)₂; O) and the auxochrome moiety. The effect of the bridging substituent on K_{L-Z} is well-established but the dependence on the auxochrome groups-especially highly electron-donating moieties has not been rigorously explored.^{15, 16,63}

TABLE 4
Spectral properties of rhodamine derivatives.

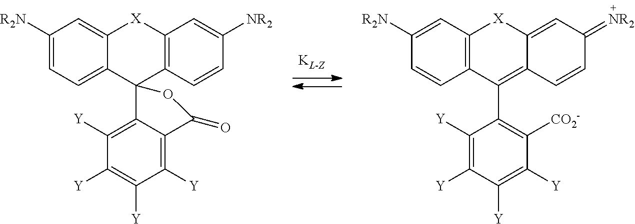

| 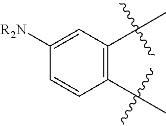 | compound | X | Y | λ_{abs} (nm) | ε (M⁻¹cm⁻¹) | λₑₘ (nm) | Φ_{f} | K_{L-Z} |
|---|---|---|---|---|---|---|---|---|
| 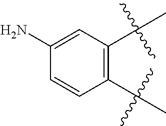 [SMILES: *c1ccc(N)cc1*] | 81 70 82 71 83 72 | O O C(CH₃)₂ C(CH₃)₂ Si(CH₃)₂ Si(CH₃)₂ | H F H F H F | 487 516 552 575 587 611 | 75,000 84,500 65,000 96,400 2,200 77,800 | 520 536 577 597 609 627 | 0.88 0.64 0.64 0.53 0.53 0.42 | 2.53 9.81 0.0127 0.832 0.0011 0.0195 |
| 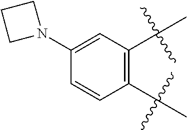 [SMILES: *c1ccc(N2CCC2)cc1*] | 84 85 86 44 1 2 | O O C(CH₃)₂ C(CH₃)₂ Si(CH₃)₂ Si(CH₃)₂ | H F H F H F | 549 571 608 632 646 669 | 101,000 101.000 99,000 139,000 5,600 112,000 | 571 590 631 649 664 662 | 0.88 0.78 0.67 0.54 0.54 0.37 | 3.47 7.93 0.0911 3.59 0.0014 0.262 |
| 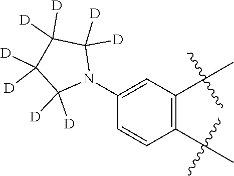 [SMILES: *c1ccc(N2C([2H])([2H])C([2H])([2H])C([2H])([2H])C2([2H])[2H])cc1*] | 87 8 88 40 89 25 | O O C(CH₃)₂ C(CH₃)₂ Si(CH₃)₂ Si(CH₃)₂ | H F H F H F | 554 576 612 637 650 673 | 184.000 124,000 130,000 147,000 17,600 156,000 | 576 597 633 654 667 690 | 0.80 0.71 0.70 0.56 0.53 0.42 | 4.96 7.50 0.880 6.12 0.0149 1.56 |
| 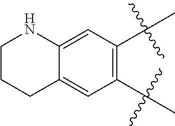 [SMILES: *c1cc2c(cc1*)NCCC2] | 93 14 94 47 95 29 | O O C(CH₃)₂ C(CH₃)₂ Si(CH₃)₂ Si(CH₃)₂ | H F H F H F | 538 558 598 621 637 689 | 99,000 118,000 101,000 124,000 77,000 124,000 | 558 576 620 638 654 675 | 0.83 0.80 0.51 0.37 0.38 0.25 | 5.74 8.57 1.51 10.8 0.0202 2.39 |
| 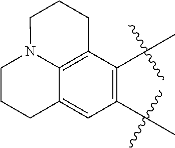 [SMILES: *c1cc2c3c(c1*)CCCN3CCC2] | 96 16 97 57 98 31 | O O C(CH₃)₂ C(CH₃)₂ Si(CH₃)₂ Si(CH₃)₂ | H F H F H F | 575 597 637 660 677 698 | 99,800 110,000 124,000 148,000 96,700 147,000 | 597 616 652 673 694 712 | 0.75 0.67 0.67 0.54 0.41 0.28 | 5.58 13.4 4.32 7.55 0.915 4.5.7 |
| 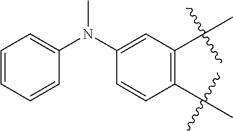 [SMILES: *c1ccc(N(C)c2ccccc2)cc1*] | 90 64 91 65 92 56 | O O C(CH₃)₂ C(CH₃)₂ Si(CH₃)₂ Si(CH₃)₂ | H F H F H F | 549 670 645 | 16,000 81,700 <200 2,700 <200 <200 | | | 0.296 4.36 <10−4 0.344 <10−4 <10−4 |

All properties were measured in 10 mM HEPES, pH 7.3 except for K_{L-Z}, which was determined in 1:1 dioxane:H₂O

Although the Hammett constants⁶⁴ for most of these functionalities are unknown, the electron-donating capability of the auxochrome groups can be ranked as: NH₂<azetidine<pyrrolidine<tetrahydroquinoline<julolidine based on data from substituted azobenzene dyes.^{65, 66} The K_{L-Z} values follow this trend with Si-rhodamines SiRh₁₁₀ (83) and JF₆₄₆ (1) showing K_{L-Z}≈10⁻³, JFX₆₅₀ (89) and SiRh_{Q} (95) exhibiting K_{L-Z}≈10⁻² and the julolidine-containing JF₆₉₈ (31) giving K_{L-Z}≈1. The correlation between the electron-donating character of the auxochrome and K_{L-Z} is also observed for the carborhodamines and rhodamine dyes at commensurately higher K_{L-Z} values (FIG. 6). N-Aryl auxochromes substantially shift the lactone-zwitterion equilibrium lower. Rhodamine 90 had a relatively small K_{L-Z}=0.296 but fluorination shifted the equilibrium constant higher; 64 showed K_{L-Z}=4.36 (Table 4).

Overall, these data show that rhodamines that typically favor the lactone form, such as SiRh₁₁₀ (83; ε=2,200 M⁻¹ cm⁻¹, K_{L-Z}, =0.0011) can be tuned through cooperative structural modifications-introduction of electron-donating auxochromes and fluorine substituents to yield JF₆₉₈ (31), a dye that exhibits high absorptivity in aqueous solution (ε=147,000 M⁻¹ cm⁻¹, K_{L-Z}, =4.57).

It is also noted that dyes containing electron-donating substituents, such as julolidine, exhibit higher and more compressed K_{L-Z} ranges. This makes such scaffolds attractive for constructing bright, highly absorbing labels-8 is correlated with K_{L-Z} (FIG. 5B)—but could limit efforts to fine-tune such fluorophores to access properties such as improved bioavailability or chromogenicity (FIG. 1A).

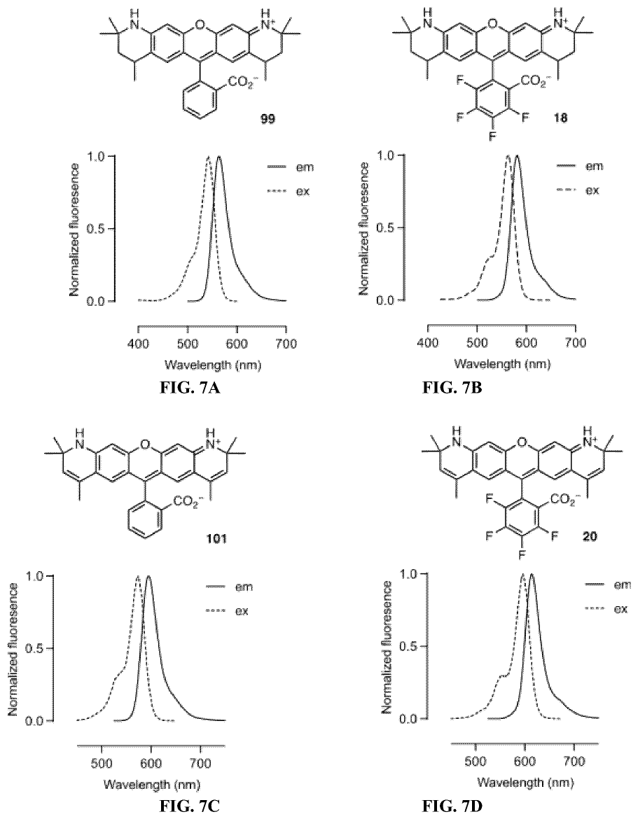

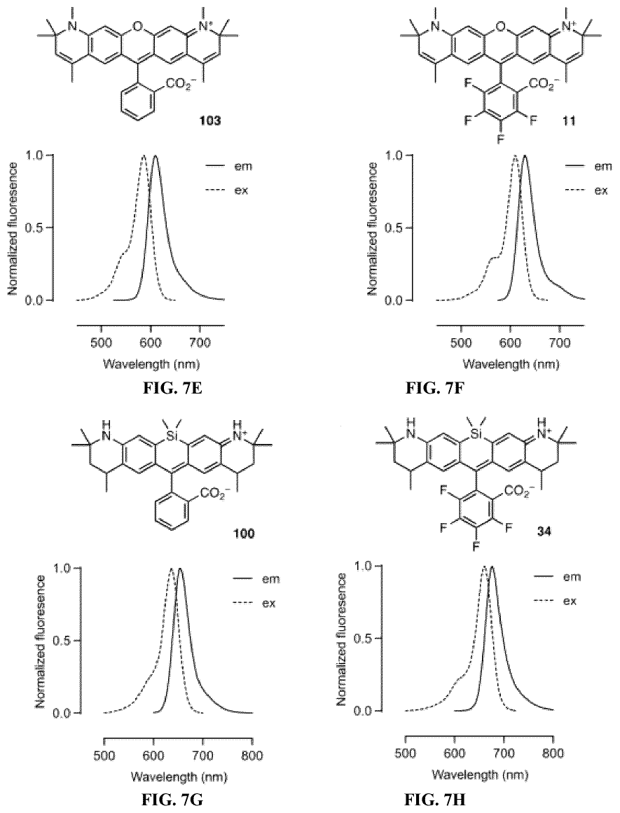

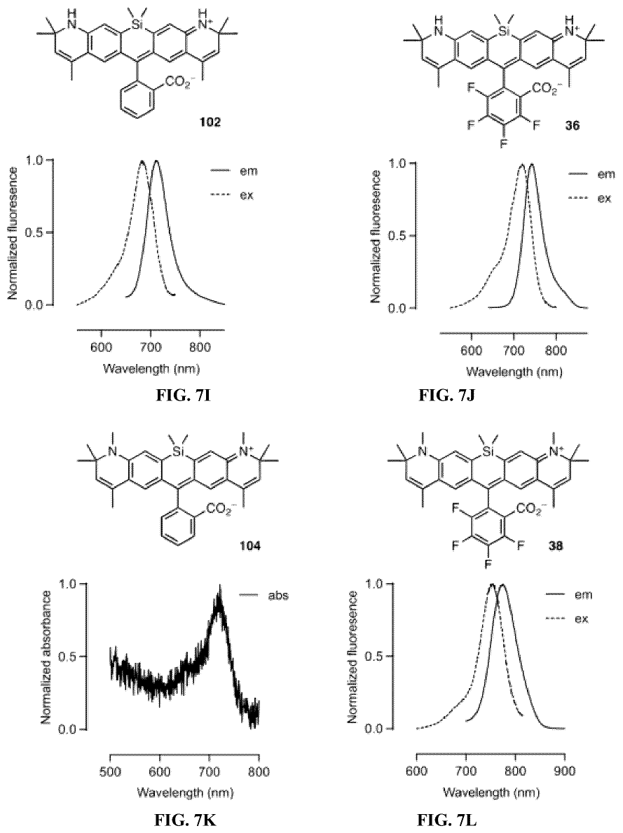

Finally, the oxygen- and silicon-containing rhodamines were compared with more complicated auxochrome substituents (Table 5, FIG. 7A-7L). Again, the fluorine modification universally elicited longer λ_{abs} and em, smaller Stokes shift, larger e and K_{L-Z}, and lower O_{f}. For example, tetrahydroquinoline rhodamine 99—the free dye form of ATTO 550 shows λ_{abs}/λₑₘ=542 nm/564 nm with ε=112,000 M⁻¹ cm⁻¹, O_{f}=0.83, and K_{L-Z}=6.30. The fluorinated analog 18 (JF₅₆₃) exhibits red-shifted spectra (λ_{abs}/λₑₘ=563 nm/581 nm), higher K_{L-Z}=15.3 and absorptivity (ε=125,000 M⁻¹ cm⁻¹), and slightly lower O_{f}=0.73. The tetrahydroquinoline-containing Si-rhodamines 100 and 34 followed this trend. The dihydroquinoline dyes (11, 20, 36, 38, and 101-104) exhibited lower K_{L-Z} values than the tetrahydroquinoline dyes (18, 34, 99 and 100) and the structurally simpler Q-rhodamines (14, 29, 93, and 95), illustrating that this extended conjugated system shifts K_{L-Z} towards the lactone form (FIG. 8). These data also revealed that Si-rhodamines containing dihydroquinoline systems (102, 36, 104, and 38) possess substantially lower O_{f}≤0.03 compared to tetrahydroquinoline Si-dyes 100 and 34 (O_{f}≈0.3), and the directly analogous oxygen-containing rhodamines 101, 20, 103, and 11 (O_{f}=0.64-0.83). This suggests the extended conjugation of the dihydroquinoline-containing dye scaffold promotes a nonradiative decay pathway in the Si-rhodamine system, making such dyes less attractive as fluorescent labels.

TABLE 5
Spectral properties of quinolinyl rhodamines.

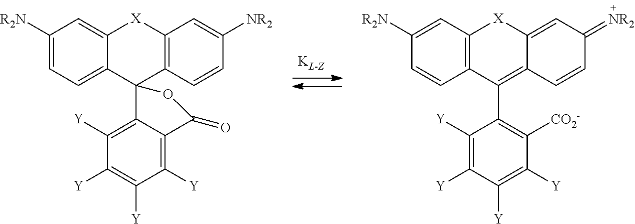

| 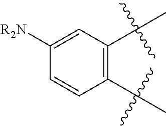 | compound | X | Y | λ_{abs} (nm) | ε (M⁻¹cm⁻¹) | λₑₘ (nm) | Φ_{f} | K_{L-Z} |
|---|---|---|---|---|---|---|---|---|
| 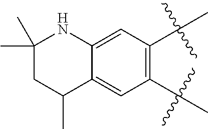 [SMILES: *c1cc2c(cc1*)C(C)CC(C)(C)N2] | 99 18 100 34 | O O Si(CH₃)₂ Si(CH₃)₂ | H F H F | 542 563 638 660 | 112,000 125,000 10,400 129,000 | 564 581 655 676 | 0.83 0.73 0.34 0.28 | 6.30 15.3 0.0220 1.90 |
| 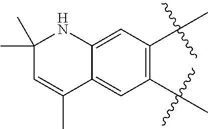 [SMILES: *c1cc2c(cc1*)C(C)=CC(C)(C)N2] | 101 20 102 36 | O O Si(CH₃)₂ Si(CH₃)₂ | H F H F | 573 596 686 713 | 94,100 117,000 4,800 85,900 | 595 614 711 742 | 0.68 0.64 0.03 0.03 | 5.14 7.65 0.0143 1.09 |
| 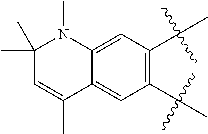 [SMILES: *c1cc2c(cc1*)N(C)C(C)(C)C=C2C] | 103 11 104 38 | O O Si(CH₃)₂ Si(CH₃)₂ | H F H F | 585 610 720 754 | 95,200 121,000 1,200 5,700 | 610 630 740 774 | 0.73 0.68 <0.01 0.02 | 4.44 7.88 0.0045 0.3251 |

All properties were measured in 10 mM HEPES, pH 7.3 except for K_{L-Z}, which was determined in 1:1 dioxane:H₂O

### Example 10: MAC Chemistry Derivatization Strategy

With this expanded collection of fluorinated dyes in hand the MAC chemistry derivatization strategy was tested, preparing the HaloTag® ligands of 8 (JFX₅₇₆), 11, 18 (JF₅₆₃), 25 (JFX₆₇₃), 29 (FSiRh_{Q}), 31 (JF₆₉₈), 36, 38, 40 (JFX₆₃₇), 44 (JF₆₃₂), 49 (FMGL), 51, 55, 57 (JF₆₆₀), 59 (JF₆₅₇), 60, 64-66, and 70-72; the benzylguanine SNAP-tag® ligands of 11, 18, 25, 31, 40, 44, 49, 55, 59, 64, 65; and the chloropyrimidine SNAP-tag® ligands of 8, 11, 18, 25, 31, 40, 44, 49, 59, 64, 65, 85. The MAC chemistry proceeded in good to excellent yield regardless of dye reactant structure (Scheme 18 and Scheme 19). This approach was successful even with carbofluoresceins 51 and 55, which bear nucleophilic phenolic substituents that could react with the electrophilic ring system or the acyl cyanide intermediate.

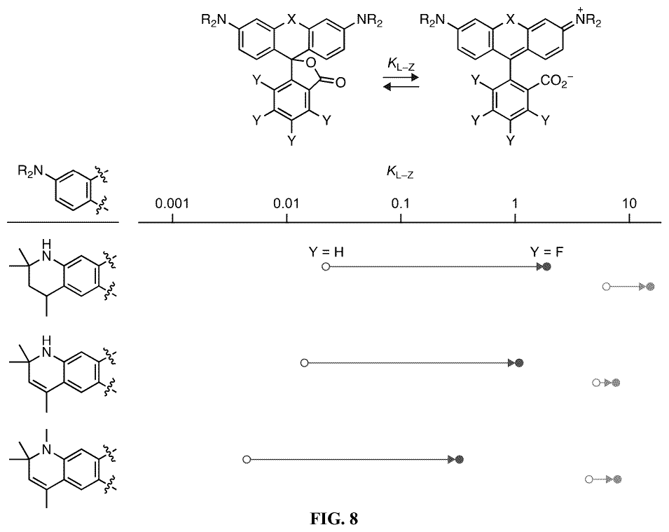

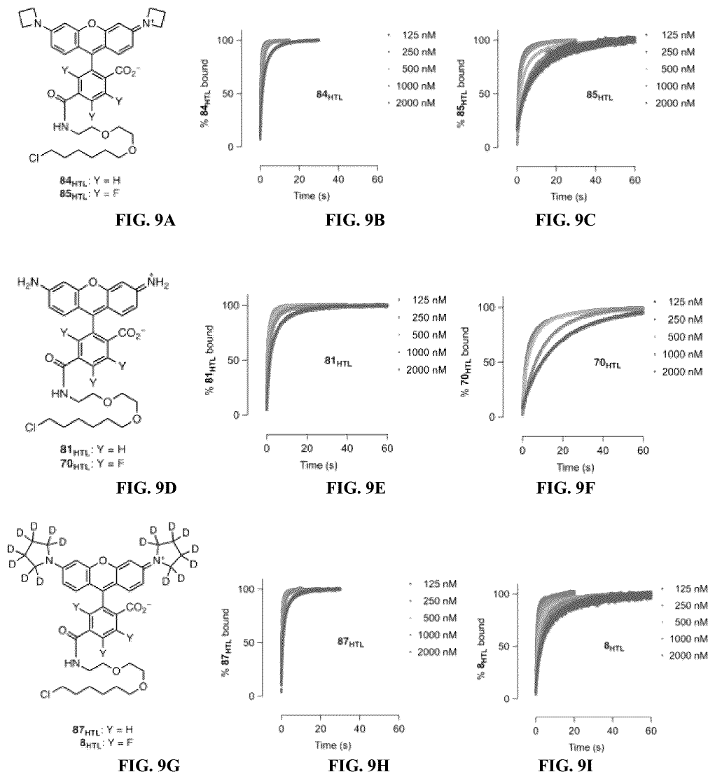

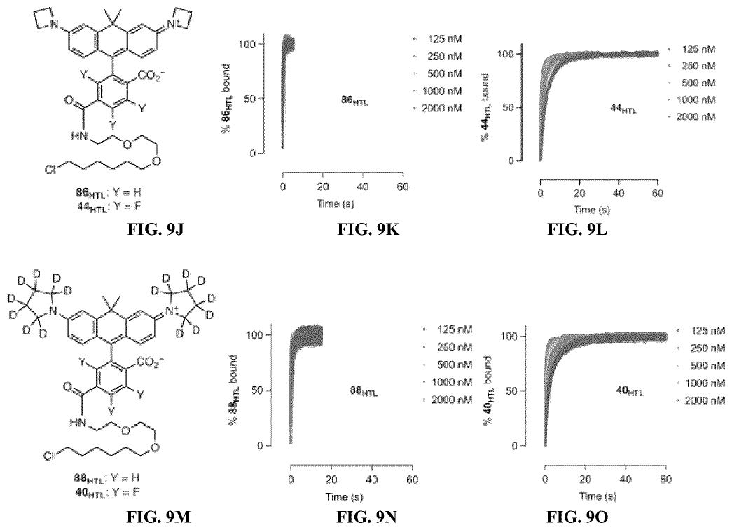

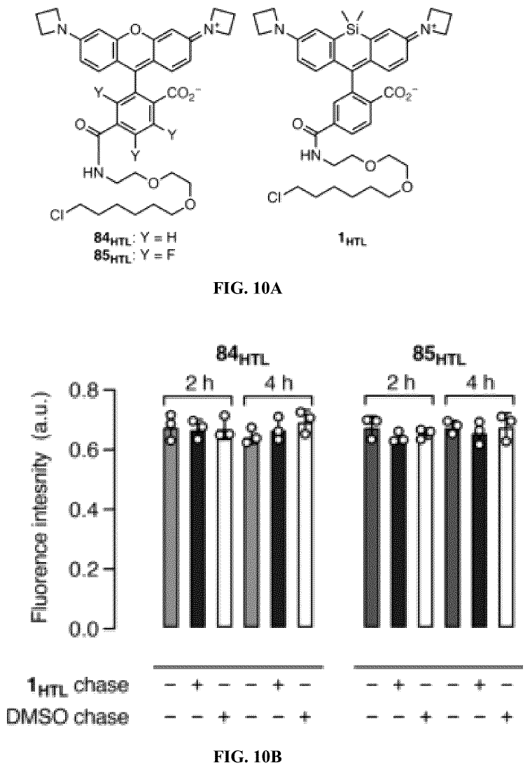

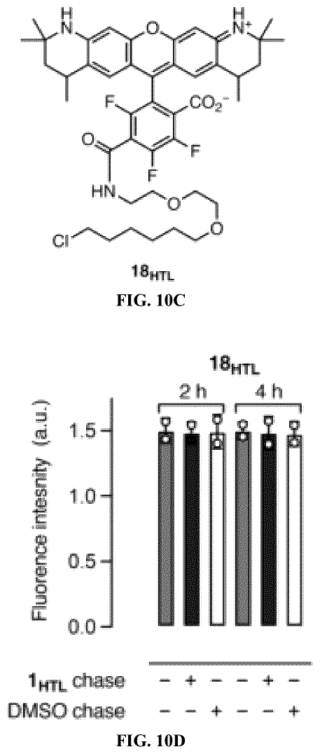

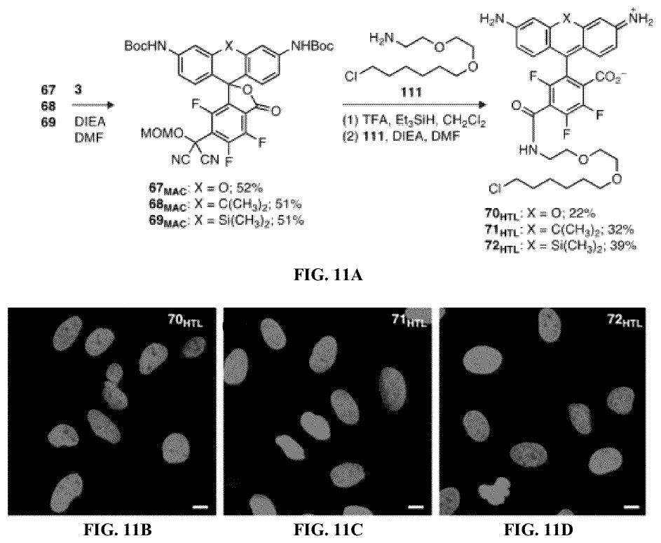

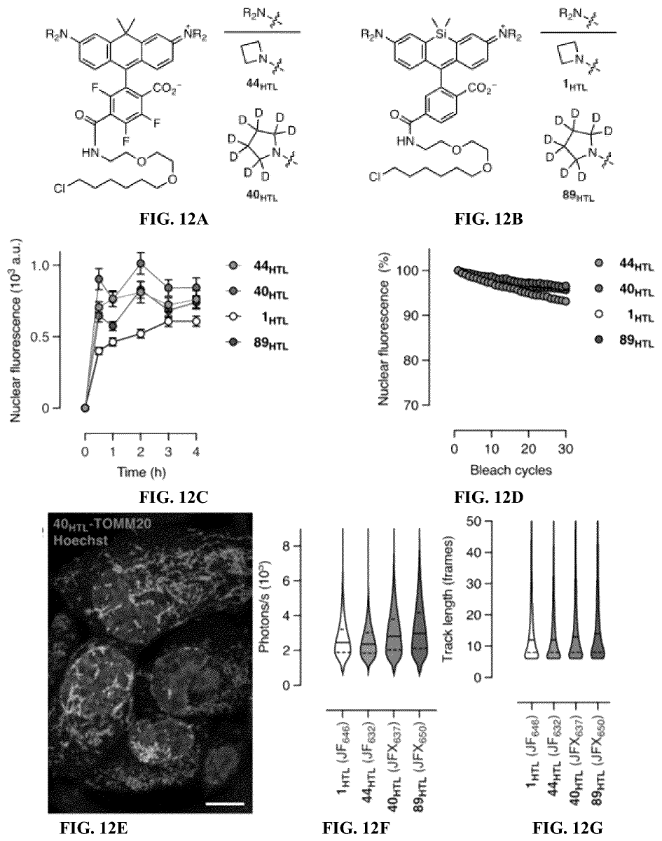

The HaloTag® labeling rates of the matched pairs of nonfluorinated/fluorinated dye ligands were evaluated: 84_{HTL}/85_{HTL}, 81_{HTL}/70_{HTL}, 86_{HTL}/44_{HTL}, 87_{HTL}/8_{HTL}, and 88_{HTL}/40_{HTL} where a modest decrease in rate of 4-14-fold was observed for the fluorinated compounds (FIG. 9A-9O and Table 6). The dye ligand-HaloTag® conjugates proved stable regardless of fluorination (FIG. 10A-10D), the rhodamine 110 analogs efficiently labeled HaloTag® fusion proteins in cells (FIG. 11A-11D), and the unique fluorinated carborhodamines 44 (JF₆₃₂) and 40 (JFX₆₃₇; Table 4) showed comparable performance to previously reported Si-rhodamine dyes JF₆₄₆ (1_{HTL}) and JFX₆₅₀ (89_{HTL}) in single-particle tracking (SPT) experiments (FIG. 12A-12G). Together these data show that fluorination does not adversely affect the performance of dyes in live-cell experiments.

TABLE 6
Labeling rates of HaloTag ® ligands: 84_{HTL}, 85_{HTL}, 81_{HTL}, 70_{HTL},
87_{HTL}, 8_{HTL}, 86_{HTL}, 44_{HTL}, 88_{HTL}, and 40_{HTL}.

| compound | base dye | labeling rate |
|---|---|---|
| 84_{HTL} 85_{HTL} | JF₅₄₉ JF₅₇₁ | 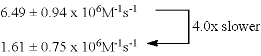 |
| 81_{HTL} 70_{HTL} | Rh₁₁₀ FRh₁₁₀ | 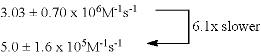 |
| 87_{HTL} 8_{HTL} | JFX₅₅₄ JFX₅₇₆ | 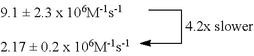 |
| 86_{HTL} 44_{HTL} | JF₆₀₈ JF₆₃₂ | 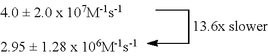 |
| 88_{HTL} 40_{HTL} | JFX₆₁₂ JFX₆₃₇ | 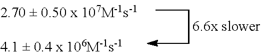 |

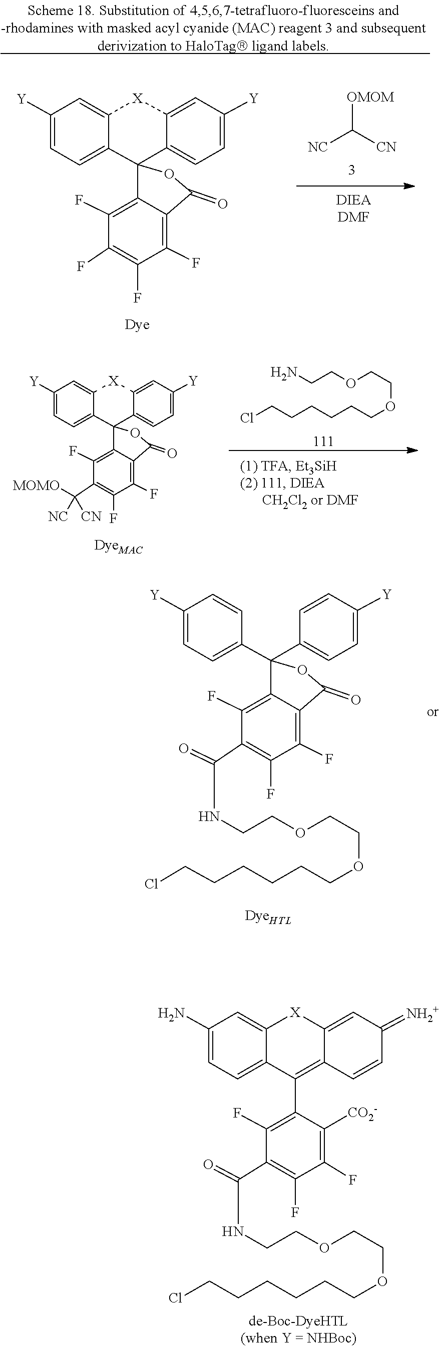

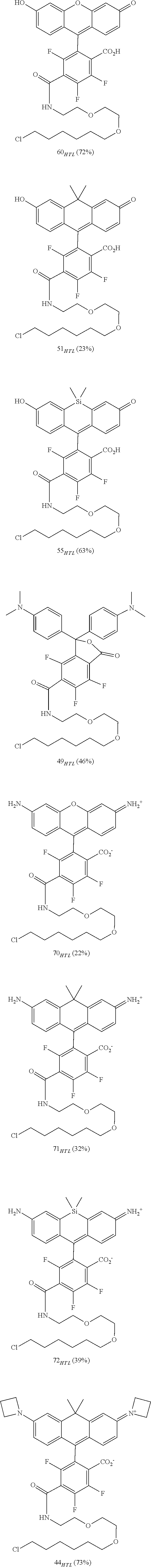

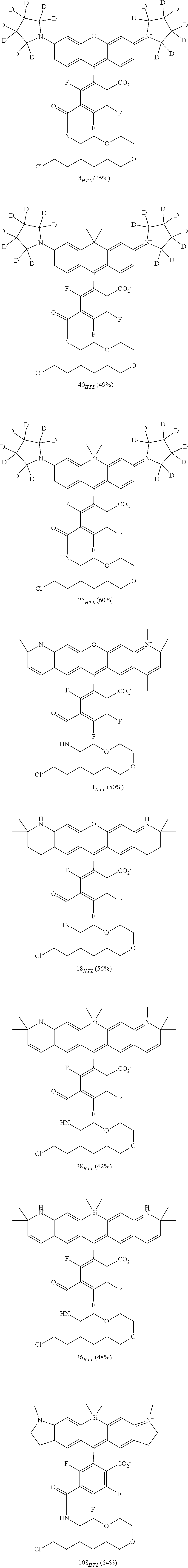

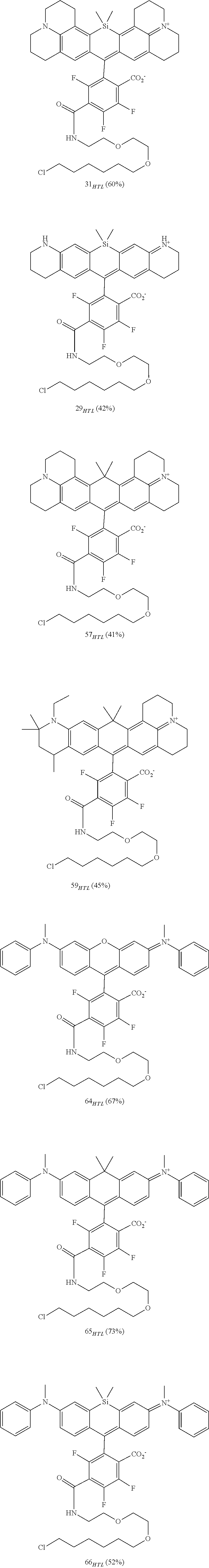

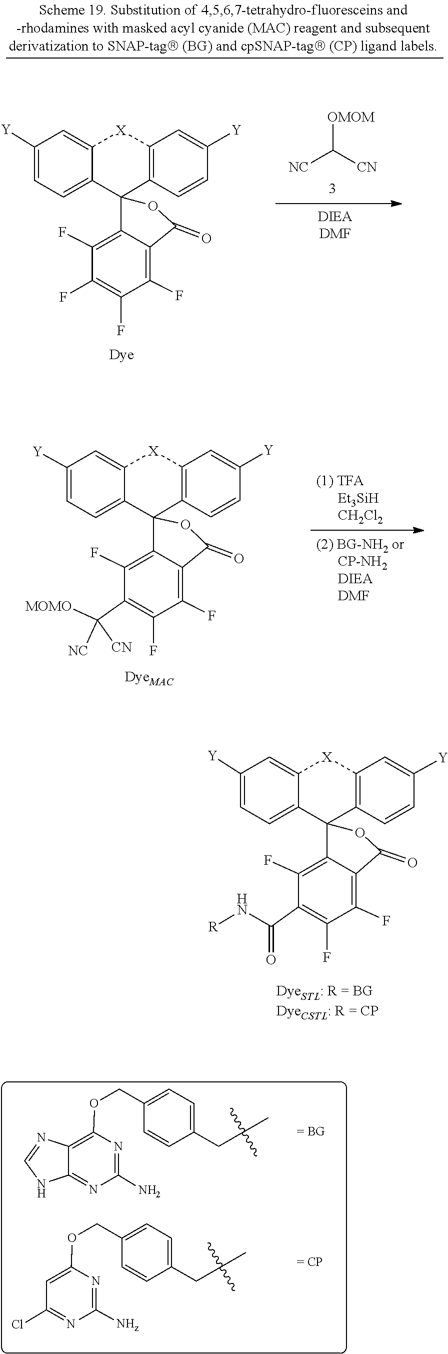

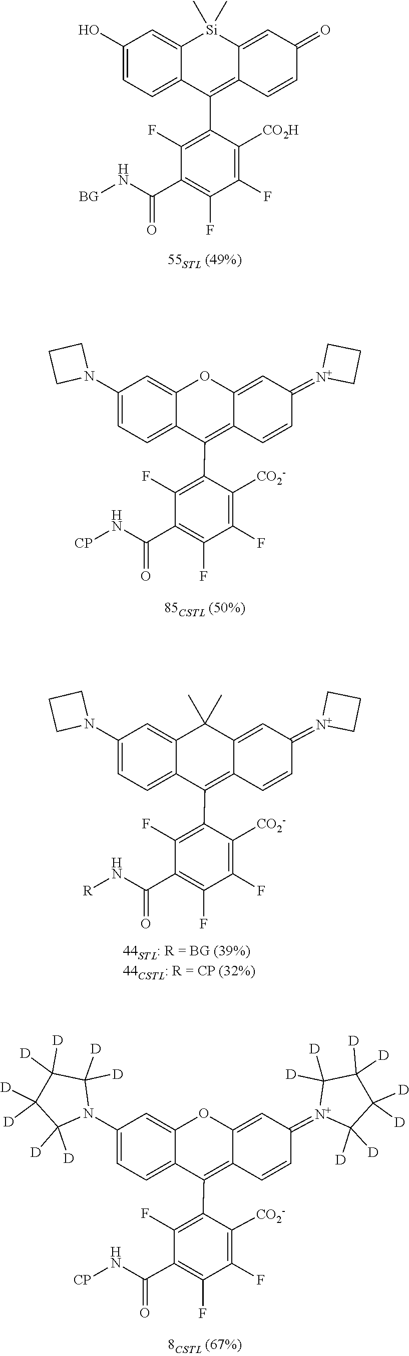

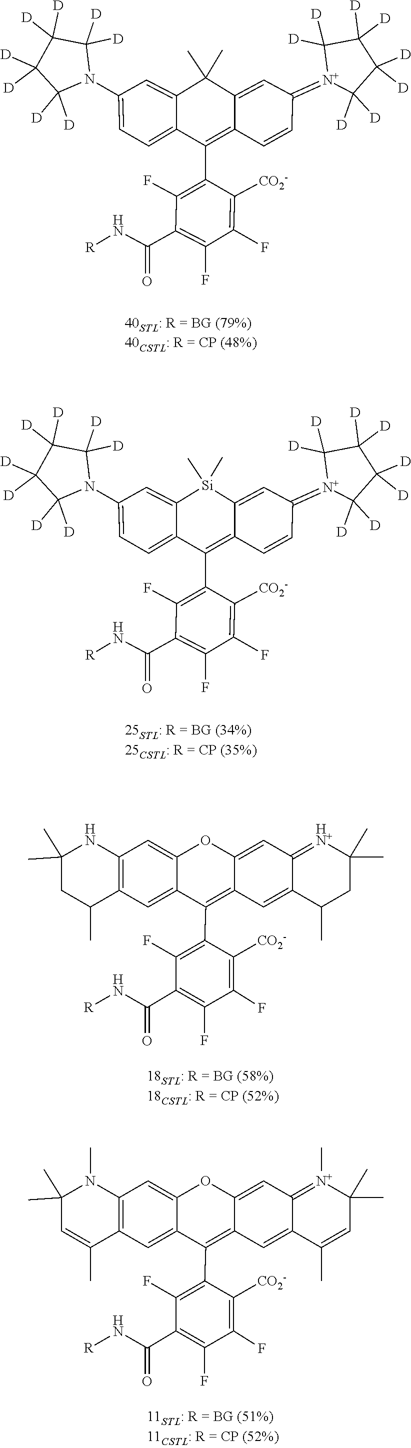

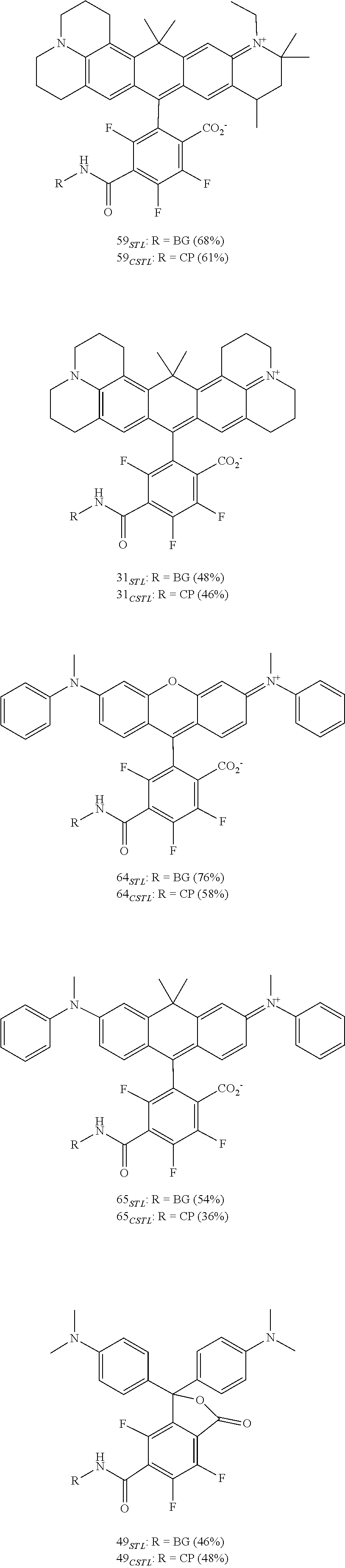

### Example 11: Evaluation for Biological Imaging Applications

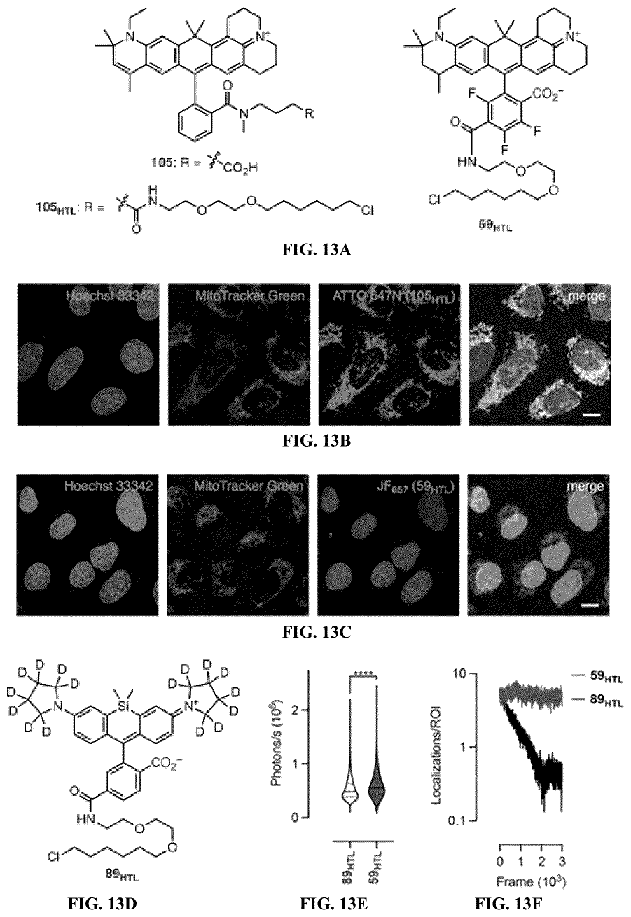

Three unique fluorinated dye compounds were selected to examine more closely for biological imaging applications. The unique carborhodamine 59 (Scheme 15) was the first compound focused upon, which is a fluorine-containing analog of ATTO 647N (105; FIG. 13A). The “famous”⁶⁷ ATTO 647N is perhaps the best dye for in vitro single-molecule imaging and has found widespread use as a label for advanced imaging experiments.^{52, 57, 68} ATTO 647N conjugates have not been used in live-cell imaging experiments, however, due to structural constraints.

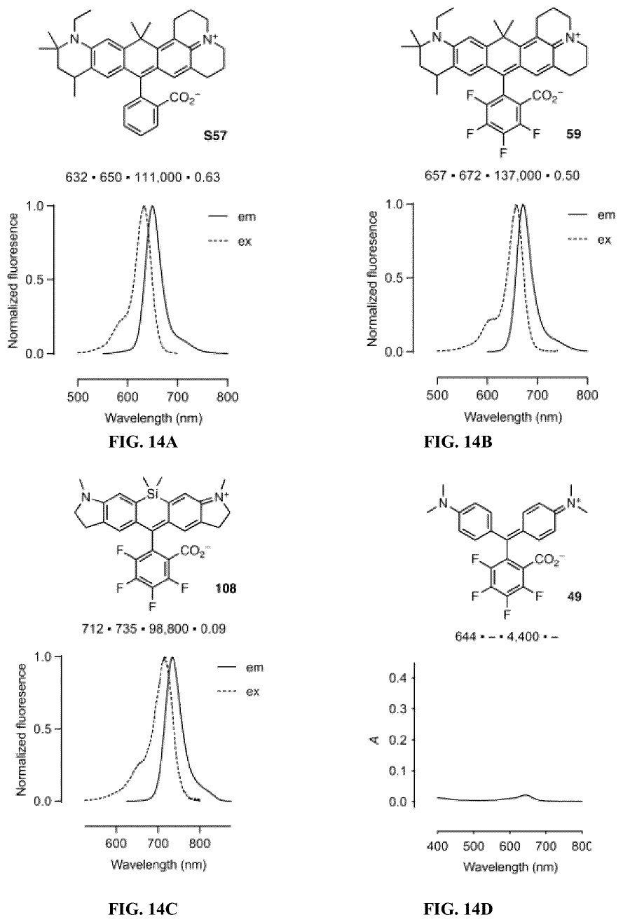

A key design principle used in many ATTO dyes involves amidation of the ortho-carboxyl group on the pendant ring with 4⁻(methylamino)butanoic acid. This conjugation strategy does three things: (i) elicits a bathochromic shift in spectral properties to give λ_{abs}>640 nm, longer than the parent, unmodified dye (λ_{abs}=632 nm; FIG. 14A-14D); (ii) enforces the visible-absorbing form by blocking lactonization; (iii) provides a carboxylic acid moiety for bioconjugation thus circumventing issues with isomeric mixtures. This strategy has an unfortunate side effect, however, as it removes the ortho-carboxylate negative charge, resulting in cationic species after derivatization. The ATTO 647N-HaloTag® ligand 69 (105_{HTL}) does not show appreciable labeling in the nucleus when applied to live cells expressing histone H2B-HaloTag® fusion proteins and instead stains mitochondria (FIG. 13B). This is consistent with the propensity of cationic rhodamine derivatives to accumulate in the mitochondria of living cells.²⁶

The approach of fluorinating fluorophores mirrors the spectral and structural advantages of using the ortho-carboxyl group for bioconjugation but does not produce a cationic species. The fluorine substitution confers a bathochromic shift in λ_{abs}/λₑₘ and works in concert with the julolidine and tetrahydroquinoline auxochromes to increase absorptivity and brightness by shifting the lactone-zwitterion equilibrium towards the zwitterionic form. Introduction of a reactive handle via the straightforward MAC chemistry approach preserves the zwitterionic character of the dye, which should minimize unwanted mitochondrial accumulation. Both diastereomers of 59 displayed identical spectral properties: λ_{abs}=657 nm, ε=137,000 M⁻¹ cm⁻¹, λₑₘ=672 nm, O_{f}=0.50, and K_{L-Z}=4.97 (FIG. 14A-14D). Based on these data the moniker “JF₆₅₇” was selected and the JF₆₅₇-HaloTag® ligand (59_{HTL}) was prepared without separating the diastereomers (Scheme 18). Incubation of cells expressing histone H2B-HaloTag® fusion proteins with 59_{HTL} showed excellent nuclear staining with little cellular background after washing (FIG. 13C). The performance of 59_{HTL} was then compared in SPT experiments to the current best-in-class far-red dye ligand JFX₆₅₀-HaloTag® ligand⁴⁵ (89_{HTL}; FIG. 13D). A significant improvement in brightness was observed as measured by photons/s (FIG. 13E). The unique JF₆₅₇ also showed consistent localizations over time whereas the JFX₆₅₀ exhibited relatively rapid bleaching (FIG. 13F). Thus, fluorination and MAC chemistry allows the venerable ATTO 647N scaffold to be used in living cells, and the resulting dye-JF₆₅₇ represents a new standard for SPT labels.

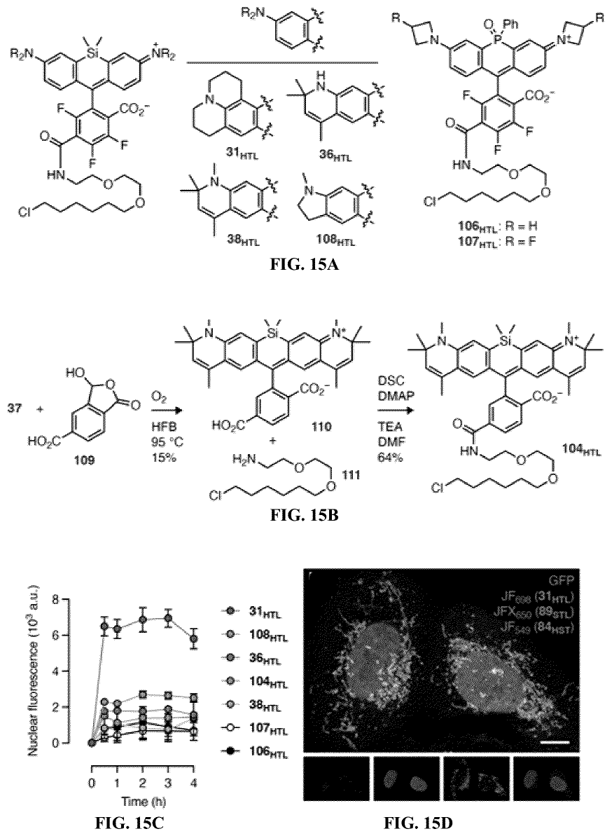

The NIR region is an attractive window for biological imaging with low autofluorescence, less scattering and relatively deep tissue penetration. Dyes that exhibit labs z 700-750 nm were considered and with particular interest in the unique fluorinated analog of Si-rhodamine 101 (JF₆₉₈; 31); its ε=147,000 M⁻¹ cm⁻¹ and Φ_{f}=0.28 (Table 4) makes it similar in brightness to Alexa Fluor 700 and Cy7⁷⁰ but with the potential for live-cell labeling applications. The JF₆₉₈-HaloTag® ligand (31_{HTL}) was compared to other NIR-excited labels (FIG. 15A), including Si-rhodamines 36_{HTL} and 38_{HTL} along with previously reported phosphine oxide-rhodamine HaloTag® ligands based on JF₇₂₂ (106_{HTL}) and JF₇₁₁ (107_{HTL}; FIG. 15A).¹⁶ Two other NIR-excited fluorophores were also prepared. The Li/H exchange approach yielded the fluorinated derivative of SiR₇₀₀^{31, 71} (108); the MAC chemistry afforded its HaloTag® derivative 108_{HTL} (Scheme 8 and Scheme 18). The spectral properties of 108 were found to be: λ_{abs}=712 nm, ε=98,800 M⁻¹ cm⁻¹, λₑₘ=735 nm, O_{f}=0.09, and K_{L-Z}=1.97 (FIG. 14A-14D); it was given the name SiRF₇12. The modified lactol condensation conditions (Scheme 6, Scheme 7) were employed to react 37 and lactol 109 yielding 110 (FIG. 15B), which is the 6⁻carboxy derivative of 104. Amidation with HaloTag® ligand amine 111 gave 104_{HTL}.

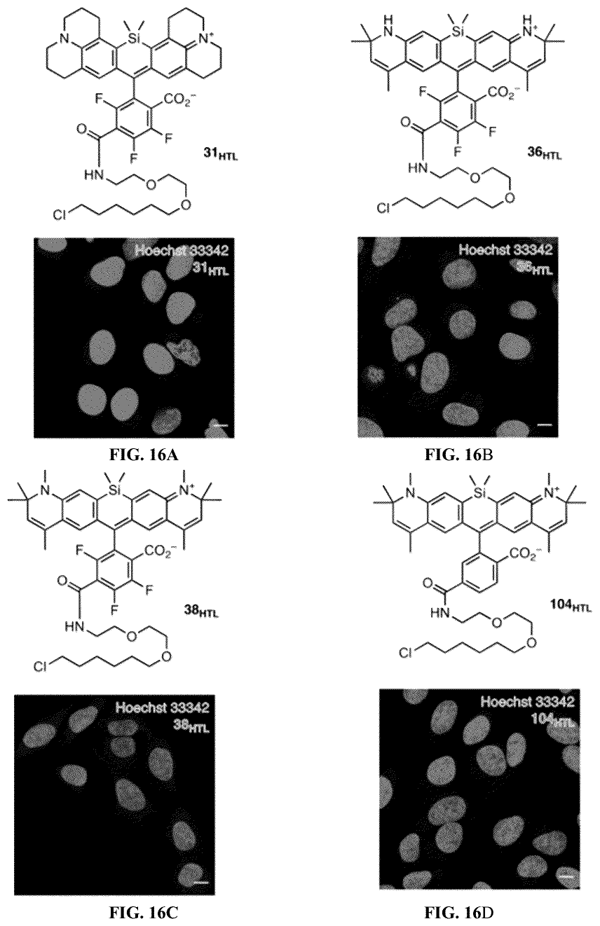

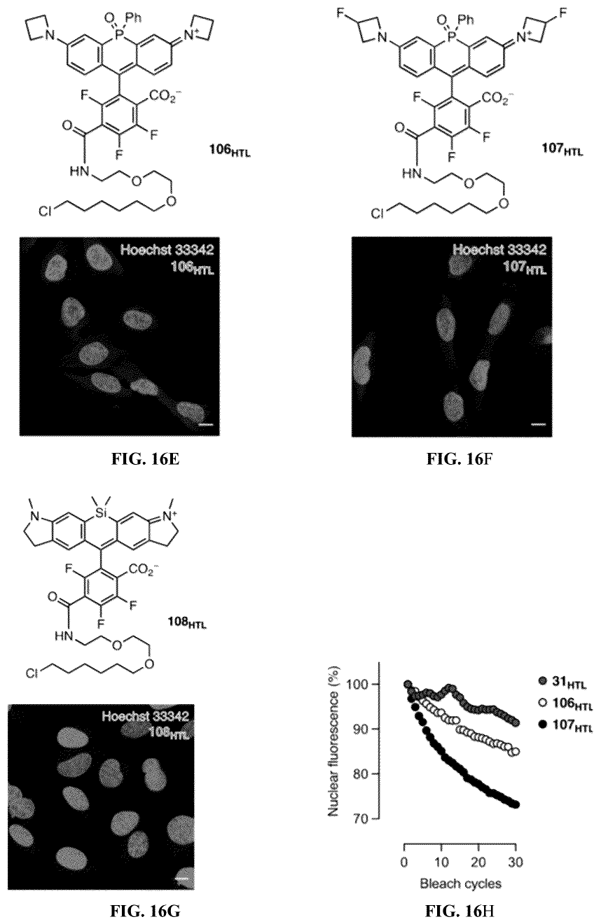

The utility of these NIR-excited HaloTag® ligands was tested in living cells. All the compounds labeled live cells expressing HaloTag® fused to histone H2B with similar kinetics but varying intensity. Consistent with the superior brightness of 31 in vitro, the HaloTag® ligand derivative 31_{HTL} gave >3-fold higher nuclear intensity compared to the other ligands when excited at λ_{abs} (FIG. 15C and FIG. 16A-16H). The HaloTag® ligand with the second highest cellular intensity was the SiRF₇₁₂ compound (108_{HTL}). The dihydroquinoline Si-rhodamines (36_{HTL}, 38_{HTL}, and 104_{HTL}) showed relatively low intensity in cells; this is expected based on their extremely small Φ_{f} values (Table 5). The JF₆₉₈-HaloTag® ligand (31_{HTL}) was also brighter and more photostable that the phosphine oxide-containing JF₇₁₁ (107_{HTL}) and JF₇₂₂ (106_{HTL}; FIG. 16J) and facilitated straightforward multicolor imaging experiments with the 650 nm-excited JFX₆₅₀-SNAP-tag® ligand (89_{STL}) and other visible-absorbing fluorescent labels (FIG. 15D).

Finally, the nonfluorescent N,N′-diaryl-carborhodamine 91 and its fluorinated analog (65; Scheme 17) were evaluated. Introduction of N-aryl groups into rhodamine dyes severely decreases fluorescence quantum yield due to increased twisted internal charge transfer (TICT),^{72, 73} making N-arylrhodamines and N-aryl-Si-rhodamines useful nonfluorescent acceptor dyes for Forster resonance energy transfer (FRET).^{26, 74, 75} These nonfluorescent rhodamine systems have not been used for intracellular labeling, however, and N-aryl-carborhodamines remain untested as FRET quenchers. Introduction of an N-aryl group into rhodamine dyes also shifts the lactone-zwitterion equilibrium toward the colorless lactone form (Table 4); commercial N-arylrhodamine quenchers typically have the ortho-carboxyl group blocked or removed, presumably to prevent unwanted lactonization.^{26, 74} The N-aryl-carborhodamine compound 91 shows extremely low visible absorption in aqueous solution (ε<200 M⁻¹ cm⁻¹) and a vanishingly small K_{L-Z}.⁴¹ Incorporation of fluorine atoms into the pendant phenyl ring system, as in compound 65, shifts the lactone-zwitterion equilibrium toward the zwitterionic form, giving a dye with a measurable absorptivity (ε=2,700 M⁻¹ cm⁻¹), K_{L-Z}=0.344, and far-red absorption (λ_{abs}=645 nm; Table 4).

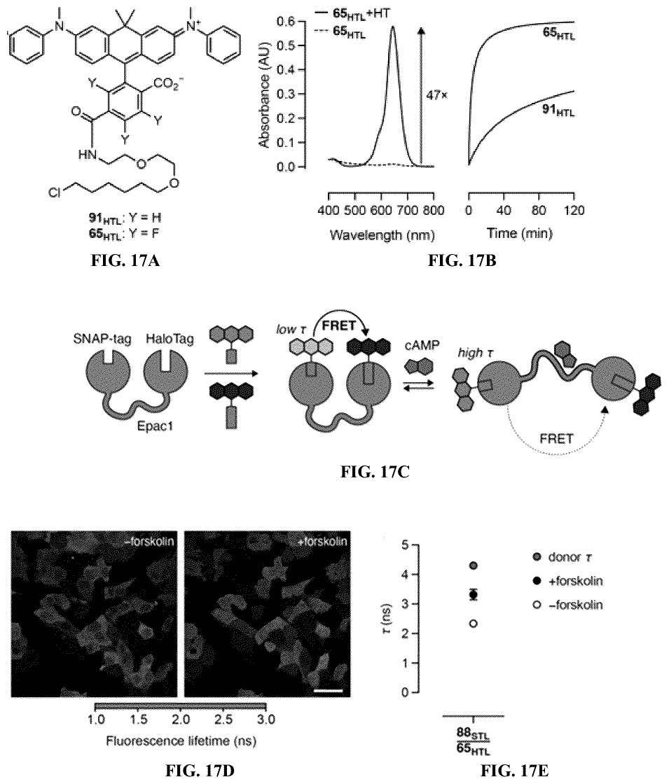

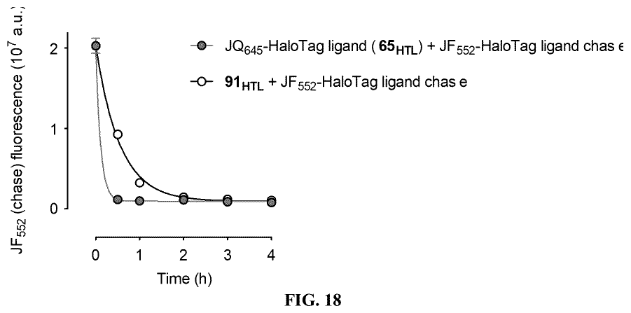

It was surmised that the properties of 65 could translate into a highly chromogenic HaloTag® ligand. Incubation of 65_{HTL} (FIG. 17A) with the HaloTag® protein gave an increase in absorptivity of 47-fold (FIG. 17B) yielding a HaloTag® conjugate with ε=110,000 M⁻¹ cm⁻¹. Unlike the fluorescent N-alkyl rhodamine ligands (FIG. 9A-9O) HaloTag® labeling of the fluorinated 65_{HTL} was substantially faster than the nonfluorinated HaloTag® ligand 91_{HTL} (Scheme 20) in vitro (FIG. 17B) and in cells (FIG. 18). Based on these properties 65 was given the name Janelia Quencher 645 (JQ₆₄₅).

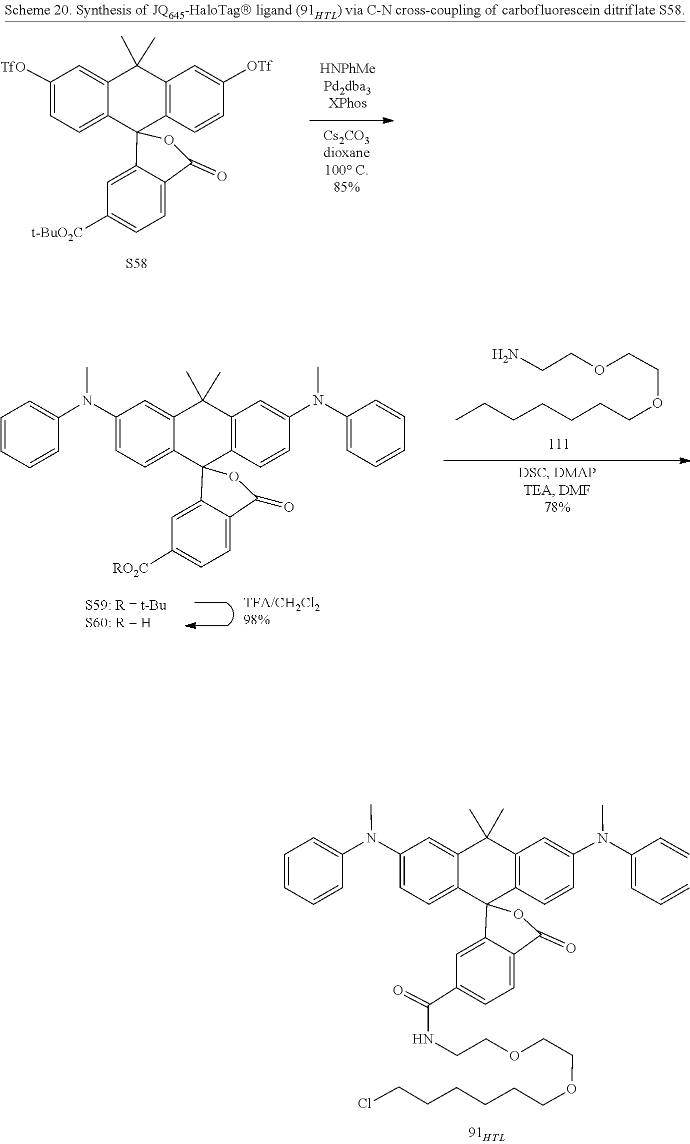

This unique, chromogenic, far-red quencher dye was employed in a semisynthetic sensor for the cyclic adenosine monophosphate (cAMP). The genes encoding the SNAP-tag® and HaloTag® proteins were linked via the sequence for the exchange protein directly activated by cAMP (Epac1),⁷⁶ basing this design on an established cAMP sensor construct that incorporates fluorescent proteins.⁷⁷ Binding of cAMP causes a conformational change that decreases FRET and increases the fluorescence lifetime (τ) of the donor (FIG. 17D); This semisynthetic cAMP indicator (“ScAMPI”) construct was expressed in cells and labeled with JFX₆₁₂⁻SNAP-tag® ligand (88_{STL}) as the donor and the unique JQ₆₄₅-HaloTag® ligand (65_{HTL}) as the acceptor. At rest, cells showed a lifetime of 2.34±0.10 ns (mean±SD) as measured by FLIM (FIG. 17E, 17F). This lifetime is substantially lower than cells labeled with the JFX₆₁₂ donor fluorophore alone (4.30±0.01 ns) showing that the JQ₆₄₅-HaloTag® ligand is an effective FRET quencher. Increasing intracellular cAMP concentration by application of forskolin elicited a substantial increase in fluorescence lifetime to 3.32±0.18 giving an overall Δτ=0.98 ns. This result demonstrates that FRET quenchers can be employed in conjunction with self-labeling tags to create useful, red-shifted FLIM indicators for intracellular analytes.

Rhodamines remain the most important fluorophores for live-cell imaging and are increasingly used in more complex systems such as intact animals. Optimizing these fluorophores for specific applications requires the synthetic chemistry methods needed to make new rhodamines and an understanding of the structure-activity relationships that govern the spectral and chemical properties of dyes. Here, the synthesis of rhodamine dyes was thoroughly surveyed, with investigation of five different methods (Scheme 1). New and improved chemistry was developed, expanding the scope of the oxidative phthalaldehydic acid (“lactol”) condensation⁴² to synthesize fluorinated and nonfluorinated Si-rhodamines (Schemes 6, 7) and establishing a unique route to fluorinated dyes using the straightforward Li/H exchange of 2,3,4,5⁻tetrafluorobenzoic acid (6; Schemes 9, 12, 13). This vetted portfolio of organic chemistry strategies will enable the synthesis of a wide range of rhodamines, particularly fluorinated derivatives.

These methods were to assemble a systematic set of rhodamine dyes and measured their properties (Tables 3-5). These data revealed structure-activity relationships governing the lactone-zwitterion equilibrium (K_{L}⁻z; FIG. 6), which is a key determinant of the performance of rhodamines in biological systems (FIG. 1A).¹⁶ Julolidine-containing dyes were found to be strongly shifted towards the zwitterionic form whereas N-aryl dyes prefer the lactone form. It was also discovered that fluorination of the pendant phenyl ring could increase K_{L-Z} regardless of chemical structure and this effect was exploited to prepare new reagents for cellular imaging, aided by previously described MAC reagent derivatization strategy.¹⁶ The zwitterionic JF₆₅₇ (59) was made, a live-cell compatible analog of the cationic ATTO 647N with performance in SPT experiments that supersedes the current best-in-class dye JFX₆₅₀ (FIG. 13A-13F). The fluorine-induced bathochromic and K_{L-Z} shifts were leveraged to make JF₆₉₈ (31), a bright, NIR-excited dye that enables multicolor imaging (FIG. 15A-15D). JQ₆₄₅ (65) was also developed, a chromogenic far-red FRET quencher compatible with self-labeling tag systems. This quencher dye was used to create ScAMPI, a semisynthetic indicator for cAMP useful for FLIM (FIG. 17A-17E).

The unique molecules disclosed herein will immediately enable new biological imaging experiments in living cells. Prior to the synthesis of this comprehensive collection of dyes and exhaustive measurement of their chemical properties, the structure-activity relationships governing absorptivity and K_{L-Z} in different classes of rhodamines appeared idiosyncratic. For example, the high Φ_{f} of julolidine-containing dyes has been appreciated for decades⁷⁸ but julolidine's effect on ε was largely unrecognized. Trends in absorptivity and K_{L-Z} can be rationalized by the electron-donating capability of the auxochrome group (FIG. 6). When making a dye, the rhodamine scaffold capable of accessing the desired spectral and chemical properties for the application should be selected. Rhodamines containing NH₂ or azetidine auxochromes can access a broad range of K_{L-Z} values and are useful for making a variety of probes including chromogenic or bioavailable reagents. Julolidine-containing rhodamines have a narrower K_{L-Z} range and are perhaps better suited for creating bright labels with enhanced absorptivity. Unique fluorinated carborhodamines as disclosed herein, such as JF₆₅₇ and JQ₆₄₅, represent a unique scaffold of far-red-absorbing dyes.

### Example 12: General Experimental Information for Synthesis

Commercial reagents were obtained from reputable suppliers and used as received. All solvents were purchased in septum-sealed bottles stored under an inert atmosphere. All reactions were sealed with septa through which a nitrogen atmosphere was introduced unless otherwise noted. Reactions were conducted in round-bottomed flasks or septum-capped crimp-top vials containing Teflon-coated magnetic stir bars. Heating of reactions was accomplished with a silicon oil bath or an aluminum reaction block on top of a stirring hotplate equipped with an electronic contact thermometer to maintain the indicated temperatures.

Reactions were monitored by thin layer chromatography (TLC) on precoated TLC glass plates (silica gel 60 F₂₅₄, 250 μm thickness) or by LC/MS (Phenomenex Kinetex 2.1 mm×30 mm 2.6 μm C18 column; 5 μL injection; 5-98% MeCN/H₂O, linear gradient, with constant 0.1% v/v HCO₂H additive; 6 min run; 0.5 mL/min flow; ESI; positive ion mode). TLC chromatograms were visualized by UV illumination or developed with p-anisaldehyde, ceric ammonium molybdate, or KMnO₄ stain. Reaction products were purified by flash chromatography on an automated purification system using pre-packed silica gel columns or by preparative HPLC (Phenomenex Gemini-NX 30×150 mm 5 m C18 column). Analytical HPLC analysis was performed with an Agilent Eclipse XDB 4.6×150 mm 5 m C18 column under the indicated conditions. High-resolution mass spectrometry was performed by the High Resolution Mass Spectrometry Facility at the University of Iowa.

NMR spectra were recorded on a 400 MHz spectrometer. ¹H and ¹³C chemical shifts were referenced to TMS or residual solvent peaks, and ¹⁹F chemical shifts were referenced to CFCl₃. Data for ¹H NMR spectra are reported as follows: chemical shift (6 ppm), multiplicity (s=singlet, d=doublet, t=triplet, q=quartet, dd=doublet of doublets, m=multiplet), coupling constant (Hz), integration. Data for ¹³C NMR spectra are reported by chemical shift (δ ppm) with hydrogen multiplicity (C, CH, CH₂, CH₃) information obtained from DEPT spectra. The ¹³C NMR spectra are not reported for compounds containing trifluoro- or tetrafluoro-substituted aryl rings, as the 3-4 distinct fluorine couplings to each of the six different carbons of the bottom ring confounded any useful interpretation of the spectra.

### Example 13: Preparation of Diarylether and Diarylsilane Intermediates (Scheme 2): 1,1′-(Oxybis(3,1⁻phenylene))bis(pyrrolidine-2,2,3,3,4,4,5,5⁻d8) (9)

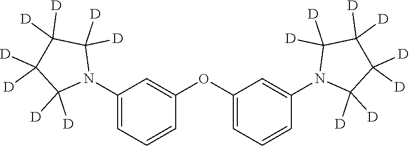 [SMILES: [2H]C1([2H])N(c2cccc(Oc3cccc(N4C([2H])([2H])C([2H])([2H])C([2H])([2H])C4([2H])[2H])c3)c2)C([2H])([2H])C([2H])([2H])C1([2H])[2H]]

A vial was charged with 3,3′-oxybis(bromobenzene)¹⁴ (S1; 1.00 g, 3.05 mmol), Pd₂dba₃ (279 mg, 0.305 mmol, 0.1 eq), XPhos (436 mg, 0.915 mmol, 0.3 eq), and Cs₂CO₃ (2.78 g, 8.54 mmol, 2.8 eq). The vial was sealed and evacuated/backfilled with nitrogen (3×). Dioxane (12 mL) was added, and the reaction was flushed again with nitrogen (3×). Following the addition of pyrrolidine-2,2,3,3,4,4,5,5⁻d₈ (612 μL, 7.32 mmol, 2.4 eq), the reaction was stirred at 100° C. for 18 h. It was then cooled to room temperature, filtered through Celite with CH₂Cl₂, and concentrated to dryness. The crude product was purified by flash chromatography (0-20% Et₂O/hexanes, linear gradient) to yield 936 mg (95%) of 9 as an off-white solid. 1H NMR (CDCl₃, 400 MHz) δ 7.16-7.10 (m, 2H), 6.35-6.24 (m, 6H); 13C NMR (CDCl₃, 101 MHz) δ 158.6 (C), 149.6 (C), 129.9 (CH), 106.7 (CH), 105.9 (CH), 102.5 (CH); HRMS (ESI) calcd for C₂₀H₉D₁₆N₂O [M+H]⁺ 325.2966, found 325.2957.

### Example 14: Preparation of Diarylether and Diarylsilane Intermediates (Scheme 2): Dimethylbis(3⁻(pyrrolidin-1⁻yl-d8)phenyl)silane (26)

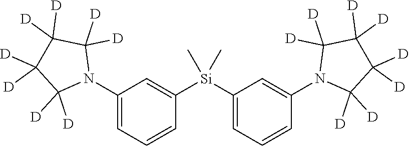 [SMILES: [2H]C1([2H])N(c2cccc([Si](C)(C)c3cccc(N4C([2H])([2H])C([2H])([2H])C([2H])([2H])C4([2H])[2H])c3)c2)C([2H])([2H])C([2H])([2H])C1([2H])[2H]]

A vial was charged with bis(3⁻bromophenyl)dimethylsilane⁷⁹ (S2; 300 mg, 0.810 mmol), Pd₂dba₃ (74.2 mg, 81.0 μmol, 0.1 eq), XPhos (116 mg, 0.243 mmol, 0.3 eq), and Cs₂CO₃ (739 mg, 2.27 mmol, 2.8 eq). The vial was sealed and evacuated/backfilled with nitrogen (3×). Dioxane (3.5 mL) was added, and the reaction was flushed again with nitrogen (3×). Following the addition of pyrrolidine-2,2,3,3,4,4,5,5⁻d₈ (163 μL, 1.95 mmol, 2.4 eq), the reaction was stirred at 100° C. for 18 h. It was then cooled to room temperature, filtered through Celite with CH₂Cl₂, and concentrated to dryness. The resulting residue was purified by flash chromatography (0-10% Et₂O/hexanes, linear gradient, with constant 0.1% v/v Et₃N additive) to provide 261 mg (88%) of 26 as an off-white solid. ¹H NMR (CDCl₃, 400 MHz) δ 7.21 (dd, J=8.2, 7.1 Hz, 2H), 6.84 (dt, J=7.1, 1.1 Hz, 2H), 6.75 (dd, J=2.6, 1.0 Hz, 2H), 6.57 (ddd, J=8.2, 2.7, 1.1 Hz, 2H), 0.52 (s, 6H); 13C NMR (CDCl₃, 101 MHz) δ 147.5 (C), 139.2 (C), 128.6 (CH), 121.5 (CH), 117.3 (CH), 112.5 (CH), −2.0 (CH₃); HRMS (ESI) calcd for C₂₂H₁₅D₁₆N₂Si [M+H]⁺ 367.3255, found 367.3259.

### Example 15: Preparation of Diarylether and Diarylsilane Intermediates (Scheme 2): 1,1′-(Oxybis(4⁻bromo-3,1⁻phenylene))bis(pyrrolidine-2,2,3,3,4,4,5,5⁻d₈) (7)

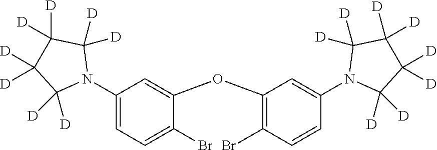 [SMILES: [2H]C1([2H])N(c2ccc(Br)c(Oc3cc(N4C([2H])([2H])C([2H])([2H])C([2H])([2H])C4([2H])[2H])ccc3Br)c2)C([2H])([2H])C([2H])([2H])C1([2H])[2H]]

1,1′-(Oxybis(3,1⁻phenylene))bis-(pyrrolidine-2,2,3,3,4,4,5,5⁻d₈) (9; 850 mg, 2.62 mmol) was taken up in DMF (20 mL). N-Bromosuccinimide (932 mg, 5.24 mmol, 2 eq) was added portion-wise over 5 min, and the reaction was then stirred at room temperature for 4 h. The reaction mixture was concentrated in vacuo; the resulting residue was diluted with water and extracted with CH₂Cl₂ (2×). The combined organic extracts were washed with water and brine, dried over anhydrous MgSO₄, filtered, and concentrated in vacuo. Silica gel chromatography (10-75% CH₂Cl₂/hexanes, linear gradient) afforded 1.04 g (82%) of 7 as a white solid. 1H NMR (CDCl₃, 400 MHz) δ 7.36 (d, J=8.7 Hz, 2H), 6.21 (dd, J=8.8, 2.8 Hz, 2H), 6.07 (d, J=2.7 Hz, 2H); 13C NMR (CDCl₃, 101 MHz) δ 153.9 (C), 148.6 (C), 133.5 (CH), 108.6 (CH), 103.0 (CH), 98.7 (C); HRMS (ESI) calcd for C₂₀H₇D₁₆Br₂N₂O [M+H]⁺ 481.1176, found 483.1149.

### Example 16: Preparation of Diarylether and Diarylsilane Intermediates (Scheme 2): Bis(2⁻bromo-5⁻(pyrrolidin-1⁻yl-d₈)phenyl)dimethylsilane (24)

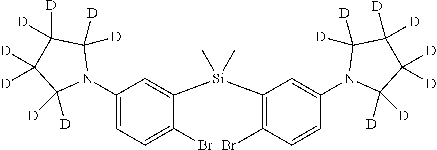 [SMILES: [2H]C1([2H])N(c2ccc(Br)c([Si](C)(C)c3cc(N4C([2H])([2H])C([2H])([2H])C([2H])([2H])C4([2H])[2H])ccc3Br)c2)C([2H])([2H])C([2H])([2H])C1([2H])[2H]]

Silane 26 (1.95 g, 5.32 mmol) was taken up in DMF (60 mL). N-Bromosuccinimide (1.89 g, 10.64 mmol, 2 eq) was added portion-wise over 2-3 min, and the reaction was then stirred at room temperature for 1 h. The reaction mixture was concentrated to remove DMF, diluted with water, and extracted with CH₂Cl₂ (2×). The combined organic extracts were washed with brine, dried over anhydrous MgSO₄, filtered, and concentrated in vacuo. The crude was dissolved in a minimum amount of CH₂Cl₂, diluted with an equivalent volume of hexanes, and gently concentrated until a white solid precipitated. The resulting suspension was filtered; the filter cake was washed with Et₂O and dried to yield 2.32 g (83%) of dibromide 24 as a white solid. ¹H NMR (CDCl₃, 400 MHz) δ 7.32 (d, J=8.7 Hz, 2H), 6.66 (d, J=3.1 Hz, 2H), 6.42 (dd, J=8.7, 3.1 Hz, 2H), 0.74 (s, 6H); 13C NMR (CDCl₃, 101 MHz) δ 146.5 (C), 139.0 (C), 133.1 (CH), 120.7 (CH), 115.4 (C), 114.3 (CH), −0.8 (CH₃); HRMS (ESI) calcd for C₂₂H₁₃D₁₆Br₂N₂Si [M+H]⁺ 523.1466, found 523.1466.

### Example 17: Preparation of Diarylether and Diarylsilane Intermediates (Scheme 2): 7,7′—Oxybis(2,2,4⁻trimethyl-1,2⁻dihydroquinoline) (19)

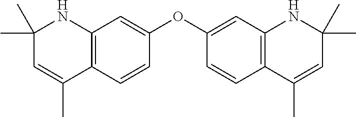

To a solution of 3,3′-oxydianiline (S3; 1.00 g, 4.99 mmol) in acetone (30 mL) was added Yb(OTf)₃ (465 mg, 0.749 mmol, 0.15 eq). After stirring the reaction at room temperature for 72 h, it was concentrated in vacuo, diluted with saturated NaHCO₃, and extracted with EtOAc (2×). The combined organic extracts were washed with brine, dried over anhydrous MgSO₄, filtered, and concentrated to dryness. Flash chromatography on silica gel (0-25% EtOAc/hexanes, linear gradient) yielded 1.27 g (71%) of 19 as a white solid. ¹H NMR (CDCl₃, 400 MHz) δ 6.98 (d, J=8.3 Hz, 2H), 6.29 (dd, J=8.3, 2.4 Hz, 2H), 6.09 (d, J=2.4 Hz, 2H), 5.23 (q, J=1.3 Hz, 2H), 3.66 (s, 2H), 1.97 (d, J=1.4 Hz, 6H), 1.26 (s, 12H); 13C NMR (CDCl₃, 101 MHz) δ 157.8 (C), 144.7 (C), 128.2 (C), 127.0 (CH), 124.8 (CH), 117.2 (C), 107.7 (CH), 103.3 (CH), 52.1 (C), 31.3 (CH₃), 18.8 (CH₃); HRMS (ESI) calcd for C₂₄H₂₉N₂O [M+H]⁺ 361.2274, found 361.2267.

### Example 18: Preparation of Diarylether and Diarylsilane Intermediates (Scheme 2): 7,7′—Oxybis(1,2,2,4⁻tetramethyl-1,2⁻dihydroquinoline) (12)

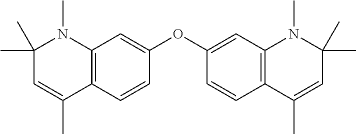

7,7′—Oxybis(2,2,4⁻trimethyl-1,2⁻dihydroquinoline) (19; 2.00 g, 5.55 mmol) was taken up in DMF (16 mL); K₂CO₃ (2.30 g, 16.64 mmol, 3 eq) and iodomethane (829 μL, 13.31 mmol, 2.4 eq) were added, and the reaction was stirred at 50° C. for 18 h. It was subsequently cooled to room temperature, diluted with water, and extracted with EtOAc (2×). The combined organic extracts were washed with brine, dried over anhydrous MgSO₄, filtered, and concentrated to dryness. Silica gel chromatography (0-20% EtOAc/hexanes, linear gradient) afforded 12 as a white solid (2.10 g, 97%). ¹H NMR (CDCl₃, 400 MHz) δ 7.00-6.93 (m, 2H), 6.31-6.23 (m, 4H), 5.22 (q, J=1.4 Hz, 2H), 2.74 (s, 6H), 1.97 (d, J=1.4 Hz, 6H), 1.30 (s, 12H); 13C NMR (CDCl₃, 101 MHz) δ 158.2 (C), 146.9 (C), 128.5 (CH), 128.0 (C), 124.2 (CH), 118.9 (C), 105.9 (CH), 102.1 (CH), 56.5 (C), 30.9 (CH₃), 27.4 (CH₃), 18.8 (CH₃); HRMS (ESI) calcd for C₂₆H₃₃N₂O [M+H]⁺389.2587, found 389.2579.

### Example 19: Preparation of Diarylether and Diarylsilane Intermediates (Scheme 2): 7,7′—Oxybis(2,2,4⁻trimethyl-1,2,3,4⁻tetrahydroquinoline) (17)

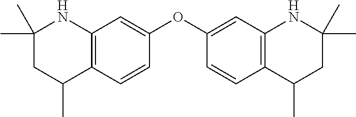

7,7′—Oxybis(2,2,4⁻trimethyl-1,2⁻dihydroquinoline) (19; 4.50 g, 12.48 mmol) was dissolved in EtOH (300 mL) in a round-bottom flask under nitrogen, and Pd/C (10%, 2.66 g, 2.50 mmol, 0.2 eq) was added. The sealed flask was evacuated/backfilled with H₂ from a balloon (4×) and then stirred under the H₂ balloon at room temperature for 18 h. The reaction mixture was filtered through Celite with EtOH and concentrated in vacuo. Silica gel chromatography (0-15% EtOAc/hexanes, linear gradient) yielded 4.47 g (98%) of 17 as a white solid (mixture of diastereomers). ¹H NMR (CDCl₃, 400 MHz) δ 7.09-7.02 (m, 2H), 6.36-6.28 (m, 2H), 6.10-6.05 (m, 2H), 3.54 (s, 2H), 2.94-2.80 (m, 2H), 1.72 (dd, J=12.9, 5.5 Hz, 2H), 1.42 (t, J=12.5 Hz, 2H), 1.31 (d, J=6.7 Hz, 6H), 1.21 (s, 6H), 1.17 (s, 6H); 13C NMR (CDCl₃, 101 MHz) δ 156.6 (C), 144.9 (C), 127.91/127.86 (CH), 120.17/120.15 (C), 107.76/107.67 (CH), 104.13/104.10 (CH), 49.5 (C), 44.7 (CH₂), 31.6 (CH), 28.04/27.96 (CH₃), 27.4 (CH₃), 20.57/20.54 (CH₃); HRMS (ESI) calcd for C₂₄H₃₃N₂O [M+H]⁺ 365.2587, found 365.2580.

### Example 20 Preparation of Diarylether and Diarylsilane Intermediates (Scheme 2): 1⁻Benzyl-7⁻bromo-1,2,3,4⁻tetrahydroquinoline (S4)

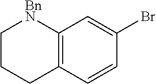 [SMILES: Brc1ccc2c(c1)N(Cc1ccccc1)CCC2]

7⁻Bromo-1,2,3,4⁻tetrahydroquinoline (1.00 g, 4.71 mmol), benzyl bromide (1.61 g, 9.43 mmol, 2 eq), K₂CO₃ (1.56 g, 11.3 mmol, 2.4 eq), and KI (157 mg, 0.943 mmol, 0.2 eq) were combined in DMF (15 mL) and stirred at 60° C. for 1 h. The reaction was subsequently diluted with water and extracted with EtOAc (2×). The combined organic extracts were washed with water and brine, dried (MgSO₄), filtered, and concentrated in vacuo. Silica gel chromatography (0-15% Et₂O/hexanes, linear gradient) yielded S4 (1.35 g, 95%) as a gum that crystallized into a colorless, low-melting solid upon standing. ¹H NMR (CDCl₃, 400 MHz) δ 7.36-7.29 (m, 2H), 7.28-7.20 (m, 3H), 6.80 (d, J=7.9 Hz, 1H), 6.66 (dd, J=7.9, 1.9 Hz, 1H), 6.62 (d, J=1.8 Hz, 1H), 4.45 (s, 2H), 3.36-3.28 (m, 2H), 2.73 (t, J=6.3 Hz, 2H), 2.02-1.91 (m, 2H); 13C NMR (CDCl₃, 101 MHz) δ 146.9 (C), 138.1 (C), 130.2 (CH), 128.8 (CH), 127.1 (CH), 126.7 (CH), 121.2 (C), 120.9 (C), 118.5 (CH), 113.4 (CH), 55.0 (CH₂), 49.6 (CH₂), 27.9 (CH₂), 22.1 (CH₂); HRMS (ESI) calcd for C₁₆H₁₆BrNNa [M+Na]+324.0358, found 324.0360.

### Example 21: Preparation of Diarylether and Diarylsilane Intermediates (Scheme 2): Bis(1⁻benzyl-1,2,3,4⁻tetrahydroquinolin-7⁻yl)dimethylsilane (27)

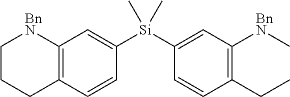 [SMILES: CCc1ccc([Si](C)(C)c2ccc3c(c2)N(Cc2ccccc2)CCC3)cc1N(C)Cc1ccccc1]

A solution of 1⁻benzyl-7⁻bromo-1,2,3,4⁻tetrahydroquinoline (S4; 4.00 g, 13.24 mmol, 2.4 eq) in THE (50 mL) was cooled to −78° C. under nitrogen. n-Butyllithium (2.5 M in hexanes, 5.29 mL, 13.24 mmol, 2.4 eq) was added, and the reaction was stirred at −78° C. for 30 min. Dichlorodimethylsilane (665 μL, 5.51 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-40% CH₂Cl₂/hexanes, linear gradient, with constant 1% v/v Et₃N additive) afforded 2.58 g (93%) of 27 as a white solid. 1H NMR (CDCl₃, 400 MHz) δ 7.30-7.24 (m, 4H), 7.23-7.18 (m, 6H), 6.90 (dt, J=7.4, 1.0 Hz, 2H), 6.67-6.62 (m, 4H), 4.38 (s, 4H), 3.37-3.31 (m, 4H), 2.78 (t, J=6.4 Hz, 4H), 2.02-1.94 (m, 4H), 0.24 (s, 6H); 13C NMR (CDCl₃, 101 MHz) δ 145.0 (C), 139.3 (C), 136.8 (C), 128.66 (CH), 128.60 (CH), 127.0 (CH), 126.8 (CH), 123.4 (C), 122.0 (CH), 116.9 (CH), 55.6 (CH₂), 50.3 (CH₂), 28.3 (CH₂), 22.5 (CH₂), −2.3 (CH₃); HRMS (ESI) calcd for C₃₄H₃₉N₂Si [M+H]⁺ 503.2877, found 503.2873.

### Example 22: Preparation of Diarylether and Diarylsilane Intermediates (Scheme 2): Dimethylbis(2,3,6,7⁻tetrahydro-1H,5H-pyrido[3,2,1⁻ij]quinolin-8-yl)silane (30)

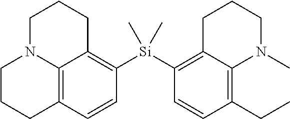 [SMILES: CCc1ccc([Si](C)(C)c2ccc3c4c2CCCN4CCC3)c2c1N(C)CCC2]

A solution of 8-bromojulolidine (S5; 3.00 g, 11.90 mmol, 2.4 eq) in THE (50 mL) was cooled to −78° C. under nitrogen. n-Butyllithium (2.5 M in hexanes, 4.76 mL, 11.90 mmol, 2.4 eq) was added, and the reaction was stirred at −78° C. for 30 min. Dichlorodimethylsilane (598 μL, 4.96 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-20% Et₂O/hexanes, linear gradient) afforded 1.54 g (77%) of 30 as a white foam. ¹H NMR (CDCl₃, 400 MHz) δ 6.77 (d, J=7.4 Hz, 2H), 6.69 (d, J=7.4 Hz, 2H), 3.16-3.10 (m, 4H), 3.10-3.04 (m, 4H), 2.76 (t, J=6.5 Hz, 4H), 2.67 (t, J=6.5 Hz, 4H), 1.96 (p, J=6.3 Hz, 4H), 1.85 (p, J=6.3 Hz, 4H), 0.51 (s, 6H); 13C NMR (CDCl₃, 101 MHz) δ 142.8 (C), 135.3 (C), 127.7 (C), 126.7 (CH), 123.3 (C), 122.9 (CH), 50.7 (CH₂), 50.2 (CH₂), 28.7 (CH₂), 28.2 (CH₂), 22.5 (CH₂), 22.2 (CH₂), 0.2 (CH₃); HRMS (ESI) calcd for C₂₆H₃₅N₂Si [M+H]⁺ 403.2564, found 403.2563.

### Example 23: Preparation of Diarylether and Diarylsilane Intermediates (Scheme 2): Bis(9⁻bromo-2,3,6,7⁻tetrahydro-1H,5H-pyrido[3,2,1⁻ij]quinolin-8-yl)dimethylsilane (S6)

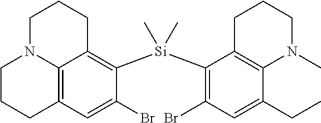 [SMILES: CCc1cc(Br)c([Si](C)(C)c2c(Br)cc3c4c2CCCN4CCC3)c2c1N(C)CCC2]

Dimethylbis(2,3,6,7⁻tetrahydro-1H,5H-pyrido[3,2,1⁻ij]quinolin-8-yl)silane (30; 980 mg, 2.43 mmol) was taken up in DMF (12 mL), and N-bromosuccinimide (866 mg, 4.87 mmol, 2 eq) was added portion-wise over 5 min. The reaction was stirred at room temperature for 2 h. It was subsequently diluted with water and extracted with EtOAc (2×). The combined organic extracts were washed with water and brine, dried over anhydrous MgSO₄, filtered, and concentrated in vacuo. Purification by silica gel chromatography (0-20% Et₂O/hexanes, linear gradient) afforded 690 mg (51%) of dibromide S6 as a white solid. ¹H NMR (DMSO-d₆, 400 MHz, 350 K) δ6.87 (s, 2H), 3.10 (t, J=6.1 Hz, 4H), 3.03 (t, J=6.0 Hz, 4H), 2.66 (t, J=6.4 Hz, 4H), 2.63 (t, J=6.4 Hz, 4H), 1.83 (p, J=6.3 Hz, 4H), 1.72 (p, J=6.2 Hz, 4H), 0.71 (s, 6H); 13C NMR (DMSO-d₆, 101 MHz, 350 K) δ 141.4 (C), 136.4 (C), 130.8 (CH), 128.6 (C), 124.1 (C), 113.9 (C), 49.4 (CH₂), 48.6 (CH₂), 28.2 (CH₂), 26.7 (CH₂), 21.4 (CH₂), 20.8 (CH₂), 7.1 (CH₃); HRMS (ESI) calcd for C₂₆H₃₃Br₂N₂Si [M+H]⁺ 561.0754, found 561.0751.

### Example 24: Preparation of Diarylether and Diarylsilane Intermediates (Scheme 2): Dimethylbis(1⁻methylindolin-6⁻yl)silane (S8)

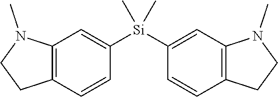 [SMILES: CN1CCc2ccc([Si](C)(C)c3ccc4c(c3)N(C)CC4)cc21]

A solution of 6⁻bromo-1⁻methylindoline (S7; 3.30 g, 15.56 mmol, 2.4 eq) in THE (40 mL) was cooled to −78° C. under nitrogen. n-Butyllithium (2.5 M in hexanes, 6.22 mL, 15.56 mmol, 2.4 eq) was added, and the reaction was stirred at −78° C. for 30 min. Dichlorodimethylsilane (782 μL, 6.48 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-25% EtOAc/hexanes, linear gradient) afforded 2.03 g (97%) of S8 as a colorless oil. ¹H NMR (CDCl₃, 400 MHz) δ 7.08 (dq, J=7.2, 1.0 Hz, 2H), 6.86 (dd, J=7.1, 0.9 Hz, 2H), 6.65 (bs, 2H), 3.27 (t, J=8.1 Hz, 4H), 2.93 (td, J=8.1, 1.1 Hz, 4H), 2.74 (s, 6H), 0.50 (s, 6H); 13C NMR (CDCl₃, 101 MHz) δ 152.9 (C), 137.4 (C), 131.8 (C), 124.4 (CH), 124.0 (CH), 112.4 (CH), 56.1 (CH₂), 36.4 (CH₃), 28.9 (CH₂), −1.7 (CH₃); HRMS (ESI) calcd for C₂₀H₂₇N₂Si [M+H]⁺ 323.1938, found 323.1946.

### Example 25: Preparation of Diarylether and Diarylsilane Intermediates (Scheme 2): Bis(5⁻bromo-1⁻methylindolin-6⁻yl)dimethylsilane (S9)

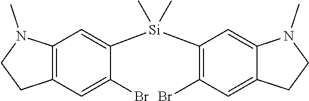 [SMILES: CN1CCc2cc(Br)c([Si](C)(C)c3cc4c(cc3Br)CCN4C)cc21]

A solution of dimethylbis(1⁻methylindolin-6⁻yl)silane (S8; 1.90 g, 5.89 mmol) in DMF (30 mL) was cooled to 0° C., and N-bromosuccinimide (2.10 g, 11.78 mmol, 2 eq) was added portion-wise over 15 min. The reaction was warmed to room temperature and stirred 2 h. It was subsequently diluted with water and extracted with EtOAc (2×). The combined organic extracts were washed with water and brine, dried over anhydrous MgSO₄, filtered, and concentrated in vacuo. Purification by silica gel chromatography (0-25% EtOAc/hexanes, linear gradient) afforded 2.46 g (87%) of dibromide S9 as a white solid. ¹H NMR (CDCl₃, 400 MHz) δ 7.20 (t, J=1.0 Hz, 2H), 6.56 (s, 2H), 3.29 (t, J=8.2 Hz, 4H), 2.92 (td, J=8.1, 1.2 Hz, 4H), 2.70 (s, 6H), 0.71 (s, 6H); 13C NMR (CDCl₃, 101 MHz) δ 152.1 (C), 137.0 (C), 134.2 (C), 128.8 (CH), 117.9 (C), 115.2 (CH), 56.2 (CH₂), 36.3 (CH₃), 28.5 (CH₂), −0.6 (CH₃); HRMS (ESI) calcd for C₂₀H₂₅Br₂N₂Si [M+H]⁺ 481.0128, found 481.0120.

### Example 26: Preparation of Diarylether and Diarylsilane Intermediates (Scheme 2): 3,3′-(Dimethylsilanediyl)bis(N,N-diallylaniline) (S11)

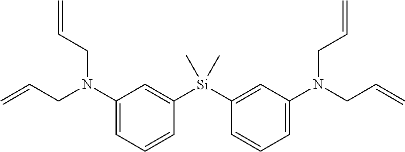 [SMILES: C=CCN(CC=C)c1cccc([Si](C)(C)c2cccc(N(CC=C)CC=C)c2)c1]

A solution of N,N-diallyl-3⁻bromoaniline (S10; 35.00 g, 138.8 mmol, 2.4 eq) in THF (300 mL) was cooled to −78° C. under nitrogen. n-Butyllithium (2.5 M in hexanes, 55.52 mL, 138.8 mmol, 2.4 eq) was added, and the reaction was stirred at −78° C. for 30 min. Dichlorodimethylsilane (6.98 mL, 57.83 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-5% Et₂O/hexanes, linear gradient) afforded 22.92 g (98%) of S11 as a colorless oil. ¹H NMR (CDCl₃, 400 MHz) δ 7.18 (dd, J=8.3, 7.1 Hz, 2H), 6.89-6.87 (m, 2H), 6.85 (dt, J=7.1, 1.0 Hz, 2H), 6.70 (ddd, J=8.3, 2.8, 1.0 Hz, 2H), 5.83 (ddt, J=17.1, 10.1, 5.0 Hz, 4H), 5.14 (dq, J=17.4, 1.8 Hz, 4H), 5.12 (dq, J=10.4, 1.7 Hz, 4H), 3.89 (dt, J=4.9, 1.7 Hz, 8H), 0.48 (s, 6H); 13C NMR (CDCl₃, 101 MHz) δ 148.1 (C), 139.0 (C), 134.3 (CH), 128.6 (CH), 122.4 (CH), 118.3 (CH), 116.2 (CH₂), 113.3 (CH), 53.0 (CH₂), −2.2 (CH₃); HRMS (ESI) calcd for C₂₆H₃₅N₂Si [M+H]⁺ 403.2564, found 403.2565.

### Example 27: Preparation of Diarylether and Diarylsilane Intermediates (Scheme 2): 3,3′-(Dimethylsilanediyl)dianiline (S12)

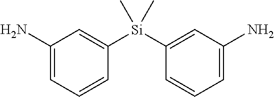 [SMILES: C[Si](C)(c1cccc(N)c1)c1cccc(N)c1]

A round-bottom flask equipped with a reflux condenser was charged with Pd(PPh₃)₄(5.74 g, 4.97 mmol, 0.1 eq) and 1,3⁻dimethylbarbituric acid (62.04 g, 397.4 mmol, 8 eq). The flask was sealed and evacuated/backfilled with nitrogen (3×). A solution of 3,3′-(dimethylsilanediyl)bis(N,N-diallylaniline) (511; 20.00 g, 49.67 mmol) in CH₂Cl₂ (500 mL) was added via cannula, and the resulting mixture was stirred at reflux for 18 h. The reaction was cooled to room temperature and slowly diluted with saturated NaHCO₃ (400 mL) while stirring vigorously. The layers were separated, and the aqueous layer was extracted again with CH₂Cl₂. The combined organic extracts were dried over anhydrous MgSO₄, filtered, and concentrated in vacuo. The residue was purified twice by silica gel chromatography (10-100% EtOAc/hexanes, linear gradient) to afford 10.34 g (86%) of S12 as an off-white solid. ¹H NMR (CDCl₃, 400 MHz) δ 7.15 (ddd, J=7.8, 7.1, 0.5 Hz, 2H), 6.92 (dt, J=7.2, 1.1 Hz, 2H), 6.83-6.79 (m, 2H), 6.68 (ddd, J=7.9, 2.5, 1.1 Hz, 2H), 3.58 (s, 4H), 0.48 (s, 6H); 13C NMR (CDCl₃, 101 MHz) δ 145.8 (C), 139.5 (C), 128.9 (CH), 124.6 (CH), 120.9 (CH), 116.1 (CH), −2.3 (CH₃); HRMS (ESI) calcd for C1₄H₁₉N₂Si [M+H]⁺ 243.1312, found 243.1315.

### Example 28: Preparation of Diarylether and Diarylsilane Intermediates (Scheme 2): Dimethylbis(2,2,4⁻trimethyl-1,2⁻dihydroquinolin-7⁻yl)silane (35)

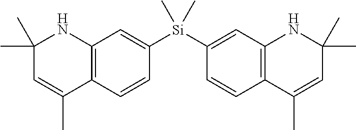

3,3′-(Dimethylsilanediyl)dianiline (512; 10.00 g, 41.25 mmol) and iodine (4.19 g, 16.50 mmol, 0.4 eq) were combined in acetone (250 mL) and stirred at reflux for 48 h. The reaction was then cooled to room temperature, concentrated to dryness, and purified by silica gel chromatography (0-30% Et₂O/hexanes, linear gradient) to afford 5.05 g (30%) of 35 as an off-white solid. ¹H NMR (CDCl₃, 400 MHz) δ 7.03 (d, J=7.5 Hz, 2H), 6.80 (dd, J=7.5, 1.1 Hz, 2H), 6.54 (d, J=1.1 Hz, 2H), 5.30 (q, J=1.5 Hz, 2H), 3.64 (s, 2H), 1.97 (d, J=1.4 Hz, 6H), 1.26 (s, 12H), 0.44 (s, 6H); 13C NMR (CDCl₃, 101 MHz) δ 142.5 (C), 138.6 (C), 128.9 (CH), 128.6 (C), 123.2 (CH), 123.0 (CH), 122.2 (C), 118.7 (CH), 52.0 (C), 31.4 (CH₃), 18.7 (CH₃),-2.2 (CH₃); HRMS (ESI) calcd for C₂₆H₃₅N₂Si [M+H]⁺ 403.2564, found 403.2562.

### Example 29: Preparation of Diarylether and Diarylsilane Intermediates (Scheme 2): Dimethylbis(2,2,4⁻trimethyl-1,2,3,4⁻tetrahydroquinolin-7⁻yl)silane (S13)

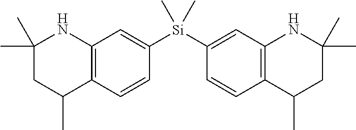

Dimethylbis(2,2,4⁻trimethyl-1,2⁻dihydroquinolin-7⁻yl)silane (35; 1.48 g, 3.68 mmol) was dissolved in THE (35 mL) in a round-bottom flask under nitrogen, and Pd/C (10%, 782 mg, 0.735 mmol, 0.2 eq) was added. The sealed flask was evacuated/backfilled with H₂ from a balloon (4×) and then stirred under the H₂ balloon at room temperature for 18 h. The reaction mixture was filtered through Celite with EtOAc and concentrated in vacuo. Silica gel chromatography (0-25% Et₂O/hexanes, linear gradient) yielded 1.25 g (84%) of S13 as a white foam (mixture of diastereomers). ¹H NMR (CDCl₃, 400 MHz) δ 7.14 (d, J=7.6 Hz, 2H), 6.83 (d, J=7.5 Hz, 2H), 6.61-6.57 (m, 2H), 3.57 (s, 2H), 2.96-2.84 (m, 2H), 1.72 (dd, J=12.9, 5.5 Hz, 2H), 1.43 (t, J=12.6 Hz, 2H), 1.32 (d, J=6.7 Hz, 6H), 1.22 (s, 6H), 1.17 (s, 6H), 0.44 (s, 6H); Analytical HPLC: t_{R}=10.0 min, >99% purity (10-95% MeCN/H₂O, linear gradient, with constant 0.1% v/v TFA additive; 20 min run; 1 mL/min flow; ESI; positive ion mode; detection at 230 nm); HRMS (ESI) calcd for C₂₆H₃₉N₂Si [M+H]⁺ 407.2877, found 407.2873.

### Example 30: Preparation of Diarylether and Diarylsilane Intermediates (Scheme 2): Bis(1⁻benzyl-2,2,4⁻trimethyl-1,2,3,4⁻tetrahydroquinolin-7⁻yl)dimethylsilane (32)

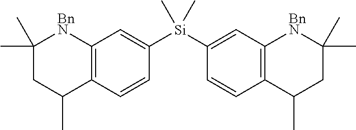

Dimethylbis(2,2,4⁻trimethyl-1,2,3,4⁻tetrahydroquinolin-7⁻yl)silane (S13; 1.15 g, 2.83 mmol), K₂CO₃ (2.34 g, 16.97 mmol, 6 eq), and KI (188 mg, 1.13 mmol, 0.4 eq) were combined in DMF (30 mL) in a round-bottom flask. Benzyl bromide (2.90 g, 16.97 mmol, 6 eq) was added, the flask was sealed, and the contents were stirred at 100° C. for 18 h. The reaction was cooled to room temperature, diluted with water, and extracted with EtOAc (2×). The combined organic extracts were washed with water and brine, dried over anhydrous MgSO₄, filtered, and concentrated in vacuo. Silica gel chromatography (0-10% Et₂O/hexanes, linear gradient) yielded 1.59 g (96%) of 32 as a white foam (mixture of diastereomers). 1H NMR (CDCl₃, 400 MHz) δ 7.28-7.15 (m, 10H), 7.03 (d, J=7.4 Hz, 2H), 6.57 (d, J=7.4 Hz, 2H), 6.42-6.37 (m, 2H), 4.62 (d, J=17.5 Hz, 2H), 4.13 (d, J=17.6 Hz, 2H), 3.04-2.92 (m, 2H), 1.80 (dd, J=13.0, 5.0 Hz, 2H), 1.72 (td, J=12.7, 2.0 Hz, 2H), 1.34 (d, J=6.6 Hz, 6H), 1.27 (s, 6H), 1.24 (s, 6H), 0.04 (s, 1.5H), 0.00 (s, 3H), −0.04 (s, 1.5H); Analytical HPLC: t_{R}=13.9 min, >99% purity (65-95% MeCN/H₂O, linear gradient, with constant 0.1% v/v TFA additive; 20 min run; 1 mL/min flow; ESI; positive ion mode; detection at 254 nm); HRMS (ESI) calcd for C₄₀H₅1N₂Si [M+H]⁺ 587.3816, found 587.3808.

### Example 31: Preparation of Diarylether and Diarylsilane Intermediates (Scheme 2): Dimethylbis(1,2,2,4⁻tetramethyl-1,2⁻dihydroquinolin-7⁻yl)silane (37)

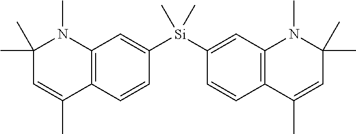

A solution of 7⁻bromo-1,2,2,4⁻tetramethyl-1,2⁻dihydroquinoline (S14; 10.26 g, 38.55 mmol, 2.5 eq) in THE (75 mL) was cooled to −78° C. under nitrogen. n-Butyllithium (2.5 M in hexanes, 15.42 mL, 38.55 mmol, 2.5 eq) was added, and the reaction was stirred at −78° C. for 30 min. Dichlorodimethylsilane (1.86 mL, 15.42 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-5% Et₂O/hexanes, linear gradient) afforded 6.00 g (90%) of 37 as a colorless oil. ¹H NMR (CDCl₃, 400 MHz) δ 7.03 (d, J=7.4 Hz, 2H), 6.84 (dd, J=7.3, 1.1 Hz, 2H), 6.68 (d, J=1.0 Hz, 2H), 5.28 (q, J=1.5 Hz, 2H), 2.76 (s, 6H), 1.96 (d, J=1.4 Hz, 6H), 1.28 (s, 12H), 0.50 (s, 6H); 13C NMR (CDCl₃, 101 MHz) δ 144.4 (C), 138.8 (C), 130.6 (CH), 128.3 (C), 124.0 (C), 122.59 (CH), 122.57 (CH), 116.1 (CH), 56.3 (C), 30.6 (CH₃), 27.4 (CH₃), 18.7 (CH₃), −2.0 (CH₃); HRMS (ESI) calcd for C₂₈H₃₉N₂Si [M+H]⁺ 431.2877, found 431.2874.

### Example 32: Preparation of Diarylether and Diarylsilane Intermediates (Scheme 2): Tetrafluorophthalaldehydic acid (5)

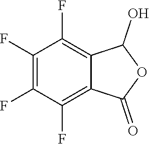 [SMILES: O=C1OC(O)c2c(F)c(F)c(F)c(F)c21]

A solution of 2,3,4,5⁻tetrafluorobenzoic acid (2.00 g, 10.31 mmol) in THE (30 mL) was cooled to −78° C. under nitrogen. N-Butyllithium (2.5 M in hexanes, 9.89 mL, 24.73 mmol, 2.4 eq) was added, and the reaction was stirred at −78° C. for 3 h. Methyl formate (3.18 mL, 51.53 mmol, 5 eq) was added in one portion; the reaction was then stirred at −78° C. for 30 min, warmed to room temperature, and stirred for 2 h. Following the addition of water (75 mL) and CH₂Cl₂ (75 mL), the pH was adjusted to 10-11 with 2 M NaOH. The aqueous layer was washed again with CH₂Cl₂, acidified to pH ˜2 with 2 M HCl, and extracted with EtOAc (2×). The combined EtOAc layers were dried over anhydrous MgSO₄, filtered, and evaporated. Purification by silica gel chromatography (5-100% EtOAc/hexanes, linear gradient, with constant 1% v/v AcOH additive) afforded 5 as a white solid (1.58 g, 69%). ¹H NMR (DMSO-d₆, 400 MHz) δ 8.77 (s, 1H), 6.88 (s, 1H); ¹⁹F NMR (DMSO-d₆, 376 MHz) δ−140.20 (td, J=20.6, 8.4 Hz, 1F), −142.72 (t, J=20.1 Hz, 1F), −143.93-−144.25 (m, 1F), −150.36 (t, J=20.5 Hz, 1F); Analytical HPLC: t_{R}=9.2 min, >99% purity (10-95% MeCN/H₂O, linear gradient, with constant 0.1% v/v TFA additive; 20 min run; 1 mL/min flow; ESI; positive ion mode; detection at 230 nm); HRMS (ESI) calcd for C₈HF₄O₃[M−H]220.9867, found 220.9861.

### Example 33: Rhodamine Synthesis Via Lactol (Phthalaldehydic Acid) Condensation (Scheme 5): 4,5,6,7⁻Tetrafluoro-Q-rhodamine (FRh_{Q}, 14)

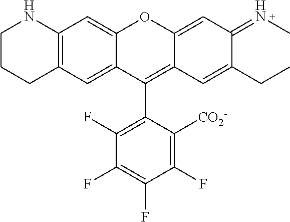

1,2,3,4⁻Tetrahydroquinolin-7⁻ol (13; 141 mg, 0.946 mmol, 2.1 eq), tetrafluorophthalaldehydic acid (5; 100 mg, 0.450 mmol), 2,2,2⁻trifluoroethanol (8 mL), and water (2 mL) were combined in a round-bottom flask. The reaction mixture was sparged with O₂ from a balloon for 10 min, then stirred at 80° C. under the O₂ balloon for 24 h. The reaction was then cooled to room temperature, diluted with MeOH, deposited onto Celite, and concentrated to dryness. Purification by silica gel chromatography (0-20% MeOH (2 M NH₃)/CH₂Cl₂, linear gradient; dry load with Celite) followed by reverse phase HPLC (20-70% MeCN/H₂O, linear gradient, with constant 0.1% v/v TFA additive) provided 14 as a dark red solid (152 mg, 57%, TFA salt). ¹H NMR (CD₃OD, 400 MHz) δ 6.98 (s, 2H), 6.64 (s, 2H), 3.51-3.44 (m, 4H), 2.78 (t, J=6.2 Hz, 4H), 1.93 (p, J=6.3 Hz, 4H); 19F NMR (CD₃OD, 376 MHz) δ−75.53 (s, 3F), −136.17-−136.31 (m, 1F), −137.42-−137.56 (m, 1F), −151.06-−151.23 (m, 1F), −152.47-−152.64 (m, 1F); Analytical HPLC: t_{R}=11.7 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₂₆H₁₉F4N₂O₃[M+H]⁺ 483.1326, found 483.1321.

### Example 34: Rhodamine Synthesis Via Lactol (Phthalaldehydic Acid) Condensation (Scheme 5): 6⁻Carboxy-Q-rhodamine (S17)

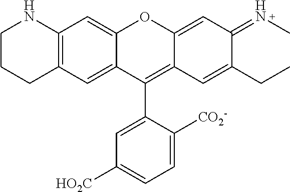

1,2,3,4⁻Tetrahydroquinolin-7⁻ol (13; 78.3 mg, 0.525 mmol, 2.1 eq), 4-carboxyphthalaldehydic acid⁴² (109; 48.6 mg, 0.250 mmol), 2,2,2⁻trifluoroethanol (4 mL), and water (1 mL) were combined in a crimp-top vial. The reaction mixture was sparged with O₂ from a balloon for 10 min, then stirred at 80° C. under the O₂ balloon for 24 h. The reaction was then cooled to room temperature, and concentrated to dryness. Purification by reverse phase HPLC (20-40% MeCN/H₂O, linear gradient, with constant 0.1% v/v TFA additive) provided S17 as a rust red solid (74.1 mg, 52%, TFA salt). ¹H NMR (DMSO-d₆, 400 MHz) δ 13.61 (s, 1H), 13.35 (s, 1H), 8.80 (s, 2H), 8.31 (d, J=8.1 Hz, 1H), 8.27 (dd, J=8.2, 1.6 Hz, 1H), 7.83 (d, J=1.5 Hz, 1H), 6.69 (s, 2H), 6.68 (s, 2H), 3.42-3.34 (m, 4H), 2.64 (t, J=6.4 Hz, 4H), 1.84-1.70 (m, 4H); 13C NMR (DMSO-d₆, 101 MHz) δ 166.1 (C), 165.7 (C), 155.7 (C), 155.4 (C), 154.8 (C), 134.5 (C), 134.2 (C), 133.8 (C), 131.4 (CH), 130.7 (CH), 130.7 (CH), 128.3 (CH), 123.4 (C), 112.8 (C), 96.4 (CH), 40.9 (CH₂), 26.1 (CH₂), 19.8 (CH₂); Analytical HPLC: t_{R}=9.4 min, >99% purity (10-95% MeCN/H₂O, linear gradient, with constant 0.1% v/v TFA additive; 20 min run; 1 mL/min flow; ESI; positive ion mode; detection at 550 nm); HRMS (ESI) calcd for C₂₇H₂₃N₂O₅ [M+H]⁺ 455.1601, found 455.1598.

### Example 35: Rhodamine Synthesis Via Lactol (Phthalaldehydic Acid) Condensation (Scheme 5): 4,5,6,7⁻Tetrafluoro-rhodamine 101 (FRh₁₀₁, 16)

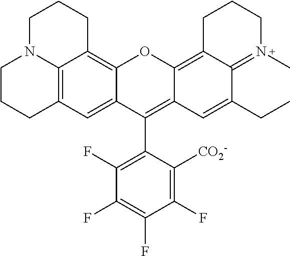

8⁻Hydroxyjulolidine (15; 358 mg, 1.89 mmol, 2.1 eq) and tetrafluorophthalaldehydic acid (5; 200 mg, 0.901 mmol) were combined in 2,2,2-trifluoroethanol (18 mL) in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH (2 M NH₃)/CH₂Cl₂, linear gradient) to provide 16 as a dark purple solid (385 mg, 76%). ¹H NMR (CD₃OD, 400 MHz) δ 6.87 (s, 2H), 3.59-3.48 (m, 8H), 3.05 (t, J=6.4 Hz, 4H), 2.86-2.70 (m, 4H), 2.09 (p, J=6.3 Hz, 4H), 1.97 (p, J=6.3 Hz, 4H); 19F NMR (CD₃OD, 376 MHz) δ-139.20 (ddd, J=21.3, 12.6, 3.4 Hz, 1F), −141.27 (ddd, J=22.5, 12.6, 3.5 Hz, 1F), −154.15 (ddd, J=22.7, 18.8, 3.8 Hz, 1F), −158.00 (ddd, J=22.4, 19.0, 3.5 Hz, 1F); Analytical HPLC: t_{R}=13.5 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₃₂H₂₇F₄N₂O₃[M+H]⁺ 563.1952, found 563.1943.

### Example 36: Rhodamine Synthesis Via Lactol (Phthalaldehydic Acid) Condensation (Scheme 5): JF₅₆₃ (18)

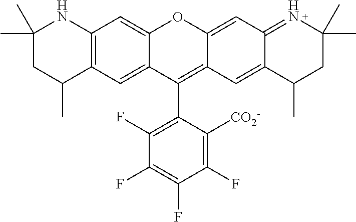

7,7′—Oxybis(2,2,4⁻trimethyl-1,2,3,4⁻tetrahydroquinoline) (17; 200 mg, 0.549 mmol) and tetrafluorophthalaldehydic acid (5; 183 mg, 0.823 mmol, 1.5 eq) were combined in 2,2,2⁻trifluoroethanol (11 mL) in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH (2 M NH₃)/CH₂Cl₂, linear gradient) to provide 84.2 mg (27%) of 18 as a dark red-purple solid (mixture of diastereomers). ¹H NMR (CD₃OD, 400 MHz) δ 7.15-7.10 (m, 2H), 6.63-6.58 (m, 2H), 2.99-2.86 (m, 2H), 1.93-1.82 (m, 2H), 1.48-1.22 (m, 20H); 19F NMR (CD₃OD, 376 MHz) δ−138.47-−139.23 (m, 1F), −139.52-−140.42 (m, 1F), −152.78-−153.26 (m, 1F), −156.57-−158.07 (m, 1F); Analytical HPLC: t_{R} (three isomers)=13.7 min, 14.0 min, 14.2 min; >99% total purity (10-95% MeCN/H₂O, linear gradient, with constant 0.1% v/v TFA additive; 20 min run; 1 mL/min flow; ESI; positive ion mode; detection at 550 nm); HRMS (ESI) calcd for C₃₂H₃₁F₄N₂O₃[M+H]⁺ 567.2265, found 567.2264.

### Example 37: Rhodamine Synthesis Via Lactol (Phthalaldehydic Acid) Condensation (Scheme 5): 2⁻(2,2,4,8,10,10⁻Hexamethyl-1,2,3,4,8,9,10,11-octahydropyrano[3,2⁻g:5,6⁻g′]diquinolin-13⁻ium-6⁻yl)benzoate (99)

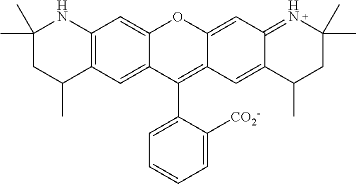

7,7′—Oxybis(2,2,4⁻trimethyl-1,2,3,4⁻tetrahydroquinoline) (17; 400 mg, 1.10 mmol) and phthalaldehydic acid (S16; 165 mg, 1.10 mmol, 1 eq) were combined in 2,2,2-trifluoroethanol (22 mL) in a round-bottom flask. The reaction mixture was sparged with O₂ from a balloon for 10 min, then stirred at 80° C. under the O₂ balloon for 18 h. The reaction was cooled to room temperature, concentrated in vacuo, and purified by silica gel chromatography (0-20% MeOH (2 M NH₃)/CH₂Cl₂, linear gradient) to provide 151 mg (28%) of 99 as a red solid (mixture of diastereomers). An analytically pure sample for spectral characterization was obtained by reverse phase HPLC (10-95% MeCN/H₂O, linear gradient, with constant 0.1% TFA). ¹H NMR (CD₃OD, 400 MHz, TFA salt) δ 8.35-8.29 (m, 1H), 7.88-7.77 (m, 2H), 7.43-7.38 (m, 1H), 6.98-6.95 (m, 1H), 6.94-6.91 (m, 1H), 6.67-6.62 (m, 2H), 2.94-2.81 (m, 2H), 1.90-1.81 (m, 2H), 1.43-1.33 (m, 8H), 1.32-1.26 (m, 6H), 1.16-1.09 (m, 6H); Analytical HPLC: t_{R} (three isomers)=13.4 min, 13.6 min, 13.8 min; >99% total purity (10-95% MeCN/H₂O, linear gradient, with constant 0.1% v/v TFA additive; 20 min run; 1 mL/min flow; ESI; positive ion mode; detection at 550 nm); HRMS (ESI) calcd for C₃₂H₃₅N₂O₃ [M+H]⁺ 495.2642, found 495.2634.

### Example 38: Rhodamine Synthesis Via Lactol (Phthalaldehydic Acid) Condensation (Scheme 5): 4⁻Carboxy-2⁻(2,2,4,8,10,10⁻hexamethyl-1,2,3,4,8,9,10,11-octahydropyrano[3,2⁻g:5,6⁻g′]diquinolin-13⁻ium-6⁻yl)benzoate (S18)

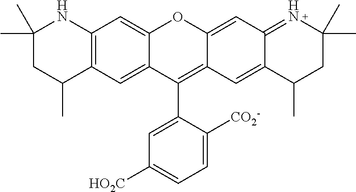

7,7′—Oxybis(2,2,4⁻trimethyl-1,2,3,4⁻tetrahydroquinoline) (17; 300 mg, 0.823 mmol) and 4⁻carboxyphthalaldehydic acid (109; 160 mg, 0.823 mmol, 1 eq) were combined in 2,2,2⁻trifluoroethanol (15 mL) in a round-bottom flask. The reaction mixture was sparged with O₂ from a balloon for 10 min, then stirred at 80° C. under the O₂ balloon for 18 h. The reaction was cooled to room temperature, diluted with MeOH, deposited onto Celite, and concentrated in vacuo. Silica gel chromatography (0-40% MeOH/CH₂Cl₂, linear gradient, with constant 1% v/v AcOH additive; dry load with Celite) produced a broad elution profile for the desired product, so the numerous fractions containing S18 were concentrated to dryness, suspended in water (50 mL), sonicated (5 min), and filtered. The resulting filter cake was washed (water, MeCN, EtOAc, Et₂O) and dried to afford 229 mg (46%, acetate salt) of S18 as a dark red-brown solid (mixture of diastereomers). ¹H NMR (CD₃OD with 1% TFA, 400 MHz) δ 8.42-8.35 (m, 2H), 8.00-7.95 (m, 1H), 6.96-6.92 (m, 1H), 6.92-6.89 (m, 1H), 6.68-6.63 (m, 2H), 2.94-2.83 (m, 2H), 1.90-1.82 (m, 2H), 1.45-1.35 (m, 8H), 1.32-1.28 (m, 6H), 1.17-1.09 (m, 6H); Analytical HPLC: t_{R} (three isomers)=12.2 min, 12.5 min, 12.8 min; >99% total purity (30-50% MeCN/H₂O, linear gradient, with constant 0.1% v/v TFA additive; 20 min run; 1 mL/min flow; ESI; positive ion mode; detection at 550 nm); HRMS (ESI) calcd for C₃₃H₃₅N₂O₅ [M+H]⁺ 539.2540, found 539.2543.

### Example 39: Rhodamine Synthesis Via Lactol (Phthalaldehydic Acid) Condensation (Scheme 5): 2,3,4,5⁻Tetrafluoro-6⁻(2,2,4,8,10,10⁻hexamethyl-1,2,10,11-tetrahydropyrano[3,2⁻g:5,6⁻g]diquinolin-13⁻ium-6⁻yl)benzoate (20)

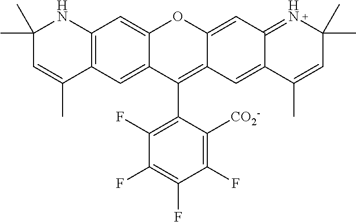

7,7′—Oxybis(2,2,4⁻trimethyl-1,2⁻dihydroquinoline) (19; 180 mg, 0.500 mmol) and tetrafluoro-phthalaldehydic acid (5; 111 mg, 0.500 mmol, 1 eq), 2,2,2⁻trifluoroethanol (8 mL), and water (2 mL) were combined in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH (2 M NH₃)/CH₂Cl₂, linear gradient) to provide 20 as a dark purple solid (82.6 mg, 29%). ¹H NMR (CD₃OD, 400 MHz) δ 6.90 (s, 2H), 6.54 (s, 2H), 5.65 (q, J=1.5 Hz, 2H), 1.91 (d, J=1.4 Hz, 6H), 1.41 (s, 6H), 1.39 (s, 6H); 19F NMR (CD₃OD, 376 MHz) δ-138.77 (ddd, J=21.2, 12.5, 4.0 Hz, 1F), −139.93-−140.09 (m, 1F), −152.85 (ddd, J=22.6, 19.1, 4.0 Hz, 1F), −157.01-−157.25 (m, 1F); Analytical HPLC: t_{R}=13.8 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₃₂H₂₇F₄N₂O₃[M+H]⁺ 563.1952, found 563.1942.

### Example 40: Rhodamine Synthesis Via Lactol (Phthalaldehydic Acid) Condensation (Scheme 5): 2⁻(2,2,4,8,10,10⁻Hexamethyl-1,2,10,11⁻tetrahydropyrano[3,2-g:5,6⁻g′]diquinolin-13⁻ium-6⁻yl)benzoate (101)

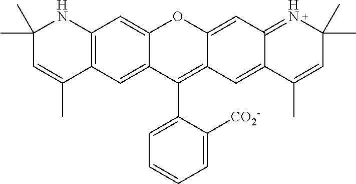

7,7′—Oxybis(2,2,4⁻trimethyl-1,2⁻dihydroquinoline) (19; 180 mg, 0.500 mmol), phthalaldehydic acid (S16; 75.1 mg, 0.500 mmol, 1 eq), 2,2,2⁻trifluoroethanol (8 mL), and water (2 mL) were combined in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH (2 M NH₃)/CH₂Cl₂, linear gradient) to provide 101 as a dark purple solid (68.4 mg, 28%).

¹H NMR (CD₃OD, 400 MHz) δ 8.15-8.10 (m, 1H), 7.70-7.62 (m, 2H), 7.30-7.24 (m, 1H), 6.88 (s, 2H), 6.53 (s, 2H), 5.58 (q, J=1.4 Hz, 2H), 1.76 (d, J=1.4 Hz, 6H), 1.38 (s, 6H), 1.37 (s, 6H); 13C NMR (CD₃OD, 101 MHz) δ 171.3 (C), 160.1 (C), 159.0 (C), 154.8 (C), 134.4 (C), 132.5 (CH), 131.97 (CH), 131.94 (C), 131.6 (CH), 131.01 (CH), 130.98 (CH), 127.4 (C), 124.6 (CH), 122.5 (C), 115.4 (C), 97.0 (CH), 55.1 (C), 31.97 (CH₃), 31.94 (CH₃), 18.1 (CH₃); Analytical HPLC: t_{R}=13.6 min, >99% purity (10-95% MeCN/H₂O, linear gradient, with constant 0.1% v/v TFA additive; 20 min run; 1 mL/min flow; ESI; positive ion mode; detection at 550 nm); HRMS (ESI) calcd for C₃₂H₃₁N₂O₃ [M+H]⁺ 491.2329, found 491.2325.

### Example 41: Rhodamine Synthesis Via Lactol (Phthalaldehydic Acid) Condensation (Scheme 5): 4⁻Carboxy-2⁻(2,2,4,8,10,10⁻hexamethyl-1,2,10,11-tetrahydropyrano[3,2⁻g:5,6⁻g′]diquinolin-13⁻ium-6⁻yl)-benzoate (S19)

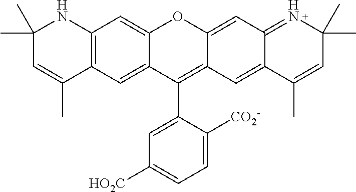

7,7′—Oxybis(2,2,4⁻trimethyl-1,2⁻dihydroquinoline) (19; 300 mg, 0.832 mmol) and 4⁻carboxyphthalaldehydic acid (109; 162 mg, 0.832 mmol, 1 eq) were combined in 2,2,2-trifluoroethanol (15 mL) in a round-bottom flask. The reaction mixture was sparged with O₂ from a balloon for 10 min, then stirred at 80° C. under the O₂ balloon for 18 h. The reaction was cooled to room temperature, diluted with MeOH, deposited onto Celite, and concentrated in vacuo. Purification by silica gel chromatography (0-20% MeOH/CH₂Cl₂, linear gradient, with constant 1% v/v AcOH additive; dry load with Celite) afforded S19 as a dark red solid (197 mg, 40%, acetate salt). An analytically pure sample for spectral characterization was obtained by reverse phase HPLC (20-50% MeCN/H₂O, linear gradient, with constant 0.1% TFA). 1H NMR (CD₃OD, 400 MHz, TFA salt) δ 8.39 (dd, J=8.2, 1.5 Hz, 1H), 8.36 (dd, J=8.2, 0.7 Hz, 1H), 8.01 (dd, J=1.6, 0.7 Hz, 1H), 6.71 (s, 2H), 6.59 (s, 2H), 5.64 (q, J=1.5 Hz, 2H), 1.74 (d, J=1.3 Hz, 6H), 1.41 (s, 12H); 13C NMR (CD₃OD, 101 MHz, TFA salt) δ 168.0 (C), 167.8 (C), 159.0 (C), 156.7 (C), 155.0 (C), 136.9 (C), 135.9 (C), 135.1 (C), 133.0 (CH), 132.51 (CH), 132.48 (CH), 132.3 (CH), 127.1 (C), 123.6 (CH), 123.0 (C), 115.1 (C), 97.3 (CH), 55.3 (C), 31.91 (CH₃), 31.89 (CH₃), 18.1 (CH₃); Analytical HPLC: t_{R}=11.3 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₃₃H₃₁N₂O₅ [M+H]⁺ 535.2227, found 535.2231.

### Example 42: Rhodamine Synthesis Via Lactol (Phthalaldehydic Acid) Condensation (Scheme 5): 2,3,4,5⁻Tetrafluoro-6⁻(1,2,2,4,8,10,10,11⁻octamethyl-1,2,10,11-tetrahydropyrano[3,2⁻g:5,6⁻g]diquinolin-13⁻ium-6⁻yl)benzoate (11)

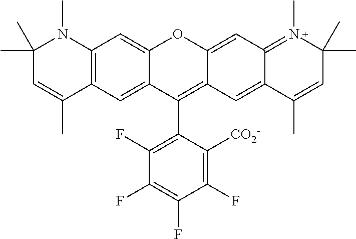

7,7′—Oxybis(1,2,2,4⁻tetramethyl-1,2⁻dihydroquinoline) (12; 1.00 g, 2.57 mmol) and tetrafluorophthalaldehydic acid (5; 572 mg, 2.57 mmol, 1 eq) were combined in 2,2,2-trifluoroethanol (50 mL) in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH (2 M NH₃)/CH₂Cl₂, linear gradient) to provide 11 as a dark blue-purple solid (1.00 g, 66%). ¹H NMR (CD₃OD, 400 MHz) δ 6.93 (s, 2H), 6.81 (s, 2H), 5.68 (q, J=1.5 Hz, 2H), 3.19 (s, 6H), 1.93 (d, J=1.4 Hz, 6H), 1.51 (s, 6H), 1.50 (s, 6H); 19F NMR (CD₃OD, 376 MHz) δ-138.84 (ddd, J=21.1, 12.6, 4.0 Hz, 1F), −140.19 (ddd, J=22.4, 12.6, 3.7 Hz, 1F), −152.79 (ddd, J=23.0, 19.2, 4.1 Hz, 1F), −157.56 (ddd, J=21.4, 19.4, 3.8 Hz, 1F); Analytical HPLC: t_{R}=14.2 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₃₄H₃₁F₄N₂O₃[M+H]⁺ 591.2265, found 591.2258.

### Example 43: Rhodamine Synthesis Via Lactol (Phthalaldehydic Acid) Condensation (Scheme 5): 2⁻(1,2,2,4,8,10,10,11-Octamethyl-1,2,10,11-tetrahydropyrano[3,2⁻g:5,6⁻g]diquinolin-13⁻ium-6⁻yl)benzoate (103)

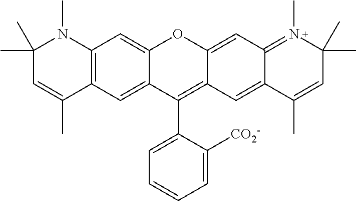

7,7′—Oxybis(1,2,2,4⁻tetramethyl-1,2⁻dihydroquinoline) (12; 200 mg, 0.515 mmol) and phthalaldehydic acid (S16; 77.3 mg, 0.515 mmol, 1 eq) were combined in 2,2,2-trifluoroethanol (10 mL) in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH (2 M NH₃)/CH₂Cl₂, linear gradient) to provide 103 as a dark purple solid (185 mg, 69%). ¹H NMR (CD₃OD, 400 MHz) δ 8.13-8.08 (m, 1H), 7.65 (td, J=7.4, 1.6 Hz, 1H), 7.62 (td, J=7.4, 1.6 Hz, 1H), 7.27-7.22 (m, 1H), 6.94 (s, 2H), 6.76 (s, 2H), 5.62-5.57 (m, 2H), 3.16 (s, 6H), 1.78 (d, J=1.4 Hz, 6H), 1.49 (s, 6H), 1.48 (s, 6H); 13C NMR (CD₃OD, 101 MHz) δ 173.3 (C), 161.5 (C), 159.6 (C), 154.5 (C), 141.9 (C), 133.6 (C), 133.4 (CH), 131.1 (CH), 130.8 (CH), 130.5 (CH), 130.4 (CH), 126.9 (C), 124.2 (C), 123.8 (CH), 115.4 (C), 96.0 (CH), 60.9 (C), 33.2 (CH₃), 29.2 (CH₃), 29.1 (CH₃), 18.3 (CH₃); Analytical HPLC: t_{R}=13.9 min, 98.5% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₃₄H₃₅N₂O₃ [M+H]⁺ 519.2642, found 519.2652.

### Example 44: Rhodamine Synthesis Via Lactol (Phthalaldehydic Acid) Condensation (Scheme 5): 4⁻Carboxy-2⁻(1,2,2,4,8,10,10,11⁻octamethyl-1,2,10,11-tetrahydropyrano[3,2⁻g:5,6⁻g′]diquinolin-13⁻ium-6⁻yl)-benzoate (S20)

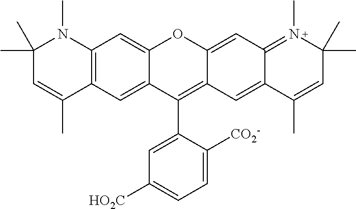

7,7′—Oxybis(1,2,2,4⁻tetramethyl-1,2⁻dihydroquinoline) (12; 300 mg, 0.772 mmol) and 4⁻carboxyphthalaldehydic acid (109; 150 mg, 0.772 mmol, 1 eq) were combined in 2,2,2-trifluoroethanol (15 mL) in a round-bottom flask. The reaction mixture was sparged with O₂ from a balloon for 10 min, then stirred at 80° C. under the O₂ balloon for 18 h. The reaction was cooled to room temperature, deposited onto Celite, and concentrated in vacuo. Purification by silica gel chromatography (0-20% MeOH/CH₂Cl₂, linear gradient, with constant 1% v/v AcOH additive; dry load with Celite) afforded S20 as a dark red-purple solid (387 mg, 80%, acetate salt). An analytically pure sample for spectral characterization was obtained by reverse phase HPLC (20-60% MeCN/H₂O, linear gradient, with constant 0.1% TFA). ¹H NMR (CD₃OD, 400 MHz, TFA salt) δ 8.40 (dd, J=8.2, 1.5 Hz, 1H), 8.38 (d, J=8.0 Hz, 1H), 8.04-8.02 (m, 1H), 6.84 (s, 2H), 6.73 (s, 2H), 5.67 (q, J=1.4 Hz, 2H), 3.20 (s, 6H), 1.75 (d, J=1.3 Hz, 6H), 1.51 (s, 12H); 13C NMR (CD₃OD, 101 MHz, TFA salt) δ 167.9 (C), 167.7 (C), 159.6 (C), 156.7 (C), 154.8 (C), 136.9 (C), 135.9 (C), 134.9 (C), 134.2 (CH), 132.54 (CH), 132.47 (CH), 132.42 (CH), 126.5 (C), 124.8 (C), 122.2 (CH), 115.1 (C), 96.4 (CH), 61.2 (C), 33.4 (CH₃), 29.14 (CH₃), 29.12 (CH₃), 18.1 (CH₃); Analytical HPLC: t_{R}=11.9 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₃₅H₃₅N₂O₅ [M+H]⁺ 563.2540, found 563.2543.

### Example 45: Rhodamine Synthesis Via Lactol (Phthalaldehydic Acid) Condensation (Scheme 5): 2⁻(1,11⁻Dibenzyl-13,13⁻dimethyl-2,3,4,8,9,10,11,13-octahydrosilino[3,2⁻g:5,6⁻g]diquinolin-6⁻ylium-6(1H)-yl)-3,4,5,6⁻tetrafluorobenzoate (28)

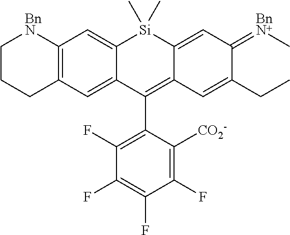

Via lactol condensation route: Bis(1⁻benzyl-1,2,3,4⁻tetrahydroquinolin-7-yl)dimethylsilane (27; 400 mg, 0.796 mmol) and tetrafluorophthalaldehydic acid (5; 265 mg, 1.19 mmol, 1.5 eq) were combined in 2,2,3,3,4,4,4⁻heptafluoro-1⁻butanol (4 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified by silica gel chromatography (0-50% EtOAc/hexanes, linear gradient) to provide 236 mg (42%) of 28 as a pale yellow-green solid.

Via tetrafluorobenzoic acid route: A solution of 2,3,4,5⁻tetrafluorobenzoic acid (6; 275 mg, 1.42 mmol, 2.5 eq) in THE (5 mL) was cooled to −78° C. under nitrogen. N-Butyllithium (2.5 M in hexanes, 1.02 mL, 2.55 mmol, 4.5 eq) was added, and the reaction was stirred at −78° C. for 3 h. A solution of 1,11⁻dibenzyl-13,13⁻dimethyl-1,2,3,4,8,9,10,11-octahydrosilino[3,2⁻g:5,6⁻g′]diquinolin-6(13H)-one³² (S56; 300 mg, 0.567 mmol) in THF (5 mL) was added; the reaction was warmed to room temperature and stirred for 72 h. It was subsequently diluted with saturated NH₄Cl and water and extracted with EtOAc (2×). The combined organic extracts were washed with brine, dried over anhydrous MgSO₄, filtered, and concentrated in vacuo. The residue was purified twice by silica gel chromatography (10-100% EtOAc/hexanes, linear gradient; then, 0-20% EtOAc/toluene, linear gradient) yielded 110 mg (28%) of 28 as a pale yellow-green solid.

¹H NMR (CDCl₃, 400 MHz) δ 7.34-7.28 (m, 4H), 7.26-7.21 (m, 6H), 6.64 (s, 2H), 6.47 (s, 2H), 4.51 (AB quartet, v_{A}=1811.4 Hz, v_{B}=1795.4 Hz, J_{AB}=16.7 Hz, 4H), 3.40-3.32 (m, 4H), 2.73-2.58 (m, 4H), 1.94 (p, J=6.1 Hz, 4H), 0.18 (s, 3H), 0.17 (s, 3H); ¹⁹F NMR (CDCl₃, 376 MHz) δ−139.43 (td, J=20.0, 8.2 Hz, 1F), −139.85 (td, J=20.4, 3.5 Hz, 1F), −143.88 (ddd, J=21.1, 18.6, 8.1 Hz, 1F), −152.53 (ddd, J=21.1, 17.9, 3.4 Hz, 1F); Analytical HPLC: t_{R}=15.5 min, >99% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₄₂H₃₇F₄N₂O₂Si [M+H]⁺ 705.2555, found 705.2547.

### Example 46: Rhodamine Synthesis Via Lactol (Phthalaldehydic Acid) Condensation (Scheme 5): 4,5,6,7⁻Tetrafluoro-Si-Q-rhodamine (FSiRh_{Q}, 29)

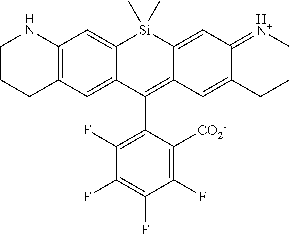

A round-bottom flask was charged with Pd/C (10%, 69.2 mg, 65.0 mol, 0.2 eq) under nitrogen, and 2⁻(1,11⁻dibenzyl-13,13⁻dimethyl-2,3,4,8,9,10,11,13⁻octahydrosilino[3,2-g:5,6⁻g′]diquinolin-6⁻ylium-6(1H)-yl)-3,4,5,6⁻tetrafluorobenzoate (28; 229 mg, 0.325 mmol) in THF (10 mL) was added. The sealed flask was evacuated/backfilled with H₂ from a balloon (4×) and then stirred under the H₂ balloon at room temperature for 24 h. The reaction mixture was filtered through Celite with MeOH, and the filtrate was concentrated in vacuo. The pale blue residue was resuspended in 3:1 CH₂Cl₂/MeOH (8 mL), and p-chloranil (160 mg, 0.650 mmol, 2 eq) was added. After stirring the mixture at room temperature for 2 h, it was deposited onto Celite and concentrated to dryness. Silica gel chromatography (20-100% EtOAc/toluene, linear gradient; dry load with Celite) yielded 133 mg (78%) of 29 as a blue solid. ¹H NMR (CD₃OD, 400 MHz) δ 6.93 (s, 2H), 6.80 (s, 2H), 3.50-3.42 (m, 4H), 2.67 (dt, J=16.0, 6.1 Hz, 2H), 2.57 (dt, J=16.1, 6.2 Hz, 2H), 1.88 (p, J=6.2 Hz, 4H), 0.45 (s, 3H), 0.44 (s, 3H); 19F NMR (CD₃OD, 376 MHz) δ−140.13 (ddd, J=21.6, 13.2, 3.2 Hz, 1F), −141.67 (ddd, J=22.3, 13.1, 3.7 Hz, 1F), −155.20-−155.37 (m, 1F), −157.47-−157.65 (m, 1F); Analytical HPLC: t_{R}=12.2 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₂₈H_{2 5}F₄N₂O₂Si [M+H]⁺ 525.1616, found 525.1613.

### Example 47: Rhodamine Synthesis Via Lactol (Phthalaldehydic Acid) Condensation (Scheme 5): 6⁻Carboxy-Si-Q-rhodamine (S22)

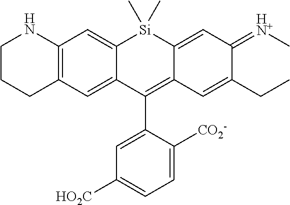

Step 1: Bis(1⁻benzyl-1,2,3,4⁻tetrahydroquinolin-7⁻yl)dimethylsilane (27; 400 mg, 0.796 mmol) and 4⁻carboxyphthalaldehydic acid (109; 232 mg, 1.19 mmol, 1.5 eq) were combined in 2,2,3,3,4,4,4⁻heptafluoro-1⁻butanol (4 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified by silica gel chromatography (5-100% EtOAc/hexanes, linear gradient, with constant 1% v/v AcOH additive) to provide 102 mg (19%) of 4⁻carboxy-2⁻(1,11⁻dibenzyl-13,13⁻dimethyl-2,3,4,8,9,10,11,13-octahydrosilino[3,2⁻g:5,6⁻g′]diquinolin-6⁻ylium-6(1H)-yl)benzoate (S21) as a green solid.

Step 2: A round-bottom flask was charged with Pd/C (10%, 55.0 mg, 51.7 mol, 0.2 eq) under nitrogen, and the product from the first step (S21; 175 mg, 0.259 mmol) in 1:1 THF/MeOH (20 mL) was added. The sealed flask was evacuated/backfilled with H₂ from a balloon (4×) and then stirred under the H₂ balloon at room temperature for 24 h. The reaction mixture was filtered through Celite with MeOH and EtOAc, and the filtrate was concentrated in vacuo. The pale blue residue was resuspended in 3:1 CH₂Cl₂/MeOH (8 mL), and p-chloranil (127 mg, 0.517 mmol, 2 eq) was added. After stirring the mixture at room temperature for 1 h, it was deposited onto Celite and concentrated to dryness. Silica gel chromatography (50-100% EtOAc/hexanes, linear gradient, with constant 1% v/v AcOH additive; dry load with Celite) yielded 98 mg (76%) of S22 as a blue solid. ¹H NMR (CD₃OD with 1% TFA, 400 MHz) δ 8.31 (d, J=8.1 Hz, 1H), 8.28 (dd, J=8.2, 1.6 Hz, 1H), 7.79 (d, J=1.5 Hz, 1H), 6.99 (s, 2H), 6.57 (s, 2H), 3.51-3.42 (m, 4H), 2.49 (t, J=6.3 Hz, 4H), 1.86 (p, J=6.2 Hz, 4H), 0.52 (s, 3H), 0.47 (s, 3H); 13C NMR (CD₃OD with 1% TFA, 101 MHz) δ 168.1 (C), 167.7 (C), 154.0 (C), 146.8 (C), 142.7 (C), 140.2 (CH), 136.1 (C), 135.1 (C), 132.5 (CH), 132.3 (CH), 130.9 (CH), 129.1 (C), 124.0 (C), 123.8 (CH), 43.1 (CH₂), 27.6 (CH₂), 21.5 (CH₂), −1.0 (CH₃), −2.1 (CH₃); Analytical HPLC: t_{R}=9.9 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₂₉H₂₉N₂O₄Si [M+H]⁺ 497.1891, found 497.1887.

### Example 48: Rhodamine Synthesis Via Lactol (Phthalaldehydic Acid) Condensation (Scheme 5): JF₆₉₈ (31)

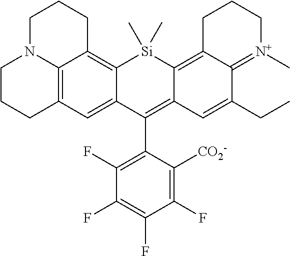

Via lactol condensation route: Reaction of 30 and 5 according to the method described for 28 (the typical Si-rhodamine lactol protocol) afforded only a very low yield (2%) of 31 as a dark green solid.

Via dibromide route: A solution of bis(9⁻bromo-2,3,6,7⁻tetrahydro-1H,5H-pyrido[3,2,1⁻ij]quinolin-8-yl)dimethylsilane (S6; 400 mg, 0.714 mmol) in THF (15 mL) was cooled to −78° C. under nitrogen. tert-Butyllithium (1.7 M in pentane, 1.85 mL, 3.14 mmol, 4.4 eq) was added, and the reaction was stirred at −78° C. for 30 min. It was then warmed to −10° C. before adding a solution of MgBr₂·OEt₂ (405 mg, 1.57 mmol, 2.2 eq) in THE (10 mL). After an additional 30 min at −10° C., a solution of tetrafluorophthalic anhydride (4; 346 mg, 1.57 mmol, 2.2 eq) in THE (10 mL) was added dropwise over 30 min via addition funnel. The reaction was then allowed to warm to room temperature overnight (18 h). It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. Silica gel chromatography (0-15% MeOH (2 M NH₃)/CH₂Cl₂, linear gradient) afforded 192 mg (44%) of 31 as a dark green solid.

¹H NMR (CDCl₃, 400 MHz) δ 6.42 (s, 2H), 3.22 (t, J=6.0 Hz, 4H), 3.20-3.15 (m, 4H), 2.95-2.82 (m, 4H), 2.68-2.51 (m, 4H), 2.00 (p, J=6.1 Hz, 4H), 1.89 (p, J=6.3 Hz, 4H), 0.67-0.63 (m, 3H), 0.59 (s, 3H); 19F NMR (CDCl₃, 376 MHz) δ−139.72 (td, J=20.1, 8.2 Hz, 1F), −142.18 (td, J=20.2, 3.3 Hz, 1F), −143.85 (ddd, J=20.4, 18.3, 8.3 Hz, 1F), −153.92 (ddd, J=21.1, 18.3, 3.3 Hz, 1F); Analytical HPLC: t_{R}=13.3 min, 98.4% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₃₄H₃₃F₄N₂O₂Si [M+H]⁺ 605.2242, found 605.2249.

### Example 49: Rhodamine Synthesis Via Lactol (Phthalaldehydic Acid) Condensation (Scheme 5): 2,3,4,5⁻Tetrafluoro-6⁻(2,2,4,8,10,10,13,13⁻octamethyl-2,3,4,8,9,10,11,13⁻octahydrosilino[3,2⁻g:5,6⁻g]diquin-olin-6⁻ylium-6(1H)-yl)benzoate (34)

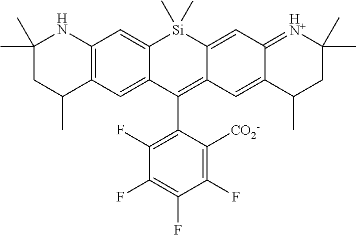

Step 1: Bis(1⁻benzyl-2,2,4⁻trimethyl-1,2,3,4⁻tetrahydroquinolin-7-yl)dimethylsilane (32; 175 mg, 0.298 mmol) and tetrafluorophthalaldehydic acid (5; 99.3 mg, 0.447 mmol, 1.5 eq) were combined in 2,2,3,3,4,4,4⁻heptafluoro-1⁻butanol (1.5 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified by silica gel chromatography (0-30% EtOAc/hexanes, linear gradient) to provide 97.5 mg (41%) of 2⁻(1,11⁻dibenzyl-2,2,4,8,10,10,13,13⁻octamethyl-2,3,4,8,9,10,11,13-octahydrosilino[3,2⁻g:5,6⁻g′]diquinolin-6⁻ylium-6(1H)-yl)-3,4,5,6⁻tetrafluorobenzoate as an off-white solid (33; mixture of diastereomers).

Step 2: A round-bottom flask was charged with Pd/C (10%, 93.6 mg, 88.0 mol, 0.2 eq) under nitrogen, and a solution of the intermediate from Step 1 (33; 347 mg, 0.440 mmol) in THE (10 mL) was added. The sealed flask was evacuated/backfilled with H₂ from a balloon (4×) and then stirred under the H₂ balloon at room temperature for 24 h. A second portion of Pd/C (10%, 93.6 mg, 88.0 mol, 0.2 eq) was added, and stirring was continued for an additional 24 h (H₂, room temperature). The reaction mixture was filtered through Celite with MeOH and THF, and the filtrate was concentrated in vacuo. The pale blue residue was resuspended in 3:1 CH₂Cl₂/MeOH (8 mL), and p-chloranil (216 mg, 0.880 mmol, 2 eq) was added. After stirring the mixture at room temperature for 1 h, it was deposited onto Celite and concentrated to dryness. Silica gel chromatography (0-50% EtOAc/toluene, linear gradient; dry load with Celite) yielded 228 mg (85%) of 34 as a blue solid (mixture of diastereomers).

¹H NMR (CD₃OD, 400 MHz) δ 7.05-7.00 (m, 2H), 6.96-6.92 (m, 2H), 2.90-2.73 (m, 2H), 1.87-1.77 (m, 2H), 1.44-1.36 (m, 2H), 1.36-1.32 (m, 6H), 1.30-1.25 (m, 6H), 1.18-1.13 (m, 3H), 1.12-1.07 (m, 3H), 0.49-0.43 (m, 6H); 19F NMR (CD₃OD, 376 MHz) δ-139.92-−140.13 (m, 1F), −140.97-−141.27 (m, 1F), −154.89-−155.43 (m, 1F), −157.23-−159.17 (m, 1F); Analytical HPLC: t_{R} (three isomers)=14.1 min, 14.4 min, 14.7 min; >99% total purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₄H₃₇F₄N₂O₂Si [M+H]⁺ 609.2555, found 609.2546.

### Example 50: Rhodamine Synthesis Via Lactol (Phthalaldehydic Acid) Condensation (Scheme 5): 2⁻(2,2,4,8,10,10,13,13-Octamethyl-2,3,4,8,9,10,11,13-octahydrosilino[3,2⁻g:5,6⁻g]diquinolin-6⁻ylium-6(1H)-yl)-benzoate (100)

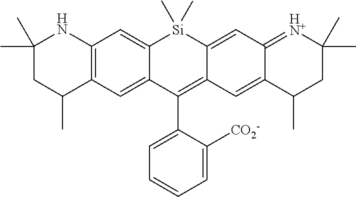

Step 1: Bis(1⁻benzyl-2,2,4⁻trimethyl-1,2,3,4⁻tetrahydroquinolin-7-yl)dimethylsilane (32; 400 mg, 0.682 mmol) and phthalaldehydic acid (S16; 128 mg, 0.852 mmol, 1.25 eq) were combined in 2,2,3,3,4,4,4⁻heptafluoro-1⁻butanol (3 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified by silica gel chromatography (0-25% EtOAc/hexanes, linear gradient) to provide 190 mg (39%) of 2⁻(1,11⁻dibenzyl-2,2,4,8,10,10,13,13⁻octamethyl-2,3,4,8,9,10,11,13-octahydrosilino[3,2⁻g:5,6⁻g′]diquinolin-6⁻ylium-6(1H)-yl)benzoate as an off-white solid (S23; mixture of diastereomers).

Step 2: A round-bottom flask was charged with Pd/C (10%, 56.4 mg, 53.0 mol, 0.2 eq) under nitrogen, and a solution of the intermediate from Step 1 (S23; 190 mg, 0.265 mmol) in THE (10 mL) was added. The sealed flask was evacuated/backfilled with H₂ from a balloon (4×) and then stirred under the H₂ balloon at room temperature for 24 h. A second portion of Pd/C (10%, 56.4 mg, 53.0 mol, 0.2 eq) was added, and stirring was continued for an additional 24 h (H₂, room temperature). The reaction mixture was filtered through Celite with MeOH, and the filtrate was concentrated in vacuo. The residue was resuspended in 3:1 CH₂Cl₂/MeOH (8 mL), and p-chloranil (130 mg, 0.530 mmol, 2 eq) was added. After stirring the mixture at room temperature for 2 h, it was deposited onto Celite and concentrated to dryness. Silica gel chromatography (0-40% EtOAc/hexanes, linear gradient; dry load with Celite) yielded 108 mg (76%) of 100 as a blue solid (mixture of diastereomers).

¹H NMR (CD₃OD, 400 MHz) δ 8.00-7.94 (m, 1H), 7.82-7.72 (m, 1H), 7.70-7.61 (m, 1H), 7.40-7.28 (m, 1H), 6.86-6.81 (m, 2H), 6.67-6.62 (m, 2H), 2.79-2.63 (m, 2H), 1.74-1.63 (m, 2H), 1.33-1.23 (m, 2H), 1.23-1.18 (m, 6H), 1.16-1.09 (m, 6H), 1.03-0.94 (m, 6H), 0.55-0.51 (m, 3H), 0.49-0.45 (m, 3H); Analytical HPLC: t_{R} (three isomers)=14.2 min, 14.4 min, 14.5 min; >99% total purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₄H₄₁N₂O₂Si [M+H]⁺ 537.2932, found 537.2927.

### Example 51: Rhodamine Synthesis Via Lactol (Phthalaldehydic Acid) Condensation (Scheme 5): 2,3,4,5⁻Tetrafluoro-6⁻(2,2,4,8,10,10,13,13⁻octamethyl-2,10,11,13-tetrahydrosilino[3,2⁻g:5,6⁻g]diquinolin-6⁻ylium-6(1H)-yl)benzoate (36)

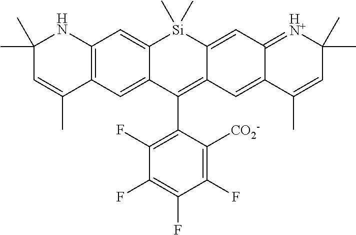

Dimethylbis(2,2,4⁻trimethyl-1,2⁻dihydroquinolin-7⁻yl)silane (35; 200 mg, 0.497 mmol) and tetrafluorophthalaldehydic acid (5; 331 mg, 1.49 mmol, 3 eq) were combined in 2,2,3,3,4,4,4⁻heptafluoro-1⁻butanol (2.5 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified by silica gel chromatography (0-50% EtOAc/hexanes, linear gradient) to provide 36 as a blue-green solid (44.7 mg, 15%). ¹H NMR (CDCl₃, 400 MHz) δ 6.64 (s, 2H), 6.53 (s, 2H), 5.29 (q, J=1.5 Hz, 2H), 3.87 (s, 2H), 1.77 (d, J=1.4 Hz, 6H), 1.274 (s, 6H), 1.272 (s, 6H), 0.48 (s, 3H), 0.48 (s, 3H); 19F NMR (CDCl₃, 376 MHz) δ−139.01-−139.26 (m, 2F), −144.21-−144.35 (m, 1F), −151.82 (t, J=18.0 Hz, 1F); Analytical HPLC: t_{R}=11.6 min, >99% purity (30-95% MeCN/H₂O, linear gradient, with constant 0.1% v/v TFA additive; 20 min run; 1 mL/min flow; ESI; positive ion mode; detection at 725 nm); HRMS (ESI) calcd for C₃₄H₃₃F₄N₂O₂Si [M+H]⁺ 605.2242, found 605.2243.

### Example 52: Rhodamine Synthesis Via Lactol (Phthalaldehydic Acid) Condensation (Scheme 5): 2⁻(2,2,4,8,10,10,13,13-Octamethyl-2,10,11,13-tetrahydrosilino[3,2⁻g:5,6⁻g]diquinolin-6⁻ylium-6(1H)-yl)-benzoate (102)

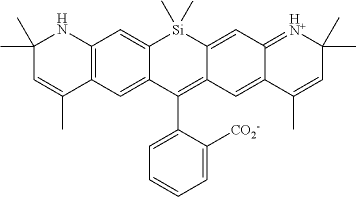

Dimethylbis(2,2,4⁻trimethyl-1,2⁻dihydroquinolin-7⁻yl)silane (35; 200 mg, 0.497 mmol) and phthalaldehydic acid (S16; 224 mg, 1.49 mmol, 3 eq) were combined in 2,2,3,3,4,4,4-heptafluoro-1⁻butanol (2.5 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified by silica gel chromatography (0-50% EtOAc/hexanes, linear gradient) to provide 102 as an off-white solid (18 mg, 7%). 1H NMR (CDCl₃, 400 MHz) δ 7.97 (dt, J=7.6, 1.0 Hz, 1H), 7.66 (td, J=7.5, 1.2 Hz, 1H), 7.56 (td, J=7.5, 1.0 Hz, 1H), 7.36 (dt, J=7.7, 1.0 Hz, 1H), 6.66 (s, 2H), 6.53 (s, 2H), 5.23 (q, J=1.1 Hz, 2H), 3.81 (s, 2H), 1.63 (d, J=1.1 Hz, 6H), 1.25 (s, 12H), 0.54 (s, 3H), 0.53 (s, 3H); 13C NMR (CDCl₃, 101 MHz) δ 170.8 (C), 154.2 (C), 142.5 (C), 136.8 (C), 133.4 (CH), 133.0 (C), 129.1 (CH), 128.9 (CH), 128.2 (C), 127.4 (C), 125.8 (CH), 124.9 (CH), 122.5 (CH), 121.9 (C), 117.5 (CH), 92.6 (C), 52.1 (C), 31.7 (CH₃), 31.6 (CH₃), 18.1 (CH₃), 0.6 (CH₃), −1.8 (CH₃); Analytical HPLC: t_{R}=11.9 min, >99% purity (30-95% MeCN/H₂O, linear gradient, with constant 0.1% v/v TFA additive; 20 min run; 1 mL/min flow; ESI; positive ion mode; detection at 700 nm); HRMS (ESI) calcd for C₃₄H₃₇N₂O₂Si [M+H]⁺ 533.2619, found 533.2616.

### Example 53: Rhodamine Synthesis Via Lactol (Phthalaldehydic Acid) Condensation (Scheme 5): 2⁻(1,2,2,4,8,10,10,11,13,13⁻Decamethyl-2,10,11,13-tetrahydrosilino[3,2⁻g:5,6⁻g]diquinolin-6⁻ylium-6(1H)-yl)-3,4,5,6⁻tetrafluorobenzoate (38)

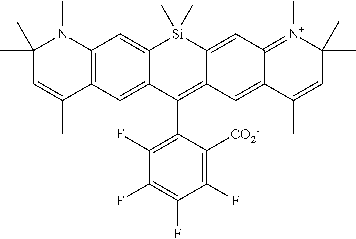

Dimethylbis(1,2,2,4⁻tetramethyl-1,2⁻dihydroquinolin-7⁻yl)silane (37; 300 mg, 0.697 mmol) and tetrafluorophthalaldehydic acid (5; 309 mg, 1.39 mmol, 2 eq) were combined in 2,2,3,3,4,4,4⁻heptafluoro-1⁻butanol (2.3 mL) in a crimp-top vial. The reaction mixture was sparged with O₂ from a balloon for 10 min, then stirred at 95° C. under the O₂ balloon for 24 h. The reaction was cooled to room temperature, diluted with saturated NaHCO₃, and extracted with EtOAc (2×). The combined organic extracts were washed with brine, dried over anhydrous MgSO₄, filtered, and concentrated in vacuo. Silica gel chromatography (0-5₀% EtOAc/hexanes, linear gradient) provided 38 as a pale yellow-green solid (206 mg, 47%). ¹H NMR (CDCl₃, 400 MHz) δ 6.66 (s, 2H), 6.53 (s, 2H), 5.28 (q, J=1.4 Hz, 2H), 2.86 (s, 6H), 1.76 (d, J=1.4 Hz, 6H), 1.31 (s, 6H), 1.30 (s, 6H), 0.55 (s, 3H), 0.52 (s, 3H); 19F NMR (CDCl₃, 376 MHz) δ−139.29 (td, J=20.0, 8.2 Hz, 1F), −140.03 (td, J=20.0, 3.5 Hz, 1F), −144.18 (ddd, J=20.7, 18.5, 8.3 Hz, 1F), −152.19 (ddd, J=21.1, 18.3, 3.6 Hz, 1F); Analytical HPLC: t_{R}=12.6 min, 98.9% purity (30-95% MeCN/H₂O, linear gradient, with constant 0.1% v/v TFA additive; 20 min run; 1 mL/min flow; ESI; positive ion mode; detection at 750 nm); HRMS (ESI) calcd for C₃₆H₃₇F₄N₂O₂Si [M+H]⁺ 633.2555, found 633.2549.

### Example 54: Rhodamine Synthesis Via Lactol (Phthalaldehydic Acid) Condensation (Scheme 5): 2⁻(1,2,2,4,8,10,10,11,13,13⁻Decamethyl-2,10,11,13-tetrahydrosilino[3,2⁻g:5,6⁻g′]diquinolin-6⁻ylium-6(1H)-yl)-benzoate (104)

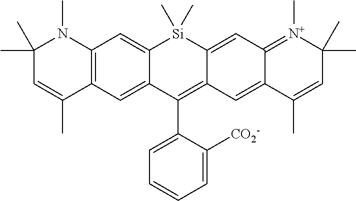

Dimethylbis(1,2,2,4⁻tetramethyl-1,2⁻dihydroquinolin-7⁻yl)silane (37; 300 mg, 0.697 mmol) and phthalaldehydic acid (S16; 157 mg, 1.04 mmol, 1.5 eq) were combined in 2,2,3,3,4,4,4⁻heptafluoro-1⁻butanol (2.3 mL) in a crimp-top vial. The reaction mixture was sparged with O₂ from a balloon for 10 min, then stirred at 95° C. under the O₂ balloon for 18 h. The reaction was cooled to room temperature, diluted with saturated NaHCO₃, and extracted with EtOAc (2×). The combined organic extracts were washed with brine, dried over anhydrous MgSO₄, filtered, and concentrated in vacuo. The crude material was purified twice by silica gel chromatography (0-2₅% EtOAc/toluene, linear gradient; then, 0-50% EtOAc/hexanes, linear gradient) to provide 104 as an off-white solid (41.8 mg, 11%). ¹H NMR (CDCl₃, 400 MHz) δ 7.96 (dt, J=7.6, 1.0 Hz, 1H), 7.61 (td, J=7.5, 1.2 Hz, 1H), 7.52 (td, J=7.5, 1.0 Hz, 1H), 7.28 (d, J=7.7 Hz, 1H), 6.69 (s, 2H), 6.53 (s, 2H), 5.21 (t, J=1.5 Hz, 2H), 2.85 (s, 6H), 1.63 (d, J=1.4 Hz, 6H), 1.29 (s, 6H), 1.27 (s, 6H), 0.61 (s, 3H), 0.57 (s, 3H); 13C NMR (CDCl₃, 101 MHz) δ 171.1 (C), 155.1 (C), 144.2 (C), 136.6 (C), 133.6 (CH), 131.9 (C), 130.8 (CH), 128.7 (CH), 127.8 (C), 127.0 (C), 125.6 (CH), 124.5 (CH), 123.8 (C), 122.2 (CH), 114.4 (CH), 92.3 (C), 56.6 (C), 30.8 (CH₃), 28.2 (CH₃), 27.6 (CH₃), 18.2 (CH₃), 0.5 (CH₃), −1.2 (CH₃); Analytical HPLC: t_{R}=12.2 min, >99% purity (30-95% MeCN/H₂O, linear gradient, with constant 0.1% v/v TFA additive; 20 min run; 1 mL/min flow; ESI; positive ion mode; detection at 725 nm); HRMS (ESI) calcd for C₃₆H₄₁N₂O₂Si [M+H]⁺ 561.2932, found 561.2921.

### Example 55: Rhodamine Synthesis Via Lactol (Phthalaldehydic Acid) Condensation (Scheme 5): 4⁻Carboxy-2⁻(1,2,2,4,8,10,10,11,13,13⁻decamethyl-2,10,11,13-tetrahydrosilino[3,2⁻g:5,6⁻g′]diquinolin-6⁻ylium-6(1H)-yl)benzoate (110)

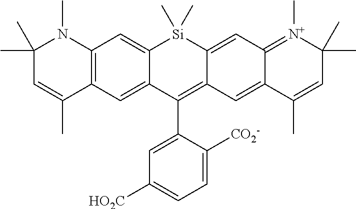

Dimethylbis(1,2,2,4⁻tetramethyl-1,2⁻dihydroquinolin-7⁻yl)silane (37; 300 mg, 0.697 mmol) and 4⁻carboxyphthalaldehydic acid (109; 203 mg, 1.04 mmol, 1.5 eq) were combined in 2,2,3,3,4,4,4⁻heptafluoro-1⁻butanol (3.5 mL) in a crimp-top vial. The reaction mixture was sparged with O₂ from a balloon for 10 min, then stirred at 95° C. under the O₂ balloon for 24 h. The reaction was cooled to room temperature, concentrated in vacuo, and purified twice by silica gel chromatography (10-100% EtOAc/hexanes, linear gradient with constant 0.1% v/v AcOH additive) to provide 110 as a green solid (63.4 mg, 15%). An analytically pure sample for spectral characterization was obtained by reverse phase HPLC (30-70% MeCN/H₂O, linear gradient, with constant 0.1% TFA). ¹H NMR (CD₃OD, 400 MHz, TFA salt) δ 8.23 (dd, J=8.0, 1.3 Hz, 1H), 8.06 (dd, J=8.0, 0.7 Hz, 1H), 7.87 (dd, J=1.3, 0.7 Hz, 1H), 6.84 (s, 2H), 6.54 (s, 2H), 5.31 (q, J=1.5 Hz, 2H), 2.91 (s, 6H), 1.61 (d, J=1.4 Hz, 6H), 1.29 (s, 6H), 1.27 (s, 6H), 0.65 (s, 3H), 0.56 (s, 3H); 13C NMR (CD₃OD, 101 MHz, TFA salt) δ 171.7 (C), 168.5 (C), 154.9 (C), 146.7 (C), 139.3 (C), 138.0 (C), 132.4 (CH), 131.8 (C), 131.25 (C), 131.21 (CH), 128.2 (C), 127.26 (CH), 127.21 (CH), 125.1 (C), 123.8 (CH), 116.4 (CH), 58.2 (C), 31.4 (CH₃), 27.9 (CH₃), 18.1 (CH₃), 0.0 (CH₃), −1.1 (CH₃); Analytical HPLC: t_{R}=12.7 min, >99% purity (10-95% MeCN/H₂O, linear gradient, with constant 0.1% v/v TFA additive; 20 min run; 1 mL/min flow; ESI; positive ion mode; detection at 725 nm); HRMS (ESI) calcd for C₃₇H₄₁N₂O₄Si [M+H]⁺ 605.2830, found 605.2816.

### Example 56: Rhodamine Synthesis Via Lactol (Phthalaldehydic Acid) Condensation (Scheme 5): SiRF₆₆₇ (22)

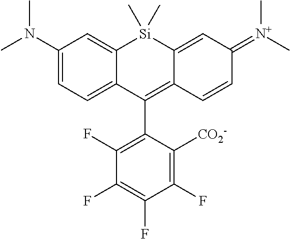

3,3′-(Dimethylsilanediyl)bis(N,N-dimethylaniline)¹ (23; 200 mg, 0.670 mmol) and tetrafluoro-phthalaldehydic acid (5; 446 mg, 2.01 mmol, 3 eq) were combined in 2,2,3,3,4,4,4-heptafluoro-1⁻butanol (2.2 mL) in a crimp-top vial. The reaction mixture was sparged with O₂ from a balloon for 10 min, then stirred at 95° C. under the O₂ balloon for 72 h. The reaction was cooled to room temperature, diluted with saturated NaHCO₃, and extracted with EtOAc (2×). The combined organic extracts were washed with brine, dried over anhydrous MgSO₄, filtered, and concentrated in vacuo. Silica gel chromatography (0-50% EtOAc/hexanes, linear gradient) provided 22 as a blue-green solid (194 mg, 58%). The characterization data for 22 matched the previously reported spectra.¹

### Example 57: Rhodamine Synthesis Via Lactol (Phthalaldehydic Acid) Condensation (Scheme 5): JFX₅₇₆ (8)

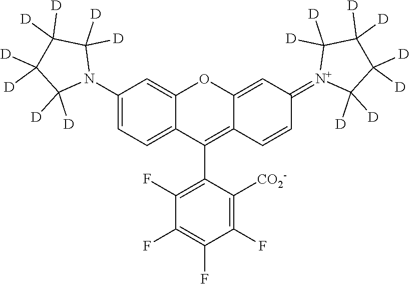

Via lactol condensation route: 1,1′-(Oxybis(3,1⁻phenylene))bis(pyrrolidine-2,2,3,3,4,4,5,5⁻d₈) (9; 248 mg, 0.764 mmol) and tetrafluorophthalaldehydic acid (5; 170 mg, 0.764 mmol, 1 eq) were combined in 2,2,2⁻trifluoroethanol (15 mL) in a round-bottom flask. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-10% MeOH (2 M NH₃)/CH₂Cl₂, linear gradient) to provide 8 as a dark purple solid (343 mg, 85%).

Via dibromide route: A solution of 1,1′-(oxybis(4⁻bromo-3,1-phenylene))bis(pyrrolidine-2,2,3,3,4,4,5,5⁻d₈) (7; 300 mg, 0.622 mmol) in THE (15 mL) was cooled to −78° C. under nitrogen. tert-Butyllithium (1.7 M in pentane, 1.61 mL, 2.74 mmol, 4.4 eq) was added, and the reaction was stirred at −78° C. for 30 min. It was then warmed to −10° C. before adding a solution of MgBr₂ OEt₂ (353 mg, 1.37 mmol, 2.2 eq) in THE (10 mL). After an additional 30 min at −10° C., a solution of tetrafluorophthalic anhydride (4; 301 mg, 1.37 mmol, 2.2 eq) in THE (10 mL) was added dropwise over 30 min via addition funnel. The reaction was then allowed to warm to room temperature overnight (18 h). Following the addition of AcOH (500 L), the mixture was diluted with MeOH, deposited onto Celite, and concentrated to dryness. Silica gel chromatography (0-10% MeOH (2 M NH₃)/CH₂Cl₂, linear gradient; dry load with Celite) afforded 113 mg (34%) of 8 as a dark purple solid.

¹H NMR (CD₃OD, 400 MHz) δ 7.34 (dd, J=9.3, 0.9 Hz, 1H), 6.93 (dd, J=9.4, 2.3 Hz, 1H), 6.76 (d, J=2.3 Hz, 1H); 19F NMR (CD₃OD, 376 MHz) δ−139.00 (ddd, J=21.1, 12.5, 4.1 Hz, 1F), −140.94 (ddd, J=22.4, 12.8, 3.7 Hz, 1F), −153.55 (ddd, J=22.7, 19.1, 4.1 Hz, 1F), −157.66-−157.87 (m, 1F); Analytical HPLC: t_{R}=12.4 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₂₈H₇D₁₆F₄N₂O₃[M+H]⁺ 527.2644, found 527.2640.

### Example 58: Rhodamine Synthesis Via Lactol (Phthalaldehydic Acid) Condensation (Scheme 5): 6⁻Carboxy-JFX554 (S24)

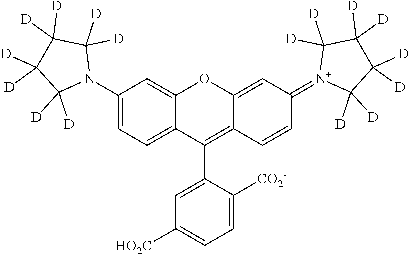

1,1′-(Oxybis(3,1⁻phenylene))bis(pyrrolidine-2,2,3,3,4,4,5,5⁻d₈) (9; 45 mg, 0.139 mmol) and 4⁻carboxyphthalaldehydic acid (109; 40.4 mg, 0.208 mmol, 1.5 eq) were combined in 2,2,2⁻trifluoroethanol (3 mL) in a crimp-top vial. The reaction mixture was sparged with O₂ from a balloon for 10 min, then stirred at 80° C. under the O₂ balloon for 24 h. The reaction was cooled to room temperature, concentrated in vacuo, and purified by silica gel chromatography (0-20% MeOH/CH₂Cl₂, linear gradient, with constant 1% v/v AcOH additive) to provide S24 as a dark red solid (61.2 mg, 79%, acetate salt). The characterization data for S24 matched the previously reported spectra.⁴⁵

### Example 59: Rhodamine Synthesis Via Lactol (Phthalaldehydic Acid) Condensation (Scheme 5): JFX₆₇₃ (25)

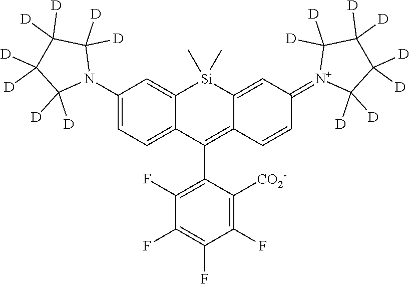

Via lactol condensation route: Dimethylbis(3⁻(pyrrolidin-1⁻yl-d₈)phenyl)silane (26; 250 mg, 0.682 mmol) and tetrafluorophthalaldehydic acid (5; 151 mg, 0.682 mmol, 1 eq) were combined in 2,2,3,3,4,4,4⁻heptafluoro-1⁻butanol (3 mL) in a crimp-top vial. The reaction mixture was sparged with O₂ from a balloon for 10 min, then stirred at 95° C. under the O₂ balloon for 48 h. The reaction was cooled to room temperature, concentrated in vacuo, and purified by silica gel chromatography (0-75% EtOAc/hexanes, linear gradient) to provide 25 as a blue solid (247 mg, 64%).

Via dibromide route: A solution of bis(2⁻bromo-5⁻(pyrrolidin-1⁻yl-d₈)phenyl)dimethylsilane (24; 600 mg, 1.14 mmol) in THE (40 mL) was cooled to −78° C. under nitrogen. tert-Butyllithium (1.7 M in pentane, 2.96 mL, 5.03 mmol, 4.4 eq) was added, and the reaction was stirred at −78° C. for 30 min. It was then warmed to −10° C. before adding a solution of MgBr₂ OEt₂ (650 mg, 2.52 mmol, 2.2 eq) in THF (10 mL). After an additional 30 min at −10° C., a solution of tetrafluorophthalic anhydride (4; 554 mg, 2.52 mmol, 2.2 eq) in THE (10 mL) was added dropwise over 30 min via addition funnel. The reaction was then allowed to warm to room temperature overnight (18 h). It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. Silica gel chromatography (0-75% EtOAc/hexanes, linear gradient) afforded 326 mg (50%) of 25 as a blue solid.

¹H NMR (CDCl₃, 400 MHz) δ 6.78 (d, J=2.9 Hz, 2H), 6.77 (d, J=8.6 Hz, 2H), 6.45 (dd, J=8.8, 2.7 Hz, 2H), 0.58 (s, 3H), 0.55 (s, 3H); 19F NMR (CDCl₃, 376 MHz) δ−139.43-−139.69 (m, 2F), −144.25-−144.45 (m, 1F), −152.39-−152.57 (m, 1F); Analytical HPLC: t_{R}=13.4 min, 98.8% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₃₀H₁₃D₁₆F₄N₂O₂Si [M+H]⁺ 569.2933, found 569.2943.

### Example 60: Rhodamine Synthesis Via Lactol (Phthalaldehydic Acid) Condensation (Scheme 5): 6⁻Carboxy-JFX₆₅₀ (S25)

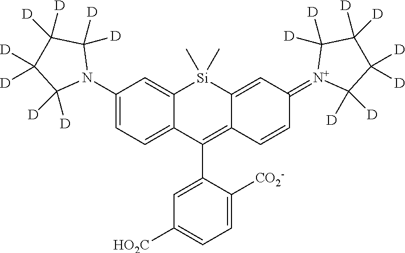

Dimethylbis(3⁻(pyrrolidin-1⁻yl-d₈)phenyl)silane (26; 250 mg, 0.682 mmol) and 4-carboxyphthalaldehydic acid (109; 132 mg, 0.682 mmol, 1 eq) were combined in 2,2,3,3,4,4,4-heptafluoro-1⁻butanol (2.5 mL) in a crimp-top vial. The reaction mixture was sparged with O₂ from a balloon for 10 min, then stirred at 95° C. under the O₂ balloon for 72 h. The reaction was cooled to room temperature, concentrated in vacuo, and purified by silica gel chromatography (10-100% EtOAc/hexanes, linear gradient, with constant 0.1% v/v AcOH additive) to provide S25 as a blue solid (139 mg, 38%). The characterization data for S25 matched the previously reported spectra.⁴⁵

### Example 61: Preparation of 2,2⁻Diarylpropane Intermediates (Scheme 10): Bis(3⁻bromophenyl)methanol (S29)

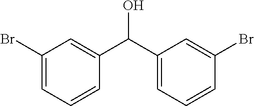 [SMILES: OC(c1cccc(Br)c1)c1cccc(Br)c1]

A solution of 1,3⁻dibromobenzene (S27; 14.02 g, 59.45 mmol, 1.1 eq) in THE (100 mL) was cooled to −78° C. under nitrogen. n-Butyllithium (2.5 M in hexanes, 23.78 mL, 59.45 mmol, 1.1 eq) was added, and the reaction was stirred at −78° C. for 30 min. 3-Bromobenzaldehye (S28; 10.00 g, 54.05 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-50% Et₂O/hexanes, linear gradient) afforded 16.15 g (92%) of S29 as a colorless oil. ¹H NMR (CDCl₃, 400 MHz) δ 7.54 (t, J=1.9 Hz, 2H), 7.42 (ddd, J=7.8, 2.0, 1.2 Hz, 2H), 7.30-7.26 (m, 2H), 7.22 (t, J=7.7 Hz, 2H), 5.76 (s, 1H), 2.25 (s, 1H); 13C NMR (CDCl₃, 101 MHz) δ 145.4 (C), 131.1 (CH), 130.4 (CH), 129.6 (CH), 125.3 (CH), 122.9 (C), 75.1 (CH); HRMS (EI) calcd for C₁₃H₁₀Br₂O [M]^{+·}339.9098, found 339.3088.

### Example 62: Preparation of 2,2⁻Diarylpropane Intermediates (Scheme 10): Bis(3⁻bromophenyl)methanone (S30)

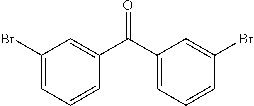 [SMILES: O=C(c1cccc(Br)c1)c1cccc(Br)c1]

To a solution of bis(3⁻bromophenyl)methanol (S29; 11.80 g, 34.50 mmol) in CH₂Cl₂ (100 mL) was added MnO₂ (50.00 g) in two portions. After stirring the reaction at room temperature for 18 h, it was filtered through Celite with CH₂Cl₂ and concentrated in vacuo. The resulting white solid was recrystallized from hot EtOAc to yield 11.20 g (95%) of S30 as white crystals. ¹H NMR (CDCl₃, 400 MHz) δ 7.93 (t, J=1.8 Hz, 2H), 7.74 (ddd, J=8.0, 2.1, 1.1 Hz, 2H), 7.71-7.67 (m, 2H), 7.38 (t, J=7.9 Hz, 2H); 13C NMR (CDCl₃, 101 MHz) δ 193.8 (C), 138.9 (C), 135.9 (CH), 132.9 (CH), 130.2 (CH), 128.7 (CH), 123.0 (C); HRMS (EI) calcd for C13H₈Br₂O [M]^{+·}337.8942, found 337.8947.

### Example 63: Preparation of 2,2⁻Diarylpropane Intermediates (Scheme 10): 3,3′-(Propane-2,2⁻diyl)bis(bromobenzene) (S31)

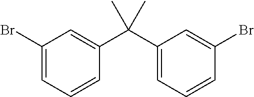 [SMILES: CC(C)(c1cccc(Br)c1)c1cccc(Br)c1]

An oven-dried 500 mL 3⁻neck round-bottom flask equipped with two addition funnels was charged with CH₂Cl₂ (100 mL) under nitrogen and cooled to −40° C. Solutions of TiCl₄ (1 M in CH₂Cl₂, 80.00 mL, 80.00 mmol, 4 eq) followed by Me₂Zn (2 M in toluene, 40.00 mL, 80.00 mmol, 4 eq) were successively added dropwise via the addition funnels. The reaction was stirred at −40° C. to −30° C. for 20 min. A solution of bis(3⁻bromophenyl)methanone (S30; 6.80 g, 20.00 mmol) in CH₂Cl₂ (100 mL) was added via cannula; the resulting suspension was then warmed to room temperature and vigorously stirred for 3 h. The mixture was carefully diluted with water (˜200 mL) and extracted with CH₂Cl₂ (2×). The combined organic extracts were dried over anhydrous MgSO₄, filtered through Celite, and concentrated in vacuo. Silica gel chromatography (100% hexanes, isocratic) afforded 6.83 g (96%) of S31 as a colorless oil. 1H NMR (CDCl₃, 400 MHz) δ 7.37 (t, J=1.8 Hz, 2H), 7.33 (ddd, J=7.6, 2.0, 1.3 Hz, 2H), 7.14 (t, J=7.8 Hz, 2H), 7.11-7.08 (m, 2H), 1.64 (s, 6H); 13C NMR (CDCl₃, 101 MHz) δ 152.4 (C), 129.90 (CH), 129.86 (CH), 129.2 (CH), 125.8 (CH), 122.6 (C), 43.3 (C), 30.6 (CH₃); HRMS (EI) calcd for C₁₅H₁₄Br₂ [M]^{+·}351.9462, found 351.9480.

### Example 64: Preparation of 2,2⁻Diarylpropane Intermediates (Scheme 10): 1,1′-(Propane-2,2⁻diylbis(3,1⁻phenylene))bis(pyrrolidine-2,2,3,3,4,4,5,5⁻d₈) (41)

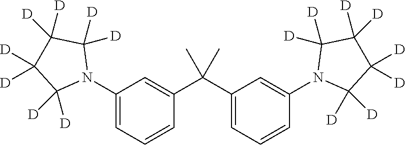 [SMILES: [2H]C1([2H])N(c2cccc(C(C)(C)c3cccc(N4C([2H])([2H])C([2H])([2H])C([2H])([2H])C4([2H])[2H])c3)c2)C([2H])([2H])C([2H])([2H])C1([2H])[2H]]

An oven-dried crimp-top vial was charged with CuI (108 mg, 0.565 mmol, 0.2 eq), L-proline (130 mg, 1.13 mmol, 0.4 eq), and K₂CO₃ (1.56 g, 11.30 mmol, 4 eq). The flask was sealed and evacuated/backfilled with nitrogen (3×). A solution of 3,3′-(propane-2,2-diyl)bis(bromobenzene) (S31; 1.00 g, 2.82 mmol) in DMSO (11 mL) was added, and the reaction was flushed again with nitrogen (3×). Following the addition of pyrrolidine-2,2,3,3,4,4,5,5⁻ds (1.42 mL, 16.94 mmol, 6 eq), the reaction was stirred at 100° C. for 18 h. It was then cooled to room temperature, diluted with saturated NH₄Cl, and extracted with EtOAc (2×). The combined organic extracts were washed with water and brine, dried over anhydrous MgSO₄, filtered, and concentrated in vacuo. Purification by flash chromatography on silica gel (0-20% Et₂O/hexanes, linear gradient) afforded 809 mg (82%) of 41 as a white solid. ¹H NMR (CDCl₃, 400 MHz) δ 7.10 (t, J=7.9 Hz, 2H), 6.58-6.52 (m, 2H), 6.51 (t, J=2.1 Hz, 2H), 6.42-6.35 (m, 2H), 1.67 (s, 6H); 13C NMR (CDCl₃, 101 MHz) δ 152.0 (C), 147.9 (C), 128.7 (CH), 114.9 (CH), 110.4 (CH), 109.0 (CH), 43.3 (C), 31.0 (CH₃); HRMS (ESI) calcd for C₂₃H₁₅D₁₆N₂[M+H]⁺ 351.3486, found 351.3478.

### Example 65: Preparation of 2,2⁻Diarylpropane Intermediates (Scheme 10): 1,1′-(Propane-2,2⁻diylbis(4⁻bromo-3,1⁻phenylene))bis(pyrrolidine-2,2,3,3,4,4,5,5⁻d₈) (39)

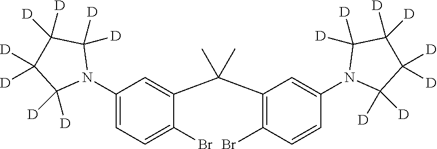 [SMILES: [2H]C1([2H])N(c2ccc(Br)c(C(C)(C)c3cc(N4C([2H])([2H])C([2H])([2H])C([2H])([2H])C4([2H])[2H])ccc3Br)c2)C([2H])([2H])C([2H])([2H])C1([2H])[2H]]

1,1′-(Propane-2,2⁻diylbis(3,1⁻phenylene))bis(pyrrolidine-2,2,3,3,4,4,5,5⁻d₈) (41; 1.19 g, 3.39 mmol) was taken up in DMF (30 mL) and cooled to 0° C. N-Bromosuccinimide (1.21 g, 6.79 mmol, 2 eq) was added portion-wise over 5 min, and the reaction was subsequently stirred at 0° C. for 2 h. After removing DMF by rotary evaporation, the resulting residue was diluted with water and extracted with CH₂Cl₂ (2×). The combined organic extracts were washed with water and brine, dried over anhydrous MgSO₄, filtered, and concentrated in vacuo. Purification by silica gel chromatography (0-100% CH₂Cl₂/hexanes, linear gradient) afforded 716 mg (41%) of 39 as a white solid. ¹H NMR (CDCl₃, 400 MHz) δ 7.24 (d, J=8.7 Hz, 2H), 6.82 (d, J=2.9 Hz, 2H), 6.29 (dd, J=8.6, 2.9 Hz, 2H), 1.81 (s, 6H); 13C NMR (CDCl₃, 101 MHz) δ 147.41 (C), 147.36 (C), 135.1 (CH), 112.5 (CH), 111.3 (CH), 108.1 (C), 46.2 (C), 29.5 (CH₃); HRMS (ESI) calcd for C₂₃H₁₃D₁₆Br₂N₂[M+H]⁺ 509.1676, found 509.1668.

### Example 66: Preparation of 2,2⁻Diarylpropane Intermediates (Scheme 10): 3,3′-(Propane-2,2⁻diyl)dianiline (S33)

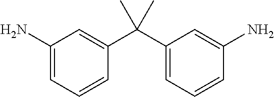 [SMILES: CC(C)(c1cccc(N)c1)c1cccc(N)c1]

Step 1: An oven-dried round-bottom flask was charged with tert-butyl carbamate (4.76 g, 40.67 mmol, 2.4 eq), CuI (3.23 g, 16.94 mmol, 1 eq), and K₃PO ₄ (10.79 g, 50.83 mmol, 3 eq). The flask was sealed and evacuated/backfilled with nitrogen (3×). A solution of 3,3′-(propane-2,2⁻diyl)bis(bromobenzene) (S31; 6.00 g, 16.94 mmol) in dioxane (60 mL) was added via cannula, and the reaction was flushed again with nitrogen (3×). Following the addition of N,N-dimethylethylenediamine (1.82 mL, 16.94 mmol, 1 eq), the reaction was stirred at 110° C. for 18 h. It was then cooled to room temperature, diluted with EtOAc (˜250 mL) and saturated NH₄Cl (˜250 mL), and vigorously stirred for 15 min while open to air. The resulting suspension was filtered; the filter cake was washed (water, EtOAc) and dried to provide a large crop of white powder that was identified as the desired dicarbamate product by NMR and LC/MS analysis. The filtrate was partitioned, and the aqueous layer was extracted again with EtOAc (3×). The combined organic extracts were washed with brine, dried over anhydrous MgSO₄, filtered, and evaporated. The white residue was triturated with Et₂O, filtered, washed with additional Et₂O, and dried to afford a second, smaller crop of the product. The two batches were combined to yield a total of 6.39 g (88%) of di-tert-butyl (propane-2,2⁻diylbis(3,1-phenylene))dicarbamate (S32) as a white solid.

Step 2: The dicarbamate (S32; 6.39 g, 14.98 mmol) was suspended in CH₂Cl₂ (60 mL), and trifluoroacetic acid (12 mL) was added. The reaction was stirred at room temperature for 4 h. It was then diluted with toluene (60 mL) and concentrated to dryness. The residue was taken up in saturated NaHCO₃ and extracted with EtOAc (2×). The combined organic extracts were washed with brine, dried over anhydrous MgSO₄, filtered, and evaporated. Flash chromatography (10-100% EtOAc/hexanes, linear gradient) afforded 2.34 g (69%) of S33 as an off-white solid. ¹H NMR (CDCl₃, 400 MHz) δ 7.06 (t, J=7.8 Hz, 2H), 6.68 (ddd, J=7.8, 1.8, 1.0 Hz, 2H), 6.54 (t, J=2.0 Hz, 2H), 6.51 (ddd, J=7.8, 2.3, 1.0 Hz, 2H), 3.56 (s, 4H), 1.60 (s, 6H); 13C NMR (CDCl₃, 101 MHz) δ 152.2 (C), 146.1 (C), 128.9 (CH), 117.4 (CH), 114.3 (CH), 112.7 (CH), 42.9 (C), 30.7 (CH₃); HRMS (ESI) calcd for C₁₅H₁₉N₂[M+H]⁺ 227.1543, found 227.1544.

### Example 67: Preparation of 2,2⁻Diarylpropane Intermediates (Scheme 10): 8,8′-(Propane-2,2⁻diyl)bis(2,3,6,7⁻tetrahydro-1H,5H-pyrido[3,2,1⁻ij]quinoline) (56)

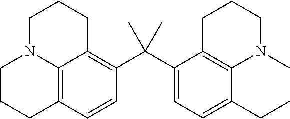 [SMILES: CCc1ccc(C(C)(C)c2ccc3c4c2CCCN4CCC3)c2c1N(C)CCC2]

A mixture of 3,3′-(propane-2,2⁻diyl)dianiline (S33; 2.30 g, 10.16 mmol), Na₂CO₃ (8.62 g, 81.30 mmol, 8 eq), and 1⁻bromo-3⁻chloropropane (24.15 mL, 243.9 mmol, 24 eq) was stirred at 140° C. for 48 h. The gummy, heterogeneous mixture that resulted was cooled to room temperature, diluted with water, and extracted with EtOAc (2×). The organic extracts were washed with brine, dried over anhydrous MgSO₄, filtered, and concentrated in vacuo. The resulting red residue was taken back up in DMF (15 mL) and stirred at 160° C. for 18 h. The reaction was cooled to room temperature, diluted with saturated NaHCO₃, and extracted with CH₂Cl₂ (2×). The CH₂Cl₂ extracts were dried over anhydrous MgSO₄, filtered, and evaporated. Silica gel chromatography (0-20% Et₂O/hexanes, linear gradient) yielded 2.04 g (52%) of 56 as a white solid. ¹H NMR (CDCl₃, 400 MHz) δ 6.76 (AB quartet, v_{A}=2710.7 Hz, v_{B}=2693.5 Hz, J_{AB}=8.0 Hz, 4H), 3.09 (t, J=5.7 Hz, 4H), 2.97 (t, J=6.3 Hz, 4H), 2.73 (t, J=6.5 Hz, 4H), 2.42-1.82 (m, 8H), 1.65-1.54 (m, 10H) ¹³C NMR (CDCl₃, 101 MHz) δ 146.0 (C), 143.5 (C), 126.7 (CH), 121.7 (C), 119.7 (C), 113.1 (CH), 51.0 (CH₂), 50.2 (CH₂), 43.6 (C), 31.4 (CH₃), 28.2 (CH₂), 25.3 (CH₂), 22.4 (CH₂); HRMS (ESI) calcd for C₂₇H₃₅N₂[M+H]⁺ 387.2795, found 387.2796.

### Example 68: Preparation of 2,2⁻Diarylpropane Intermediates (Scheme 10): (3⁻Bromophenyl)(3⁻methoxyphenyl)methanol (S35)

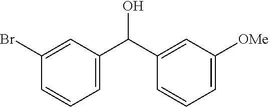 [SMILES: COc1cccc(C(O)c2cccc(Br)c2)c1]

A solution of 1,3⁻dibromobenzene (S27; 25.00 g, 106.0 mmol, 1.2 eq) in THE (200 mL) was cooled to −78° C. under nitrogen. n-Butyllithium (2.7 M in hexanes, 39.25 mL, 106.0 mmol, 1.2 eq) was added, and the reaction was stirred at −78° C. for 30 min. m-Anisaldehyde (S34; 10.76 mL, 88.31 mmol) was then added. The dry ice bath was removed, and the reaction was stirred at room temperature for 2 h. It was subsequently quenched with saturated NH₄Cl, diluted with water, and extracted with EtOAc (2×). The combined organic extracts were washed with brine, dried over anhydrous MgSO₄, filtered, and concentrated in vacuo. Purification by flash chromatography on silica gel (0-50% Et₂O/hexanes, linear gradient) afforded 22.23 g (86%) of S35 as a pale yellow oil. ¹H NMR (CDCl₃, 400 MHz) δ 7.56 (t, J=1.9 Hz, 1H), 7.39 (ddd, J=7.9, 2.1, 1.2 Hz, 1H), 7.31-7.27 (m, 1H), 7.26 (t, J=8.2 Hz, 1H), 7.19 (t, J=7.8 Hz, 1H), 6.95-6.90 (m, 2H), 6.82 (ddd, J=8.4, 2.5, 1.2 Hz, 1H), 5.77 (d, J=3.4 Hz, 1H), 3.79 (s, 3H), 2.22 (d, J=3.4 Hz, 1H); 13C NMR (CDCl₃, 101 MHz) δ 160.0 (C), 146.0 (C), 145.0 (C), 130.8 (CH), 130.2 (CH), 129.9 (CH), 129.6 (CH), 125.2 (CH), 122.8 (C), 119.0 (CH), 113.4 (CH), 112.4 (CH), 75.7 (CH), 55.4 (CH₃); HRMS (EI) calcd for C₁₄H₁₃BrO₂ [M]^{+·}292.0094, found 292.0088.

### Example 69: Preparation of 2,2⁻Diarylpropane Intermediates (Scheme 10): (3⁻Bromophenyl)(3⁻methoxyphenyl)methanone (S36)

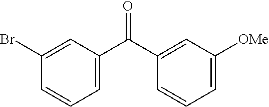 [SMILES: COc1cccc(C(=O)c2cccc(Br)c2)c1]

To a solution of (3⁻bromophenyl)(3⁻methoxyphenyl)-methanol (S35; 21.75 g, 74.19 mmol) in CH₂Cl₂ (400 mL) was added MnO₂ (100 g, portion-wise over 10 min). After stirring the reaction at room temperature for 18 h, it was filtered through Celite with CH₂Cl₂ and concentrated in vacuo. Silica gel chromatography (0-40% Et₂O/hexanes, linear gradient) yielded 20.34 g (94%) of S36 as a viscous, colorless oil that crystallized into a white solid upon standing.

¹H NMR (CDCl₃, 400 MHz) δ 7.94 (t, J=1.8 Hz, 1H), 7.74-7.69 (m, 2H), 7.40 (t, J=7.2 Hz, 1H), 7.38-7.33 (m, 2H), 7.31 (dt, J=7.6, 1.3 Hz, 1H), 7.16 (ddd, J=8.2, 2.7, 1.1 Hz, 1H), 3.87 (s, 3H); 13C NMR (CDCl₃, 101 MHz) δ 195.1 (C), 159.9 (C), 139.7 (C), 138.4 (C), 135.4 (CH), 132.9 (CH), 130.0 (CH), 129.5 (CH), 128.7 (CH), 123.0 (CH), 122.7 (C), 119.5 (CH), 114.4 (CH), 55.6 (CH₃); HRMS (ESI) calcd for C₁₄H₁₂BrO₂ [M+H]⁺ 291.0015, found 291.0021.

### Example 70: Preparation of 2,2⁻Diarylpropane Intermediates (Scheme 10): 1⁻Bromo-3⁻(2⁻(3⁻methoxyphenyl)propan-2⁻yl)benzene (S37)

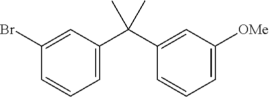 [SMILES: COc1cccc(C(C)(C)c2cccc(Br)c2)c1]

An oven-dried 1 L 3⁻neck round-bottom flask equipped with two addition funnels was charged with CH₂Cl₂ (150 mL) under nitrogen and cooled to −30° C. Solutions of TiCl₄ (1 M in CH₂Cl₂, 100.0 mL, 100.0 mmol, 4 eq) followed by Me₂Zn (1.2 M in toluene, 83.33 mL, 100.0 mmol, 4 eq) were successively added dropwise via the addition funnels. The reaction was stirred at −30° C. for 20 min. A solution of (3⁻bromophenyl)(3⁻methoxyphenyl)methanone (S36; 7.28 g, 25.00 mmol) in CH₂Cl₂ (50 mL) was added dropwise via cannula; the resulting brown suspension was then warmed to room temperature and vigorously stirred for 3 h. The mixture was carefully diluted with water (˜400 mL) and extracted with CH₂Cl₂ (2×). The combined organic extracts were dried over anhydrous MgSO₄, filtered through Celite, and concentrated in vacuo. Silica gel chromatography (0-10% Et₂O/hexanes, linear gradient) afforded 7.23 g (95%) of S37 as a viscous, colorless oil. ¹H NMR (CDCl₃, 400 MHz) δ 7.41-7.37 (m, 1H), 7.33-7.27 (m, 1H), 7.23-7.17 (m, 1H), 7.14-7.09 (m, 2H), 6.81-6.76 (m, 2H), 6.75-6.71 (m, 1H), 3.77 (s, 3H), 1.65 (s, 6H); 13C NMR (CDCl₃, 101 MHz) δ 159.5 (C), 153.1 (C), 151.7 (C), 129.9 (CH), 129.7 (CH), 129.2 (CH), 129.0 (CH), 125.8 (CH), 122.5 (C), 119.5 (CH), 113.6 (CH), 110.5 (CH), 55.3 (CH₃), 43.2 (C), 30.7 (CH₃); HRMS (EI) calcd for C₁₆H₁₇BrO [M]^{+·}304.0458, found 304.0469.

### Example 71: Preparation of 2,2⁻Diarylpropane Intermediates (Scheme 10)

### tert-Butyl (3⁻(2⁻(3⁻methoxyphenyl)propan-2⁻yl)phenyl)carbamate (S38)

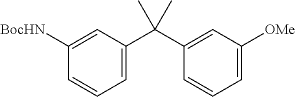 [SMILES: COc1cccc(C(C)(C)c2cccc(NC(=O)OC(C)(C)C)c2)c1]

An oven-dried round-bottom flask was charged with tert-butyl carbamate (4.61 g, 39.32 mmol, 1.2 eq), CuI (3.12 g, 16.38 mmol, 0.5 eq), and K₃PO ₄ (10.43 g, 49.14 mmol, 1.5 eq). The flask was sealed and evacuated/backfilled with nitrogen (3×). A solution of 1⁻bromo-3-(2⁻(3⁻methoxyphenyl)propan-2⁻yl)benzene (S37; 10.00 g, 32.76 mmol) in dioxane (100 mL) was added via cannula, and the reaction was flushed again with nitrogen (3×). Following the addition of N,N′-dimethylethylenediamine (1.76 mL, 16.38 mmol, 0.5 eq), the reaction was stirred at 110° C. for 18 h. It was then cooled to room temperature, stirred open to air for 3 h, filtered through Celite with EtOAc, and concentrated in vacuo. Purification of the crude residue by flash chromatography (0-25% EtOAc/hexanes, linear gradient) yielded 10.42 g (93%) of S38 as a viscous, colorless gum that crystallized into a white solid upon standing. ¹H NMR (CDCl₃, 400 MHz) δ 7.33 (d, J=8.0 Hz, 1H), 7.18 (td, J=8.1, 2.0 Hz, 2H), 7.05 (t, J=2.0 Hz, 1H), 6.90 (ddd, J=7.8, 1.9, 1.0 Hz, 1H), 6.83-6.77 (m, 2H), 6.71 (ddd, J=8.1, 2.4, 1.1 Hz, 1H), 6.40 (s, 1H), 3.75 (s, 3H), 1.64 (s, 6H), 1.49 (s, 9H); 13C NMR (CDCl₃, 101 MHz) δ 159.4 (C), 152.9 (C), 152.4 (C), 151.6 (C), 138.2 (C), 129.0 (CH), 128.8 (CH), 121.8 (CH), 119.6 (CH), 117.3 (CH), 116.2 (CH), 113.5 (CH), 110.4 (CH), 80.5 (C), 55.3 (CH₃), 43.1 (C), 30.8 (CH₃), 28.5 (CH₃); HRMS (ESI) calcd for C₂1H₂₇NO₃Na [M+Na]⁺364.1883, found 364.1884.

### Example 72: Preparation of 2,2⁻Diarylpropane Intermediates (Scheme 10): 3⁻(2⁻(3⁻Methoxyphenyl)propan-2⁻yl)aniline (S39)

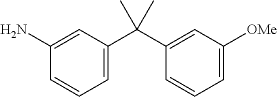 [SMILES: COc1cccc(C(C)(C)c2cccc(N)c2)c1]

tert-Butyl (3⁻(2⁻(3⁻methoxyphenyl)propan-2⁻yl)phenyl)-carbamate (S38; 10.30 g, 30.17 mmol) was taken up in CH₂Cl₂ (200 mL), and trifluoroacetic acid (40 mL) was added. The reaction was stirred at room temperature for 2 h. It was then diluted with toluene (100 mL) and concentrated to dryness. The residue was taken up in saturated NaHCO₃ and extracted with EtOAc (2×). The combined organic extracts were washed with brine, dried over anhydrous MgSO₄, filtered, and concentrated in vacuo. Flash chromatography (0-4₀% EtOAc/hexanes, linear gradient) afforded 6.72 g (92%) of S39 as an off-white solid. ¹H NMR (CDCl₃, 400 MHz) δ 7.18 (t, J=8.2 Hz, 1H), 7.05 (t, J=7.8 Hz, 1H), 6.84-6.80 (m, 2H), 6.71 (ddd, J=8.2, 2.5, 1.0 Hz, 1H), 6.66 (ddd, J=7.8, 1.9, 1.0 Hz, 1H), 6.53 (t, J=2.1 Hz, 1H), 6.50 (ddd, J=7.8, 2.4, 1.0 Hz, 1H), 3.76 (s, 3H), 3.45 (s, 2H), 1.63 (s, 6H); 13C NMR (CDCl₃, 101 MHz) δ 159.4 (C), 152.7 (C), 151.9 (C), 146.1 (C), 128.97 (CH), 128.96 (CH), 119.7 (CH), 117.4 (CH), 114.2 (CH), 113.5 (CH), 112.8 (CH), 110.3 (CH), 55.3 (CH₃), 43.0 (C), 30.7 (CH₃); HRMS (ESI) calcd for C₁₆H₂₀NO [M+H]⁺ 242.1539, found 242.1541.

### Example 73: Preparation of 2,2⁻Diarylpropane Intermediates (Scheme 10): 7⁻(2⁻(3⁻Methoxyphenyl)propan-2⁻yl)-2,2,4⁻trimethyl-1,2⁻dihydroquinoline (S40)

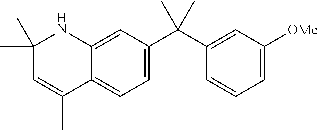 [SMILES: COc1cccc(C(C)(C)c2ccc3c(c2)NC(C)(C)C=C3C)c1]

3⁻(2⁻(3⁻Methoxyphenyl)-propan-2⁻yl)aniline (S39; 2.20 g, 9.12 mmol) and iodine (463 mg, 1.82 mmol, 0.2 eq) were combined in acetone (50 mL) and stirred at reflux for 18 h. The reaction was then cooled to room temperature, concentrated to dryness, and purified by silica gel chromatography (0-20% Et₂O/hexanes, linear gradient) to afford 1.91 g (65%) of S40 as a colorless oil that crystallized into an off-white solid upon standing. ¹H NMR (CDCl₃, 400 MHz) δ 7.17 (t, J=8.2 Hz, 1H), 6.95 (d, J=8.0 Hz, 1H), 6.85-6.81 (m, 2H), 6.70 (ddd, J=8.1, 2.5, 1.0 Hz, 1H), 6.52 (dd, J=8.0, 1.9 Hz, 1H), 6.27 (d, J=1.9 Hz, 1H), 5.24 (q, J=1.5 Hz, 1H), 3.76 (s, 3H), 3.60 (s, 1H), 1.95 (d, J=1.4 Hz, 3H), 1.61 (s, 6H), 1.24 (s, 6H); 13C NMR (CDCl₃, 101 MHz) δ 159.4 (C), 152.7 (C), 151.1 (C), 142.9 (C), 128.9 (CH), 128.5 (C), 127.8 (CH), 123.3 (CH), 119.7 (CH), 119.3 (C), 115.9 (CH), 113.5 (CH), 111.8 (CH), 110.3 (CH), 55.3 (CH₃), 52.0 (C), 42.9 (C), 31.3 (CH₃), 30.7 (CH₃), 18.6 (CH₃); HRMS (ESI) calcd for C₂₂H₂₈NO [M+H]⁺ 322.2165, found 322.2168.

### Example 74: Preparation of 2,2⁻Diarylpropane Intermediates (Scheme 10): 1⁻Ethyl-7⁻(2⁻(3⁻methoxyphenyl)propan-2⁻yl)-2,2,4⁻trimethyl-1,2⁻dihydroquinoline (S41)

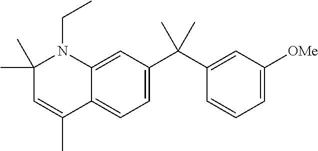 [SMILES: CCN1c2cc(C(C)(C)c3cccc(OC)c3)ccc2C(C)=CC1(C)C]

7⁻(2⁻(3⁻Methoxy-phenyl)propan-2⁻yl)-2,2,4⁻trimethyl-1,2⁻dihydroquinoline (S40; 1.50 g, 4.67 mmol), K₂CO₃ (3.87 g, 28.00 mmol, 6 eq), and iodoethane (4.50 mL, 55.99 mmol, 12 eq) were combined in DMF (24 mL) in a heavy-wall pressure flask. The vessel was sealed (PTFE bushing, Viton O-ring) and stirred at 120° C. for 4 h. After cooling to room temperature, the mixture was diluted with water and extracted with EtOAc (2×). The organic extracts were washed with water and brine, dried over anhydrous MgSO₄, filtered, and concentrated in vacuo. Purification by flash chromatography on silica gel (0-10% Et₂O/hexanes, linear gradient) afforded 1.57 g (96%) of S41 as a colorless gum. ¹H NMR (CDCl₃, 400 MHz) δ 7.20-7.14 (m, 1H), 6.94 (d, J=7.9 Hz, 1H), 6.87 (d, J=1.7 Hz, 1H), 6.87-6.84 (m, 1H), 6.74-6.66 (m, 1H), 6.48 (dd, J=7.9, 1.8 Hz, 1H), 6.26 (d, J=1.8 Hz, 1H), 5.13 (q, J=1.5 Hz, 1H), 3.76 (s, 3H), 3.19 (q, J=7.0 Hz, 2H), 1.93 (d, J=1.4 Hz, 3H), 1.65 (s, 6H), 1.28 (s, 6H), 1.02 (t, J=7.0 Hz, 3H); 13C NMR (CDCl₃, 101 MHz) δ 159.4 (C), 152.9 (C), 151.1 (C), 143.1 (C), 128.9 (CH), 128.7 (CH), 127.6 (C), 123.3 (CH), 120.3 (C), 119.8 (CH), 113.6 (CH), 113.5 (CH), 110.2 (CH), 109.7 (CH), 57.0 (C), 55.3 (CH₃), 43.2 (C), 38.1 (CH₂), 30.7 (CH₃), 28.9 (CH₃), 18.8 (CH₃), 14.2 (CH₃); HRMS (ESI) calcd for C₂₄H₃₂NO [M+H]⁺ 350.2478, found 350.2481.

### Example 75: Preparation of 2,2⁻Diarylpropane Intermediates (Scheme 10): 1⁻Ethyl-7⁻(2⁻(3⁻methoxyphenyl)propan-2⁻yl)-2,2,4⁻trimethyl-1,2,3,4⁻tetrahydroquinoline (S42)

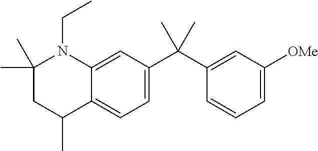 [SMILES: CCN1c2cc(C(C)(C)c3cccc(OC)c3)ccc2C(C)CC1(C)C]

A round-bottom flask was charged with Pd/C (10%, 1.52 g, 1.43 mmol, 0.2 eq) under nitrogen, and 1-ethyl-7⁻(2⁻(3⁻methoxyphenyl)propan-2⁻yl)-2,2,4⁻trimethyl-1,2-dihydroquinoline (S41; 2.50 g, 7.15 mmol) in THE (70 mL) was added. The sealed flask was evacuated/backfilled with H₂ from a balloon (4×) and then stirred under the H₂ balloon at room temperature for 4 h. The reaction mixture was filtered through Celite with EtOAc and concentrated in vacuo. Silica gel chromatography (0-10% Et₂O/hexanes, linear gradient) yielded 2.46 g (98%) of S42 as a colorless oil. ¹H NMR (CDCl₃, 400 MHz) δ 7.18 (t, J=8.2 Hz, 1H), 7.01 (dd, J=7.9, 1.2 Hz, 1H), 6.90-6.85 (m, 2H), 6.70 (ddd, J=8.1, 2.4, 1.0 Hz, 1H), 6.49 (dd, J=7.9, 1.8 Hz, 1H), 6.35 (d, J=1.9 Hz, 1H), 3.77 (s, 3H), 3.33 (dq, J=14.2, 7.0 Hz, 1H), 3.04 (dq, J=13.9, 6.9 Hz, 1H), 2.90-2.78 (m, 1H), 1.67 (dd, J=12.9, 4.8 Hz, 1H), 1.66 (s, 6H), 1.54 (t, J=12.8 Hz, 1H), 1.29 (d, J=6.5 Hz, 3H), 1.28 (s, 3H), 1.14 (s, 3H), 1.02 (t, J=6.9 Hz, 3H); 13C NMR (CDCl₃, 101 MHz) δ 159.4 (C), 153.0 (C), 149.0 (C), 144.2 (C), 128.8 (CH), 125.3 (CH), 125.0 (C), 119.9 (CH), 113.6 (CH), 113.5 (CH), 110.4 (CH), 110.1 (CH), 55.3 (CH₃), 54.4 (C), 47.2 (CH₂), 43.0 (C), 39.1 (CH₂), 30.9 (CH₃), 30.8 (CH₃), 29.9 (CH₃), 27.1 (CH), 25.4 (CH₃), 20.0 (CH₃), 15.0 (CH₃); HRMS (ESI) calcd for C₂₄H₃₄NO [M+H]⁺ 352.2635, found 352.2636.

### Example 76: Preparation of 2,2⁻Diarylpropane Intermediates (Scheme 10): 3⁻(2⁻(1⁻Ethyl-2,2,4⁻trimethyl-1,2,3,4⁻tetrahydroquinolin-7⁻yl)propan-2⁻yl)phenol (S43)

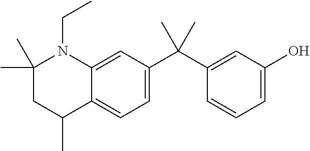 [SMILES: CCN1c2cc(C(C)(C)c3cccc(O)c3)ccc2C(C)CC1(C)C]

1⁻Ethyl-7⁻(2⁻(3⁻methoxyphenyl)propan-2⁻yl)-2,2,4⁻trimethyl-1,2,3,4-tetrahydroquinoline (S42; 2.00 g, 5.69 mmol) was dissolved in glacial AcOH (12 mL); 48% aqueous HBr (12 mL) was added, and the mixture was stirred at reflux for 4 h. After cooling to room temperature, the solution was carefully diluted into water (100 mL), adjusted to pH 5-6 with 25% w/w NaOH, and extracted with EtOAc (2×). The combined organic extracts were washed with saturated NaHCO₃ and brine, dried over anhydrous MgSO₄, filtered, and concentrated in vacuo. Purification by flash chromatography on silica gel (0-4₀% EtOAc/hexanes, linear gradient) afforded 1.83 g (95%) of S43 as a colorless gum. 1H NMR (CDCl₃, 400 MHz) δ 7.13 (t, J=7.9 Hz, 1H), 7.02 (dd, J=7.9, 1.2 Hz, 1H), 6.92-6.87 (m, 1H), 6.73 (t, J=2.1 Hz, 1H), 6.65-6.61 (m, 1H), 6.50 (dd, J=7.9, 1.9 Hz, 1H), 6.34 (d, J=1.9 Hz, 1H), 4.52 (s, 1H), 3.33 (dq, J=14.2, 7.0 Hz, 1H), 3.05 (dq, J=14.1, 6.9 Hz, 1H), 2.90-2.79 (m, 1H), 1.67 (dd, J=12.9, 4.8 Hz, 1H), 1.64 (s, 6H), 1.54 (t, J=12.8 Hz, 1H), 1.29 (d, J=7.3 Hz, 3H), 1.28 (s, 3H), 1.15 (s, 3H), 1.02 (t, J=7.0 Hz, 3H); ¹³C NMR (CDCl₃, 101 MHz) δ 155.2 (C), 153.4 (C), 149.0 (C), 144.2 (C), 129.1 (CH), 125.3 (CH), 125.0 (C), 119.5 (CH), 114.3 (CH), 113.4 (CH), 112.4 (CH), 110.5 (CH), 54.4 (C), 47.2 (CH₂), 42.9 (C), 39.1 (CH₂), 30.8 (CH₃), 29.9 (CH₃), 27.0 (CH), 25.4 (CH₃), 20.0 (CH₃), 15.0 (CH₃); HRMS (ESI) calcd for C₂₃H₃₂NO [M+H]⁺ 338.2478, found 338.2478.

### Example 77: Preparation of 2,2⁻Diarylpropane Intermediates (Scheme 10): 3⁻(2⁻(1⁻Ethyl-2,2,4⁻trimethyl-1,2,3,4⁻tetrahydroquinolin-7⁻yl)propan-2⁻yl)phenyl trifluoromethanesulfonate (S44)

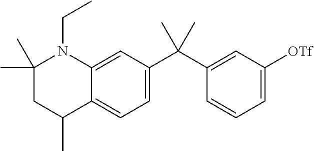 [SMILES: CCN1c2cc(C(C)(C)c3cccc(OS(=O)(=O)C(F)(F)F)c3)ccc2C(C)CC1(C)C]

3⁻(2⁻(1⁻Ethyl-2,2,4⁻trimethyl-1,2,3,4⁻tetrahydroquinolin-7⁻yl)propan-2⁻yl)phenol (S43; 1.70 g, 5.04 mmol) was taken up in CH₂Cl₂ (50 mL). DIEA (2.63 mL, 15.11 mmol, 3 eq) and N-phenyl-bis(trifluoromethanesulfonimide) (3.60 g, 10.07 mmol, 2 eq) were added, and the reaction was stirred at room temperature for 18 h. The solvent was removed by rotary evaporation, and the crude residue was purified by silica gel chromatography (0-40% CH₂Cl₂/hexanes, linear gradient) to afford 2.24 g (95%) of S44 as a white solid. 1H NMR (CDCl₃, 400 MHz) δ 7.35-7.27 (m, 2H), 7.19 (t, J=2.0 Hz, 1H), 7.07 (dt, J=7.4, 2.2 Hz, 1H), 7.04 (d, J=7.9 Hz, 1H), 6.48 (dd, J=7.9, 1.9 Hz, 1H), 6.23 (d, J=1.9 Hz, 1H), 3.31 (dq, J=14.3, 7.1 Hz, 1H), 3.01 (dq, J=14.1, 6.9 Hz, 1H), 2.90-2.79 (m, 1H), 1.68 (dd, J=12.9, 4.7 Hz, 1H), 1.67 (s, 6H), 1.53 (t, J=12.8 Hz, 1H), 1.30 (d, J=6.6 Hz, 3H), 1.28 (s, 3H), 1.15 (s, 3H), 0.98 (t, J=7.0 Hz, 3H); 19F NMR (CDCl₃, 376 MHz) δ−73.41 (s); 13C NMR (CDCl₃, 101 MHz) δ 154.7 (C), 149.7 (C), 147.9 (C), 144.4 (C), 129.6 (CH), 127.4 (CH), 125.6 (CH), 119.9 (CH), 118.9 (q, ¹J_{CF}=320.8 Hz, CF₃), 118.3 (CH), 113.1 (CH), 110.4 (CH), 54.5 (C), 47.1 (CH₂), 43.2 (C), 39.0 (CH₂), 30.64 (CH₃), 30.63 (CH₃), 29.8 (CH₃), 27.1 (CH), 25.3 (CH₃), 20.0 (CH₃), 14.8 (CH₃); HRMS (ESI) calcd for C₂₄H₃₁F₃NO₃S [M+H]⁺ 470.1971, found 470.1974.

### Example 78: Preparation of 2,2⁻Diarylpropane Intermediates (Scheme 10): tert-Butyl (3⁻(2⁻(1-ethyl-2,2,4⁻trimethyl-1,2,3,4⁻tetrahydroquinolin-7⁻yl)propan-2-yl)phenyl)carbamate (S45)

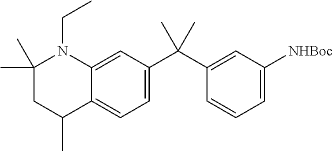 [SMILES: CCN1c2cc(C(C)(C)c3cccc(NC(=O)OC(C)(C)C)c3)ccc2C(C)CC1(C)C]

An oven-dried crimp-top vial was charged with tert-butyl carbamate (299 mg, 2.56 mmol, 1.2 eq), Pd₂dba₃ (97.5 mg, 0.106 mmol, 0.05 eq), XPhos (152 mg, 0.319 mmol, 0.15 eq), and Cs₂CO₃ (971 mg, 2.98 mmol, 1.4 eq). The vial was sealed and evacuated/backfilled with nitrogen (3×). A solution of 3⁻(2⁻(1-ethyl-2,2,4⁻trimethyl-1,2,3,4⁻tetrahydroquinolin-7-yl)propan-2⁻yl)phenyl trifluoromethanesulfonate (S44; 1.00 g, 2.13 mmol) in dioxane (10 mL) was added; after flushing the reaction again with nitrogen (3×), it was stirred at 100° C. for 4 h. It was then cooled to room temperature, filtered through Celite with CH₂Cl₂, and evaporated. The residue was purified by silica gel chromatography (0-20% Et₂O/hexanes, linear gradient) to afford S45 (906 mg, 97%) as pale yellow gum. ¹H NMR (CDCl₃, 400 MHz) δ 7.43-7.30 (m, 1H), 7.19 (t, J=7.9 Hz, 1H), 7.05 (t, J=2.0 Hz, 1H), 7.01 (dd, J=7.9, 1.2 Hz, 1H), 6.97 (ddd, J=7.8, 1.9, 1.0 Hz, 1H), 6.48 (dd, J=7.9, 1.9 Hz, 1H), 6.38 (s, 1H), 6.34 (d, J=1.9 Hz, 1H), 3.33 (dq, J=14.2, 7.0 Hz, 1H), 3.05 (dq, J=13.9, 6.9 Hz, 1H), 2.90-2.78 (m, 1H), 1.67 (dd, J=12.9, 4.7 Hz, 1H), 1.65 (s, 6H), 1.54 (t, J=12.9 Hz, 1H), 1.49 (s, 9H), 1.29 (d, J=7.0 Hz, 3H), 1.28 (s, 3H), 1.15 (s, 3H), 1.02 (t, J=7.0 Hz, 3H); 13C NMR (CDCl₃, 101 MHz) δ 152.9 (C), 152.2 (C), 149.0 (C), 144.2 (C), 138.0 (C), 128.6 (CH), 125.3 (CH), 125.0 (C), 121.9 (CH), 117.5 (CH), 116.0 (CH), 113.5 (CH), 110.4 (CH), 80.4 (C), 54.4 (C), 47.2 (CH₂), 43.0 (C), 39.0 (CH₂), 30.9 (CH₃), 29.9 (CH₃), 28.5 (CH₃), 27.1 (CH), 25.4 (CH₃), 20.0 (CH₃), 15.0 (CH₃); HRMS (ESI) calcd for C₂₈H₄₁N₂O₂ [M+H]⁺ 437.3163, found 437.3163.

### Example 79: Preparation of 2,2⁻Diarylpropane Intermediates (Scheme 10): 3⁻(2⁻(1⁻Ethyl-2,2,4⁻trimethyl-1,2,3,4⁻tetrahydroquinolin-7⁻yl)propan-2⁻yl)aniline (S46)

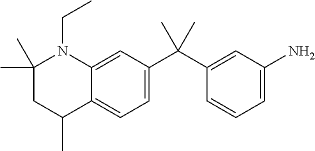 [SMILES: CCN1c2cc(C(C)(C)c3cccc(N)c3)ccc2C(C)CC1(C)C]

tert-Butyl (3⁻(2⁻(1-ethyl-2,2,4⁻trimethyl-1,2,3,4⁻tetrahydroquinolin-7⁻yl)propan-2⁻yl)phenyl)carbamate (S45; 1.90 g, 4.35 mmol) was taken up in CH₂Cl₂ (35 mL), and trifluoroacetic acid (7 mL) was added. The reaction was stirred at room temperature for 2 h. It was then diluted with toluene (30 mL) and concentrated to dryness. The residue was taken up in saturated NaHCO₃ and extracted with EtOAc (2×). The combined organic extracts were washed with brine, dried over anhydrous MgSO₄, filtered, concentrated in vacuo. Flash chromatography (0-30% EtOAc/hexanes, linear gradient) afforded 1.29 g (88%) of S46 as an off-white solid. 1H NMR (CDCl₃, 400 MHz) δ 7.05 (t, J=7.8 Hz, 1H), 7.01 (dd, J=7.9, 1.0 Hz, 1H), 6.73 (ddd, J=7.8, 1.7, 0.9 Hz, 1H), 6.61 (t, J=2.1 Hz, 1H), 6.53-6.47 (m, 2H), 6.38 (d, J=1.9 Hz, 1H), 3.53 (s, 2H), 3.33 (dq, J=14.2, 7.0 Hz, 1H), 3.06 (dq, J=14.2, 6.9 Hz, 1H), 2.90-2.78 (m, 1H), 1.67 (dd, J=12.9, 4.8 Hz, 1H), 1.63 (s, 6H), 1.54 (t, J=12.8 Hz, 1H), 1.29 (d, J=6.7 Hz, 3H), 1.28 (s, 3H), 1.15 (s, 3H), 1.04 (t, J=7.0 Hz, 3H); 13C NMR (CDCl₃, 101 MHz) δ 152.5 (C), 149.3 (C), 146.0 (C), 144.1 (C), 128.8 (CH), 125.3 (CH), 124.9 (C), 117.5 (CH), 114.4 (CH), 113.5 (CH), 112.6 (CH), 110.5 (CH), 54.4 (C), 47.2 (CH₂), 42.8 (C), 39.1 (CH₂), 30.86 (CH₃), 30.84 (CH₃), 29.9 (CH₃), 27.0 (CH), 25.4 (CH₃), 20.0 (CH₃), 15.0 (CH₃); HRMS (ESI) calcd for C₂₃H₃₃N₂[M+H]⁺ 337.2638, found 337.2637.

### Example 80: Preparation of 2,2⁻Diarylpropane Intermediates (Scheme 10): 8-(2⁻(1⁻Ethyl-2,2,4⁻trimethyl-1,2,3,4⁻tetrahydroquinolin-7⁻yl)propan-2⁻yl)-2,3,6,7-tetrahydro-1H,5H-pyrido-[3,2,1⁻ij]quinoline (58)

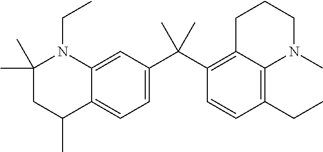

A mixture of 3⁻(2⁻(1-ethyl-2,2,4⁻trimethyl-1,2,3,4⁻tetrahydroquinolin-7-yl)propan-2⁻yl)aniline (S46; 700 mg, 2.08 mmol), Na₂CO₃ (2.20 g, 20.80 mmol, 10 eq), and 1-bromo-3⁻chloropropane (15.44 mL, 156.0 mmol, 75 eq) was stirred at 140° C. for 18 h. The reaction was subsequently cooled to room temperature, diluted with water, and extracted with EtOAc (2×). The combined organic extracts were dried over anhydrous MgSO₄, filtered, and evaporated. Silica gel chromatography (0-10% Et₂O/hexanes, linear gradient) yielded 453 mg (52%) of 58 as a colorless gum. ¹H NMR (CDCl₃, 400 MHz) δ 6.95 (d, J=7.9 Hz, 1H), 6.79 (s, 2H), 6.40 (dd, J=7.9, 1.8 Hz, 1H), 6.36 (d, J=1.8 Hz, 1H), 3.32 (dq, J=14.2, 7.0 Hz, 1H), 3.13-3.08 (m, 2H), 3.04 (dq, J=15.0, 7.0 Hz, 1H), 2.99 (t, J=6.3 Hz, 2H), 2.89-2.77 (m, 1H), 2.74 (t, J=6.4 Hz, 2H), 2.25-2.12 (m, 2H), 1.97-1.88 (m, 2H), 1.67 (dd, J=12.9, 4.8 Hz, 1H), 1.614 (s, 3H), 1.607 (s, 3H), 1.57-1.48 (m, 3H), 1.272 (s, 3H), 1.271 (d, J=6.7 Hz, 3H), 1.14 (s, 3H), 1.01 (t, J=6.9 Hz, 3H); 13C NMR (CDCl₃, 101 MHz) δ 150.3 (C), 145.3 (C), 144.3 (C), 143.7 (C), 125.9 (CH), 125.3 (CH), 124.6 (C), 122.4 (C), 120.4 (C), 114.3 (CH), 112.9 (CH), 109.3 (CH), 54.3 (C), 50.9 (CH₂), 50.3 (CH₂), 47.5 (CH₂), 43.4 (C), 39.0 (CH₂), 32.0 (CH₃), 31.3 (CH₃), 29.8 (CH₃), 28.2 (CH₂), 27.1 (CH), 26.2 (CH₂), 25.2 (CH₃), 22.4 (CH₂), 22.1 (CH₂), 20.2 (CH₃), 15.0 (CH₃); HRMS (ESI) calcd for C₂₉H₄₁N₂[M+H]⁺ 417.3264, found 417.3264.

### Example 81: Preparation of Anthrone Intermediates (Scheme 11): 9,9-Dimethyl-10⁻oxo-9,10⁻dihydroanthracene-2,7⁻diyl bis(trifluoromethanesulfonate) (S48)

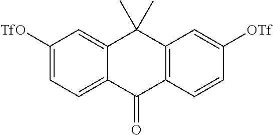 [SMILES: CC1(C)c2cc(OS(=O)(=O)C(F)(F)F)ccc2C(=O)c2ccc(OS(=O)(=O)C(F)(F)F)cc21]

3,6⁻Dihydroxy-10,10⁻dimethylanthracen-9(10)-one⁴¹ (S47; 747 mg, 2.94 mmol) was taken up in CH₂Cl₂ (15 mL) and cooled to 0° C. Pyridine (1.90 mL, 23.50 mmol, 8 eq) and trifluoromethanesulfonic anhydride (1.98 mL, 11.75 mmol, 4 eq) were added, and the ice bath was removed. The reaction was stirred at room temperature for 2 h. It was subsequently diluted with water and extracted with CH₂Cl₂ (2×). The combined organic extracts were washed with saturated CuSO₄ and brine, dried over anhydrous MgSO₄, filtered, and concentrated in vacuo. Flash chromatography on silica gel (0-30% EtOAc/hexanes, linear gradient) afforded 1.46 g (96%) of S48 as a white solid. ¹H NMR (CDCl₃, 400 MHz) δ 8.47 (d, J=8.7 Hz, 2H), 7.58 (d, J=2.4 Hz, 2H), 7.38 (dd, J=8.8, 2.4 Hz, 2H), 1.78 (s, 6H); 19F NMR (CDCl₃, 376 MHz) δ−73.13 (s); 13C NMR (CDCl₃, 101 MHz) δ 180.9 (C), 153.4 (C), 152.7 (C), 130.9 (CH), 129.4 (C), 120.5 (CH), 120.0 (CH), 118.9 (q, ¹J_{CF}=320.8 Hz, CF₃) 38.8 (C), 33.0 (CH₃); HRMS (ESI) calcd for C₁₈H₁₃F₆O₇S₂[M+H]⁺ 519.0001, found 518.9993.

### Example 82: Preparation of Anthrone Intermediates (Scheme 11): 10,10-Dimethyl-3,6⁻bis(pyrrolidin-1⁻yl-d₈)anthracen-9(10H)-one (42)

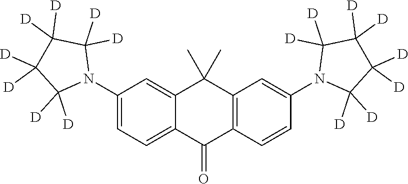 [SMILES: [2H]C1([2H])N(c2ccc3c(c2)C(C)(C)c2cc(N4C([2H])([2H])C([2H])([2H])C([2H])([2H])C4([2H])[2H])ccc2C3=O)C([2H])([2H])C([2H])([2H])C1([2H])[2H]]

A vial was charged with 9,9⁻dimethyl-10⁻oxo-9,10⁻dihydroanthracene-2,7⁻diyl bis(trifluoromethanesulfonate) (S48; 1.00 g, 1.93 mmol), Pd₂dba₃ (177 mg, 0.193 mmol, 0.1 eq), XPhos (276 mg, 0.579 mmol, 0.3 eq), and Cs₂CO₃ (1.76 g, 5.40 mmol, 2.8 eq). The vial was sealed and evacuated/backfilled with nitrogen (3×). Dioxane (10 mL) was added, and the reaction was flushed again with nitrogen (3×). Following the addition of pyrrolidine-2,2,3,3,4,4,5,5⁻d₈(387 μL, 4.63 mmol, 2.4 eq), the reaction was stirred at 100° C. for 3 h. It was then cooled to room temperature, filtered through Celite with CH₂Cl₂, and concentrated to dryness. Purification by silica gel chromatography (0-40% EtOAc/hexanes, linear gradient, with constant 40% v/v CH₂Cl₂ additive) afforded 42 (498 mg, 69%) as a yellow solid. 1H NMR (CDCl₃, 400 MHz) δ 8.28-8.25 (m, 2H), 6.64-6.60 (m, 4H), 1.71 (s, 6H); 13C NMR (CDCl₃, 101 MHz) δ 181.3 (C), 152.6 (C), 150.8 (C), 129.5 (CH), 119.6 (C), 111.0 (CH), 107.6 (CH), 38.2 (C), 33.9 (CH₃); HRMS (ESI) calcd for C₂₄H₁₃D₁₆N₂O [M+H]⁺ 377.3279, found 377.3264.

### Example 83: Preparation of Anthrone Intermediates (Scheme 11): 3,6-Di(azetidin-1⁻yl)-10,10⁻dimethylanthracen-9(10H)-one (43)

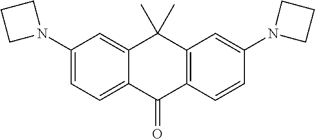 [SMILES: CC1(C)c2cc(N3CCC3)ccc2C(=O)c2ccc(N3CCC3)cc21]

A vial was charged with 9,9⁻dimethyl-10⁻oxo-9,10⁻dihydroanthracene-2,7⁻diyl bis(trifluoromethanesulfonate) (S48; 1.25 g, 2.41 mmol), Pd₂dba₃ (221 mg, 0.241 mmol, 0.1 eq), XPhos (345 mg, 0.723 mmol, 0.3 eq), and Cs₂CO₃ (2.20 g, 6.75 mmol, 2.8 eq). The vial was sealed and evacuated/backfilled with nitrogen (3×). Dioxane (12 mL) was added, and the reaction was flushed again with nitrogen (3×). Following the addition of azetidine (390 μL, 5.79 mmol, 2.4 eq), the reaction was stirred at 100° C. for 4 h. It was then cooled to room temperature, filtered through Celite with CH₂Cl₂, and concentrated to dryness. Purification by silica gel chromatography (0-40% EtOAc/hexanes, linear gradient, with constant 40% v/v CH₂Cl₂ additive) afforded 43 (645 mg, 80%) as a yellow solid. ¹H NMR (CDCl₃, 400 MHz) δ 8.26-8.21 (m, 2H), 6.46-6.40 (m, 4H), 4.03 (t, J=7.3 Hz, 8H), 2.43 (p, J=7.2 Hz, 4H), 1.66 (s, 6H); 13C NMR (CDCl₃, 101 MHz) δ 181.4 (C), 154.3 (C), 152.4 (C), 129.3 (CH), 120.7 (C), 109.8 (CH), 106.5 (CH), 51.8 (CH₂), 38.1 (C), 33.6 (CH₃), 16.7 (CH₂); HRMS (ESI) calcd for C₂₂H_{2 5}N₂O [M+H]⁺ 333.1961, found 333.1959.

### Example 84: Preparation of Anthrone Intermediates (Scheme 11): Methyl 1-benzyl-1,2,3,4⁻tetrahydroquinoline-6⁻carboxylate (S50)

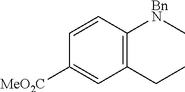 [SMILES: CCc1cc(C(=O)OC)ccc1N(C)Cc1ccccc1]

Methyl 1,2,3,4⁻tetrahydroquinoline-6⁻carboxylate (S49; 1.90 g, 9.94 mmol), benzyl bromide (4.25 g, 24.8 mmol, 2.5 eq), K₂CO₃ (4.12 g, 29.8 mmol, 3 eq), and KI (412 mg, 2.49 mmol, 0.25 eq) were combined in DMF (25 mL) and stirred at 60° C. for 4 h. The reaction was subsequently diluted with water and extracted with EtOAc (2×). The combined organic extracts were washed with water and brine, dried (MgSO₄), filtered, and concentrated in vacuo. Silica gel chromatography (0-25% EtOAc/hexanes, linear gradient) afforded 2.60 g (93%) of S50 as a gum that crystallized into a colorless solid upon standing. ¹H NMR (CDCl₃, 400 MHz) δ 7.69-7.62 (m, 2H), 7.36-7.29 (m, 2H), 7.28-7.24 (m, 1H), 7.24-7.19 (m, 2H), 6.49-6.43 (m, 1H), 4.55 (s, 2H), 3.82 (s, 3H), 3.47-3.40 (m, 2H), 2.84 (t, J=6.2 Hz, 2H), 2.07-1.96 (m, 2H); 13C NMR (CDCl₃, 101 MHz) δ 167.6 (C), 149.3 (C), 137.7 (C), 130.7 (CH), 129.8 (CH), 128.9 (CH), 127.2 (CH), 126.5 (CH), 121.3 (C), 116.8 (C), 109.8 (CH), 54.8 (CH₂), 51.5 (CH₃), 50.1 (CH₂), 28.2 (CH₂), 22.0 (CH₂); HRMS (ESI) calcd for C₁₈H₂₀NO₂ [M+H]⁺ 282.1489, found 282.1488.

### Example 85: Preparation of Anthrone Intermediates (Scheme 11): (1⁻Benzyl-1,2,3,4⁻tetrahydroquinolin-6⁻yl)methanol (S51)

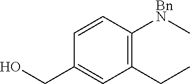 [SMILES: CCc1cc(CO)ccc1N(C)Cc1ccccc1]

A solution of methyl 1⁻benzyl-1,2,3,4⁻tetrahydroquinoline-6⁻carboxylate (S50; 4.25 g, 15.1 mmol) in THE (50 mL) was cooled to 0° C. under nitrogen, and LiAlH₄ (1.0 M in THF, 30.2 mL, 30.2 mmol, 2 eq) was added. The reaction was stirred for 2 h at 0° C. The Fieser workup was performed by the sequential addition of (1) H₂O (1.15 mL), (2) 15% NaOH (1.15 mL), and (3) H₂O (3×1.15 mL). The resulting suspension was vigorously stirred for 10 min. It was then diluted with Et₂O (100 mL), and MgSO₄ (˜5 g) was added. After stirring for another 5 min, the mixture was filtered through Celite with Et₂O and concentrated to dryness. Flash chromatography on silica gel (10-50% EtOAc/hexanes, linear gradient) provided S51 (3.43 g, 90%) as a colorless gum. Note: Alcohol S51 is photosensitive and decomposes rapidly in chlorinated solvents. ¹H NMR (acetone-d₆, 400 MHz) δ 7.34-7.25 (m, 4H), 7.25-7.19 (m, 1H), 6.93-6.90 (m, 1H), 6.90-6.85 (m, 1H), 6.45 (d, J=8.3 Hz, 1H), 4.49 (s, 2H), 4.41 (d, J=5.8 Hz, 2H), 3.71 (t, J=5.7 Hz, 1H), 3.41-3.35 (m, 2H), 2.77 (t, J=6.3 Hz, 2H), 2.02-1.93 (m, 2H); 13C NMR (acetone-d₆, 101 MHz) δ 145.5 (C), 140.2 (C), 130.5 (C), 129.3 (CH), 129.0 (CH), 127.5 (CH), 127.4 (CH), 126.9 (CH), 122.6 (C), 111.6 (CH), 64.9 (CH₂), 55.6 (CH₂), 50.7 (CH₂), 28.9 (CH₂), 23.20 (CH₂); HRMS (ESI) calcd for C₁₇H₁₉NONa [M+Na]⁺276.1359, found 276.1354.

### Example 86: Preparation of Anthrone Intermediates (Scheme 11): 1⁻Benzyl-6⁻((1⁻benzyl-1,2,3,4⁻tetrahydroquinolin-6⁻yl)methyl)-7⁻bromo-1,2,3,4⁻tetrahydroquinoline (S52)

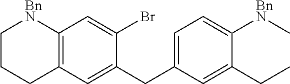 [SMILES: CCc1cc(Cc2cc3c(cc2Br)N(Cc2ccccc2)CCC3)ccc1N(C)Cc1ccccc1]

1⁻Benzyl-7⁻bromo-1,2,3,4⁻tetrahydroquinoline (S4; 2.16 g, 7.15 mmol) and (1-benzyl-1,2,3,4⁻tetrahydroquinolin-6⁻yl)methanol (S51; 1.90 g, 7.50 mmol, 1.05 eq) were combined in CH₂Cl₂ (50 mL) and cooled to 0° C. under nitrogen. BCl₃ (1 M in CH₂Cl₂, 21.4 mL, 21.4 mmol, 3 eq) was added over 5 min; the reaction was then allowed to warm to room temperature overnight while stirring (18 h). The reaction was carefully neutralized by the slow addition of saturated NaHCO₃ with vigorous stirring. The mixture was then extracted with CH-₂Cl₂ (2×). The combined organics were dried (MgSO₄), filtered, and evaporated. Flash chromatography (0-20% Et₂O/hexanes, linear gradient) afforded 3.15 g (82%) of S52 as a colorless, gummy foam. ¹H NMR (CDCl₃, 400 MHz) δ 7.35-7.19 (m, 10H), 6.83 (d, J=2.0 Hz, 1H), 6.80 (dd, J=8.3, 2.2 Hz, 1H), 6.74 (s, 1H), 6.69 (s, 1H), 6.43 (d, J=8.3 Hz, 1H), 4.43 (s, 2H), 4.42 (s, 2H), 3.79 (s, 2H), 3.35-3.30 (m, 2H), 3.30-3.24 (m, 2H), 2.78 (t, J=6.4 Hz, 2H), 2.67 (t, J=6.3 Hz, 2H), 2.04-1.89 (m, 4H); 13C NMR (CDCl₃, 101 MHz) δ 145.1 (C), 144.1 (C), 139.4 (C), 138.5 (C), 131.1 (CH), 129.6 (CH), 128.8 (CH), 128.7 (CH), 128.3 (C), 128.1 (C), 127.5 (CH), 127.1 (CH), 126.83 (CH), 126.81 (CH), 122.8 (C), 122.4 (C), 122.0 (C), 114.4 (CH), 111.2 (CH), 55.6 (CH₂), 55.2 (CH₂), 50.1 (CH₂), 49.5 (CH₂), 39.7 (CH₂), 28.4 (CH₂), 27.9 (CH₂), 22.7 (CH₂), 22.3 (CH₂); HRMS (ESI) calcd for C₃₃H₃₄BrN₂ [M+H]⁺ 537.1900, found 537.1914.

### Example 87: Preparation of Anthrone Intermediates (Scheme 11): 2⁻(1-Benzyl-6⁻((1⁻benzyl-1,2,3,4⁻tetrahydroquinolin-6⁻yl)methyl)-1,2,3,4⁻tetrahydroquinolin-7-yl)propan-2⁻ol (S53)

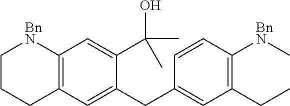 [SMILES: CCc1cc(Cc2cc3c(cc2C(C)(C)O)N(Cc2ccccc2)CCC3)ccc1N(C)Cc1ccccc1]

A solution of 1⁻benzyl-6⁻((1⁻benzyl-1,2,3,4⁻tetrahydroquinolin-6⁻yl)methyl)-7-bromo-1,2,3,4⁻tetrahydro-quinoline (S52; 2.35 g, 4.37 mmol) in THE (40 mL) was cooled to −78° C. under nitrogen. n-Butyllithium (2.5 M in hexanes, 4.37 mL, 10.9 mmol, 2.5 eq) was added, and the reaction was stirred for 15 min at −78° C. Acetone (3.21 mL, 43.7 mmol, 10 eq) was then added. The dry ice bath was removed, and the reaction was stirred at room temperature for 4 h. It was subsequently quenched with saturated NH₄Cl and extracted with EtOAc (2×). The combined organic extracts were washed with brine, dried (MgSO₄), filtered, and concentrated in vacuo. The residue was purified by silica gel chromatography (10-40% Et₂O/hexanes, linear gradient) to provide S53 as a colorless gum that crystallized into a white solid upon standing (2.07 g, 92%). ¹H NMR (CDCl₃, 400 MHz) δ 7.34-7.18 (m, 10H), 6.77 (d, J=1.9 Hz, 1H), 6.75 (s, 1H), 6.69 (dd, J=8.4, 2.1 Hz, 1H), 6.59 (s, 1H), 6.41 (d, J=8.4 Hz, 1H), 4.44 (s, 2H), 4.42 (s, 2H), 4.07 (s, 2H), 3.37-3.27 (m, 4H), 2.79-2.68 (m, 4H), 2.03-1.93 (m, 4H), 1.63 (bs, 1H), 1.45 (s, 6H); 13C NMR (CDCl₃, 101 MHz) δ 144.7 (C), 143.9 (C), 143.6 (C), 139.5 (C), 139.4 (C), 133.9 (CH), 130.5 (C), 129.4 (CH), 128.7 (CH), 128.6 (CH), 127.4 (CH), 126.96 (CH), 126.91 (CH), 126.85 (CH), 126.82 (CH), 126.0 (C), 122.5 (C), 121.3 (C), 111.3 (CH), 109.1 (CH), 74.2 (C), 56.0 (CH₂), 55.7 (CH₂), 50.4 (CH₂), 50.1 (CH₂), 38.0 (CH₂), 31.9 (CH₃), 28.4 (CH₂), 27.6 (CH₂), 22.7 (CH₂); HRMS (ESI) calcd for C₃₆H₄₀N₂ONa [M+Na]⁺539.3033, found 539.3024.

### Example 88: Preparation of Anthrone Intermediates (Scheme 11): 1,11-Dibenzyl-13,13⁻dimethyl-1,2,3,4,8,9,10,11⁻octahydrobenzo[1,2⁻g:5,4⁻g′]diquinolin-6(13H)-one (45)

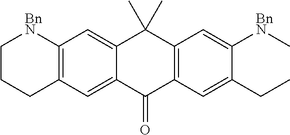 [SMILES: CCc1cc2c(cc1N(C)Cc1ccccc1)C(C)(C)c1cc3c(cc1C2=O)CCCN3Cc1ccccc1]

2⁻(1⁻Benzyl-6⁻((1⁻benzyl-1,2,3,4⁻tetrahydroquinolin-6⁻yl)methyl)-1,2,3,4-tetrahydroquinolin-7⁻yl)propan-2⁻ol (S53; 1.85 g, 3.58 mmol) was dissolved in CH₂Cl₂ (50 mL) and cooled to 0° C. under nitrogen. BBr₃ (1.0 M in CH₂Cl₂, 14.3 mL, 14.3 mmol, 4 eq) was added dropwise; the reaction was then allowed to warm to room temperature overnight while stirring (18 h). It was carefully neutralized through the sequential addition of H₂O (40 mL) and saturated NaHCO₃. The mixture was diluted with CH₂Cl₂ (50 mL), filtered through a hydrophobic phase separator column, and concentrated to give a blue residue. The crude material was taken up in acetone (35 mL) and cooled to −15° C. KMnO₄ (1.13 g, 7.16 mmol, 2 eq) was added in 4 equal portions 15 min apart (45 min total). After stirring for another 30 min at −15° C., the brown mixture was diluted with CH₂Cl₂, filtered through Celite, and concentrated in vacuo. Flash chromatography on silica gel (10-50% EtOAc/hexanes, linear gradient) provided 1.093 g (60%, 2 steps) of 45 as a photosensitive yellow solid. ¹H NMR (CDCl₃, 400 MHz) δ 7.96 (s, 2H), 7.34-7.29 (m, 4H), 7.27-7.21 (m, 6H), 6.48 (s, 2H), 4.57 (s, 4H), 3.52-3.43 (m, 4H), 2.89 (t, J=6.2 Hz, 4H), 2.10-1.97 (m, 4H), 1.27 (s, 6H); 13C NMR (CDCl₃, 101 MHz) δ 181.1 (C), 150.9 (C), 148.9 (C), 138.1 (C), 128.9 (CH), 127.8 (CH), 127.2 (CH), 126.6 (CH), 121.3 (C), 119.9 (C), 107.4 (CH), 55.5 (CH₂), 50.7 (CH₂), 37.5 (C), 33.3 (CH₃), 27.8 (CH₂), 22.3 (CH₂); HRMS (ESI) calcd for C₃₆H₃₇N₂O [M+H]⁺ 513.2900, found 513.2894.

### Example 89: Preparation of Anthrone Intermediates (Scheme 11): 2⁻(1,11-Dibenzyl-13,13⁻dimethyl-2,3,4,8,9,10,11,13⁻octahydrobenzo[1,2⁻g:5,4⁻g′]diquinolin-6⁻ylium-6(1H)-yl)-benzoate (S55)

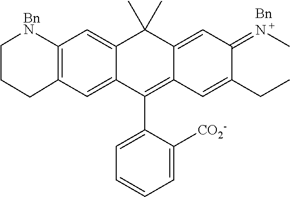

Step 1: A solution of 1⁻(2⁻bromophenyl)-4⁻methyl-2,6,7-trioxabicyclo[2.2.2]octane³² (S54; 556 mg, 1.95 mmol, 5 eq) in THE (30 mL) was cooled to −78° C. under nitrogen. tert-Butyllithium (1.7 M in pentane, 2.29 mL, 3.90 mmol, 10 eq) was added dropwise, and the reaction was stirred for 30 min at −78° C. A solution of 1,11⁻dibenzyl-13,13-dimethyl-1,2,3,4,8,9,10,11⁻octahydrobenzo[1,2⁻g:5,4⁻g′]diquinolin-6(13H)-one (45; 200 mg, 0.390 mmol) in THE (10 mL) was then added dropwise. The reaction was allowed to slowly warm to room temperature while stirring overnight (18 h). It was subsequently quenched with 1 N HCl (˜20 mL), stirred for 10 min, diluted with water, and extracted with 15% i-PrOH/CHCl₃ (2×). The combined organic extracts were dried over anhydrous MgSO₄, filtered, and evaporated. Silica gel chromatography (0-15% MeOH/CH₂Cl₂, linear gradient, with constant 1% v/v AcOH additive) afforded the 2,2⁻bis(hydroxymethyl)propyl ester intermediate as a dark blue solid (280 mg, 92%, acetate salt).

Step 2: The ester intermediate from Step 1 (280 mg, 0.359 mmol) was taken up in 2,2,2⁻trifluoroethanol (18 mL), and 25% w/w NaOH (6 mL) was added. The reaction was stirred at room temperature for 7 days. It was then diluted with water and extracted with CH₂Cl₂ (3×). The combined organic extracts were dried over anhydrous MgSO₄, filtered, and concentrated in vacuo. Purification by flash chromatography on silica gel (5-75% EtOAc/toluene, linear gradient) afforded 175 mg (79%) of S55 as a pale blue solid. ¹H NMR (CDCl₃, 400 MHz) δ 7.99 (d, J=7.4 Hz, 1H), 7.58 (td, J=7.5, 1.4 Hz, 1H), 7.53 (td, J=7.2, 1.2 Hz, 1H), 7.35-7.29 (m, 4H), 7.28-7.20 (m, 6H), 7.07 (d, J=7.3 Hz, 1H), 6.53 (s, 2H), 6.23 (s, 2H), 4.49 (AB quartet, v_{A}=1809.4 Hz, v_{B}=1781.5 Hz, J_{AB}=16.8 Hz, 4H), 3.42-3.30 (m, 4H), 2.65-2.47 (m, 4H), 1.98-1.86 (m, 4H), 1.41 (s, 3H), 1.31 (s, 3H); 13C NMR (CDCl₃, 101 MHz) δ 171.2 (C), 155.8 (C), 145.9 (C), 144.9 (C), 138.8 (C), 134.5 (CH), 128.75 (CH), 128.70 (CH), 128.0 (CH), 127.3 (C), 127.0 (CH), 126.7 (CH), 124.8 (CH), 124.1 (CH), 121.6 (C), 118.6 (C), 108.2 (CH), 88.7 (C), 55.6 (CH₂), 50.3 (CH₂), 37.7 (C), 34.9 (CH₃), 33.0 (CH₃), 27.7 (CH₂), 22.3 (CH₂); Analytical HPLC: t_{R}=12.0 min, >99% purity (30-95% MeCN/H₂O, linear gradient, with constant 0.1% v/v TFA additive; 20 min run; 1 mL/min flow; ESI; positive ion mode; detection at 625 nm); HRMS (ESI) calcd for C₄₃H₄₁N₂O₂ [M+H]⁺ 617.3163, found 617.3156.

### Example 90: Preparation of Anthrone Intermediates (Scheme 11): Carbo-Q-rhodamine (CRh_{Q}, 94)

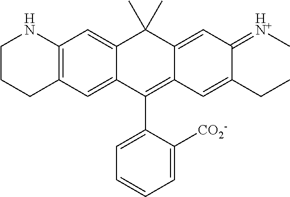

A round-bottom flask was charged with Pd/C (10%, 34.5 mg, 32.4 mol, 0.2 eq) under nitrogen, and a solution of carborhodamine S55 (100 mg, 0.162 mmol) in 1:1 THF/MeOH (15 mL) was added. The sealed flask was evacuated/backfilled with H₂ from a balloon (4×) and then stirred under the H₂ balloon at room temperature for 24 h. The reaction mixture was filtered through Celite with MeOH, and the filtrate was concentrated in vacuo. The blue residue was resuspended in 3:1 CH₂Cl₂/MeOH (8 mL), and p-chloranil (79.7 mg, 0.324 mmol, 2 eq) was added. After stirring the mixture at room temperature for 2 h, it was deposited onto Celite and concentrated to dryness. Silica gel chromatography (0-10% MeOH (2 M NH₃)/CH₂Cl₂, linear gradient; dry load with Celite) yielded 49.5 mg (70%) of 94 as a blue-purple solid. 1H NMR (DMSO-d₆, 400 MHz) δ 7.91 (d, J=7.6 Hz, 1H), 7.71 (td, J=7.4, 1.1 Hz, 1H), 7.62 (td, J=7.4, 1.0 Hz, 1H), 7.07 (d, J=7.6 Hz, 1H), 6.68 (s, 2H), 6.01 (s, 2H), 5.92 (s, 2H), 3.20-3.06 (m, 4H), 2.45 (dt, J=15.9, 5.9 Hz, 2H), 2.35 (dt, J=15.8, 6.1 Hz, 2H), 1.68 (p, J=6.1 Hz, 4H), 1.64 (s, 3H), 1.54 (s, 3H); 13C NMR (DMSO-d₆, 101 MHz) δ 170.0 (C), 155.0 (C), 146.0 (C), 144.1 (C), 135.0 (CH), 129.1 (CH), 127.4 (CH), 126.3 (C), 124.4 (CH), 123.9 (CH), 119.1 (C), 117.9 (C), 109.7 (CH), 88.5 (C), 40.6 (CH₂), 36.8 (C), 35.0 (CH₃), 32.7 (CH₃), 26.3 (CH₂), 21.3 (CH₂); Analytical HPLC: t_{R}=11.8 min, 98.3% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₂₉H₂₉N₂O₂ [M+H]⁺ 437.2224, found 437.2219.

### Example 91: Xanthene Synthesis Via Lithiation of Tetrafluorobenzoic Acid (Scheme 14): 4,5,6,7⁻Tetrafluoro-carbofluorescein (51)

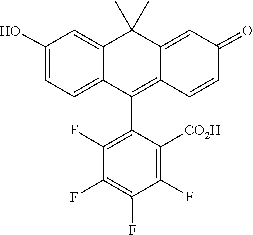 [SMILES: CC1(C)C2=CC(=O)C=CC2=C(c2c(F)c(F)c(F)c(F)c2C(=O)O)c2ccc(O)cc21]

A solution of 2,3,4,5⁻tetrafluorobenzoic acid (6; 281 mg, 1.45 mmol, 2 eq) in THF (5 mL) was cooled to −78° C. under nitrogen. N-Butyllithium (2.5 M in hexanes, 1.16 mL, 2.90 mmol, 4 eq) was added, and the reaction was stirred at −78° C. for 3 h. A solution of 3,6-bis((tert-butyldimethylsilyl)oxy)-10,10⁻dimethylanthracen-9(10)-one⁴¹ (50; 350 mg, 0.725 mmol) in THE (4 mL) was added; the reaction was warmed to room temperature and stirred for 1 h. It was subsequently diluted with saturated NH₄Cl and water and extracted with EtOAc (2×). The combined organic extracts were washed with brine, dried over anhydrous MgSO₄, filtered, and evaporated. The resulting yellow-orange residue was taken up in THE (10 mL) before adding TBAF (1.0 M in THF, 2.90 mL, 2.90 mmol, 4 eq). After stirring the reaction at room temperature for 30 min, it was acidified with 1 M HCl, diluted with water, and extracted with EtOAc (2×). The organic extracts were washed with brine, dried over anhydrous MgSO₄, filtered, and concentrated in vacuo. Flash chromatography (0-75% EtOAc/hexanes, linear gradient) afforded 51 (258 mg, 83%) as a pale yellow solid. ¹H NMR (DMSO-d₆, 400 MHz) δ 9.81 (s, 2H), 7.10 (d, J=2.4 Hz, 2H), 6.84 (d, J=8.6 Hz, 2H), 6.66 (dd, J=8.6, 2.4 Hz, 2H), 1.69 (s, 3H), 1.60 (s, 3H); 19F NMR (DMSO-d₆, 376 MHz) δ−139.94-−140.16 (m, 1F), −143.10-−143.30 (m, 1F), −143.78 (td, J=21.4, 20.8, 8.4 Hz, 1F), −152.01-−152.23 (m, 1F); Analytical HPLC: t_{R}=12.8 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₂₃H₁₅F₄O₄ [M+H]⁺ 431.0901, found 431.0899.

### Example 92: Xanthene Synthesis Via Lithiation of Tetrafluorobenzoic Acid (Scheme 14): 2′,4,5,6,7,7′-Hexafluorocarbofluorescein (53)

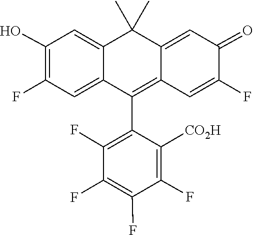 [SMILES: CC1(C)C2=CC(=O)C(F)=CC2=C(c2c(F)c(F)c(F)c(F)c2C(=O)O)c2cc(F)c(O)cc21]

A solution of 2,3,4,5⁻tetrafluorobenzoic acid (6; 281 mg, 1.45 mmol, 1.5 eq) in THF (5 mL) was cooled to −78° C. under nitrogen. N-Butyllithium (2.5 M in hexanes, 1.16 mL, 2.89 mmol, 3 eq) was added, and the reaction was stirred at −78° C. for 3 h. A solution of 3,6-bis((tert-butyldimethylsilyl)oxy)-2,7⁻difluoro-10,10⁻dimethylanthracen-9(10H)-one⁵⁴ (52; 500 mg, 0.964 mmol) in THF (5 mL) was added; the reaction was warmed to room temperature and stirred for 1 h. It was subsequently diluted with saturated NH₄Cl and water and extracted with EtOAc (2×). The combined organic extracts were washed with brine, dried over anhydrous MgSO₄, filtered, and evaporated. The resulting yellow-orange residue was taken up in THE (10 mL) before adding TBAF (1.0 M in THF, 3.86 mL, 3.86 mmol, 4 eq). After stirring the reaction at room temperature for 30 min, it was acidified with 1 M HCl, diluted with water, and extracted with EtOAc (2×). The organic extracts were washed with brine, dried over anhydrous MgSO₄, filtered, and concentrated in vacuo. Flash chromatography (0-75% EtOAc/hexanes, linear gradient, with constant 0.1% v/v AcOH additive) afforded 53 (345 mg, 77%) as a pale yellow solid. ¹H NMR (DMSO-d₆, 400 MHz) δ 10.35 (s, 2H), 7.28 (d, 4J_{HF}=8.7 Hz, 2H), 6.87 (d, 3J_{HF}=12.0 Hz, 2H), 1.66 (s, 3H), 1.58 (s, 3H); 19F NMR (DMSO-d₆, 376 MHz) δ−136.55-−136.80 (m, 2F), −139.04-−139.27 (m, 1F), −143.52-−143.86 (m, 2F), −152.25 (t, J=19.8 Hz, 1F); Analytical HPLC: t_{R}=13.4 min, 98.9% purity (10-95% MeCN/H₂O, linear gradient, with constant 0.1% v/v TFA additive; 20 min run; 1 mL/min flow; ESI; positive ion mode; detection at 280 nm); HRMS (ESI) calcd for C₂₃H₁₃F₆O₄ [M+H]⁺ 467.0713, found 467.0705.

### Example 93: Xanthene Synthesis Via Lithiation of Tetrafluorobenzoic Acid (Scheme 14): 4,5,6,7⁻Tetrafluoro-Si-fluorescein (55)

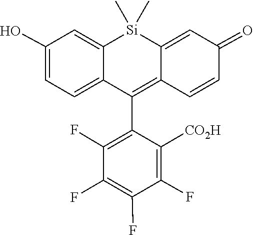 [SMILES: C[Si]1(C)C2=CC(=O)C=CC2=C(c2c(F)c(F)c(F)c(F)c2C(=O)O)c2ccc(O)cc21]

A solution of 2,3,4,5⁻tetrafluorobenzoic acid (6; 292 mg, 1.50 mmol, 1.5 eq) in THF (5 mL) was cooled to −78° C. under nitrogen. N-Butyllithium (2.5 M in hexanes, 1.20 mL, 3.01 mmol, 3 eq) was added, and the reaction was stirred at −78° C. for 3 h. A solution of 3,7-bis((tert-butyldimethylsilyl)oxy)-5,5⁻dimethyldibenzo[b,e]silin-10(5H)-one⁸⁰ (54; 500 mg, 1.00 mmol) in THE (5 mL) was added; the reaction was warmed to room temperature and stirred for 1 h. It was subsequently diluted with saturated NH₄Cl and water and extracted with EtOAc (2×). The combined organic extracts were washed with brine, dried over anhydrous MgSO₄, filtered, and evaporated. The resulting red-orange residue was taken up in THE (10 mL) before adding TBAF (1.0 M in THF, 4.00 mL, 4.00 mmol, 4 eq). After stirring the reaction at room temperature for 30 min, it was acidified with 1 M HCl, diluted with water, and extracted with EtOAc (2×). The organic extracts were washed with brine, dried over anhydrous MgSO₄, filtered, and concentrated in vacuo. Flash chromatography (10-100% EtOAc/hexanes, linear gradient) afforded 55 (422 mg, 94%) as an orange foam. The characterization data for 55 matched the previously reported spectra.¹

### Example 94: Xanthene Synthesis Via Lithiation of Tetrafluorobenzoic Acid (Scheme 14): 4,5,6,7⁻Tetrafluoro-Malachite Green lactone (FMGL, 49)

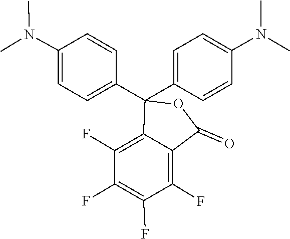

A solution of 2,3,4,5⁻tetrafluorobenzoic acid (6; 868 mg, 4.47 mmol, 6 eq) in THF (15 mL) was cooled to −78° C. under nitrogen. N-Butyllithium (2.5 M in hexanes, 3.58 mL, 8.94 mmol, 12 eq) was added, and the reaction was stirred at −78° C. for 3 h. A solution of Michler's ketone (48; 200 mg, 0.745 mmol) in THE (10 mL) was added; the reaction was warmed to room temperature and stirred for 18 h. It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. Purification by silica gel chromatography (0-5₀% EtOAc/hexanes, linear gradient) yielded 266 mg (80%) of 49 as a blue-green foam. ¹H NMR (CDCl₃, 400 MHz) δ 7.15 (d, J=9.0 Hz, 4H), 6.64 (d, J=9.0 Hz, 4H), 2.96 (s, 12H); 19F NMR (CDCl₃, 376 MHz) δ−137.82-−137.98 (m, 1F), −138.99 (td, J=20.0, 8.7 Hz, 1F), −143.58 (ddd, J=21.1, 18.2, 8.7 Hz, 1F), −152.13 (ddd, J=20.8, 18.3, 4.3 Hz, 1F); Analytical HPLC: t_{R}=12.5 min, >99% purity (10-95% MeCN/H₂O, linear gradient, with constant 0.1% v/v TFA additive; 20 min run; 1 mL/min flow; ESI; positive ion mode; detection at 280 nm); HRMS (ESI) calcd for C₂₄H₂₁F₄N₂O₂[M+H]⁺ 445.1534, found 445.1531.

### Example 95: Xanthene Synthesis Via Lithiation of Tetrafluorobenzoic Acid (Scheme 14): JF₆₃₂ (44)

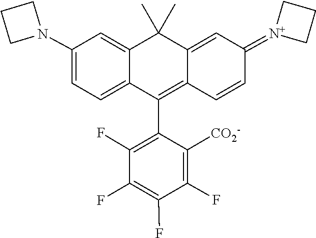

A solution of 2,3,4,5⁻tetrafluorobenzoic acid (6; 525 mg, 2.71 mmol, 6 eq) in THF (9 mL) was cooled to −78° C. under nitrogen. N-Butyllithium (2.5 M in hexanes, 2.17 mL, 5.41 mmol, 12 eq) was added, and the reaction was stirred at −78° C. for 3 h. A solution of 3,6-di(azetidin-1⁻yl)-10,10⁻dimethylanthracen-9(10H)-one (43; 150 mg, 0.451 mmol) in THF (40 mL) was added; the reaction was warmed to room temperature and stirred for 18 h. It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. Purification by silica gel chromatography (0-10% MeOH (2 M NH₃)/CH₂Cl₂, linear gradient) yielded 121 mg (53%) of 44 as a deep blue solid. 1H NMR (CDCl₃, 400 MHz) δ 6.66 (d, J=8.6 Hz, 2H), 6.54 (d, J=2.4 Hz, 2H), 6.27 (dd, J=8.6, 2.3 Hz, 2H), 3.93 (t, J=7.4 Hz, 8H), 2.39 (p, J=7.2 Hz, 4H), 1.75 (s, 3H), 1.70 (s, 3H); 19F NMR (CDCl₃, 376 MHz) δ−139.76 (td, J=20.1, 8.6 Hz, 1F), −141.87 (td, J=20.1, 3.9 Hz, 1F),-143.57 (ddd, J=20.4, 18.0, 8.4 Hz, 1F), −152.12 (ddd, J=20.4, 18.6, 3.8 Hz, 1F); Analytical HPLC: t_{R}=12.0 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₂₉H_{2 5}F₄N₂O₂[M+H]⁺ 509.1847, found 509.1839.

### Example 96: Xanthene Synthesis Via Lithiation of Tetrafluorobenzoic Acid (Scheme 14): JFX₆₃₇ (40)

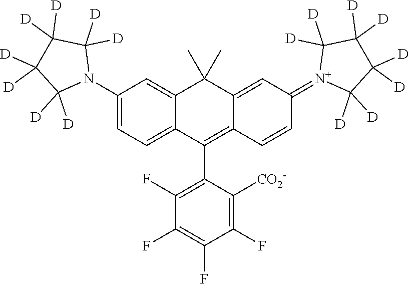

Via lithiation of tetrafluorobenzoic acid: A solution of 2,3,4,5⁻tetrafluorobenzoic acid (6; 557 mg, 2.87 mmol, 6 eq) in THF (10 mL) was cooled to −78° C. under nitrogen. N-Butyllithium (2.5 M in hexanes, 2.29 mL, 5.74 mmol, 12 eq) was added, and the reaction was stirred at −78° C. for 3 h. A solution of 10,10⁻dimethyl-3,6⁻bis(pyrrolidin-1⁻yl-d₈)anthracen-9(10H)-one (42; 180 mg, 0.478 mmol) in THE (15 mL) was added; the reaction was warmed to room temperature and stirred for 18 h. It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. Purification by silica gel chromatography (0-10% MeOH (2 M NH₃)/CH₂Cl₂, linear gradient) yielded 150 mg (57%) of 40 as a dark blue solid.

Via dibromide route: A solution of 1,1′-(propane-2,2⁻diylbis(4⁻bromo-3,1-phenylene))bis(pyrrolidine-2,2,3,3,4,4,5,5⁻d₈) (39; 600 mg, 1.18 mmol) in THE (30 mL) was cooled to −78° C. under nitrogen. tert-Butyllithium (1.7 M in pentane, 3.05 mL, 5.19 mmol, 4.4 eq) was added, and the reaction was stirred at −78° C. for 30 min. It was then warmed to −10° C. before adding a solution of MgBr₂ OEt₂ (670 mg, 2.60 mmol, 2.2 eq) in THE (20 mL). After an additional 30 min at −10° C., a solution of tetrafluorophthalic anhydride (4; 571 mg, 2.60 mmol, 2.2 eq) in THE (20 mL) was added dropwise over 30 min via addition funnel. The reaction was then allowed to warm to room temperature overnight (18 h). It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. Silica gel chromatography (0-10% MeOH (2 M NH₃)/CH₂Cl₂, linear gradient) afforded 257 mg (39%) of 40 as a deep blue solid.

¹H NMR (CDCl₃, 400 MHz) δ 6.694 (d, J=2.5 Hz, 2H), 6.691 (d, J=8.7 Hz, 2H), 6.41 (dd, J=8.7, 2.5 Hz, 2H), 1.81 (s, 3H), 1.75 (s, 3H); 19F NMR (CDCl₃, 376 MHz) δ-140.08 (td, J=20.1, 8.3 Hz, 1F), −141.94 (td, J=20.1, 3.8 Hz, 1F), −143.94-−144.24 (m, 1F),-152.52 (ddd, J=21.8, 18.5, 3.6 Hz, 1F); Analytical HPLC: t_{R}=13.0 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₁H₁₃D₁₆F₄N₂O₂[M+H]⁺ 553.3164, found 553.3154.

### Example 97: Xanthene Synthesis Via Lithiation of Tetrafluorobenzoic Acid (Scheme 14): 4,5,6,7⁻Tetrafluoro-carbo-Q-rhodamine (FCRh_{Q}, 47)

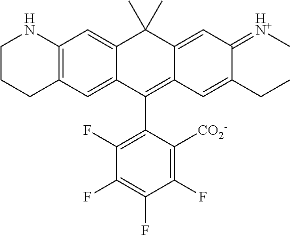

Step 1: A solution of 2,3,4,5⁻tetrafluorobenzoic acid (6; 2.27 g, 11.70 mmol, 6 eq) in THE (40 mL) was cooled to −78° C. under nitrogen. N-Butyllithium (2.5 M in hexanes, 9.36 mL, 23.41 mmol, 12 eq) was added, and the reaction was stirred at −78° C. for 3 h. A solution of 1,11⁻dibenzyl-13,13⁻dimethyl-1,2,3,4,8,9,10,11⁻octahydrobenzo[1,2⁻g:5,4⁻g′]diquinolin-6(13H)-one (45; 1.00 g, 1.95 mmol) in THF (40 mL) was added via cannula; the reaction was immediately warmed to room temperature and stirred for 48 h. It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. The residue was purified by silica gel chromatography (0-20% MeOH (2 M NH₃)/CH₂Cl₂, linear gradient) to yield 145 mg (11%) of 2⁻(1,11⁻dibenzyl-13,13⁻dimethyl-2,3,4,8,9,10,11,13⁻octahydrobenzo[1,2⁻g:5,4⁻g′]diquinolin-6⁻ylium-6(1)-yl)-3,4,5,6-tetrafluorobenzoate (46) as a blue solid.

Step 2: A round-bottom flask was charged with Pd/C (10%, 44.8 mg, 42.1 mol, 0.2 eq) under nitrogen, and a solution of the intermediate from Step 1 (46; 145 mg, 0.211 mmol) in MeOH (15 mL) was added. The sealed flask was evacuated/backfilled with H₂ from a balloon (4×) and then stirred under the H₂ balloon at room temperature for 18 h. The reaction mixture was filtered through Celite with MeOH and EtOAc, and the filtrate was concentrated in vacuo. The blue residue was resuspended in 3:1 CH₂Cl₂/MeOH (8 mL), and p-chloranil (104 mg, 0.421 mmol, 2 eq) was added. After stirring the mixture at room temperature for 4 h, it was deposited onto Celite and concentrated to dryness. Silica gel chromatography (0-20% MeOH (2 M NH₃)/CH₂Cl₂, linear gradient; dry load with Celite) yielded 54.5 mg (51%) of 47 as a dark blue solid. ¹H NMR (CD₃OD, 400 MHz) δ 6.96 (s, 2H), 6.82 (s, 2H), 3.53-3.46 (m, 4H), 2.67 (t, J=6.3 Hz, 4H), 1.91 (p, J=6.2 Hz, 4H), 1.69 (s, 3H), 1.57 (s, 3H); 19F NMR (CD₃OD, 376 MHz) δ -75.44 (s, 3F), −137.26 (ddd, J=19.9, 11.9, 6.9 Hz, 1F), −138.11 (ddd, J=22.2, 12.0, 4.4 Hz, 1F), −151.72-−151.90 (m, 1F), −153.58-−153.75 (m, 1F); Analytical HPLC: t_{R}=12.1 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₂₉H_{2 5}F₄N₂O₂[M+H]⁺ 509.1847, found 509.1847.

### Example 98: Xanthene Synthesis Via Lithiation of Tetrafluorobenzoic Acid (Scheme 14): SiRF₇₁₂ (108)

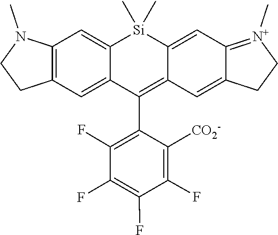

Via tetrafluorobenzoic acid route: A solution of 2,3,4,5⁻tetrafluorobenzoic acid (6; 835 mg, 4.30 mmol, 6 eq) in THE (14 mL) was cooled to −78° C. under nitrogen. N-Butyllithium (2.5 M in hexanes, 3.44 mL, 8.61 mmol, 12 eq) was added, and the reaction was stirred at −78° C. for 3 h. A solution of 1,9,11,11⁻tetramethyl-2,3,7,8,9,11⁻hexahydrosilino[3,2-f5,6⁻f′]diindol-5(1H)-one³¹ (S26; 250 mg, 0.717 mmol) in THE (100 mL) was added via cannula; the reaction was immediately warmed to room temperature and stirred for 48 h. It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. The residue was purified twice by silica gel chromatography (10-100% EtOAc/hexanes, linear gradient; then, 5-100% EtOAc/hexanes, linear gradient, with constant 20% v/v CH₂Cl₂ additive) to yield 45.9 mg (12%) of 108 as a pale blue solid.

Via dibromide route: A solution of bis(5⁻bromo-1⁻methylindolin-6-yl)dimethylsilane (S9; 500 mg, 1.04 mmol) in THF (25 mL) was cooled to −78° C. under nitrogen. tert-Butyllithium (1.7 M in pentane, 2.69 mL, 4.58 mmol, 4.4 eq) was added, and the reaction was stirred at −78° C. for 30 min. It was then warmed to −10° C. before adding a solution of MgBr₂ OEt₂ (591 mg, 2.29 mmol, 2.2 eq) in THF (10 mL). After an additional 30 min at −10° C., a solution of tetrafluorophthalic anhydride (4; 504 mg, 2.29 mmol, 2.2 eq) in THE (10 mL) was added dropwise over 30 min via addition funnel. The reaction was then allowed to warm to room temperature overnight (18 h). It was subsequently diluted with saturated NH₄Cl and water and extracted with EtOAc (2×). The combined organic extracts were washed with saturated NaHCO₃ and brine, dried over anhydrous MgSO₄, filtered, and concentrated in vacuo. The crude material was redissolved in MeOH (10 mL); 1 M HCl (0.5 mL) was added, and the solution was aged 15 min. After adding 1 N NaOH (0.5 mL), the mixture was deposited onto Celite and concentrated to dryness. Silica gel chromatography (20-100% EtOAc/hexanes, linear gradient; dry load with Celite) afforded 232 mg (42%) of 108 as a light green solid.

¹H NMR (CDCl₃, 400 MHz) δ 6.63 (s, 2H), 6.60 (s, 2H), 3.33 (t, J=8.2 Hz, 4H), 2.94-2.84 (m, 4H), 2.82 (s, 6H), 0.52 (s, 3H), 0.51 (s, 3H); 19F NMR (CDCl₃, 376 MHz) δ-139.28 (td, J=20.1, 8.5 Hz, 1F), −140.87 (td, J=20.2, 3.8 Hz, 1F), −143.48 (ddd, J=21.0, 18.3, 8.6 Hz, 1F), −152.48 (ddd, J=21.6, 18.4, 3.7 Hz, 1F); Analytical HPLC: t_{R}=12.3 min, >99% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₂₈H_{2 5}F₄N₂O₂Si [M+H]⁺ 525.1616, found 525.1614.

### Example 99: Synthesis of Carborhodamines via AlCl₃ Condensation (Scheme 16): JF₆₆₀ (57)

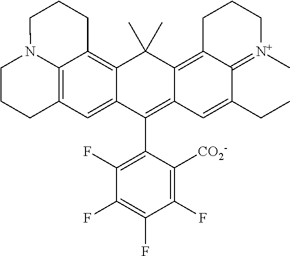

A solution of tetrafluorophthalic anhydride (4; 85.4 mg, 0.388 mmol, 1.5 eq) and AlCl₃ (103 mg, 0.776 mmol, 3 eq) in 1,2⁻dichloroethane (5 mL) was added to a stirred solution of 8,8′-(propane-2,2⁻diyl)bis(2,3,6,7⁻tetrahydro-1H,5H-pyrido[3,2,1⁻ij]quinoline) (56; 100 mg, 0.259 mmol) in 1,2⁻dichloroethane (2 mL). After stirring the reaction at room temperature for 1 h, it was treated with Et₃N (108 μL, 0.776 mmol, 3 eq). A second portion of tetrafluorophthalic anhydride (57.5 mg, 0.388 mmol, 1.5 eq) and AlCl₃ (103 mg, 0.776 mmol, 3 eq) in 1,2-dichloroethane (5 mL) was added, and the reaction was stirred at room temperature an additional 1 h. It was then poured into ice water (50 mL), diluted with 1 M HCl (10 mL), and extracted with CH₂Cl₂ (2×). The organic extracts were washed with saturated NaHCO₃, dried over anhydrous MgSO₄, filtered, and concentrated in vacuo. Flash chromatography on silica gel (0-20% MeOH (2 M NH₃)/CH₂Cl₂, linear gradient) afforded 103 mg (68%) of 57 as a deep blue solid. 1H NMR (CD₃OD, 400 MHz) δ 6.67 (s, 2H), 3.56 (t, J=6.2 Hz, 4H), 3.51 (t, J=5.9 Hz, 4H), 3.10-3.01 (m, 4H), 2.69 (dt, J=15.9, 6.2 Hz, 2H), 2.59 (dt, J=15.4, 6.3 Hz, 2H), 2.07-1.99 (m, 4H), 2.01 (s, 3H), 1.98 (s, 3H), 1.92 (p, J=6.2 Hz, 4H); 19F NMR (CD₃OD, 376 MHz) δ−139.88 (ddd, J=21.8, 12.4, 3.2 Hz, 1F), −141.77 (ddd, J=22.4, 12.5, 3.1 Hz, 1F), −155.38 (ddd, J=22.4, 19.3, 3.1 Hz, 1F), −158.93 (ddd, J=22.2, 19.2, 3.2 Hz, 1F); Analytical HPLC: t_{R}=13.4 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₅H₃₃F₄N₂O₂[M+H]⁺ 589.2473, found 589.2476.

### Example 100: Synthesis of Carborhodamines via AlCl₃ Condensation (Scheme 16): Carborhodamine 101 (CRh₁₀₁, 97)

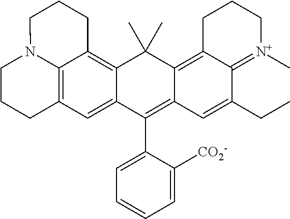

A solution of phthalic anhydride (S15; 57.5 mg, 0.388 mmol, 1.5 eq) and AlCl₃ (103 mg, 0.776 mmol, 3 eq) in 1,2⁻dichloroethane (5 mL) was added to a stirred solution of 8,8′-(propane-2,2⁻diyl)bis(2,3,6,7⁻tetrahydro-1H,5H-pyrido[3,2,1⁻ij]quinoline) (56; 100 mg, 0.259 mmol) in 1,2⁻dichloroethane (2 mL). After stirring the reaction at room temperature for 1 h, it was treated with Et₃N (108 μL, 0.776 mmol, 3 eq). A second portion of phthalic anhydride (57.5 mg, 0.388 mmol, 1.5 eq) and AlCl₃ (103 mg, 0.776 mmol, 3 eq) in 1,2⁻dichloroethane (5 mL) was added, and the reaction was stirred at room temperature an additional 1 h. It was then poured into ice water (50 mL), diluted with 1 M HCl (10 mL), and extracted with CH₂Cl₂ (2×). The organic extracts were washed with saturated NaHCO₃, dried over anhydrous MgSO₄, filtered, and concentrated in vacuo. Flash chromatography on silica gel (0-20% MeOH (2 M NH₃)/CH₂Cl₂, linear gradient) afforded 116 mg (87%) of 97 as a deep blue solid. 1H NMR (CDCl₃, 400 MHz) δ 7.97-7.92 (m, 1H), 7.52 (td, J=7.4, 1.4 Hz, 1H), 7.47 (td, J=7.4, 1.2 Hz, 1H), 7.05-7.01 (m, 1H), 6.11 (s, 2H), 3.20 (t, J=6.7 Hz, 4H), 3.16 (t, J=5.8 Hz, 4H), 2.98-2.85 (m, 4H), 2.56-2.37 (m, 4H), 2.07 (s, 3H), 2.02 (s, 3H), 1.95 (p, J=6.4 Hz, 4H), 1.88-1.79 (m, 4H); 13C NMR (CDCl₃, 101 MHz) δ 171.4 (C), 157.0 (C), 146.4 (C), 145.0 (C), 134.5 (CH), 128.4 (CH), 127.1 (C), 126.1 (CH), 124.7 (CH), 123.8 (CH), 121.3 (C), 119.6 (C), 116.4 (C), 50.8 (CH₂), 50.1 (CH₂), 37.0 (C), 34.6 (CH₃), 33.3 (CH₃), 28.4 (CH₂), 28.2 (CH₂), 22.3 (CH₂), 21.8 (CH₂); Analytical HPLC: t_{R}=13.1 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₅H₃₇N₂O₂ [M+H]⁺ 517.2850, found 517.2856.

### Example 101: Synthesis of Carborhodamines via AlCl₃ Condensation (Scheme 16): JF₆₅₇ (59)

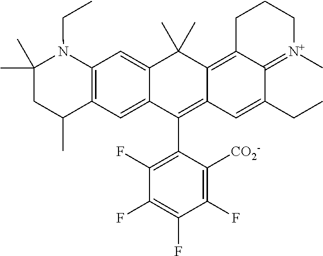

A solution of tetrafluorophthalic anhydride (4; 79.2 mg, 0.360 mmol, 1.5 eq) and AlCl₃ (96.0 mg, 0.720 mmol, 3 eq) in 1,2⁻dichloroethane (5 mL) was added to a stirred solution of 8-(2⁻(1-ethyl-2,2,4⁻trimethyl-1,2,3,4⁻tetrahydroquinolin-7⁻yl)propan-2⁻yl)-2,3,6,7⁻tetrahydro-1H,5H-pyrido[3,2,1⁻ij]quinoline (58; 100 mg, 0.240 mmol) in 1,2⁻dichloroethane (4 mL). After stirring the reaction at room temperature for 1 h, it was treated with Et₃N (100 μL, 0.720 mmol, 3 eq). A second portion of phthalic anhydride (79.2 mg, 0.360 mmol, 1.5 eq) and AlCl₃ (96.0 mg, 0.720 mmol, 3 eq) in 1,2⁻dichloroethane (5 mL) was added, and the reaction was stirred at room temperature an additional 2 h. It was then poured into ice water (50 mL), diluted with 1 M HCl (10 mL), and extracted with CH₂Cl₂ (2×). The organic extracts were washed with saturated NaHCO₃, dried over anhydrous MgSO₄, filtered, and concentrated in vacuo. Flash chromatography on silica gel (0-20% MeOH (2 M NH₃)/CH₂Cl₂, linear gradient) afforded 119 mg (80%) of 59 as a deep blue solid (mixture of two diastereomers). ¹H NMR (CD₃OD, 400 MHz) δ 6.91 (s, 1H), 6.88-6.83 (m, 2H), 3.82 (dq, J=14.3, 7.1 Hz, 1H), 3.70-3.62 (m, 1H), 3.60 (t, J=6.0 Hz, 2H), 3.55 (t, J=5.9 Hz, 2H), 3.23-3.15 (m, 2H), 2.89-2.67 (m, 2H), 2.62 (dt, J=15.5, 6.2 Hz, 1H), 2.10-2.00 (m, 2H), 1.98-1.90 (m, 2H), 1.90-1.82 (m, 1H), 1.89 (s, 1.5H), 1.87 (s, 1.5H), 1.86 (s, 1.5H), 1.85 (s, 1.5H), 1.55 (t, J=13.3 Hz, 0.5H), 1.54 (t, J=13.4 Hz, 0.5H), 1.48 (s, 1.5H), 1.47 (s, 1.5H), 1.36 (s, 1.5H), 1.35 (t, J=7.1 Hz, 3H), 1.34 (s, 1.5H), 1.18 (d, J=6.5 Hz, 1.5H), 1.14 (d, J=6.5 Hz, 1.5H); 19F NMR (CD₃OD, 376 MHz) δ−139.60 (ddd, J=21.9, 12.4, 3.3 Hz, 0.5F), −139.68 (ddd, J=21.6, 12.7, 3.2 Hz, 0.5F), −141.14 (ddd, J=22.4, 12.3, 3.4 Hz, 0.5F), −141.31 (ddd, J=22.5, 12.4, 3.2 Hz, 0.5F), −154.88 (ddd, J=22.4, 19.2, 3.2 Hz, 0.5F), −155.04 (ddd, J=22.5, 19.2, 3.0 Hz, 0.5F), −158.72 (ddd, J=22.5, 18.8, 3.3 Hz, 0.5F), −159.07 (ddd, J=22.0, 19.2, 3.2 Hz, 0.5F); Analytical HPLC: t_{R} (two isomers)=14.1 min, 14.4 min; >99% total purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₃₉F₄N₂O₂[M+H]⁺ 619.2942, found 619.2948.

A portion of this material (60 mg) was further purified by reverse phase HPLC (30-60% MeCN/H₂O, linear gradient, with constant 0.1% v/v TFA additive) to separate the isomeric mixture. The pooled fractions of each isomer were partially concentrated to remove MeCN, diluted with saturated NaHCO₃, and extracted with CH₂Cl₂ (3×). The organic extracts were dried over anhydrous MgSO₄, filtered, and evaporated to provide the two individual diastereomers as blue solids: 15.7 mg of the faster-eluting isomer (“diastereomer 1”) and 26.3 mg of the slower-eluting isomer (“diastereomer 2”).

Diastereomer 1: ¹H NMR (CD₃OD, 400 MHz) δ 6.91 (s, 1H), 6.86 (s, 1H), 6.85 (s, 1H), 3.82 (dq, J=14.2, 7.1 Hz, 1H), 3.66 (dq, J=14.1, 7.4 Hz, 1H), 3.60 (t, J=6.1 Hz, 2H), 3.55 (t, J=5.8 Hz, 2H), 3.23-3.15 (m, 2H), 2.82-2.67 (m, 2H), 2.62 (dt, J=15.4, 6.2 Hz, 1H), 2.10-2.00 (m, 2H), 1.98-1.90 (m, 2H), 1.90-1.82 (m, 1H), 1.89 (s, 3H), 1.85 (s, 3H), 1.55 (t, J=13.3 Hz, 1H), 1.47 (s, 3H), 1.36 (s, 3H), 1.35 (t, J=7.1 Hz, 3H), 1.18 (d, J=6.6 Hz, 3H); 19F NMR (CD₃OD, 376 MHz) δ−139.61 (ddd, J=21.9, 12.4, 3.1 Hz, 1F), −141.17 (ddd, J=22.6, 12.3, 3.3 Hz, 1F), −155.05 (ddd, J=22.3, 19.0, 3.2 Hz, 1F), −158.78 (ddd, J=22.4, 19.3, 3.2 Hz, 1F); Analytical HPLC: t_{R}=14.1 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₃₉F₄N₂O₂[M+H]⁺ 619.2942, found 619.2945.

Diastereomer 2: ¹H NMR (CD₃OD, 400 MHz) δ 6.91 (s, 1H), 6.88-6.83 (m, 2H), 3.82 (dq, J=14.2, 7.0 Hz, 1H), 3.64 (dq, J=14.2, 7.4 Hz, 1H), 3.60 (t, J=5.9 Hz, 2H), 3.55 (t, J=5.9 Hz, 2H), 3.23-3.15 (m, 2H), 2.89-2.78 (m, 1H), 2.73 (dt, J=15.8, 6.1 Hz, 1H), 2.62 (dt, J=15.4, 6.2 Hz, 1H), 2.10-2.00 (m, 2H), 1.98-1.90 (m, 2H), 1.90-1.83 (m, 1H), 1.87 (s, 3H), 1.86 (s, 3H), 1.54 (t, J=13.4 Hz, 1H), 1.48 (s, 3H), 1.35 (t, J=7.1 Hz, 3H), 1.34 (s, 3H), 1.14 (d, J=6.5 Hz, 3H); 19F NMR (CD₃OD, 376 MHz) δ−139.69 (ddd, J=21.8, 12.6, 3.3 Hz, 1F), −141.32 (ddd, J=22.5, 12.5, 3.1 Hz, 1F), −154.88 (ddd, J=22.5, 19.3, 3.2 Hz, 1F), −159.09 (ddd, J=21.7, 19.2, 3.1 Hz, 1F); Analytical HPLC: t_{R}=14.4 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₃₉F₄N₂O₂[M+H]⁺ 619.2942, found 619.2948.

### Example 102: Synthesis of Carborhodamines via AlCl₃ Condensation (Scheme 16): ATTO 647N free dye (S57)

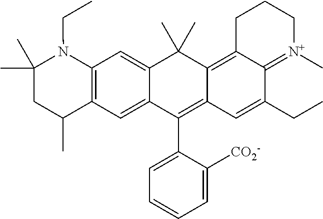

A solution of phthalic anhydride (S15; 53.3 mg, 0.360 mmol, 1.5 eq) and AlCl₃ (96.0 mg, 0.720 mmol, 3 eq) in 1,2⁻dichloroethane (5 mL) was added to a stirred solution of 8-(2⁻(1-ethyl-2,2,4⁻trimethyl-1,2,3,4⁻tetrahydroquinolin-7⁻yl)propan-2⁻yl)-2,3,6,7⁻tetrahydro-1H,5H-pyrido[3,2,1⁻ij]quinoline (58; 100 mg, 0.240 mmol) in 1,2⁻dichloroethane (4 mL). After stirring the reaction at room temperature for 1 h, it was treated with Et₃N (100 μL, 0.720 mmol, 3 eq). A second portion of phthalic anhydride (53.3 mg, 0.360 mmol, 1.5 eq) and AlCl₃ (96.0 mg, 0.720 mmol, 3 eq) in 1,2⁻dichloroethane (5 mL) was added, and the reaction was stirred at room temperature an additional 2 h. It was then poured into ice water (50 mL), diluted with 1 M HCl (10 mL), and extracted with CH₂Cl₂ (2×). The organic extracts were washed with saturated NaHCO₃, dried over anhydrous MgSO₄, filtered, and concentrated in vacuo. Flash chromatography on silica gel (0-20% MeOH (2 M NH₃)/CH₂Cl₂, linear gradient) afforded 110 mg (84%) of S57 as a blue solid (mixture of two diastereomers). ¹H NMR (CD₃OD, 400 MHz) δ 8.10-8.01 (m, 1H), 7.61-7.52 (m, 2H), 7.13-7.07 (m, 1H), 6.881 (s, 0.5H), 6.876 (s, 0.5H), 6.85-6.78 (m, 2H), 3.78 (dq, J=14.5, 7.1 Hz, 1H), 3.65-3.56 (m, 1H), 3.56-3.51 (m, 2H), 3.51-3.45 (m, 2H), 3.23-3.14 (m, 2H), 2.80-2.63 (m, 1H), 2.62-2.45 (m, 2H), 2.04 (p, J=6.1 Hz, 2H), 1.93-1.85 (m, 2H), 1.910 (s, 1.5H), 1.901 (s, 1.5H), 1.892 (s, 1.5H), 1.885 (s, 1.5H), 1.85-1.78 (m, 1H), 1.52-1.45 (m, 1H), 1.45 (s, 1.5H), 1.44 (s, 1.5H), 1.36-1.30 (m, 3H), 1.31 (s, 1.5H), 1.30 (s, 1.5H), 1.01 (d, J=6.6 Hz, 1.5H), 0.98 (d, J=6.5 Hz, 1.5H); Analytical HPLC: t_{R} (two isomers)=14.0 min, 14.3 min; >99% total purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₄₃N₂O₂ [M+H]⁺ 547.3319, found 547.3325.

A portion of this material (80 mg) was further purified by reverse phase HPLC (30-60% MeCN/H₂O, linear gradient, with constant 0.1% v/v TFA additive) to separate the isomeric mixture. The pooled fractions of each isomer were partially concentrated to remove MeCN, diluted with saturated NaHCO₃, and extracted with CH₂Cl₂ (2×). The organic extracts were dried over anhydrous MgSO₄, filtered, and evaporated to provide the two individual diastereomers as blue solids: 30.3 mg of the faster-eluting isomer (“diastereomer 1”) and 25.4 mg of the slower-eluting isomer (“diastereomer 2”).

Diastereomer 1: ¹H NMR (CD₃OD, 400 MHz) δ 8.10-8.05 (m, 1H), 7.57 (td, J=7.4, 1.6 Hz, 1H), 7.54 (td, J=7.4, 1.6 Hz, 1H), 7.11-7.07 (m, 1H), 6.87 (s, 1H), 6.82 (d, J=1.6 Hz, 1H), 6.81-6.78 (m, 1H), 3.78 (dq, J=14.1, 7.0 Hz, 1H), 3.59 (dq, J=14.2, 7.1 Hz, 1H), 3.53 (t, J=6.3 Hz, 2H), 3.47 (t, J=5.8 Hz, 2H), 3.22-3.14 (m, 2H), 2.74-2.63 (m, 1H), 2.62-2.45 (m, 2H), 2.03 (p, J=6.3 Hz, 2H), 1.92-1.85 (m, 2H), 1.91 (s, 3H), 1.89 (s, 3H), 1.80 (dd, J=13.2, 4.3 Hz, 1H), 1.49 (d, J=13.2 Hz, 1H), 1.44 (s, 3H), 1.33 (t, J=7.0 Hz, 3H), 1.31 (s, 3H), 1.01 (d, J=6.5 Hz, 3H); 13C NMR (CD₃OD, 101 MHz) δ 173.3 (C), 165.2 (C), 158.7 (C), 153.5 (C), 152.8 (C), 151.0 (C), 140.5 (C), 139.8 (C), 135.6 (CH), 131.5 (CH), 130.6 (CH), 130.3 (CH), 130.1 (CH), 129.7 (CH), 129.3 (C), 123.7 (C), 123.4 (C), 122.2 (C), 120.5 (C), 111.8 (CH), 58.4 (C), 52.8 (CH₂), 52.2 (CH₂), 46.6 (CH₂), 41.5 (C), 40.7 (CH₂), 31.4 (CH₃), 31.0 (CH₃), 29.5 (CH₃), 28.7 (CH₂), 27.6 (CH), 26.2 (CH₃), 22.0 (CH₂), 21.9 (CH₂), 19.1 (CH₃), 14.7 (CH₃); Analytical HPLC: t_{R}=14.1 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₄₃N₂O₂ [M+H]⁺ 547.3319, found 547.3328.

Diastereomer 2: ¹H NMR (CD₃OD, 400 MHz) δ 8.06-8.01 (m, 1H), 7.57 (td, J=7.4, 1.6 Hz, 1H), 7.54 (td, J=7.4, 1.7 Hz, 1H), 7.13-7.09 (m, 1H), 6.88 (s, 1H), 6.84 (d, J=1.6 Hz, 1H), 6.83-6.80 (m, 1H), 3.78 (dq, J=14.2, 7.0 Hz, 1H), 3.60 (dq, J=14.2, 7.1 Hz, 1H), 3.54 (t, J=6.2 Hz, 2H), 3.48 (t, J=5.8 Hz, 2H), 3.23-3.15 (m, 2H), 2.80-2.68 (m, 1H), 2.62-2.45 (m, 2H), 2.04 (p, J=6.2 Hz, 2H), 1.93-1.85 (m, 2H), 1.90 (s, 3H), 1.89 (s, 3H), 1.82 (dd, J=13.3, 4.4 Hz, 1H), 1.46 (t, J=13.2 Hz, 1H), 1.45 (s, 3H), 1.34 (t, J=7.0 Hz, 3H), 1.30 (s, 3H), 0.98 (d, J=6.5 Hz, 3H); 13C NMR (CD₃OD, 101 MHz) δ 173.8 (C), 166.4 (C), 158.9 (C), 153.6 (C), 153.0 (C), 151.2 (C), 140.9 (C), 139.1 (C), 135.8 (CH), 132.1 (CH), 130.5 (CH), 130.4 (CH), 130.1 (CH), 129.7 (CH), 129.4 (C), 123.8 (C), 123.5 (C), 122.3 (C), 120.6 (C), 111.9 (CH), 58.4 (C), 52.9 (CH₂), 52.2 (CH₂), 46.6 (CH₂), 41.6 (C), 40.8 (CH₂), 31.3 (CH₃), 31.0 (CH₃), 29.5 (CH₃), 28.67 (CH₂), 28.65 (CH₂), 27.5 (CH), 26.1 (CH₃), 22.0 (CH₂), 21.9 (CH₂), 19.4 (CH₃), 14.7 (CH₃); Analytical HPLC: t_{R}=14.4 min, 98.7% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₄₃N₂O₂ [M+H]⁺ 547.3319, found 547.3319.

### c 4,5,6,7⁻Tetrafluoro-fluorescein ditriflate (61)

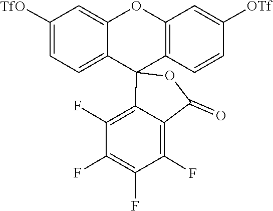 [SMILES: O=C1OC2(c3ccc(OS(=O)(=O)C(F)(F)F)cc3Oc3cc(OS(=O)(=O)C(F)(F)F)ccc32)c2c(F)c(F)c(F)c(F)c21]

4,5,6,7⁻Tetrafluoro-fluorescein¹⁹ (60; 1.00 g, 2.47 mmol) was taken up in CH₂Cl₂ (25 mL) and cooled to 0° C. Pyridine (1.60 mL, 19.79 mmol, 8 eq) and trifluoromethanesulfonic anhydride (1.66 mL, 9.89 mmol, 4 eq) were added, and the reaction was allowed to warm to room temperature overnight (18 h). It was subsequently diluted with water and extracted with CH₂Cl₂ (2×). The combined organic extracts were washed with saturated CuSO₄ and brine, dried over anhydrous MgSO₄, filtered, and concentrated in vacuo. Flash chromatography on silica gel (0-40% EtOAc/hexanes, linear gradient) afforded 1.10 g (67%) of 61 as a white foam. 1H NMR (CDCl₃, 400 MHz) δ 7.35-7.32 (m, 2H), 7.16-7.10 (m, 4H); 19F NMR (CDCl₃, 376 MHz) δ-73.07 (s, 6F), −136.01 (td, J=19.8, 10.3 Hz, 1F), −139.59 (ddd, J=20.7, 18.0, 10.3 Hz, 1F),-141.78 (td, J=19.9, 5.1 Hz, 1F), −147.64 (ddd, J=20.2, 18.0, 5.1 Hz, 1F); Analytical HPLC: t_{R}=16.2 min, >99% purity (30-95% MeCN/H₂O, linear gradient, with constant 0.1% v/v TFA additive; 20 min run; 1 mL/min flow; ESI; positive ion mode; detection at 280 nm); HRMS (ESI) calcd for C₂₂H₇F₁₀₀₉S₂ [M+H]⁺ 668.9366, found 668.9361.

### c 4,5,6,7⁻Tetrafluoro-carbofluorescein ditriflate (62)

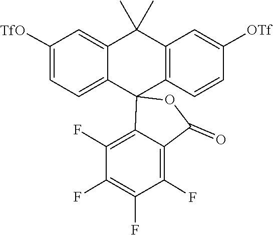 [SMILES: CC1(C)c2cc(OS(=O)(=O)C(F)(F)F)ccc2C2(OC(=O)c3c(F)c(F)c(F)c(F)c32)c2ccc(OS(=O)(=O)C(F)(F)F)cc21]

To a solution of 4,5,6,7⁻tetrafluoro-carbofluorescein (51; 445 mg, 1.03 mmol) in CH₂Cl₂ (10 mL) were added pyridine (669 μL, 8.27 mmol, 8 eq) and trifluoromethanesulfonic anhydride (696 μL, 4.14 mmol, 4 eq). The reaction was stirred at room temperature for 1 h. It was subsequently diluted with water and extracted with CH₂Cl₂ (2×). The combined organic extracts were washed with saturated CuSO₄ and brine, dried over anhydrous MgSO₄, filtered, and concentrated in vacuo. Flash chromatography on silica gel (0-30% EtOAc/hexanes, linear gradient) afforded 684 mg (95%) of 62 as a white solid. ¹H NMR (CDCl₃, 400 MHz) δ 7.56 (d, J=2.6 Hz, 2H), 7.19 (dd, J=8.8, 2.5 Hz, 2H), 7.02 (d, J=8.8 Hz, 2H), 1.84 (s, 3H), 1.79 (s, 3H); 19F NMR (CDCl₃, 376 MHz) δ−73.10 (s, 6F), −136.52 (td, J=19.9, 10.1 Hz, 1F), −140.09 (ddd, J=20.4, 18.1, 10.1 Hz, 1F), −141.51 (td, J=19.9, 4.8 Hz, 1F), −148.41 (ddd, J=20.5, 18.2, 4.8 Hz, 1F); Analytical HPLC: t_{R}=16.4 min, 97.7% purity (30-95% MeCN/H₂O, linear gradient, with constant 0.1% v/v TFA additive; 20 min run; 1 mL/min flow; ESI; positive ion mode; detection at 280 nm); HRMS (EI) calcd for C₂₅H₁₂F₁₀O₈S₂ [M^{·}]⁺693.9808, found 693.9820.

### Example 105: Rhodamine 110 and N-Aryl Rhodamine Synthesis via Cross-Coupling (Scheme 11): 4,5,6,7⁻Tetrafluoro-Si-fluorescein ditriflate (63)

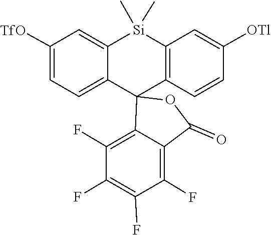

To a solution of 4,5,6,7⁻tetrafluoro-Si-fluorescein (55; 170 mg, 0.381 mmol) in CH₂Cl₂ (5 mL) were added pyridine (246 μL, 3.05 mmol, 8 eq) and trifluoromethanesulfonic anhydride (256 μL, 1.52 mmol, 4 eq). The reaction was stirred at room temperature for 1 h. It was subsequently diluted with water and extracted with CH₂Cl₂ (2×). The combined organic extracts were washed with saturated CuSO₄ and brine, dried over anhydrous MgSO₄, filtered, and concentrated in vacuo. Flash chromatography on silica gel (0-25% EtOAc/hexanes, linear gradient) afforded 261 mg (96%) of 63 as a white foam. ¹H NMR (CDCl₃, 400 MHz) δ 7.58 (d, J=2.7 Hz, 2H), 7.27 (dd, J=8.9, 2.7 Hz, 2H), 7.15 (dd, J=8.9, 1.4 Hz, 2H), 0.69 (s, 3H), 0.67 (s, 3H); 19F NMR (CDCl₃, 376 MHz) δ−73.17 (s, 6F), −135.84 (td, J=19.8, 10.1 Hz, 1F), −137.58-−137.80 (m, 1F), −140.68 (ddd, J=21.0, 18.3, 10.1 Hz, 1F), −148.14 (ddd, J=20.5, 18.1, 4.9 Hz, 1F); Analytical HPLC: t_{R}=16.9 min, 97.8% purity (30-95% MeCN/H₂O, linear gradient, with constant 0.1% v/v TFA additive; 20 min run; 1 mL/min flow; ESI; positive ion mode; detection at 280 nm); HRMS (ESI) calcd for C₂₄H₁₃F₁₀O₈S₂Si [M+H]⁺ 710.9656, found 710.9660.

### Example 106: Rhodamine 110 and N-Aryl Rhodamine Synthesis via Cross-Coupling (Scheme 11): 2⁻(3,6⁻Bis(methyl(phenyl)amino)xanthylium-9⁻yl)-3,4,5,6-tetrafluorobenzoate (64)

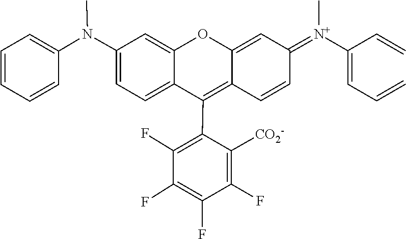

A vial was charged with 4,5,6,7⁻tetrafluoro-fluorescein ditriflate (61; 90 mg, 0.135 mmol), Pd₂dba₃ (12.3 mg, 13.5 mol, 0.1 eq), XPhos (19.3 mg, 40.4 mol, 0.3 eq), and Cs₂CO₃ (123 mg, 0.377 mmol, 2.8 eq). The vial was sealed and evacuated/backfilled with nitrogen (3×). Dioxane (1 mL) was added, and the reaction was flushed again with nitrogen (3×). Following the addition of N-methylaniline (32.1 μL, 0.296 mmol, 2.2 eq), the reaction was stirred at 80° C. for 4 h. It was then cooled to room temperature, diluted with CH₂Cl₂, deposited onto Celite, and concentrated to dryness. Purification by silica gel chromatography (10-100% EtOAc/hexanes, linear gradient; dry load with Celite) afforded 64 (70.7 mg, 90%) as a pale purple solid. ¹H NMR (CDCl₃, 400 MHz) δ 7.41-7.35 (m, 4H), 7.22-7.16 (m, 6H), 6.67 (d, J=8.8 Hz, 2H), 6.60 (d, J=2.4 Hz, 2H), 6.52 (dd, J=8.8, 2.5 Hz, 2H), 3.33 (s, 6H); 19F NMR (CDCl₃, 376 MHz) δ−139.02 (td, J=20.0, 8.7 Hz, 1F), −141.95 (td, J=20.0, 4.0 Hz, 1F),-143.05 (ddd, J=20.4, 18.0, 8.6 Hz, 1F), −150.82-−151.01 (m, 1F); Analytical HPLC: t_{R}=11.0 min, >99% purity (30-95% MeCN/H₂O, linear gradient, with constant 0.1% v/v TFA additive; 20 min run; 1 mL/min flow; ESI; positive ion mode; detection at 550 nm); HRMS (ESI) calcd for C₃₄H_{2 3}F₄N₂O₃[M+H]⁺ 583.1639, found 583.1631.

### Example 107: Rhodamine 110 and N-Aryl Rhodamine Synthesis via Cross-Coupling (Scheme 11): JQ₆₄₅ (65)

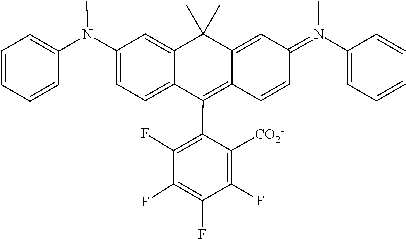

A vial was charged with 4,5,6,7⁻tetrafluoro-carbofluorescein ditriflate (62; 250 mg, 0.360 mmol), Pd₂dba₃ (33.0 mg, 36.0 mol, 0.1 eq), XPhos (51.5 mg, 0.108 mmol, 0.3 eq), and Cs₂CO₃ (328 mg, 1.01 mmol, 2.8 eq). The vial was sealed and evacuated/backfilled with nitrogen (3×). Dioxane (2 mL) was added, and the reaction was flushed again with nitrogen (3×). Following the addition of N-methylaniline (85.8 μL, 0.792 mmol, 2.2 eq), the reaction was stirred at 80° C. for 8 h. It was then cooled to room temperature, filtered through Celite with CH₂Cl₂, and concentrated to dryness. Purification by silica gel chromatography (0-2₅% EtOAc/hexanes, linear gradient) afforded 65 (167 mg, 76%) as an off-white solid. 1H NMR (CDCl₃, 400 MHz) δ 7.37-7.31 (m, 4H), 7.17-7.06 (m, 8H), 6.74 (dd, J=8.8, 2.4 Hz, 2H), 6.69 (d, J=8.8 Hz, 2H), 3.36 (s, 6H), 1.65 (s, 3H), 1.63 (s, 3H); 19F NMR (CDCl₃, 376 MHz) δ-139.31 (td, J=20.0, 8.6 Hz, 1F), −141.79 (td, J=20.1, 3.9 Hz, 1F), −143.16 (ddd, J=21.0, 18.6, 8.7 Hz, 1F), −151.67 (ddd, J=21.2, 18.2, 3.7 Hz, 1F); Analytical HPLC: t_{R}=16.4 min, >99% purity (30-95% MeCN/H₂O, linear gradient, with constant 0.1% v/v TFA additive; 20 min run; 1 mL/min flow; ESI; positive ion mode; detection at 650 nm); HRMS (ESI) calcd for C₃₇H₂₉F₄N₂O₂[M+H]⁺ 609.2160, found 609.2149.

### Example 108: Rhodamine 110 and N-Aryl Rhodamine Synthesis via Cross-Coupling (Scheme 11): 5,5⁻Dimethyl-3,7⁻bis(methyl(phenyl)amino)-3′H,5H-spiro[dibenzo[b,e]siline-10,1′-isobenzofuran]-3′-one (92)

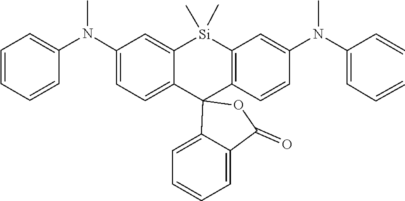 [SMILES: C=C/C=C(\C=C)N(C)c1ccc2c(c1)[Si](C)(C)c1cc(N(C)c3ccccc3)ccc1C21OC(=O)c2ccccc21]

A vial was charged with Si-fluorescein ditriflate¹⁷ (120 mg, 0.188 mmol), Pd₂dba₃ (17.2 mg, 18.8 mol, 0.1 eq), XPhos (26.9 mg, 56.4 mol, 0.3 eq), and Cs₂CO₃ (171 mg, 0.526 mmol, 2.8 eq). The vial was sealed and evacuated/backfilled with nitrogen (3×). Dioxane (1.5 mL) was added, and the reaction was flushed again with nitrogen (3×). Following the addition of N-methylaniline (44.8 μL, 0.413 mmol, 2.2 eq), the reaction was stirred at 100° C. for 3 h. It was then cooled to room temperature, filtered through Celite with CH₂Cl₂, and concentrated to dryness. Purification by silica gel chromatography (0-40% EtOAc/hexanes, linear gradient) afforded 92 (71.1 mg, 68%) as a white solid. ¹H NMR (CDCl₃, 400 MHz) δ 7.97 (dt, J=7.7, 1.0 Hz, 1H), 7.67 (td, J=7.5, 1.2 Hz, 1H), 7.56 (td, J=7.5, 0.9 Hz, 1H), 7.37 (dt, J=7.7, 1.0 Hz, 1H), 7.32-7.26 (m, 4H), 7.24 (d, J=2.6 Hz, 2H), 7.11-7.05 (m, 4H), 7.05-7.00 (m, 2H), 6.81 (d, J=8.7 Hz, 2H), 6.76 (dd, J=8.8, 2.6 Hz, 2H), 3.32 (s, 6H), 0.56 (s, 3H), 0.53 (s, 3H);

¹³C NMR (CDCl₃, 101 MHz) δ 170.5 (C), 153.7 (C), 148.4 (C), 148.2 (C), 137.4 (C), 135.6 (C), 133.8 (CH), 129.5 (CH), 129.1 (CH), 128.1 (CH), 127.1 (C), 126.0 (CH), 124.8 (CH), 122.92 (CH), 122.89 (CH), 122.5 (CH), 119.3 (CH), 91.4 (C), 40.2 (CH₃), 0.4 (CH₃), −1.7 (CH₃); Analytical HPLC: t_{R}=15.3 min, >99% purity (50-95% MeCN/H₂O, linear gradient, with constant 0.1% v/v TFA additive; 20 min run; 1 mL/min flow; ESI; positive ion mode; detection at 254 nm); HRMS (ESI) calcd for C₃₆H₃₃N₂O₂Si [M+H]⁺ 553.2306, found 553.2304.

### Example 109: Rhodamine 110 and N-Aryl Rhodamine Synthesis via Cross-Coupling (Scheme 11): 2⁻(5,5⁻Dimethyl-3,7⁻bis(methyl(phenyl)amino)dibenzo[b,e]silin-10-ylium-10(5H)-yl)-3,4,5,6⁻tetrafluoro-benzoate (66)

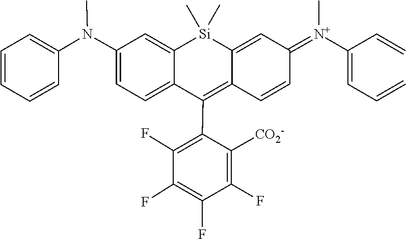

A vial was charged with 4,5,6,7⁻tetrafluoro-Si-fluorescein ditriflate (63; 175 mg, 0.246 mmol), Pd₂dba₃ (22.6 mg, 24.6 mol, 0.1 eq), XPhos (35.2 mg, 73.9 mol, 0.3 eq), and Cs₂CO₃ (225 mg, 0.690 mmol, 2.8 eq). The vial was sealed and evacuated/backfilled with nitrogen (3×). Dioxane (2 mL) was added, and the reaction was flushed again with nitrogen (3×). Following the addition of N-methylaniline (58.7 μL, 0.542 mmol, 2.2 eq), the reaction was stirred at 80° C. for 3 h. It was then cooled to room temperature, filtered through Celite with CH₂Cl₂, and concentrated to dryness. Purification by silica gel chromatography (0-2₅% EtOAc/hexanes, linear gradient) afforded 66 (144 mg, 94%) as an off-white solid. 1H NMR (CDCl₃, 400 MHz) δ 7.36-7.30 (m, 4H), 7.20-7.06 (m, 8H), 6.82-6.75 (m, 4H), 3.35 (s, 6H), 0.50 (s, 3H), 0.47 (s, 3H); 19F NMR (CDCl₃, 376 MHz) δ−138.60 (td, J=19.8, 3.0 Hz, 1F),-138.76 (td, J=19.4, 7.8 Hz, 1F), −143.54-−143.71 (m, 1F), −151.30-−151.47 (m, 1F); Analytical HPLC: t_{R}=16.3 min, >99% purity (50-95% MeCN/H₂O, linear gradient, with constant 0.1% v/v TFA additive; 20 min run; 1 mL/min flow; ESI; positive ion mode; detection at 675 nm); HRMS (ESI) calcd for C₃₆H₂₉F₄N₂O₂Si [M+H]⁺ 625.1929, found 625.1922.

### Example 110: Rhodamine 110 and N-Aryl Rhodamine Synthesis via Cross-Coupling (Scheme 11): 4,5,6,7⁻Tetrafluoro-rhodamine 110 bis(tert-butyl carbamate) (67)

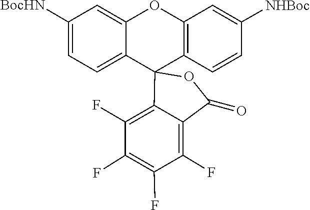 [SMILES: CC(C)(C)OC(=O)Nc1ccc2c(c1)Oc1cc(NC(=O)OC(C)(C)C)ccc1C21OC(=O)c2c(F)c(F)c(F)c(F)c21]

A vial was charged with 4,5,6,7⁻tetrafluoro-fluorescein ditriflate (61; 200 mg, 0.299 mmol), tert-butyl carbamate (84.1 mg, 0.718 mmol, 2.4 eq), Pd₂dba₃ (27.4 mg, 29.9 μmol, 0.1 eq), XPhos (42.8 mg, 89.8 μmol, 0.3 eq), and Cs₂CO₃ (273 mg, 0.838 mmol, 2.8 eq). The vial was sealed and evacuated/backfilled with nitrogen (3×). Dioxane (2 mL) was added; after flushing the reaction again with nitrogen (3×), it was stirred at 80° C. for 4 h. It was then cooled to room temperature, filtered through Celite with CH₂Cl₂, and evaporated. The residue was purified by silica gel chromatography (0-30% EtOAc/hexanes, linear gradient) to afford 67 (172 mg, 95%) as an off-white solid. ¹H NMR (CDCl₃, 400 MHz) δ 7.49 (d, J=2.2 Hz, 2H), 7.01 (dd, J=8.7, 2.3 Hz, 2H), 6.82 (d, J=8.7 Hz, 2H), 6.61 (s, 2H), 1.53 (s, 18H); 19F NMR (CDCl₃, 376 MHz) δ-138.19 (td, J=19.8, 9.4 Hz, 1F), −141.93 (ddd, J=20.5, 17.6, 9.2 Hz, 1F), −142.12 (td, J=19.8, 4.0 Hz, 1F), −149.98 (ddd, J=20.5, 17.7, 3.9 Hz, 1F); Analytical HPLC: t_{R}=15.6 min, >99% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₀H₂₇F₄N₂O₇[M+H]⁺ 603.1749, found 603.1750.

### Example 111: Rhodamine 110 and N-Aryl Rhodamine Synthesis via Cross-Coupling (Scheme 11): 4,5,6,7⁻Tetrafluoro-carborhodamine 110 bis(tert-butyl carbamate) (68)

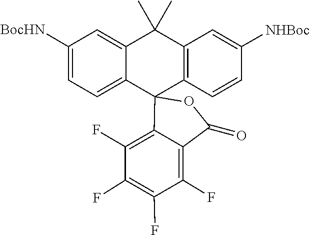 [SMILES: CC(C)(C)OC(=O)Nc1ccc2c(c1)C(C)(C)c1cc(NC(=O)OC(C)(C)C)ccc1C21OC(=O)c2c(F)c(F)c(F)c(F)c21]

A vial was charged with 4,5,6,7⁻tetrafluoro-carbofluorescein ditriflate (62; 350 mg, 0.504 mmol), tert-butyl carbamate (142 mg, 1.21 mmol, 2.4 eq), Pd₂dba₃ (46.1 mg, 50.4 μmol, 0.1 eq), XPhos (72.1 mg, 0.151 mmol, 0.3 eq), and Cs₂CO₃ (460 mg, 1.41 mmol, 2.8 eq). The vial was sealed and evacuated/backfilled with nitrogen (3×). Dioxane (3.5 mL) was added; after flushing the reaction again with nitrogen (3×), it was stirred at 80° C. for 4 h. It was then cooled to room temperature, filtered through Celite with CH₂Cl₂, and evaporated. The residue was purified by silica gel chromatography (0-30% EtOAc/hexanes, linear gradient) to afford 68 (305 mg, 96%) as an off-white solid. ¹H NMR (CDCl₃, 400 MHz) δ 7.79 (d, J=2.2 Hz, 2H), 7.11 (dd, J=8.6, 2.2 Hz, 2H), 6.77 (d, J=8.6 Hz, 2H), 6.59 (s, 2H), 1.81 (s, 3H), 1.74 (s, 3H), 1.53 (s, 18H); 19F NMR (CDCl₃, 376 MHz) δ−138.65 (td, J=20.0, 9.1 Hz), −141.98 (td, J=20.0, 4.0 Hz), −142.47 (td, J=19.5, 8.8 Hz), −150.82-−151.01 (m); Analytical HPLC: t_{R}=15.7 min, 98.2% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₃H₃₃F₄N₂O₆[M+H]⁺ 629.2269, found 629.2280.

### Example 112: Rhodamine 110 and N-Aryl Rhodamine Synthesis via Cross-Coupling (Scheme 11): 4,5,6,7⁻Tetrafluoro-Si-rhodamine 110 bis(tert-butyl carbamate) (69)

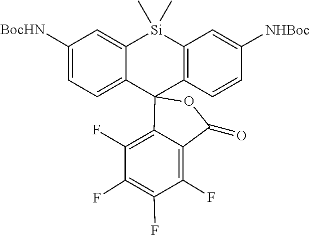 [SMILES: CC(C)(C)OC(=O)Nc1ccc2c(c1)[Si](C)(C)c1cc(NC(=O)OC(C)(C)C)ccc1C21OC(=O)c2c(F)c(F)c(F)c(F)c21]

A vial was charged with 4,5,6,7⁻tetrafluoro-Si-fluorescein ditriflate (63; 245 mg, 0.345 mmol), tert-butyl carbamate (96.9 mg, 0.828 mmol, 2.4 eq), Pd₂dba₃ (31.6 mg, 34.5 μmol, 0.1 eq), XPhos (49.3 mg, 0.103 mmol, 0.3 eq), and Cs₂CO₃ (315 mg, 0.965 mmol, 2.8 eq). The vial was sealed and evacuated/backfilled with nitrogen (3×). Dioxane (3 mL) was added; after flushing the reaction again with nitrogen (3×), it was stirred at 80° C. for 5 h. It was then cooled to room temperature, filtered through Celite with CH₂Cl₂, and evaporated. The residue was purified by silica gel chromatography (0-30% EtOAc/hexanes, linear gradient) to afford 69 (212 mg, 95%) as an off-white solid. ¹H NMR (CDCl₃, 400 MHz) δ 7.70 (d, J=2.5 Hz, 2H), 7.30 (dd, J=8.7, 2.5 Hz, 2H), 6.90 (d, J=8.7 Hz, 2H), 6.56 (s, 2H), 1.52 (s, 18H), 0.60 (s, 3H), 0.56 (s, 3H); 19F NMR (CDCl₃, 376 MHz) δ−138.04 (td, J=20.0, 8.8 Hz, 1F), −139.27-−139.47 (m, 1F), −142.79 (td, J=19.4, 8.8 Hz, 1F), −150.67-−150.88 (m, 1F); Analytical HPLC: t_{R}=16.1 min, 98.4% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₂H₃₃F₄N₂O₆Si [M+H]⁺ 645.2039, found 645.2049.

### Example 113: Rhodamine 110 and N-Aryl Rhodamine Synthesis via Cross-Coupling (Scheme 11): 4,5,6,7⁻Tetrafluoro-rhodamine 110 (FRh₁₁₀, 70)

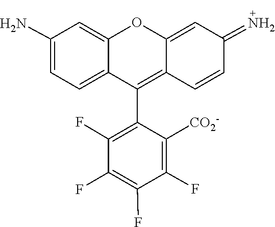

4,5,6,7⁻Tetrafluoro-rhodamine 110 bis(tert-butyl carbamate) (67; 100 mg, 0.166 mmol) was taken up in CH₂Cl₂ (5 mL), and trifluoroacetic acid (1 mL) was added. The reaction was stirred at room temperature for 3 h. It was then diluted with toluene (5 mL) and concentrated to dryness. Purification by reverse phase HPLC (10-40% MeCN/H₂O, linear gradient, with constant 0.1% v/v TFA additive) yielded 82.6 mg (96%, TFA salt) of 70 as a red solid. 1H NMR (CD₃OD, 400 MHz) δ 7.29 (dd, J=9.2, 0.9 Hz, 2H), 6.90 (dd, J=9.2, 2.1 Hz, 2H), 6.83 (d, J=2.1 Hz, 2H); 19F NMR (CD₃OD, 376 MHz) δ−75.77 (s, 3F), −135.40 (ddd, J=20.4, 11.9, 8.1 Hz, 1F), −137.30 (ddd, J=21.7, 11.9, 5.1 Hz, 1F), −150.52 (ddd, J=21.8, 18.9, 7.9 Hz, 1F), −151.93 (ddd, J=21.1, 19.0, 5.3 Hz, 1F); Analytical HPLC: t_{R}=9.5 min, >99% purity (10-95% MeCN/H₂O, linear gradient, with constant 0.1% v/v TFA additive; 20 min run; 1 mL/min flow; ESI; positive ion mode; detection at 525 nm); HRMS (ESI) calcd for C₂₀H₁₁F₄N₂O₃[M+H]⁺ 403.0700, found 403.0703.

### Example 114: Rhodamine 110 and N-Aryl Rhodamine Synthesis via Cross-Coupling (Scheme 11): 4,5,6,7⁻Tetrafluoro-carborhodamine 110 (FCRh₁₁₀, 71)

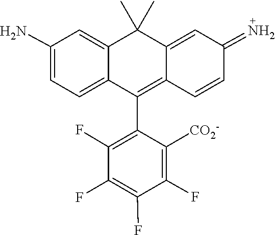

4,5,6,7⁻Tetrafluoro-carborhodamine 110 bis(tert-butyl carbamate) (68; 90 mg, 0.143 mmol) was taken up in CH₂Cl₂ (5 mL), and trifluoroacetic acid (1 mL) was added. The reaction was stirred at room temperature for 4 h. It was then diluted with toluene (5 mL) and concentrated to dryness. The residue was taken up in saturated NaHCO₃ and extracted with EtOAc (2×). The combined organic extracts were washed with brine, dried over anhydrous MgSO₄, filtered, deposited onto Celite, and concentrated to dryness. Flash chromatography (10-100% EtOAc/hexanes, linear gradient; dry load with Celite) afforded 52.2 mg (85%) of 71 as a dark purple solid. ¹H NMR (CD₃OD, 400 MHz) δ 7.09 (d, J=2.2 Hz, 2H), 7.06 (dd, J=8.9, 0.8 Hz, 2H), 6.62 (dd, J=8.9, 2.2 Hz, 2H), 1.72 (s, 3H), 1.66 (s, 3H); 19F NMR (CD₃OD, 376 MHz) δ-140.30 (ddd, J=21.2, 13.8, 3.5 Hz, 1F), −141.26 (ddd, J=21.9, 13.9, 4.6 Hz, 1F), −154.43 (ddd, J=22.4, 18.8, 3.5 Hz, 1F), −155.33-−155.56 (m, 1F); Analytical HPLC: t_{R}=9.9 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₂₃H₁₇F₄N₂O₂[M+H]⁺ 429.1221, found 429.1223.

### Example 115: Rhodamine 110 and N-Aryl Rhodamine Synthesis via Cross-Coupling (Scheme 11): 4,5,6,7⁻Tetrafluoro-Si-rhodamine 110 (FSiRh₁₁₀, 72)

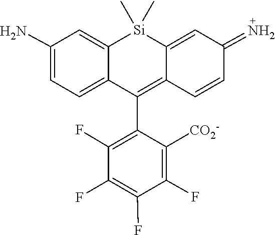

4,5,6,7⁻Tetrafluoro-Si-rhodamine 110 bis(tert-butyl carbamate) (69; 50 mg, 77.6 μmol) was taken up in CH₂Cl₂ (4 mL), and trifluoroacetic acid (0.8 mL) was added. The reaction was stirred at room temperature for 4 h. It was then diluted with toluene (4 mL) and concentrated to dryness. The residue was taken up in saturated NaHCO₃ and extracted with EtOAc (2×). The combined organic extracts were washed with brine, dried over anhydrous MgSO₄, filtered, deposited onto Celite, and concentrated to dryness. Flash chromatography (10-100% EtOAc/hexanes, linear gradient; dry load with Celite) afforded 32.2 mg (93%) of 72 as a light blue solid. ¹H NMR (CDCl₃, 400 MHz) δ 6.94 (d, J=2.6 Hz, 2H), 6.72 (dd, J=8.5, 1.2 Hz, 2H), 6.58 (dd, J=8.5, 2.7 Hz, 2H), 3.82 (s, 4H), 0.54 (s, 3H), 0.52 (s, 3H); 19F NMR (CDCl₃, 376 MHz) δ-138.90 (td, J=19.8, 8.4 Hz, 1F), −139.14 (td, J=19.9, 3.4 Hz, 1F), −143.59 (ddd, J=20.5, 18.3, 8.3 Hz, 1F), −151.59 (ddd, J=20.2, 18.3, 3.8 Hz, 1F); Analytical HPLC: t_{R}=9.9 min, 98.6% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₂₂H₁₇F₄N₂O₂Si [M+H]⁺ 445.0990, found 445.0998.

### Example 116: MAC Substitution of 4,5,6,7⁻Tetrafluoroxanthenes: 4,5,7-Trifluoro-6⁻(MOM-MAC)-fluorescein (60_{MAC})

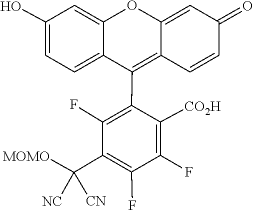

4,5,6,7⁻Tetrafluoro-fluorescein¹⁹ (60; 100 mg, 0.247 mmol) and 2-(methoxymethoxy)malononitrile (3; 31.2 mg, 0.247 mmol, 1 eq) were combined in DMF (5 mL), and DIEA (129 μL, 0.742 mmol, 3 eq) was added. After stirring the reaction at room temperature for 18 h, it was evaporated to dryness and purified by reverse phase HPLC (30-60% MeCN/H₂O, linear gradient, with constant 0.1% v/v TFA additive). The pooled product fractions were partially concentrated to remove MeCN and extracted with CH₂Cl₂ (3×). The organic extracts were dried over anhydrous MgSO₄, filtered, and evaporated to provide 60_{MAC} (69 mg, 55%) as a yellow-orange solid. ¹H NMR (DMSO-d₆, 400 MHz) δ 10.26 (s, 2H), 7.05 (d, J=8.7 Hz, 2H), 6.70 (d, J=2.3 Hz, 2H), 6.60 (dd, J=8.7, 2.4 Hz, 2H), 5.11 (s, 2H), 3.34 (s, 3H); 19F NMR (DMSO-d₆, 376 MHz) δ−118.57 (d, J=21.4 Hz, 1F), −129.27 (d, J=21.1 Hz, 1F),-140.99 (t, J=21.3 Hz, 1F); Analytical HPLC: t_{R}=12.9 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₂₅H₁₄F₃N₂O₇[M+H]⁺ 511.0748, found 511.0746.

### Example 117: MAC Substitution of 4,5,6,7⁻Tetrafluoroxanthenes: 4,5,7-Trifluoro-6⁻(MOM-MAC)-carbofluorescein (51_{MAC})

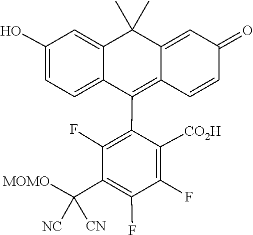

4,5,6,7⁻Tetrafluoro-carbofluorescein (51; 125 mg, 0.290 mmol) and 2-(methoxymethoxy)malononitrile (3; 36.6 mg, 0.290 mmol, 1 eq) were combined in DMF (4 mL), and DIEA (152 μL, 0.871 mmol, 3 eq) was added. After stirring the reaction at room temperature for 2 h, it was diluted with 10% citric acid and extracted with EtOAc (2×). The combined organic extracts were washed with water and brine, dried over anhydrous MgSO₄, filtered, and concentrated in vacuo. Flash chromatography on silica gel (0-75% EtOAc/toluene, linear gradient) afforded 92 mg (59%) of 51_{MAC} as an orange solid. ¹H NMR (DMSO-d₆, 400 MHz) δ 9.83 (s, 2H), 7.10 (d, J=2.5 Hz, 2H), 6.87 (d, J=8.6 Hz, 2H), 6.66 (dd, J=8.7, 2.4 Hz, 2H), 5.10 (s, 2H), 3.32 (s, 3H), 1.68 (s, 3H), 1.60 (s, 3H); 19F NMR (DMSO-d₆, 376 MHz) δ−117.77 (d, J=21.4 Hz, 1F), −129.80 (d, J=21.4 Hz, 1F), −141.28 (t, J=21.5 Hz, 1F); Analytical HPLC: t_{R}=13.1 min, 99.0% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₂₈H₂₀F₃N₂O₆[M+H]⁺ 537.1268, found 537.1268.

### Example 118: MAC Substitution of 4,5,6,7⁻Tetrafluoroxanthenes: 4,5,7-Trifluoro-6⁻(MOM-MAC)—Si-fluorescein (55MAC)

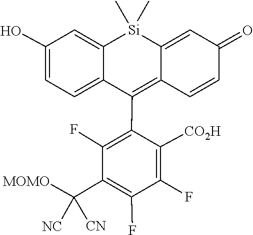

4,5,6,7⁻Tetrafluoro-Si-fluorescein (55; 45 mg, 0.101 mmol) and 2-(methoxymethoxy)malononitrile (3; 12.7 mg, 0.101 mmol, 1 eq) were combined in DMF (2 mL), and DIEA (52.7 μL, 0.302 mmol, 3 eq) was added. After stirring the reaction at room temperature for 3 h, it was diluted with 10% citric acid and extracted with EtOAc (2×). The combined organic extracts were washed with water and brine, dried over anhydrous MgSO₄, filtered, and concentrated in vacuo. Flash chromatography on silica gel (0-60% EtOAc/toluene, linear gradient) afforded 31.6 mg (57%) of 55Mac as an off-white solid. ¹H NMR (CDCl₃, 400 MHz) δ 7.14 (d, J=2.6 Hz, 2H), 6.82 (d, J=8.8 Hz, 2H), 6.78 (dd, J=8.7, 2.6 Hz, 2H), 5.17 (s, 2H), 5.01 (s, 2H), 3.52 (s, 3H), 0.57 (s, 3H), 0.54 (s, 3H); 19F NMR (CDCl₃, 376 MHz) δ−113.71 (d, J=22.6 Hz, 1F), −126.48 (d, J=19.9 Hz, 1F), −138.58 (dd, J=22.7, 20.1 Hz, 1F); Analytical HPLC: t_{R}=13.5 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₂₇H₂₀F₃N₂O₆Si [M+H]⁺ 553.1037, found 553.1039.

### Example 119: MAC Substitution of 4,5,6,7⁻Tetrafluoroxanthenes: 6⁻(MOM-MAC)-FMGL (49MAC)

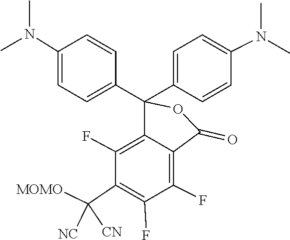

4,5,6,7⁻Tetrafluoro-MGL (49; 300 mg, 0.675 mmol) and 2⁻(methoxymethoxy)-malononitrile (3; 85.1 mg, 0.675 mmol, 1 eq) were combined in DMF (10 mL), and DIEA (235 L, 1.35 mmol, 2 eq) was added. After stirring the reaction at room temperature for 2 h, it was concentrated in vacuo and purified by flash chromatography on silica gel (0-50% EtOAc/hexanes, linear gradient) to provide 49_{MAC} (225 mg, 61%) as a yellow foam. 1H NMR (CDCl₃, 400 MHz) δ 7.16-7.09 (m, 4H), 6.65 (d, J=9.0 Hz, 4H), 5.18 (s, 2H), 3.53 (s, 3H), 2.97 (s, 12H); 19F NMR (CDCl₃, 376 MHz) δ−112.40 (d, J=22.4 Hz, 1F), −127.57 (d, J=20.4 Hz, 1F), −139.50 (dd, J=22.5, 20.3 Hz, 1F); Analytical HPLC: t_{R}=13.3 min, >99% purity (10-95% MeCN/H₂O, linear gradient, with constant 0.1% v/v TFA additive; 20 min run; 1 mL/min flow; ESI; positive ion mode; detection at 280 nm); HRMS (ESI) calcd for C₂₉H₂₆F₃N₄O₄ [M+H]⁺ 551.1901, found 551.1910.

### Example 120: MAC Substitution of 4,5,6,7⁻Tetrafluoroxanthenes: 4,5,7-Trifluoro-6⁻(MOM-MAC)-rhodamine 110 bis(tert-butyl carbamate) (67_{MAC})

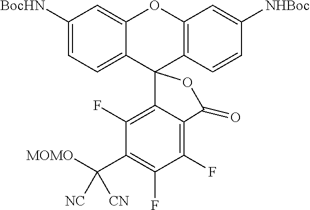

4,5,6,7⁻Tetrafluoro-rhodamine 110 bis(tert-butyl carbamate) (67; 300 mg, 0.498 mmol) and 2⁻(methoxymethoxy)malononitrile (3; 62.8 mg, 0.498 mmol, 1 eq) were combined in DMF (5 mL), and DIEA (173 μL, 0.996 mmol, 2 eq) was added. After stirring the reaction at room temperature for 2 h, it was concentrated in vacuo and purified by silica gel chromatography (0-50% EtOAc/hexanes, linear gradient) to yield 183 mg (52%) of 67_{MAC} as an off-white solid.

¹H NMR (CDCl₃, 400 MHz) δ 7.48 (d, J=2.2 Hz, 2H), 7.06 (dd, J=8.7, 2.2 Hz, 2H), 6.82 (d, J=8.6 Hz, 2H), 6.63 (s, 2H), 5.13 (s, 2H), 3.51 (s, 3H), 1.53 (s, 18H); 19F NMR (CDCl₃, 376 MHz) δ-116.95 (d, J=22.9 Hz, 1F), −125.48 (d, J=19.9 Hz, 1F), −138.65 (dd, J=23.0, 20.2 Hz, 1F); Analytical HPLC: t_{R}=15.2 min, >99% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₅H₃₂F₃N₄O₉[M+H]⁺ 709.2116, found 709.2130.

### Example 121: MAC Substitution of 4,5,6,7⁻Tetrafluoroxanthenes: 4,5,7-Trifluoro-6⁻(MOM-MAC)-carborhodamine 110 bis(tert-butyl carbamate) (68_{MAC})

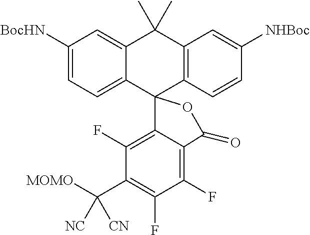

4,5,6,7⁻Tetrafluoro-carborhodamine 110 bis(tert-butyl carbamate) (68; 180 mg, 0.286 mmol) and 2⁻(methoxymethoxy)malononitrile (3; 36.1 mg, 0.286 mmol, 1 eq) were combined in DMF (3 mL), and DIEA (99.7 μL, 0.573 mmol, 2 eq) was added. After stirring the reaction at room temperature for 2 h, it was concentrated in vacuo and purified by silica gel chromatography (0-50% EtOAc/hexanes, linear gradient) to yield 107 mg (51%) of 68_{MAC} as an off-white solid. ¹H NMR (CDCl₃, 400 MHz) δ 7.78 (d, J=2.2 Hz, 2H), 7.16 (dd, J=8.6, 2.2 Hz, 2H), 6.75 (d, J=8.6 Hz, 2H), 6.61 (s, 2H), 5.12 (s, 2H), 3.50 (s, 3H), 1.81 (s, 3H), 1.75 (s, 3H), 1.53 (s, 18H); 19F NMR (CDCl₃, 376 MHz) δ−116.84 (d, J=22.7 Hz, 1F), −126.32 (d, J=19.7 Hz, 1F), −139.19 (dd, J=22.9, 20.1 Hz, 1F); Analytical HPLC: t_{R}=15.2 min, >99% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₈H₃₈F₃N₄O₈ [M+H]⁺ 735.2636, found 735.2641.

### Example 122: MAC Substitution of 4,5,6,7⁻Tetrafluoroxanthenes: 4,5,7-Trifluoro-6⁻(MOM-MAC)—Si-rhodamine 110 bis(tert-butyl carbamate) (69_{MAC})

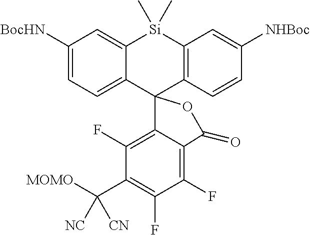

4,5,6,7⁻Tetrafluoro-Si-rhodamine 110 bis(tert-butyl carbamate) (69; 150 mg, 0.233 mmol) and 2⁻(methoxymethoxy)malononitrile (3; 29.3 mg, 0.233 mmol, 1 eq) were combined in DMF (3 mL), and DIEA (81.1 μL, 0.465 mmol, 2 eq) was added. After stirring the reaction at room temperature for 2 h, it was concentrated in vacuo and purified by silica gel chromatography (0-50% EtOAc/hexanes, linear gradient) to yield 89 mg (51%) of 69_{MAC} as an off-white solid. ¹H NMR (CDCl₃, 400 MHz) δ 7.68 (d, J=2.5 Hz, 2H), 7.36 (dd, J=8.8, 2.5 Hz, 2H), 6.88 (d, J=8.7 Hz, 2H), 6.58 (s, 2H), 5.16 (s, 2H), 3.52 (s, 3H), 1.52 (s, 18H), 0.60 (s, 3H), 0.56 (s, 3H); 19F NMR (CDCl₃, 376 MHz) δ−114.17 (d, J=22.5 Hz, 1F), −126.44 (d, J=20.0 Hz, 1F), −138.55 (t, J=21.4 Hz, 1F); Analytical HPLC: t_{R}=15.7 min, >99% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₇H₃₈F₃N₄O₈Si [M+H]⁺ 751.2406, found 751.2413.

### Example 123: MAC Substitution of 4,5,6,7⁻Tetrafluoroxanthenes: 6⁻(MOM-MAC)-JF₆₃₂ (44_{MAC})

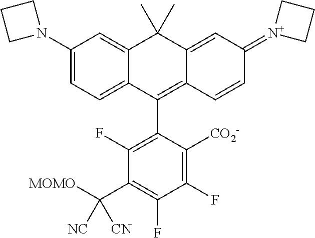

JF₆₃₂ (44; 45.0 mg, 88.5 mol) and 2⁻(methoxymethoxy)malononitrile (3; 11.2 mg, 88.5 mol, 1 eq) were combined in DMF (2 mL), and DIEA (30.8 μL, 0.177 mmol, 2 eq) was added. After stirring the reaction at room temperature for 3 h, it was concentrated in vacuo and purified by reverse phase HPLC (30-50% MeCN/H₂O, linear gradient, with constant 0.1% v/v TFA additive). The pooled HPLC product fractions were partially concentrated to remove MeCN, diluted with saturated NaHCO₃, and extracted with CH₂Cl₂ (2×). The organic extracts were dried over anhydrous MgSO₄, filtered, and evaporated to yield 23.5 mg (43%) of 44_{MAC} as a blue solid. ¹H NMR (CDCl₃, 400 MHz) δ 6.63 (d, J=8.6 Hz, 2H), 6.55 (d, J=2.3 Hz, 2H), 6.28 (dd, J=8.6, 2.4 Hz, 2H), 5.11 (s, 2H), 3.95 (t, J=7.3 Hz, 8H), 3.50 (s, 3H), 2.40 (p, J=7.2 Hz, 4H), 1.75 (s, 3H), 1.70 (s, 3H); 19F NMR (CDCl₃, 376 MHz) δ−116.34 (d, J=22.7 Hz, 1F),-127.69 (d, J=20.0 Hz, 1F), −140.39 (dd, J=22.8, 20.1 Hz, 1F); Analytical HPLC: t_{R}=12.5 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₃₄H₃₀F₃N₄O₄[M+H]⁺ 615.2214, found 615.2207.

### Example 124: MAC Substitution of 4,5,6,7⁻Tetrafluoroxanthenes: 6⁻(MOM-MAC)-JFX₅₇₆ (8_{MAC})

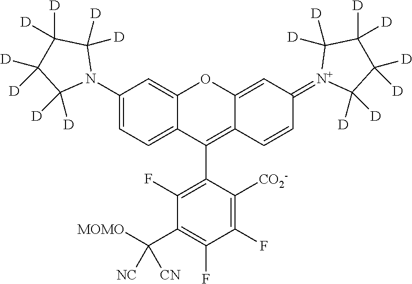

JFX₅₇₆ (8; 250 mg, 0.475 mmol) and 2⁻(methoxymethoxy)malononitrile (3; 59.9 mg, 0.475 mmol, 1 eq) were combined in DMF (9 mL), and DIEA (165 μL, 0.950 mmol, 2 eq) was added. After stirring the reaction at room temperature for 2 h, it was concentrated in vacuo and purified by silica gel chromatography (0-15% MeOH/CH₂Cl₂, linear gradient, with constant 1% v/v AcOH additive) to yield 8_{MAC} as a dark purple solid (188 mg, 57%, acetate salt). 1H NMR (CD₃OD, 400 MHz) δ 7.32 (dd, J=9.4, 0.8 Hz, 2H), 6.94 (dd, J=9.4, 2.3 Hz, 2H), 6.75 (d, J=2.3 Hz, 2H), 5.20 (s, 2H), 3.53 (s, 3H); 19F NMR (CD₃OD, 376 MHz) δ−112.06 (d, J=14.9 Hz, 1F), −128.39 (d, J=21.8 Hz, 1F), −141.73 (dd, J=21.7, 14.9 Hz, 1F); Analytical HPLC: t_{R}=12.7 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₃₃H₁₂D₁₆F₃N₄O₅[M+H]⁺ 633.3011, found 633.3006.

### Example 125: MAC Substitution of 4,5,6,7⁻Tetrafluoroxanthenes: 6⁻(MOM-MAC)-JFX₆₃₇ (40_{MAC})

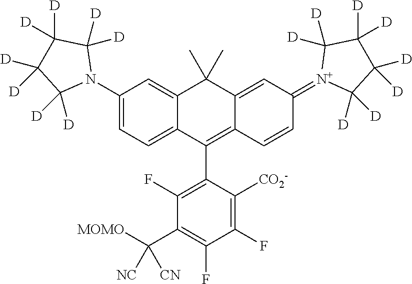

JFX₆₃₇ (40; 40 mg, 72.4 mol) and 2⁻(methoxymethoxy)malononitrile (3; 9.1 mg, 72.4 mol, 1 eq) were combined in DMF (2 mL), and DIEA (25.2 μL, 0.145 mmol, 2 eq) was added. After stirring the reaction at room temperature for 1 h, it was concentrated in vacuo and purified by reverse phase HPLC (30-50% MeCN/H₂O, linear gradient, with constant 0.1% v/v TFA additive). The pooled HPLC product fractions were partially concentrated to remove MeCN, diluted with saturated NaHCO₃, and extracted with CH₂Cl₂ (2×). The organic extracts were dried over anhydrous MgSO₄, filtered, and evaporated to yield 19.2 mg (40%) of 40_{MAC} as a blue solid. ¹H NMR (CD₃OD, 400 MHz) δ 7.20 (dd, J=9.3, 0.9 Hz, 2H), 7.08 (d, J=2.4 Hz, 2H), 6.72 (dd, J=9.3, 2.4 Hz, 2H), 5.19 (s, 2H), 3.53 (s, 3H), 1.80 (s, 3H), 1.74 (s, 3H); 19F NMR (CD₃OD, 376 MHz) δ−112.49 (d, J=14.6 Hz, 1F), −129.79 (d, J=22.3 Hz, 1F), −142.26 (dd, J=21.8, 14.9 Hz, 1F); Analytical HPLC: t_{R}=13.0 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₃₆H₁₈D₁₆F₃N₄O₄[M+H]⁺ 659.3531, found 659.3522.

### Example 126: MAC Substitution of 4,5,6,7⁻Tetrafluoroxanthenes: 6⁻(MOM-MAC)-JFX₆₇₃ (25_{MAC})

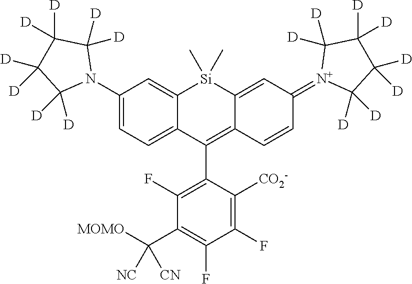

JFX₆₇₃ (25; 300 mg, 0.527 mmol) and 2⁻(methoxymethoxy)malononitrile (3; 66.5 mg, 0.527 mmol, 1 eq) were combined in DMF (7 mL), and DIEA (184 μL, 1.05 mmol, 2 eq) was added. After stirring the reaction at room temperature for 2 h, it was concentrated in vacuo and purified by silica gel chromatography (10-100% EtOAc/hexanes, linear gradient) to yield 166 mg (47%) of 25_{MAC} as a blue-green solid. ¹H NMR (CDCl₃, 400 MHz) δ 6.77 (d, J=2.7 Hz, 2H), 6.73 (dd, J=8.8, 1.1 Hz, 2H), 6.46 (dd, J=8.8, 2.8 Hz, 2H), 5.16 (s, 2H), 3.53 (s, 3H), 0.57 (s, 3H), 0.55 (s, 3H); 19F NMR (CDCl₃, 376 MHz) δ−113.90 (d, J=22.5 Hz, 1F), −128.13 (d, J=20.2 Hz, 1F), −140.21 (dd, J=22.7, 20.2 Hz, 1F); Analytical HPLC: t_{R}=13.2 min, 97.5% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₃₅H₁₈D₁₆F₃N₄O₄Si [M+H]⁺ 675.3300, found 675.3302.

### Example 127: MAC Substitution of 4,5,6,7⁻Tetrafluoroxanthenes: 4-(Dicyano(methoxymethoxy)methyl)-2,3,5⁻trifluoro-6⁻(1,2,2,4,8,10,10,11⁻octamethyl-1,2,10,11⁻tetrahydro-pyrano[3,2⁻g:5,6⁻g]diquinolin-13⁻ium-6⁻yl)benzoate (11_{MAC})

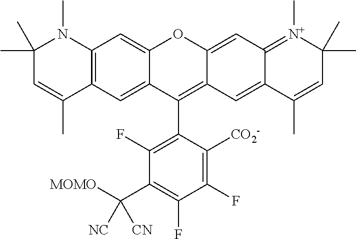

Rhodamine 11 (100 mg, 0.169 mmol) and 2⁻(methoxymethoxy)malononitrile (3; 21.4 mg, 0.169 mmol, 1 eq) were combined in DMF (4 mL), and DIEA (59.0 μL, 0.339 mmol, 2 eq) was added. After stirring the reaction at room temperature for 4 h, it was evaporated to dryness. Flash chromatography on silica gel (0-10% MeOH/CH₂Cl₂, linear gradient, with constant 1% v/v AcOH additive) afforded 11_{MAC} as a dark blue solid (93.8 mg, 73%, acetate salt). ¹H NMR (CD₃OD, 400 MHz) δ 6.94 (d, J=1.1 Hz, 2H), 6.81 (s, 2H), 5.68 (q, J=1.4 Hz, 2H), 5.19 (s, 2H), 3.51 (s, 3H), 3.20 (s, 6H), 1.94 (d, J=1.4 Hz, 6H), 1.52 (s, 6H), 1.51 (s, 6H); 19F NMR (CD₃OD, 376 MHz) δ−111.06 (d, J=14.9 Hz, 1F), −127.94 (d, J=21.9 Hz, 1F),-140.94 (dd, J=21.8, 14.9 Hz, 1F); Analytical HPLC: t_{R}=14.2 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₃₉H₃₆F₃N₄O₅ [M+H]⁺ 697.2632, found 697.2622.

### Example 128: MAC Substitution of 4,5,6,7⁻Tetrafluoroxanthenes: 6⁻(MOM-MAC)-JF₅₆₃ (18_{MAC})

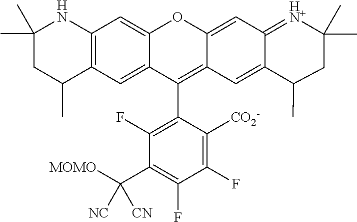

JF₅₆₃ (18; 50 mg, 88.2 μmol) and 2⁻(methoxymethoxy)malononitrile (3; 11.1 mg, 88.2 μmol, 1 eq) were combined in DMSO (5 mL), and DIEA (30.7 μL, 0.176 mmol, 2 eq) was added. The resulting dark red solution was stirred at room temperature for 4 h. A second portion of 2⁻(methoxymethoxy)malononitrile (3; 2.8 mg, 22.1 μmol, 0.25 eq) was added, and stirring was continued for an additional 2 h at room temperature. It was subsequently diluted with water and extracted with CH₂Cl₂ (2×). The combined organic extracts were dried over anhydrous MgSO₄, filtered, and concentrated in vacuo. Purification by silica gel chromatography (0-20% MeOH/CH₂Cl₂, linear gradient, with constant 1% v/v AcOH additive) provided 30.5 mg (47%, acetate salt) of 18_{MAC} as a dark red-purple solid (mixture of diastereomers). ¹H NMR (CD₃OD, 400 MHz) δ 7.16-7.09 (m, 2H), 6.64-6.59 (m, 2H), 5.21-5.18 (m, 2H), 3.55-3.50 (m, 3H), 3.00-2.85 (m, 2H), 1.91-1.84 (m, 2H), 1.49-1.23 (m, 20H); 19F NMR (CD₃OD, 376 MHz) δ -111.22-−111.37 (m, 1F), −128.02-−128.46 (m, 1F), −140.92-−141.30 (m, 1F); Analytical HPLC: t_{R} (three isomers)=13.2 min, 13.4 min, 13.6 min; 95.1% total purity (10-95% MeCN/H₂O, linear gradient, with constant 0.1% v/v TFA additive; 20 min run; 1 mL/min flow; ESI; positive ion mode; detection at 550 nm); HRMS (ESI) calcd for C₃₇H₃₆F₃N₄O₅[M+H]⁺ 673.2632, found 673.2623.

### Example 129: MAC Substitution of 4,5,6,7⁻Tetrafluoroxanthenes: 2-(1,2,2,4,8,10,10,11,13,13⁻decamethyl-2,10,11,13⁻Tetrahydrosilino[3,2⁻g:5,6⁻g′]diquinolin-6-ylium-6(1H)-yl)-4⁻(dicyano(methoxymethoxy)methyl)-3,5,6⁻trifluorobenzoate (38_{MAC})

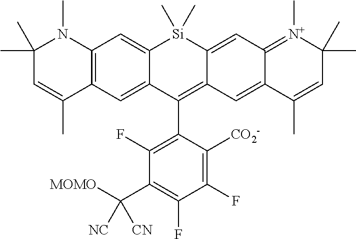

Si-rhodamine 38 (425 mg, 0.672 mmol) and 2⁻(methoxymethoxy)malononitrile (3; 84.7 mg, 0.672 mmol, 1 eq) were combined in DMF (13 mL); DIEA (234 μL, 1.34 mmol, 2 eq) was added, and the reaction was stirred at room temperature for 1 h. LC/MS analysis indicated that the reaction was incomplete, so additional 2⁻(methoxymethoxy)malononitrile (3; 25.4 mg, 0.201 mmol, 1 eq) was added. After stirring the reaction for another 1 h at room temperature, it was concentrated in vacuo and purified by silica gel chromatography (10-100% EtOAc/hexanes, linear gradient) to yield 152 mg (31%) of 38_{MAC} as a green solid. 1H NMR (CDCl₃, 400 MHz) δ 6.69 (s, 2H), 6.46 (s, 2H), 5.27 (q, J=1.4 Hz, 2H), 5.16 (s, 2H), 3.52 (s, 3H), 2.87 (s, 6H), 1.75 (d, J=1.5 Hz, 6H), 1.33 (s, 6H), 1.30 (s, 6H), 0.56 (s, 3H), 0.55 (s, 3H); 19F NMR (CDCl₃, 376 MHz) δ−112.08 (d, J=22.4 Hz, 1F), −128.21 (d, J=19.7 Hz, 1F),-139.61 (dd, J=22.5, 20.3 Hz, 1F); Analytical HPLC: t_{R}=11.5 min, 98.0% purity (30-95% MeCN/H₂O, linear gradient, with constant 0.1% v/v TFA additive; 20 min run; 1 mL/min flow; ESI; positive ion mode; detection at 750 nm); HRMS (ESI) calcd for C₄₁H₄₂F₃N₄O₄Si [M+H]⁺ 739.2922, found 739.2917.

### Example 130: MAC Substitution of 4,5,6,7⁻Tetrafluoroxanthenes: 4-(Dicyano(methoxymethoxy)methyl)-2,3,5⁻trifluoro-6⁻(2,2,4,8,10,10,13,13⁻octamethyl-2,10,11,13⁻tetrahydro-silino[3,2⁻g:5,6⁻g′]diquinolin-6⁻ylium-6(1H)-yl)benzoate (36Mac)

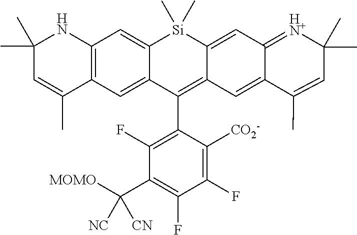

Si-rhodamine 36 (150 mg, 0.248 mmol) and 2⁻(methoxymethoxy)malononitrile (3; 31.3 mg, 0.248 mmol, 1 eq) were combined in DMF (5 mL); DIEA (86.4 μL, 0.496 mmol, 2 eq) was added, and the reaction was stirred at room temperature for 1 h. A second portion of 2-(methoxymethoxy)malononitrile (3; 15.6 mg, 0.124 mmol, 0.5 eq) was added, and stirring was continued for an additional 1 h at room temperature. The reaction was then concentrated in vacuo and purified by silica gel chromatography (25-100% EtOAc/toluene, linear gradient) to yield 30 mg (17%) of 36_{MAC} as a green solid. ¹H NMR (CD₃OD, 400 MHz) δ 6.94 (s, 2H), 6.83 (s, 2H), 5.51 (q, J=1.5 Hz, 2H), 5.19 (s, 2H), 3.53 (s, 3H), 1.77 (d, J=1.4 Hz, 6H), 1.40 (s, 6H), 1.39 (s, 6H), 0.52 (s, 3H), 0.48 (s, 3H); 19F NMR (CD₃OD, 376 MHz) δ−112.09 (dd, J=14.6, 2.3 Hz, 1F), −130.38 (dd, J=21.9, 2.5 Hz, 1F), −141.65 (dd, J=21.9, 14.7 Hz, 1F); Analytical HPLC: t_{R}=13.9 min, >99% purity (10-95% MeCN/H₂O, linear gradient, with constant 0.1% v/v TFA additive; 20 min run; 1 mL/min flow; ESI; positive ion mode; detection at 725 nm); HRMS (ESI) calcd for C₃₉H₃₈F₃N₄O₄Si [M+H]⁺ 711.2609, found 711.2614.

### Example 131: MAC Substitution of 4,5,6,7⁻Tetrafluoroxanthenes: 6⁻(MOM-MAC)—SiRF₇₁₂ (108_{MAC})

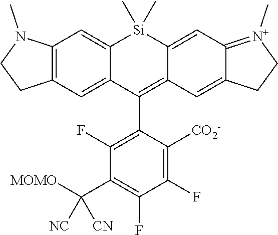

SiRF₇₁₂(108; 180 mg, 0.343 mmol) and 2⁻(methoxymethoxy)malononitrile (3; 43.3 mg, 0.343 mmol, 1 eq) were combined in DMF (5 mL), and DIEA (120 μL, 0.686 mmol, 2 eq) was added. After stirring the reaction at room temperature for 1 h, it was concentrated in vacuo and purified by silica gel chromatography (10-100% acetone/CH₂Cl₂, linear gradient) followed by reverse phase HPLC (30-50% MeCN/H₂O, linear gradient, with constant 0.1% v/v TFA additive). The pooled HPLC product fractions were partially concentrated to remove MeCN, diluted with saturated NaHCO₃, and extracted with CH₂Cl₂ (2×). The organic extracts were dried over anhydrous MgSO₄, filtered, and evaporated to yield 99 mg (46%) of 108_{MAC} as a green solid. ¹H NMR (CD₃OD, 400 MHz) δ 7.09 (s, 2H), 6.81 (s, 2H), 5.18 (s, 2H), 3.83 (t, J=8.1 Hz, 4H), 3.51 (s, 3H), 3.21 (s, 6H), 3.06 (dt, J=16.3, 8.0 Hz, 2H), 2.95 (dt, J=16.8, 8.0 Hz, 2H), 0.54 (s, 3H), 0.53 (s, 3H); 19F NMR (CD₃OD, 376 MHz) δ−112.97 (d, J=14.8 Hz, 1F),-130.78 (d, J=21.8 Hz, 1F), −142.67 (dd, J=21.7, 14.9 Hz, 1F); Analytical HPLC: t_{R}=12.4 min, >99% purity (10-95% MeCN/H₂O, linear gradient, with constant 0.1% v/v TFA additive; 20 min run; 1 mL/min flow; ESI; positive ion mode; detection at 725 nm); HRMS (ESI) calcd for C₃₃H₃₀F₃N₄O₄Si [M+H]⁺ 631.1983, found 631.1989.

### Example 132: MAC Substitution of 4,5,6,7⁻Tetrafluoroxanthenes: 6⁻(MOM-MAC)-JF₆₉₈ (31_{MAC})

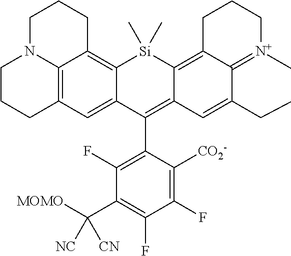

JF₆₉₈ (31; 150 mg, 0.248 mmol) and 2⁻(methoxymethoxy)malononitrile (3; 31.3 mg, 0.248 mmol, 1 eq) were combined in DMF (4 mL), and DIEA (86.4 μL, 0.496 mmol, 2 eq) was added. After stirring the reaction at room temperature for 2 h, it was concentrated in vacuo and purified by silica gel chromatography (0-15% MeOH/CH₂Cl₂, linear gradient, with constant 1% v/v AcOH additive) followed by reverse phase HPLC (40-50% MeCN/H₂O, linear gradient, with constant 0.1% v/v TFA additive). The pooled HPLC product fractions were partially concentrated to remove MeCN, diluted with saturated NaHCO₃, and extracted with CH₂Cl₂ (2×). The organic extracts were dried over anhydrous MgSO₄, filtered, and evaporated to yield 46 mg (26%) of 31_{MAC} as a dark green solid. ¹H NMR (CD₃OD, 400 MHz) δ 6.70 (s, 2H), 5.15 (s, 2H), 3.56 (t, J=6.0 Hz, 4H), 3.53 (t, J=5.9 Hz, 4H), 3.50 (s, 3H), 3.00-2.92 (m, 4H), 2.72-2.46 (m, 4H), 2.10-2.01 (m, 4H), 1.96-1.88 (m, 4H), 0.68 (s, 3H), 0.68 (s, 3H); 19F NMR (CD₃OD, 376 MHz) δ−112.34 (dd, J=14.3, 2.3 Hz, 1F), −130.94 (dd, J=22.0, 2.3 Hz, 1F), −142.74 (dd, J=22.0, 14.6 Hz, 1F); Analytical HPLC: t_{R}=13.2 min, >99% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₃₉H₃₈F₃N₄O₄Si [M+H]⁺ 711.2609, found 711.2615.

### Example 133: MAC Substitution of 4,5,6,7⁻Tetrafluoroxanthenes: 6⁻(MOM-MAC)—FSiRh_{Q} (29Mac)

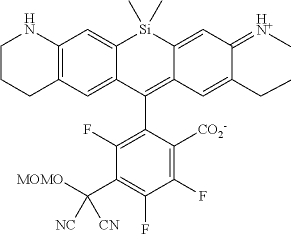

FSiRh_{Q} (29; 170 mg, 0.324 mmol) and 2⁻(methoxymethoxy)malononitrile (3; 40.9 mg, 0.324 mmol, 1 eq) were combined in DMF (5 mL), and DIEA (113 μL, 0.648 mmol, 2 eq) was added. After stirring the reaction at room temperature for 1 h, it was concentrated in vacuo and purified by silica gel chromatography (0-20% MeOH/CH₂Cl₂, linear gradient, with constant 1% v/v AcOH additive) followed by reverse phase HPLC (30-50% MeCN/H₂O, linear gradient, with constant 0.1% v/v TFA additive). The pooled HPLC product fractions were partially concentrated to remove MeCN, diluted with saturated NaHCO₃, and extracted with 15% i-PrOH/CHCl₃ (2×) followed by 20% MeOH/CH₂Cl₂ (3×). The combined organic extracts were dried over anhydrous MgSO₄, filtered, and evaporated to yield 86 mg (42%) of 29_{MAC} as a dark blue solid. ¹H NMR (CD₃OD, 400 MHz) δ 6.95 (s, 2H), 6.84 (s, 2H), 5.17 (s, 2H), 3.51 (s, 3H), 3.50-3.45 (m, 4H), 2.68 (dt, J=16.2, 6.0 Hz, 2H), 2.56 (dt, J=16.1, 6.3 Hz, 2H), 1.89 (p, J=6.2 Hz, 4H), 0.47 (s, 3H), 0.44 (s, 3H); 19F NMR (CD₃OD, 376 MHz) δ−112.41 (dd, J=15.0, 2.0 Hz, 1F), −130.57 (dd, J=21.8, 2.0 Hz, 1F), −142.51 (dd, J=21.9, 14.8 Hz, 1F); Analytical HPLC: t_{R}=12.2 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₃₃H₃₀F₃N₄O₄Si [M+H]⁺ 631.1983, found 631.1988.

### Example 134: MAC Substitution of 4,5,6,7⁻Tetrafluoroxanthenes: 6⁻(MOM-MAC)-JF₆₆₀ (57_{MAC})

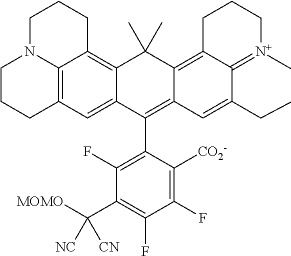

JF₆₆₀ (57; 275 mg, 0.467 mmol) and 2⁻(methoxymethoxy)malononitrile (3; 58.9 mg, 0.467 mmol, 1 eq) were combined in DMF (8 mL), and DIEA (163 μL, 0.934 mmol, 2 eq) was added. After stirring the reaction at room temperature for 2 h, it was evaporated to dryness. Flash chromatography on silica gel (0-20% MeOH/CH₂Cl₂, linear gradient, with constant 0.1% v/v AcOH additive) afforded 57_{MAC} as a dark blue solid (133 mg, 41%). ¹H NMR (CD₃OD, 400 MHz) δ 6.63 (s, 2H), 5.20 (s, 2H), 3.58 (t, J=6.1 Hz, 4H), 3.55-3.51 (m, 4H), 3.51 (s, 3H), 3.11-3.05 (m, 4H), 2.68-2.58 (m, 4H), 2.08-2.00 (m, 4H), 2.05 (s, 3H), 1.97 (s, 3H), 1.93 (p, J=6.1 Hz, 4H); 19F NMR (CD₃OD, 376 MHz) δ−111.21 (d, J=14.7 Hz, 1F), −129.21 (d, J=20.6 Hz, 1F), −139.69 (dd, J=20.5, 14.5 Hz, 1F); Analytical HPLC: t_{R}=13.1 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₄₀H₃₈F₃N₄O₄[M+H]⁺ 695.2840, found 695.2842.

### Example 135: MAC Substitution of 4,5,6,7⁻Tetrafluoroxanthenes: 6⁻(MOM-MAC)-JF₆₅₇ (59MAC)

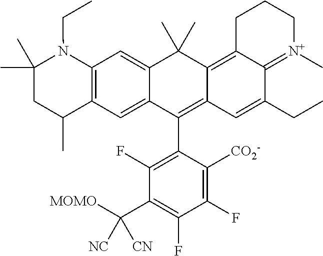

JF₆₅₇ (59; 150 mg, 0.242 mmol) and 2⁻(methoxymethoxy)malononitrile (3; 30.6 mg, 0.242 mmol, 1 eq) were combined in DMF (5 mL), and DIEA (84.5 μL, 0.485 mmol, 2 eq) was added. After stirring the reaction at room temperature for 2 h, it was concentrated in vacuo and purified by flash chromatography on silica gel (0-20% MeOH/CH₂Cl₂, linear gradient, with constant 0.1% v/v AcOH additive) to provide 83.8 mg (48%) of 59_{MAC} as a dark blue solid (mixture of two diastereomers). ¹H NMR (CD₃OD, 400 MHz) δ 6.92 (s, 1H), 6.89-6.80 (m, 2H), 5.22-5.14 (m, 2H), 3.82 (dq, J=14.2, 7.0 Hz, 1H), 3.66 (dq, J=14.5, 7.3 Hz, 1H), 3.60 (t, J=5.8 Hz, 2H), 3.56 (t, J=6.0 Hz, 2H), 3.53 (s, 1.5H), 3.52 (s, 1.5H), 3.24-3.16 (m, 2H), 2.87-2.69 (m, 2H), 2.61 (dt, J=15.4, 6.2 Hz, 1H), 2.11-2.00 (m, 2H), 1.97-1.92 (m, 2H), 1.91-1.83 (m, 1H), 1.90 (s, 1.5H), 1.88 (s, 1.5H), 1.86 (s, 1.5H), 1.85 (s, 1.5H), 1.60-1.49 (m, 1H), 1.48 (s, 1.5H), 1.47 (s, 1.5H), 1.36 (s, 1.5H), 1.35 (t, J=7.1 Hz, 3H), 1.33 (s, 1.5H), 1.19 (d, J=6.6 Hz, 1.5H), 1.15 (d, J=6.5 Hz, 1.5H); 19F NMR (CD₃OD, 376 MHz) δ−111.78 (d, J=14.7 Hz, 1F), −129.97 (dd, J=21.8, 2.0 Hz, 0.5F), −130.09 (dd, J=21.8, 2.0 Hz, 0.5F), −141.98 (dd, J=21.9, 14.5 Hz, 0.5F), −142.00 (dd, J=22.1, 14.6 Hz, 0.5F); Analytical HPLC: t_{R} (two isomers)=13.8 min, 14.1 min; >99% total purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₄₂H₄₄F₃N₄O₄[M+H]⁺ 725.3309, found 725.3310.

### Example 136: MAC Substitution of 4,5,6,7⁻Tetrafluoroxanthenes: 2⁻(3,6-Bis(methyl(phenyl)amino)xanthylium-9⁻yl)-4⁻(dicyano(methoxymethoxy)methyl)-3,5,6-trifluoro-benzoate (64_{MAC})

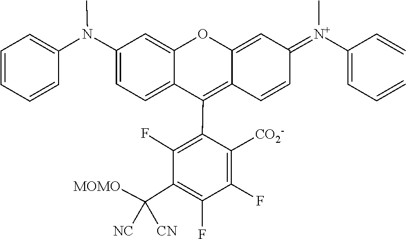

Rhodamine 64 (165 mg, 0.283 mmol) and 2⁻(methoxymethoxy)malononitrile (3; 35.7 mg, 0.283 mmol, 1 eq) were combined in DMF (6 mL), and DIEA (98.7 μL, 0.566 mmol, 2 eq) was added. After stirring the reaction at room temperature for 2 h, it was evaporated to dryness. Flash chromatography on silica gel (25-100% EtOAc/hexanes, linear gradient) afforded 64_{MAC} (115 mg, 59%) as a dark purple solid. ¹H NMR (CDCl₃, 400 MHz) δ 7.45-7.37 (m, 4H), 7.26-7.19 (m, 6H), 6.78 (d, J=8.9 Hz, 2H), 6.61 (d, J=2.4 Hz, 2H), 6.58 (dd, J=8.9, 2.5 Hz, 2H), 5.14 (s, 2H), 3.53 (s, 3H), 3.38 (s, 6H); 19F NMR (CDCl₃, 376 MHz) δ−116.29 (d, J=21.6 Hz, 1F), −126.47 (d, J=20.8 Hz, 1F), −139.41 (t, J=21.2 Hz, 1F); Analytical HPLC: t_{R}=11.4 min, >99% purity (30-95% MeCN/H₂O, linear gradient, with constant 0.1% v/v TFA additive; 20 min run; 1 mL/min flow; ESI; positive ion mode; detection at 550 nm); HRMS (ESI) calcd for C₃₉H₂₈F₃N₄O₅[M+H]⁺ 689.2006, found 689.1999.

### Example 137: MAC Substitution of 4,5,6,7⁻Tetrafluoroxanthenes: 6⁻(MOM-MAC)-JQ₆₄₅ (65_{MAC})

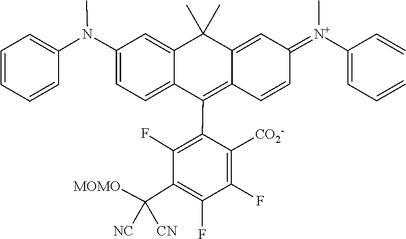

Carborhodamine 65 (150 mg, 0.246 mmol) and 2-(methoxymethoxy)malononitrile (3; 31.1 mg, 0.246 mmol, 1 eq) were combined in DMF (6 mL), and DIEA (85.8 μL, 0.493 mmol, 2 eq) was added. After stirring the reaction at room temperature for 2 h, it was evaporated to dryness. Flash chromatography on silica gel (0-75% EtOAc/hexanes, linear gradient) afforded 65_{MAC} as a yellow-green solid (93.3 mg, 53%). 1H NMR (CDCl₃, 400 MHz) δ 7.39-7.32 (m, 4H), 7.20-7.15 (m, 4H), 7.14-7.09 (m, 2H), 7.09 (d, J=2.5 Hz, 2H), 6.74 (dd, J=8.8, 2.5 Hz, 2H), 6.64 (d, J=8.8 Hz, 2H), 5.14 (s, 2H), 3.51 (s, 3H), 3.37 (s, 6H), 1.65 (s, 3H), 1.63 (s, 3H); 19F NMR (CDCl₃, 376 MHz) δ−116.51 (d, J=22.7 Hz, 1F), −127.06 (d, J=20.5 Hz, 1F), −140.00 (dd, J=22.9, 20.3 Hz, 1F); Analytical HPLC: t_{R}=14.1 min, >99% purity (30-95% MeCN/H₂O, linear gradient, with constant 0.1% v/v TFA additive; 20 min run; 1 mL/min flow; ESI; positive ion mode; detection at 650 nm); HRMS (ESI) calcd for C₄₂H₃₄F₃N₄O₄[M+H]⁺ 715.2527, found 715.2518.

### Example 138: MAC Substitution of 4,5,6,7⁻Tetrafluoroxanthenes: 4-(Dicyano(methoxymethoxy)methyl)-2⁻(5,5⁻dimethyl-3,7-bis(methyl(phenyl)amino)dibenzo[b,e]silin-10⁻ylium-10(5H)-yl)-3,5,6⁻trifluorobenzoate (661_{MAC})

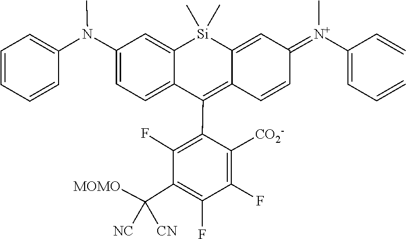

Si-rhodamine 66 (125 mg, 0.200 mmol) and 2⁻(methoxymethoxy)malononitrile (3; 25.2 mg, 0.200 mmol, 1 eq) were combined in DMF (5 mL), and DIEA (69.7 μL, 0.400 mmol, 2 eq) was added. After stirring the reaction at room temperature for 2 h, it was evaporated to dryness. Flash chromatography on silica gel (0-40% EtOAc/hexanes, linear gradient) afforded 66_{MAC} as an off-white solid (79.9 mg, 55%). ¹H NMR (CDCl₃, 400 MHz) δ 7.38-7.31 (m, 4H), 7.19-7.09 (m, 8H), 6.79 (dd, J=8.9, 2.7 Hz, 2H), 6.71 (dd, J=8.9, 0.7 Hz, 2H), 5.18 (s, 2H), 3.54 (s, 3H), 3.36 (s, 6H), 0.50 (s, 3H), 0.47 (s, 3H); 19F NMR (CDCl₃, 376 MHz) δ−113.25 (d, J=22.4 Hz, 1F), −127.11 (d, J=20.2 Hz, 1F), −139.39 (dd, J=22.7, 20.2 Hz, 1F); Analytical HPLC: t_{R}=15.0 min, >99% purity (50-95% MeCN/H₂O, linear gradient, with constant 0.1% v/v TFA additive; 20 min run; 1 mL/min flow; ESI; positive ion mode; detection at 675 nm); HRMS (ESI) calcd for C₄₁H₃₄F₃N₄O₄Si [M+H]⁺ 731.2296, found 731.2290.

### Example 139: Conversion of MAC Xanthenes to HaloTag® Ligands (Scheme 18): 4,5,7⁻Trifluoro-fluorescein-HaloTag® ligand (60_{HTL})

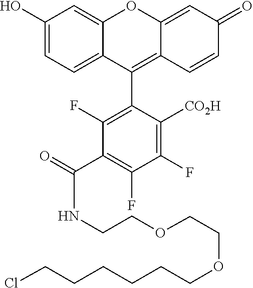 [SMILES: O=C(NCCOCCOCCCCCCCl)c1c(F)c(F)c(C(=O)O)c(-c2c3ccc(=O)cc-3oc3cc(O)ccc23)c1F]

4,5,7⁻Trifluoro-6⁻(MOM-MAC)-fluorescein (60_{MAC}; 30 mg, 58.8 mol) was taken up in CH₂Cl₂ (4 mL); triethylsilane (400 μL) was added, followed by trifluoroacetic acid (800 L). The reaction was stirred at room temperature for 24 h. Toluene (5 mL) was added, and the reaction mixture was concentrated to dryness. The residue was combined with a premixed solution of HaloTag®(O2)amine (HTL-NH₂, 111; TFA salt; 39.7 mg, 0.118 mmol, 2 eq) and DIEA (102 μL, 0.588 mmol, 10 eq) in DMF (4 mL), and the reaction was stirred at room temperature for 18 h. The solvent was removed by rotary evaporation, and the crude material was purified by reverse phase HPLC (20-70% MeCN/H₂O, linear gradient, with constant 0.1% v/v TFA additive) to yield 27.1 mg (72%) of 60_{HTL} as an orange solid. ¹H NMR (CD₃OD, 400 MHz) δ 9.05 (t, J=5.1 Hz, 1H), 6.89 (d, J=8.7 Hz, 2H), 6.70 (d, J=2.4 Hz, 2H), 6.63 (dd, J=8.7, 2.4 Hz, 2H), 3.61-3.47 (m, 10H), 3.40 (t, J=6.5 Hz, 2H), 1.78-1.69 (m, 2H), 1.55-1.46 (m, 2H), 1.46-1.38 (m, 2H), 1.37-1.28 (m, 2H); 19F NMR (CD₃OD, 376 MHz) δ−122.09-−122.44 (m, 1F), −133.85 (d, J=20.3 Hz, 1F), −142.82-−143.23 (m, 1F); Analytical HPLC: t_{R}=13.7 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₃₁H₃₀C₁F₃NO₈ [M+H]⁺ 636.1607, found 636.1611.

### Example 140: Conversion of MAC Xanthenes to HaloTag® Ligands (Scheme 18): 4,5,7⁻Trifluoro-carbofluorescein-HaloTag® ligand (51_{HTL})

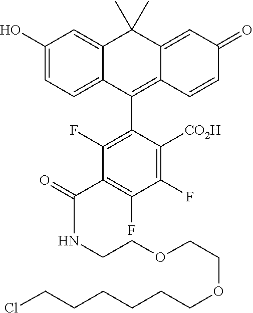 [SMILES: CC1(C)C2=CC(=O)C=CC2=C(c2c(F)c(C(=O)NCCOCCOCCCCCCCl)c(F)c(F)c2C(=O)O)c2ccc(O)cc21]

4,5,7⁻Trifluoro-6⁻(MOM-MAC)-carbofluorescein (51_{MAC}; 50 mg, 93.2 mol) was taken up in CH₂Cl₂ (3 mL); triethylsilane (300 μL) was added, followed by trifluoroacetic acid (600 L). The reaction was stirred at room temperature for 18 h. Toluene (4 mL) was added, and the reaction mixture was concentrated to dryness. The residue was combined with a premixed solution of HaloTag®(O2)amine (HTL-NH₂, 111; TFA salt; 63.0 mg, 0.186 mmol, 2 eq) and DIEA (162 μL, 0.932 mmol, 10 eq) in DMF (3 mL), and the reaction was stirred at room temperature for 2 h. It was then diluted with 10% citric acid and extracted with EtOAc (2×). The combined organic extracts were dried over anhydrous MgSO₄, filtered, and concentrated in vacuo. Purification by reverse phase HPLC (30-70% MeCN/H₂O, linear gradient, with constant 0.1% v/v TFA additive) yielded 14.2 mg (23%) of 51_{HTL} as a light pink solid. ¹H NMR (CD₃OD, 400 MHz) δ 9.07 (t, J=5.6 Hz, 1H), 7.09 (d, J=2.5 Hz, 2H), 6.77 (d, J=8.6 Hz, 2H), 6.67 (dd, J=8.6, 2.5 Hz, 2H), 3.60-3.46 (m, 10H), 3.40 (t, J=6.5 Hz, 2H), 1.78-1.68 (m, 2H), 1.74 (s, 3H), 1.69 (s, 3H), 1.56-1.46 (m, 2H), 1.46-1.38 (m, 2H), 1.38-1.28 (m, 2H); 19F NMR (CD₃OD, 376 MHz) δ−122.22 (d, J=22.5 Hz, 1F), −134.87 (d, J=20.4 Hz, 1F), −143.66 (t, J=21.6 Hz, 1F); Analytical HPLC: t_{R}=13.9 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₃₄H₃₆C₁F₃NO₇ [M+H]⁺ 662.2127, found 662.2129.

### Example 141: Conversion of MAC Xanthenes to HaloTag® Ligands (Scheme 18): 4,5,7⁻Trifluoro-Si-fluorescein-HaloTag® ligand (55_{HTL})

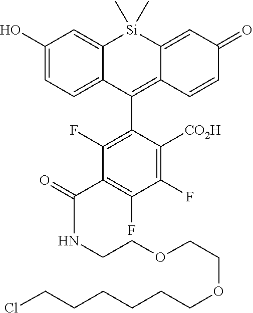 [SMILES: C[Si]1(C)C2=CC(=O)C=CC2=C(c2c(F)c(C(=O)NCCOCCOCCCCCCCl)c(F)c(F)c2C(=O)O)c2ccc(O)cc21]

4,5,7⁻Trifluoro-6⁻(MOM-MAC)—Si-fluorescein (55_{MAC}; 30 mg, 54.3 mol) was taken up in CH₂Cl₂ (3 mL); triethylsilane (300 μL) was added, followed by trifluoroacetic acid (600 L). The reaction was stirred at room temperature for 24 h. Toluene (4 mL) was added, and the reaction mixture was concentrated to dryness. The residue was combined with a premixed solution of HaloTag®(O2)amine (HTL-NH₂, 111; TFA salt; 36.7 mg, 0.109 mmol, 2 eq) and DIEA (94.6 μL, 0.543 mmol, 10 eq) in DMF (3 mL), and the reaction was stirred at room temperature for 2 h. The solvent was removed by rotary evaporation, and the crude material was purified by reverse phase HPLC (20-70% MeCN/H₂O, linear gradient, with constant 0.1% v/v TFA additive) to yield 20.4 mg (55%) of 55_{HTL} as a white solid. ¹H NMR (CD₃OD, 400 MHz) δ 9.15 (t, J=5.3 Hz, 1H), 7.14 (d, J=2.7 Hz, 2H), 6.87 (dd, J=8.7, 1.0 Hz, 2H), 6.74 (dd, J=8.7, 2.8 Hz, 2H), 3.65-3.52 (m, 8H), 3.50 (t, J=6.7 Hz, 2H), 3.42 (t, J=6.6 Hz, 2H), 1.74-1.65 (m, 2H), 1.55-1.46 (m, 2H), 1.44-1.26 (m, 4H), 0.56 (s, 3H), 0.52 (s, 3H); 19F NMR (CD₃OD, 376 MHz) δ−118.77 (d, J=22.2 Hz, 1F), −134.57-−134.69 (m, 1F), −143.21 (t, J=21.6 Hz, 1F); Analytical HPLC: t_{R}=14.2 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₃₃H₃₆ClF₃NO₇Si [M+H]⁺ 678.1896, found 678.1907.

### Example 142: Conversion of MAC Xanthenes to HaloTag® Ligands (Scheme 18): FMGL-HaloTag® ligand (49_{HTL})

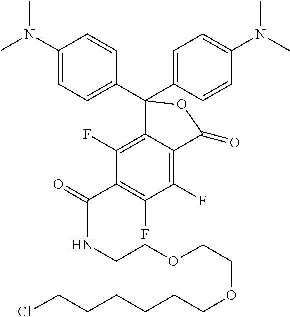

4,5,7⁻Trifluoro-6⁻(MOM-MAC)-MGL (49_{MAC}; 75 mg, 0.136 mmol) was taken up in CH₂Cl₂ (5 mL); triethylsilane (500 μL) was added, followed by trifluoroacetic acid (1 mL). The reaction was stirred at room temperature for 18 h. Toluene (5 mL) was added, and the reaction mixture was concentrated to dryness. The residue was combined with a premixed solution of HaloTag®(O2)amine (HTL-NH₂, 111; TFA salt; 92.0 mg, 0.272 mmol, 2 eq) and DIEA (237 μL, 1.36 mmol, 10 eq) in CH₂Cl₂ (4 mL), and the reaction was stirred at room temperature for 6 h. The solvent was removed by rotary evaporation, and the crude material was purified by reverse phase HPLC (20-80% MeCN/H₂O, linear gradient, with constant 0.1% v/v TFA additive) to yield 49.5 mg (46%, TFA salt) of 49_{HTL} as a blue solid. ¹H NMR (CD₃OD, 400 MHz) δ 9.09 (t, J=5.4 Hz, 1H), 7.21 (d, J=9.0 Hz, 4H), 6.92 (d, J=8.9 Hz, 4H), 3.66-3.54 (m, 8H), 3.52 (t, J=6.7 Hz, 2H), 3.45 (t, J=6.5 Hz, 2H), 3.03 (s, 12H), 1.77-1.68 (m, 2H), 1.58-1.50 (m, 2H), 1.47-1.29 (m, 4H); 19F NMR (CD₃OD, 376 MHz) δ−75.79 (s, 3F), −117.70 (d, J=22.0 Hz, 1F), −134.45 (d, J=21.0 Hz, 1F), −142.89 (t, J=21.5 Hz, 1F); Analytical HPLC: t_{R}=13.5 min, >99% purity (10-95% MeCN/H₂O, linear gradient, with constant 0.1% v/v TFA additive; 20 min run; 1 mL/min flow; ESI; positive ion mode; detection at 280 nm); HRMS (ESI) calcd for C₃₅H₄₂C₁F₃N₃O₅ [M+H]⁺ 676.2760, found 676.2767.

### Example 143: Conversion of MAC Xanthenes to HaloTag® Ligands (Scheme 18): FRh₁₁₀-HaloTag® ligand (70_{HTL})

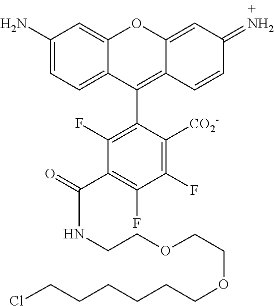

4,5,7⁻Trifluoro-6⁻(MOM-MAC)-rhodamine 110 bis(tert-butyl carbamate) (67_{MAC}; 90 mg, 0.127 mmol) was taken up in CH₂Cl₂ (5 mL); triethylsilane (500 μL) was added, followed by trifluoroacetic acid (1 mL). The reaction was stirred at room temperature for 8 h. Toluene (5 mL) was added, and the reaction mixture was concentrated to dryness. The residue was combined with a premixed solution of HaloTag®(O2)amine (HTL-NH₂, 111; TFA salt; 85.8 mg, 0.254 mmol, 2 eq) and DIEA (221 μL, 1.27 mmol, 10 eq) in DMF (3 mL), and the reaction was stirred at room temperature for 18 h. The solvent was removed by rotary evaporation, and the crude material was purified by reverse phase HPLC (30-40% MeCN/H₂O, linear gradient, with constant 0.1% v/v TFA additive) to afford 70_{HTL} as a red-orange solid (20.5 mg, 22%, TFA salt). ¹H NMR (CD₃OD, 400 MHz) δ 9.12 (t, J=5.5 Hz, 1H), 7.29 (dd, J=9.2, 0.9 Hz, 2H), 6.88 (dd, J=9.2, 2.1 Hz, 2H), 6.80 (d, J=2.1 Hz, 2H), 3.68-3.54 (m, 8H), 3.51 (t, J=6.6 Hz, 2H), 3.42 (t, J=6.5 Hz, 2H), 1.76-1.66 (m, 2H), 1.54-1.45 (m, 2H), 1.44-1.27 (m, 4H); 19F NMR (CD₃OD, 376 MHz) δ−75.34 (s, 3F), −116.65 (d, J=15.3 Hz, 1F), −132.55 (d, J=21.7 Hz, 1F), −140.18-−140.53 (m, 1F); Analytical HPLC: t_{R}=11.0 min, >99% purity (10-95% MeCN/H₂O, linear gradient, with constant 0.1% v/v TFA additive; 20 min run; 1 mL/min flow; ESI; positive ion mode; detection at 525 nm); HRMS (ESI) calcd for C₃₁H₃₂C₁F₃N₃O₆ [M+H]⁺ 634.1926, found 634.1926.

### Example 144: Conversion of MAC Xanthenes to HaloTag® Ligands (Scheme 18): FCRh₁₁₀-HaloTag@ligand (71_{HTL})

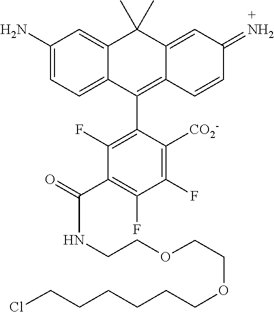

4,5,7⁻Trifluoro-6⁻(MOM-MAC)-carborhodamine 110 bis(tert-butyl carbamate) (68_{MAC}; 90 mg, 0.122 mmol) was taken up in CH₂Cl₂ (5 mL); triethylsilane (500 μL) was added, followed by trifluoroacetic acid (1 mL). The reaction was stirred at room temperature for 6 h. Toluene (5 mL) was added, and the reaction mixture was concentrated to dryness. The residue was combined with a premixed solution of HaloTag®(O2)amine (HTL-NH₂, 111; TFA salt; 82.7 mg, 0.245 mmol, 2 eq) and DIEA (213 μL, 1.22 mmol, 10 eq) in DMF (3 mL), and the reaction was stirred at room temperature for 18 h. The solvent was removed by rotary evaporation, and the crude material was purified by reverse phase HPLC (30-50% MeCN/H₂O, linear gradient, with constant 0.1% v/v TFA additive) to provide 71_{HTL} as a purple solid (30.5 mg, 32%, TFA salt). ¹H NMR (CD₃OD, 400 MHz) δ 9.14 (t, J=5.4 Hz, 1H), 7.153 (dd, J=9.1, 0.9 Hz, 2H), 7.148 (d, J=2.2 Hz, 2H), 6.67 (dd, J=9.0, 2.2 Hz, 2H), 3.68-3.55 (m, 8H), 3.52 (t, J=6.6 Hz, 2H), 3.44 (t, J=6.5 Hz, 2H), 1.75 (s, 3H), 1.74-1.67 (m, 2H), 1.64 (s, 3H), 1.56-1.47 (m, 2H), 1.45-1.28 (m, 4H); 19F NMR (CD₃OD, 376 MHz) δ−75.34 (s, 3F), −117.19 (d, J=15.3 Hz, 1F), −133.70 (d, J=22.2 Hz, 1F), −140.45 (dd, J=22.1, 15.6 Hz, 1F); Analytical HPLC: t_{R}=11.3 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₃₄H₃₈C₁F₃N₃O₅ [M+H]⁺ 660.2447, found 660.2459.

### Example 145: Conversion of MAC Xanthenes to HaloTag® Ligands (Scheme 18): FSiRh₁₁₀-HaloTag® ligand (72_{HTL})

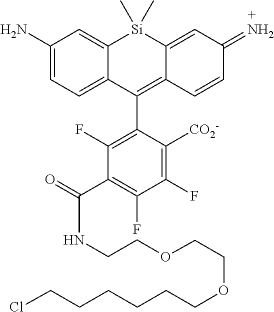

4,5,7⁻Trifluoro-6⁻(MOM-MAC)—Si-rhodamine 110 bis(tert-butyl carbamate) (69_{MAC}; 75 mg, 0.100 mmol) was taken up in CH₂Cl₂ (4 mL); triethylsilane (400 μL) was added, followed by trifluoroacetic acid (800 L). The reaction was stirred at room temperature for 6 h. Toluene (4 mL) was added, and the reaction mixture was concentrated to dryness. The residue was combined with a premixed solution of HaloTag®(O2)amine (HTL-NH₂, 111; TFA salt; 67.5 mg, 0.200 mmol, 2 eq) and DIEA (174 μL, 1.00 mmol, 10 eq) in DMF (3 mL), and the reaction was stirred at room temperature for 18 h. The solvent was removed by rotary evaporation, and the crude material was purified by reverse phase HPLC (30-50% MeCN/H₂O, linear gradient, with constant 0.1% v/v TFA additive) to provide 72_{HTL} as a bright blue solid (30.7 mg, 39%, TFA salt). ¹H NMR (CD₃OD, 400 MHz) δ 9.17 (t, J=5.0 Hz, 1H), 7.21 (d, J=2.5 Hz, 2H), 6.99 (dd, J=8.9, 1.3 Hz, 2H), 6.76 (dd, J=8.9, 2.5 Hz, 2H), 3.67-3.54 (m, 8H), 3.51 (t, J=6.6 Hz, 2H), 3.44 (t, J=6.5 Hz, 2H), 1.75-1.66 (m, 2H), 1.56-1.47 (m, 2H), 1.45-1.28 (m, 4H), 0.57 (s, 3H), 0.51 (s, 3H); 19F NMR (CD₃OD, 376 MHz) δ−75.40 (s, 3F), −118.23 (d, J=19.4 Hz, 1F), −134.29 (d, J=21.2 Hz, 1F), −141.77-−142.06 (m, 1F); Analytical HPLC: t_{R}=11.7 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₃₃H₃₈C₁F₃N₃O₅Si [M+H]⁺ 676.2216, found 676.2222.

### Example 146: Conversion of MAC Xanthenes to HaloTag® Ligands (Scheme 18): JF₆₃₂-HaloTag® ligand (44_{HTL})

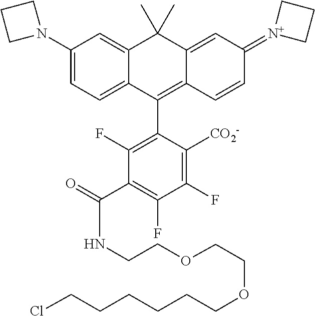

6⁻(MOM-MAC)-JF₆₃₂ (44_{MAC}; 20.0 mg, 32.5 mol) was taken up in CH₂Cl₂ (2 mL); triethylsilane (200 μL) was added, followed by trifluoroacetic acid (400 L). The reaction was stirred at room temperature for 6 h. Toluene (3 mL) was added, and the reaction mixture was concentrated to dryness. The residue was combined with a premixed solution of HaloTag®(O2)amine (HTL-NH₂, 111; TFA salt; 22.0 mg, 65.1 mol, 2 eq) and DIEA (56.7 μL, 0.325 mmol, 10 eq) in CH₂Cl₂ (2 mL), and the reaction was stirred at room temperature for 18 h. The solvent was removed by rotary evaporation, and the crude material was purified by reverse phase HPLC (20-80% MeCN/H₂O, linear gradient, with constant 0.1% v/v TFA additive) to yield 20.2 mg (73%, TFA salt) of 44_{HTL} as a deep blue solid. ¹H NMR (CD₃OD, 400 MHz) δ 9.09 (t, J=5.4 Hz, 1H), 7.15 (dd, J=9.1, 0.5 Hz, 2H), 6.81 (d, J=2.2 Hz, 2H), 6.45 (dd, J=9.1, 2.2 Hz, 2H), 4.37 (t, J=7.7 Hz, 8H), 3.67-3.55 (m, 8H), 3.52 (t, J=6.7 Hz, 2H), 3.44 (t, J=6.5 Hz, 2H), 2.56 (p, J=7.8 Hz, 4H), 1.77 (s, 3H), 1.75-1.68 (m, 2H), 1.66 (s, 3H), 1.56-1.48 (m, 2H), 1.44-1.30 (m, 4H); 19F NMR (CD₃OD, 376 MHz) δ−75.31 (s, 3F), −117.35 (d, J=15.2 Hz, 1F), −133.92 (d, J=22.5 Hz, 1F), −140.91 (dd, J=22.1, 15.3 Hz, 1F); Analytical HPLC: t_{R}=12.9 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₄₀H₄₆C₁F₃N₃O₅ [M+H]⁺ 740.3073, found 740.3068.

### Example 147: Conversion of MAC Xanthenes to HaloTag® Ligands (Scheme 18): JFX₅₇₆-HaloTag® ligand (8_{HTL})

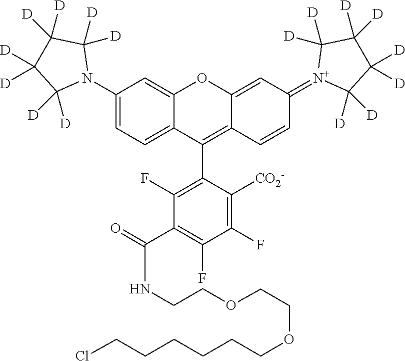

6⁻(MOM-MAC)-JFX₅₇₆ (8_{MAC}; 60 mg, 86.6 mol) was taken up in CH₂Cl₂ (5 mL); triethylsilane (500 μL) was added, followed by trifluoroacetic acid (1 mL). The reaction was stirred at room temperature for 6 h. Toluene (5 mL) was added, and the reaction mixture was concentrated to dryness. The residue was combined with a premixed solution of HaloTag®(O2)amine (HTL-NH₂, 111; TFA salt; 58.5 mg, 0.173 mmol, 2 eq) and DIEA (151 L, 0.866 mmol, 10 eq) in CH₂Cl₂ (4 mL), and the reaction was stirred at room temperature for 18 h. The solvent was removed by rotary evaporation, and the crude material was purified by reverse phase HPLC (30-60% MeCN/H₂O, linear gradient, with constant 0.1% v/v TFA additive) to afford 8_{HTL} as a dark red-purple solid (49.2 mg, 65%, TFA salt). ¹H NMR (CD₃OD, 400 MHz) δ 9.13 (t, J=5.5 Hz, 1H), 7.33 (d, J=9.4 Hz, 2H), 6.97 (dd, J=9.4, 2.3 Hz, 2H), 6.82 (d, J=2.3 Hz, 2H), 3.67-3.55 (m, 8H), 3.51 (t, J=6.6 Hz, 2H), 3.42 (t, J=6.5 Hz, 2H), 1.74-1.66 (m, 2H), 1.50 (p, J=6.8 Hz, 2H), 1.43-1.26 (m, 4H); 19F NMR (CD₃OD, 376 MHz) δ-75.36 (s, 3F), −116.57 (d, J=15.1 Hz, 1F), −132.50 (d, J=22.3 Hz, 1F), −140.03-−140.23 (m, 1F); Analytical HPLC: t_{R}=13.1 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₃₉H₂₈D₁₆C₁F₃N₃O₆ [M+H]⁺ 758.3870, found 758.3861.

### Example 148: Conversion of MAC Xanthenes to HaloTag® Ligands (Scheme 18): JFX₆₃₇-HaloTag® ligand (40_{HTL})

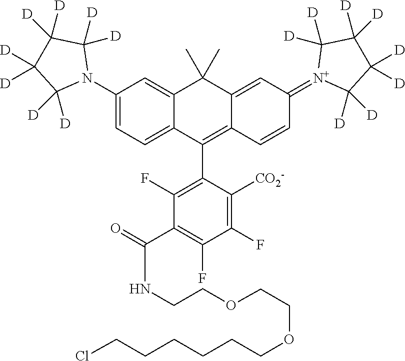

6⁻(MOM-MAC)-JFX₆₃₇ (40_{MAC}; 28.0 mg, 42.5 mol) was taken up in CH₂Cl₂ (3 mL); triethylsilane (300 μL) was added, followed by trifluoroacetic acid (600 L). The reaction was stirred at room temperature for 6 h. Toluene (4 mL) was added, and the reaction mixture was concentrated to dryness. The residue was combined with a premixed solution of HaloTag®(O2)amine (HTL-NH₂, 111; TFA salt; 28.7 mg, 85.0 mol, 2 eq) and DIEA (74.0 μL, 0.425 mmol, 10 eq) in CH₂Cl₂ (3 mL), and the reaction was stirred at room temperature for 18 h. The solvent was removed by rotary evaporation, and the crude material was purified by reverse phase HPLC (30-70% MeCN/H₂O, linear gradient, with constant 0.1% v/v TFA additive) to yield 18.9 mg (49%, TFA salt) of 40_{HTL} as a deep blue solid. ¹H NMR (CD₃OD, 400 MHz) δ 9.15 (t, J=5.4 Hz, 1H), 7.21 (dd, J=9.3, 1.0 Hz, 2H), 7.10 (d, J=2.4 Hz, 2H), 6.74 (dd, J=9.3, 2.4 Hz, 2H), 3.68-3.55 (m, 8H), 3.51 (t, J=6.6 Hz, 2H), 3.43 (t, J=6.5 Hz, 2H), 1.83 (s, 3H), 1.75-1.66 (m, 2H), 1.71 (s, 3H), 1.55-1.47 (m, 2H), 1.44-1.26 (m, 4H); 19F NMR (CD₃OD, 376 MHz) δ−75.29 (s, 3F), −117.12 (d, J=15.0 Hz, 1F), −133.84 (d, J=22.1 Hz, 1F),-140.64-−140.81 (m, 1F); Analytical HPLC: t_{R}=13.4 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₄₂H₃₄D₁₆C₁F₃N₃O₅ [M+H]⁺ 784.4390, found 784.4385.

### Example 149: Conversion of MAC Xanthenes to HaloTag® Ligands (Scheme 18): JFX₆₇₃-HaloTag® ligand (25_{HTL})

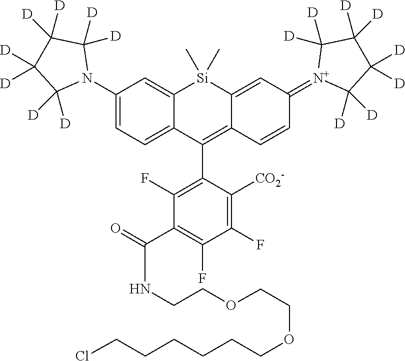

6⁻(MOM-MAC)-JFX₆₇₃ (25_{MAC}; 50 mg, 74.1 mol) was taken up in CH₂Cl₂ (3 mL); triethylsilane (300 μL) was added, followed by trifluoroacetic acid (600 L). The reaction was stirred at room temperature for 6 h. Toluene (4 mL) was added, and the reaction mixture was concentrated to dryness. The residue was combined with a premixed solution of HaloTag®(O2)amine (HTL-NH₂, 111; TFA salt; 50.0 mg, 0.148 mmol, 2 eq) and DIEA (129 L, 0.741 mmol, 10 eq) in CH₂Cl₂ (3 mL), and the reaction was stirred at room temperature for 18 h. The solvent was removed by rotary evaporation, and the crude material was purified by reverse phase HPLC (20-70% MeCN/H₂O, linear gradient, with constant 0.1% v/v TFA additive) to afford 25_{HTL} as a blue solid (40.4 mg, 60%, TFA salt). ¹H NMR (CD₃OD, 400 MHz) δ 9.12 (t, J=5.4 Hz, 1H), 7.16 (dd, J=9.4, 1.0 Hz, 2H), 7.14 (d, J=2.7 Hz, 2H), 6.64 (dd, J=9.4, 2.7 Hz, 2H), 3.67-3.54 (m, 8H), 3.50 (t, J=6.6 Hz, 2H), 3.43 (t, J=6.5 Hz, 2H), 1.75-1.66 (m, 2H), 1.55-1.47 (m, 2H), 1.44-1.27 (m, 4H), 0.58 (s, 3H), 0.54 (s, 3H); 19F NMR (CD₃OD, 376 MHz) δ−75.24 (s, 3F), −118.19 (d, J=16.4 Hz, 1F), −135.10 (d, J=22.6 Hz, 1F),-142.57-−142.90 (m, 1F); Analytical HPLC: t_{R}=13.5 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₄1H₃₄D₁₆ClF₃N₃O₅Si [M+H]⁺ 800.4159, found 800.4167.

### Example 150: Conversion of MAC Xanthenes to HaloTag® Ligands (Scheme 18): 4⁻((2⁻(2⁻((6⁻Chlorohexyl)oxy)ethoxy)ethyl)carbamoyl)-2,3,5⁻trifluoro-6-(1,2,2,4,8,10,10,11⁻octamethyl-1,2,10,11⁻tetrahydropyrano[3,2⁻g:5,6⁻g′]diquinolin-13⁻ium-6⁻yl)benzoate (11_{HTL})

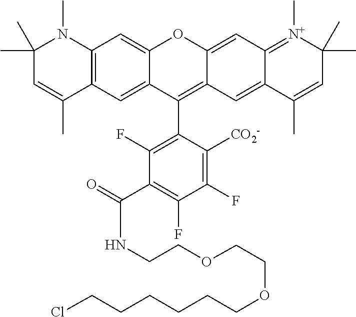

Rhodamine 11_{MAC} (acetate salt; 60 mg, 79.3 μmol) was taken up in CH₂Cl₂ (5 mL), and trifluoroacetic acid (1 mL) was added. The reaction was stirred at room temperature for 18 h. Toluene (5 mL) was added, and the reaction mixture was concentrated to dryness. The residue was combined with a premixed solution of HaloTag®(O2)amine (HTL-NH₂, 111; TFA salt; 53.6 mg, 0.159 mmol, 2 eq) and DIEA (138 μL, 0.793 mmol, 10 eq) in DMF (4 mL), and the reaction was stirred at room temperature for 2 h. The solvent was removed by rotary evaporation, and the crude material was purified by reverse phase HPLC (40-60% MeCN/H₂O, linear gradient, with constant 0.1% v/v TFA additive) to afford 11H_{TL} as a purple solid (37.3 mg, 50%, TFA salt). ¹H NMR (CD₃OD, 400 MHz) δ 9.12 (t, J=5.4 Hz, 1H), 6.89 (s, 2H), 6.84 (s, 2H), 5.74-5.70 (m, 2H), 3.67-3.55 (m, 8H), 3.50 (t, J=6.7 Hz, 2H), 3.42 (t, J=6.5 Hz, 2H), 3.21 (s, 6H), 1.92 (d, J=1.4 Hz, 6H), 1.73-1.66 (m, 2H), 1.525 (s, 6H), 1.523 (s, 6H), 1.50-1.45 (m, 2H), 1.41-1.28 (m, 4H); 19F NMR (CD₃OD, 376 MHz) δ−75.36 (s, 3F), −115.98 (d, J=15.4 Hz, 1F), −131.78 (d, J=22.2 Hz, 1F), −139.85-−140.17 (m, 1F); Analytical HPLC: t_{R}=14.1 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₄₅H₅₂C₁F₃N₃O₆ [M+H]⁺ 822.3491, found 822.3490.

### Example 151: Conversion of MAC Xanthenes to HaloTag® Ligands (Scheme 18): JF₅₆₃-HaloTag® ligand (18_{HTL})

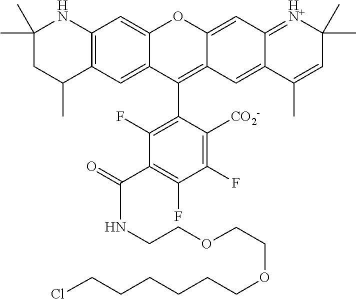

6⁻(MOM-MAC)-JF₅₆₃ (18_{MAC}, acetate salt; 25 mg, 34.1 mol) was taken up in CH₂Cl₂ (2 mL); anisole (200 μL) was added, followed by trifluoroacetic acid (400 L). The reaction was stirred at room temperature for 24 h. Toluene (3 mL) was added, and the reaction mixture was concentrated to dryness. The residue was combined with a premixed solution of HaloTag®(O2)amine (HTL-NH₂, 111; TFA salt; 23.0 mg, 68.2 mol, 2 eq) and DIEA (59.4 μL, 0.341 mmol, 10 eq) in DMF (2 mL), and the reaction was stirred at room temperature for 3 h. The solvent was removed by rotary evaporation, and the crude material was purified by reverse phase HPLC (35-65% MeCN/H₂O, linear gradient, with constant 0.1% v/v TFA additive) to yield 17.3 mg (56%, TFA salt) of 18_{HTL} as a purple solid (mixture of diastereomers). 1H NMR (CD₃OD, 400 MHz) δ 9.19-9.08 (m, 1H), 7.15-7.05 (m, 2H), 6.67-6.61 (m, 2H), 3.70-3.52 (m, 8H), 3.53-3.47 (m, 2H), 3.45-3.40 (m, 2H), 2.98-2.87 (m, 2H), 1.95-1.86 (m, 2H), 1.75-1.66 (m, 2H), 1.54-1.19 (m, 26H); 19F NMR (CD₃OD, 376 MHz) δ−75.35 (s, 3F),-116.07-−116.23 (m, 1F), −132.05-−132.22 (m, 1F), −139.69-−140.86 (m, 1F); Analytical HPLC: t_{R} (three isomers)=13.7 min, 13.8 min, 14.0 min; >99% total purity (10-95% MeCN/H₂O, linear gradient, with constant 0.1% v/v TFA additive; 20 min run; 1 mL/min flow; ESI; positive ion mode; detection at 550 nm); HRMS (ESI) calcd for C₄₃H₅₂C₁F₃N₃O₆ [M+H]⁺ 798.3491, found 798.3488.

### Example 152: Conversion of MAC Xanthenes to HaloTag® Ligands (Scheme 18): 4⁻((2⁻(2⁻((6⁻Chlorohexyl)oxy)ethoxy)ethyl)carbamoyl)-2⁻(1,2,2,4,8,10,10,11,13,13-decamethyl-2,10,11,13⁻tetra-hydrosilino[3,2⁻g:5,6⁻g′]diquinolin-6⁻ylium-6(1H)-yl)-3,5,6-trifluorobenzoate (38_{HTL})

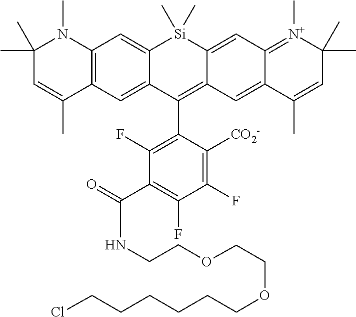

Si-rhodamine 38_{MAC} (50 mg, 67.7 mol) was taken up in CH₂Cl₂ (4 mL), and trifluoroacetic acid (800 μL) was added. The reaction was stirred at room temperature for 18 h. Toluene (5 mL) was added, and the reaction mixture was concentrated to dryness. The residue was combined with a premixed solution of HaloTag®(O2)amine (HTL-NH₂, 111; TFA salt; 45.7 mg, 0.135 mmol, 2 eq) and DIEA (118 μL, 0.677 mmol, 10 eq) in CH₂Cl₂ (4 mL), and the reaction was stirred at room temperature for 1 h. The solvent was removed by rotary evaporation, and the crude material was purified by flash chromatography on silica gel (10-100% EtOAc/hexanes, linear gradient) to afford 38_{HTL} as a yellow-green solid (36.3 mg, 62%). 1H NMR (CDCl₃, 400 MHz) δ 6.622 (s, 2H), 6.621 (s, 1H), 6.59 (s, 2H), 5.28 (q, J=1.3 Hz, 2H), 3.64-3.56 (m, 6H), 3.53-3.47 (m, 4H), 3.33 (t, J=6.6 Hz, 2H), 2.86 (s, 6H), 1.79 (d, J=1.4 Hz, 6H), 1.77-1.69 (m, 2H), 1.50-1.35 (m, 4H), 1.31 (s, 12H), 1.30-1.25 (m, 2H), 0.51 (s, 3H), 0.51 (s, 3H); 19F NMR (CDCl₃, 376 MHz) δ−119.92 (d, J=22.8 Hz, 1F), −134.05 (d, J=21.5 Hz, 1F), −142.39 (t, J=22.2 Hz, 1F); Analytical HPLC: t_{R}=11.9 min, 98.2% purity (30-95% MeCN/H₂O, linear gradient, with constant 0.1% v/v TFA additive; 20 min run; 1 mL/min flow; ESI; positive ion mode; detection at 750 nm); HRMS (ESI) calcd for C₄₇H₅₈C₁F₃N₃O₅Si [M+H]⁺ 864.3781, found 864.3777.

### Example 153: Conversion of MAC Xanthenes to HaloTag® Ligands (Scheme 18): 4⁻((2⁻(2⁻((6⁻Chlorohexyl)oxy)ethoxy)ethyl)carbamoyl)-2,3,5⁻trifluoro-6-(2,2,4,8,10,10,13,13⁻octamethyl-2,10,11,13⁻tetrahydrosilino [3,2⁻g:5,6⁻g′]diquinolin-6⁻ylium-6(1H)-yl)benzoate (36_{HTL})

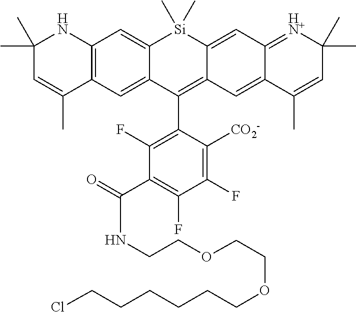

Si-rhodamine 36_{MAC} (20 mg, 28.1 mol) was taken up in CH₂Cl₂ (2 mL); anisole (200 μL) was added, followed by trifluoroacetic acid (400 L). The reaction was stirred at room temperature for 18 h. Toluene (2 mL) was added, and the reaction mixture was concentrated to dryness. The residue was combined with a premixed solution of HaloTag®(O2)amine (HTL-NH₂, 111; TFA salt; 19.0 mg, 56.3 mol, 2 eq) and DIEA (49.0 μL, 0.281 mmol, 10 eq) in DMF (2 mL), and the reaction was stirred at room temperature for 2 h. The solvent was removed by rotary evaporation, and the crude material was purified by reverse phase HPLC (30-60% MeCN/H₂O, linear gradient, with constant 0.1% v/v TFA additive) to afford 36_{HTL} as a green solid (12.9 mg, 48%, TFA salt). ¹H NMR (CD₃OD, 400 MHz) δ 9.16 (t, J=5.5 Hz, 1H), 6.96 (s, 2H), 6.82 (s, 2H), 5.55 (q, J=1.2 Hz, 2H), 3.68-3.54 (m, 8H), 3.51 (t, J=6.6 Hz, 2H), 3.43 (t, J=6.5 Hz, 2H), 1.78 (d, J=1.4 Hz, 6H), 1.75-1.67 (m, 2H), 1.51 (p, J=6.8 Hz, 2H), 1.45-1.28 (m, 4H), 1.414 (s, 6H), 1.405 (s, 6H), 0.52 (s, 3H), 0.48 (s, 3H); 19F NMR (CD₃OD, 376 MHz) δ-75.33 (s, 3F), −116.54 (d, J=15.6 Hz, 1F), −133.68 (d, J=21.7 Hz, 1F), −141.14 (dd, J=22.1, 15.5 Hz, 1F); Analytical HPLC: t_{R}=14.0 min, 99.0% purity (10-95% MeCN/H₂O, linear gradient, with constant 0.1% v/v TFA additive; 20 min run; 1 mL/min flow; ESI; positive ion mode; detection at 725 nm); HRMS (ESI) calcd for C₄₅H₅₄C₁F₃N₃O₅Si [M+H]⁺ 836.3468, found 836.3472.

### Example 154: Conversion of MAC Xanthenes to HaloTag® Ligands (Scheme 18): SiRF₇₁₂-HaloTag® ligand (108_{HTL})

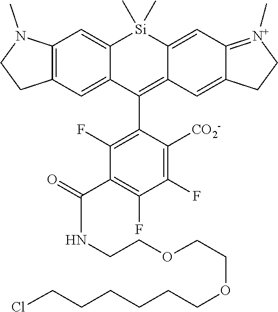

6⁻(MOM-MAC)—SiRF₇₁₂(108_{MAC}; 40 mg, 63.4 mol) was taken up in CH₂Cl₂ (3 mL); triethylsilane (300 μL) was added, followed by trifluoroacetic acid (600 L). The reaction was stirred at room temperature for 6 h. Toluene (4 mL) was added, and the reaction mixture was concentrated to dryness. The residue was combined with a premixed solution of HaloTag®(O2)amine (HTL-NH₂, 111; TFA salt; 42.8 mg, 0.127 mmol, 2 eq) and DIEA (110 L, 0.634 mmol, 10 eq) in DMF (3 mL), and the reaction was stirred at room temperature for 18 h. The solvent was removed by rotary evaporation, and the crude material was purified by reverse phase HPLC (30-50% MeCN/H₂O, linear gradient, with constant 0.1% v/v TFA additive) to afford 108_{HTL} as a blue-green solid (29.9 mg, 54%, TFA salt). ¹H NMR (CD₃OD, 400 MHz) δ 7.10 (s, 2H), 6.81 (s, 2H), 3.83 (t, J=8.0 Hz, 4H), 3.68-3.55 (m, 8H), 3.51 (t, J=6.7 Hz, 2H), 3.44 (t, J=6.5 Hz, 2H), 3.22 (s, 6H), 3.08-2.93 (m, 4H), 1.76-1.67 (m, 2H), 1.56-1.48 (m, 2H), 1.45-1.28 (m, 4H), 0.57 (s, 3H), 0.51 (s, 3H); 19F NMR (CD₃OD, 376 MHz) δ-75.29 (s, 3F), −118.27 (d, J=15.5 Hz, 1F), −135.07 (d, J=22.5 Hz, 1F), −141.70 (dd, J=22.3, 15.9 Hz, 1F); Analytical HPLC: t_{R}=12.8 min, >99% purity (10-95% MeCN/H₂O, linear gradient, with constant 0.1% v/v TFA additive; 20 min run; 1 mL/min flow; ESI; positive ion mode; detection at 725 nm); HRMS (ESI) calcd for C₃₉H₄₆C₁F₃N₃O₅Si [M+H]⁺ 756.2842, found 756.2845.

### Example 155: Conversion of MAC Xanthenes to HaloTag® Ligands (Scheme 18): JF₆₉₈-HaloTag® ligand (31_{HTL})

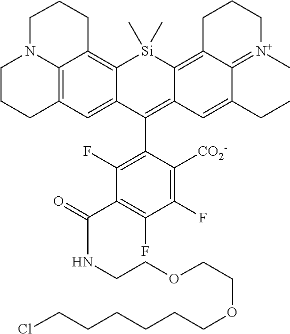

6⁻(MOM-MAC)-JF₆₉₈ (3_{MAC}; 35 mg, 49.2 mol) was taken up in CH₂Cl₂ (3 mL); triethylsilane (300 μL) was added, followed by trifluoroacetic acid (600 L). The reaction was stirred at room temperature for 6 h. Toluene (4 mL) was added, and the reaction mixture was concentrated to dryness. The residue was combined with a premixed solution of HaloTag®(O2)amine (HTL-NH₂, 111; TFA salt; 33.3 mg, 98.5 mol, 2 eq) and DIEA (85.8 μL, 0.492 mmol, 10 eq) in DMF (3 mL), and the reaction was stirred at room temperature for 18 h. The solvent was removed by rotary evaporation, and the crude material was purified by reverse phase HPLC (30-60% MeCN/H₂O, linear gradient, with constant 0.1% v/v TFA additive) to afford 31_{HTL} as a blue-green solid (27.9 mg, 60%, TFA salt). ¹H NMR (CD₃OD, 400 MHz) δ 9.17 (t, J=5.3 Hz, 1H), 6.66 (s, 2H), 3.68-3.54 (m, 16H), 3.52 (t, J=6.7 Hz, 2H), 3.45 (t, J=6.5 Hz, 2H), 3.01-2.94 (m, 4H), 2.63-2.56 (m, 4H), 2.13-2.02 (m, 4H), 1.98-1.88 (m, 4H), 1.77-1.68 (m, 2H), 1.57-1.49 (m, 2H), 1.46-1.30 (m, 4H), 0.71 (s, 3H), 0.67 (s, 3H); 19F NMR (CD₃OD, 376 MHz) δ−75.37 (s, 3F), −117.65 (dd, J=15.0, 2.0 Hz, 1F), −134.78 (dd, J=21.9, 2.0 Hz, 1F), −141.18 (dd, J=22.3, 15.1 Hz, 1F); Analytical HPLC: t_{R}=13.6 min, >99% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₄₅H₅₄C₁F₃N₃O₅Si [M+H]⁺ 836.3468, found 836.3476.

### Example 156: Conversion of MAC Xanthenes to HaloTag® Ligands (Scheme 18): FSiRh_{Q}-HaloTag® ligand (29_{HTL})

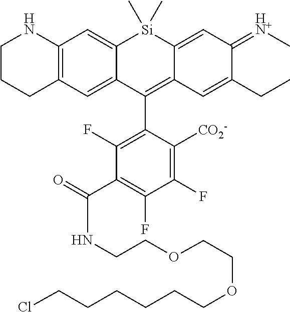

6⁻(MOM-MAC)—FSiRh_{Q} (29_{MAC}; 40 mg, 63.4 mol) was taken up in CH₂Cl₂ (3 mL); triethylsilane (300 μL) was added, followed by trifluoroacetic acid (600 L). The reaction was stirred at room temperature for 8 h. Toluene (4 mL) was added, and the reaction mixture was concentrated to dryness. The residue was combined with a premixed solution of HaloTag®(O2)amine (HTL-NH₂, 111; TFA salt; 42.8 mg, 0.127 mmol, 2 eq) and DIEA (110 L, 0.634 mmol, 10 eq) in DMF (3 mL), and the reaction was stirred at room temperature for 18 h. The solvent was removed by rotary evaporation, and the crude material was purified by reverse phase HPLC (30-5₀% MeCN/H₂O, linear gradient, with constant 0.1% v/v TFA additive) to afford 29_{HTL} as a deep blue solid (23.2 mg, 42%, TFA salt). ¹H NMR (CD₃OD, 400 MHz) δ 9.16 (t, J=5.3 Hz, 1H), 6.98 (s, 2H), 6.82 (s, 2H), 3.68-3.56 (m, 8H), 3.54-3.48 (m, 6H), 3.44 (t, J=6.5 Hz, 2H), 2.66-2.59 (m, 4H), 1.95-1.87 (m, 4H), 1.76-1.68 (m, 2H), 1.56-1.48 (m, 2H), 1.44-1.29 (m, 4H), 0.49 (s, 3H), 0.43 (s, 3H); 19F NMR (CD₃OD, 376 MHz) δ-75.32 (s, 3F), −117.66 (d, J=15.2 Hz, 1F), −134.57 (d, J=22.5 Hz, 1F), −141.14 (dd, J=22.3, 15.2 Hz, 1F); Analytical HPLC: t_{R}=12.6 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₃₉H₄₆C₁F₃N₃O₅Si [M+H]⁺ 756.2842, found 756.2849.

### Example 157: Conversion of MAC Xanthenes to HaloTag® Ligands (Scheme 18): JF₆₆₀-HaloTag® ligand (57_{HTL})

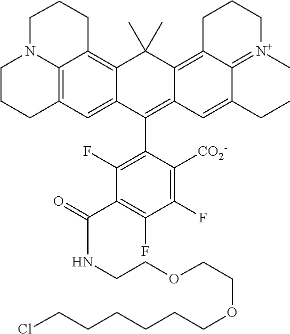

6⁻(MOM-MAC)-JF₆₆₀ (57_{MAC}; 60 mg, 86.4 mol) was taken up in CH₂Cl₂ (3 mL); triethylsilane (300 μL) was added, followed by trifluoroacetic acid (600 L). The reaction was stirred at room temperature for 8 h. Toluene (4 mL) was added, and the reaction mixture was concentrated to dryness. The residue was combined with a premixed solution of HaloTag®(O2)amine (HTL-NH₂, 111; TFA salt; 58.3 mg, 0.173 mmol, 2 eq) and DIEA (150 L, 0.864 mmol, 10 eq) in CH₂Cl₂ (3 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive) to afford 57_{HTL} as a blue solid (33.0 mg, 41%, TFA salt). ¹H NMR (CD₃OD, 400 MHz) δ 9.15 (t, J=5.2 Hz, 1H), 6.64 (s, 2H), 3.68-3.50 (m, 18H), 3.44 (t, J=6.5 Hz, 2H), 3.13-3.02 (m, 4H), 2.67-2.60 (m, 4H), 2.10-1.99 (m, 4H), 2.04 (s, 3H), 1.98-1.90 (m, 4H), 1.96 (s, 3H), 1.76-1.68 (m, 2H), 1.57-1.49 (m, 2H), 1.45-1.30 (m, 4H); 19F NMR (CD₃OD, 376 MHz) δ-75.36 (s, 3F), −117.34 (d, J=15.1 Hz, 1F), −134.31 (d, J=22.5 Hz, 1F), −140.92 (dd, J=22.2, 15.3 Hz, 1F); Analytical HPLC: t_{R}=13.5 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₄₆H₅₄C₁F₃N₃O₅ [M+H]⁺ 820.3699, found 820.3688.

### Example 158: Conversion of MAC Xanthenes to HaloTag® Ligands (Scheme 18): JF₆₅₇-HaloTag® ligand (59_{HTL})

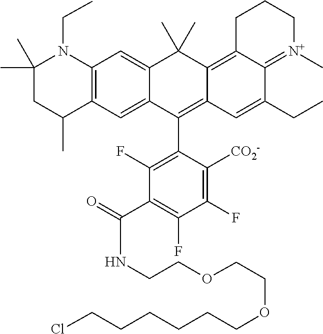

6⁻(MOM-MAC)-JF₆₅₇ (59_{MAC}; 55 mg, 75.9 mol) was taken up in CH₂Cl₂ (3 mL); triethylsilane (300 μL) was added, followed by trifluoroacetic acid (600 L). The reaction was stirred at room temperature for 8 h. Toluene (4 mL) was added, and the reaction mixture was concentrated to dryness. The residue was combined with a premixed solution of HaloTag®(O2)amine (HTL-NH₂, 111; TFA salt; 51.3 mg, 0.152 mmol, 2 eq) and DIEA (132 L, 0.759 mmol, 10 eq) in CH₂Cl₂ (3 mL), and the reaction was stirred at room temperature for 18 h. The solvent was removed by rotary evaporation, and the crude material was purified by reverse phase HPLC (30-60% MeCN/H₂O, linear gradient, with constant 0.1% v/v TFA additive) to provide 33.0 mg (45%, TFA salt) of 59_{HTL} as a blue solid (mixture of two diastereomers). ¹H NMR (CD₃OD, 400 MHz) δ 9.18 (t, J=5.1 Hz, 0.5H), 9.12 (t, J=5.4 Hz, 0.5H), 6.95 (s, 1H), 6.86-6.79 (m, 2H), 3.84 (dq, J=14.4, 6.8 Hz, 1H), 3.71-3.56 (m, 13H), 3.51 (t, J=6.6 Hz, 2H), 3.45 (t, J=6.5 Hz, 1H), 3.44 (t, J=6.6 Hz, 1H), 3.25-3.17 (m, 2H), 2.86-2.75 (m, 1H), 2.72-2.63 (m, 2H), 2.14-2.02 (m, 2H), 1.96 (s, 2H), 1.93-1.86 (m, 1H), 1.92 (s, 1.5H), 1.91 (s, 1.5H), 1.84 (s, 1.5H), 1.83 (s, 1.5H), 1.72 (p, J=6.9 Hz, 2H), 1.59-1.47 (m, 3H), 1.49 (s, 3H), 1.46-1.28 (m, 4H), 1.362 (s, 1.5H), 1.361 (t, J=6.9 Hz, 3H), 1.35 (s, 1.5H), 1.174 (d, J=6.6 Hz, 1.5H), 1.165 (d, J=6.4 Hz, 1.5H); 19F NMR (CD₃OD, 376 MHz) δ-75.38 (s, 3F), −116.63 (d, J=15.1 Hz, 0.5F), −116.78 (d, J=15.4 Hz, 0.5F), −133.56 (d, J=22.2 Hz, 0.5F), −133.70 (d, J=22.5 Hz, 0.5F), −140.50 (dd, J=22.0, 15.3 Hz, 0.5F), −140.72 (dd, J=22.0, 14.9 Hz, 0.5F); Analytical HPLC: t_{R} (two isomers)=14.2 min, 14.4 min; >99% total purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₄₈H₆₀C₁F₃N₃O₅ [M+H]⁺ 850.4168, found 850.4160.

### Example 159: Conversion of MAC Xanthenes to HaloTag® Ligands (Scheme 18): 2⁻(3,6⁻Bis(methyl(phenyl)amino)xanthylium-9⁻yl)-4⁻((2⁻(2⁻((6-chlorohexyl)oxy)ethoxy)ethyl)carbamoyl)-3,5,6⁻trifluorobenzoate (64_{HTL})

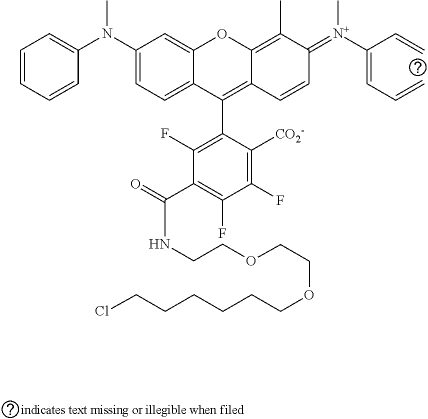

2⁻(3,6⁻Bis(methyl(phenyl)amino)xanthylium-9⁻yl)-4⁻(dicyano(methoxymethoxy)-methyl)-3,5,6⁻trifluorobenzoate (64_{MAC}; 100 mg, 0.145 mmol) was taken up in CH₂Cl₂ (10 mL); triethylsilane (1 mL) was added, followed by trifluoroacetic acid (2 mL). The reaction was stirred at room temperature for 6 h. Toluene (10 mL) was added, and the reaction mixture was concentrated to dryness. The residue was combined with a premixed solution of HaloTag®(O2)amine (HTL-NH₂, 111; TFA salt; 98.1 mg, 0.290 mmol, 2 eq) and DIEA (253 L, 1.45 mmol, 10 eq) in CH₂Cl₂ (7 mL), and the reaction was stirred at room temperature for 1 h. The solvent was removed by rotary evaporation, and the crude material was purified by silica gel chromatography (25-100% EtOAc/toluene, linear gradient) to yield 78.7 mg (67%) of 64_{HTL} as a dark purple solid. ¹H NMR (CDCl₃, 400 MHz) δ 7.41-7.35 (m, 4H), 7.22-7.16 (m, 6H), 6.77 (s, 1H), 6.72 (d, J=8.9 Hz, 2H), 6.59 (d, J=2.4 Hz, 2H), 6.54 (dd, J=8.8, 2.5 Hz, 2H), 3.65-3.57 (m, 6H), 3.54-3.48 (m, 4H), 3.37 (t, J=6.7 Hz, 2H), 3.34 (s, 6H), 1.77-1.69 (m, 2H), 1.53-1.45 (m, 2H), 1.44-1.36 (m, 2H), 1.34-1.26 (m, 2H); 19F NMR (CDCl₃, 376 MHz) δ-121.01 (d, J=22.5 Hz, 1F), −132.34 (d, J=21.6 Hz, 1F), −142.02 (t, J=22.0 Hz, 1F); Analytical HPLC: t_{R}=11.5 min, >99% purity (30-95% MeCN/H₂O, linear gradient, with constant 0.1% v/v TFA additive; 20 min run; 1 mL/min flow; ESI; positive ion mode; detection at 550 nm); HRMS (ESI) calcd for C₄₅H₄₄C₁F₃N₃O₆ [M+H]⁺ 814.2865, found 814.2858.

### Example 160: Conversion of MAC Xanthenes to HaloTag® Ligands (Scheme 18): JQ₆₄₅-HaloTag® ligand (65_{HTL})

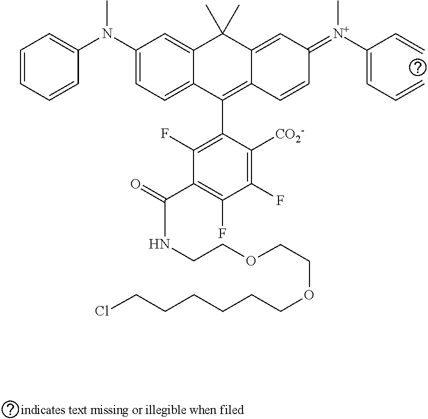

4⁻(Dicyano(methoxymethoxy)methyl)-2⁻(10,10⁻dimethyl-3,6⁻bis(methyl(phenyl)-amino)anthracen-9⁻ylium-9(10H)-yl)-3,5,6⁻trifluorobenzoate (65_{MAC}; 50 mg, 70.0 mol) was taken up in CH₂Cl₂ (4 mL); triethylsilane (400 μL) was added, followed by trifluoroacetic acid (800 L). The reaction was stirred at room temperature for 6 h. Toluene (4 mL) was added, and the reaction mixture was concentrated to dryness. The residue was combined with a premixed solution of HaloTag®(O2)amine (HTL-NH₂, 111; TFA salt; 47.3 mg, 0.140 mmol, 2 eq) and DIEA (122 μL, 0.700 mmol, 10 eq) in CH₂Cl₂ (4 mL), and the reaction was stirred at room temperature for 18 h. The solvent was removed by rotary evaporation, and the crude material was purified by silica gel chromatography (5-75% EtOAc/hexanes, linear gradient) to yield 43.0 mg (73%) of 65_{HTL} as a pale blue-green solid. ¹H NMR (CDCl₃, 400 MHz) δ 7.37-7.30 (m, 4H), 7.18-7.12 (m, 4H), 7.11-7.05 (m, 4H), 6.75 (dd, J=8.7, 2.4 Hz, 2H), 6.72 (bs, 1H), 6.71 (d, J=8.7 Hz, 2H), 3.65-3.57 (m, 6H), 3.54-3.47 (m, 4H), 3.37 (t, J=6.6 Hz, 2H), 3.36 (s, 6H), 1.78-1.69 (m, 2H), 1.63 (s, 6H), 1.54-1.46 (m, 2H), 1.45-1.37 (m, 2H), 1.34-1.27 (m, 2H); 19F NMR (CDCl₃, 376 MHz) δ−120.93 (d, J=22.7 Hz, 1F), −133.20 (d, J=21.8 Hz, 1F),-142.37 (t, J=22.2 Hz, 1F); Analytical HPLC: t_{R}=15.0 min, >99% purity (30-95% MeCN/H₂O, linear gradient, with constant 0.1% v/v TFA additive; 20 min run; 1 mL/min flow; ESI; positive ion mode; detection at 650 nm); HRMS (ESI) calcd for C₄₈H₅₀ClF₃N₃O₅ [M+H]⁺ 840.3386, found 840.3377.

### Example 161: Conversion of MAC Xanthenes to HaloTag® Ligands (Scheme 18): 4⁻((2⁻(2⁻((6⁻Chlorohexyl)oxy)ethoxy)ethyl)carbamoyl)-2⁻(5,5⁻dimethyl-3,7-bis(methyl(phenyl)amino)dibenzo-[b,e]silin-10⁻ylium-10(5H)-yl)-3,5,6⁻trifluorobenzoate (66_{HTL})

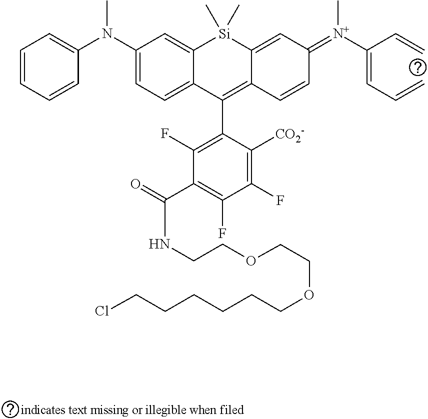

4⁻(Dicyano(methoxymethoxy)methyl)-2⁻(5,5⁻dimethyl-3,7-bis(methyl(phenyl)amino)dibenzo[b,e]silin-10⁻ylium-10(5H)-yl)-3,5,6⁻trifluorobenzoate (66_{MAC}; 40 mg, 54.7 mol) was taken up in CH₂Cl₂ (3 mL); triethylsilane (300 μL) was added, followed by trifluoroacetic acid (600 L). The reaction was stirred at room temperature for 6 h. Toluene (3 mL) was added, and the reaction mixture was concentrated to dryness. The residue was combined with a premixed solution of HaloTag®(O2)amine (HTL-NH₂, 111; TFA salt; 37.0 mg, 0.109 mmol, 2 eq) and DIEA (95.3 μL, 0.547 mmol, 10 eq) in CH₂Cl₂ (3 mL), and the reaction was stirred at room temperature for 18 h. The solvent was removed by rotary evaporation, and the crude material was purified by silica gel chromatography (5-75% EtOAc/hexanes, linear gradient) to yield 24.4 mg (52%) of 66_{HTL} as an off-white solid. ¹H NMR (CDCl₃, 400 MHz) δ 7.36-7.29 (m, 4H), 7.19-7.11 (m, 6H), 7.11-7.05 (m, 2H), 6.86 (s, 1H), 6.84-6.77 (m, 4H), 3.67-3.58 (m, 6H), 3.55-3.51 (m, 2H), 3.49 (t, J=6.7 Hz, 2H), 3.37 (t, J=6.7 Hz, 2H), 3.35 (s, 6H), 1.76-1.68 (m, 2H), 1.52-1.44 (m, 2H), 1.43-1.35 (m, 2H), 1.32-1.25 (m, 2H), 0.49 (s, 3H), 0.45 (s, 3H); 19F NMR (CDCl₃, 376 MHz) δ−118.19 (d, J=22.7 Hz, 1F), −133.12 (d, J=21.4 Hz, 1F), −141.91 (t, J=22.1 Hz, 1F); Analytical HPLC: t_{R}=15.9 min, 98.8% purity (50-95% MeCN/H₂O, linear gradient, with constant 0.1% v/v TFA additive; 20 min run; 1 mL/min flow; ESI; positive ion mode; detection at 675 nm); HRMS (ESI) calcd for C₄₇H₅₀ClF₃N₃O₅Si [M+H]⁺ 856.3155, found 856.3150.

### Example 162: Synthesis of Other HaloTag® and SNAP-tag® Ligands: 4⁻((2-(2⁻((6⁻Chlorohexyl)oxy)ethoxy)ethyl)carbamoyl)-2⁻(1,2,2,4,8,10,10,11,13,13⁻decamethyl-2,10,11,13⁻tetra-hydrosilino[3,2⁻g:5,6⁻g′]diquinolin-6⁻ylium-6(1H)-yl)benzoate (104_{HTL})

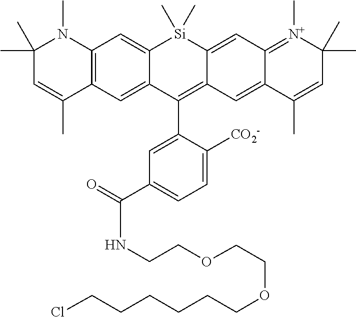

4⁻Carboxy-2⁻(1,2,2,4,8,10,10,11,13,13⁻decamethyl-2,10,11,13-tetrahydrosilino[3,2⁻g:5,6⁻g′]diquinolin-6⁻ylium-6(1H)-yl)benzoate (110; 40 mg, 66.1 mol) was combined with DSC (40.7 mg, 0.159 mmol, 2.4 eq) in DMF (3 mL). After adding Et₃N (55.3 μL, 0.397 mmol, 6 eq) and DMAP (0.8 mg, 6.6 mol, 0.1 eq), the reaction was stirred at room temperature for 30 min. A solution of HaloTag®(O2)amine (HTL-NH₂, 111; TFA salt; 53.6 mg, 0.159 mmol, 2.4 eq) in DMF (200 μL) was then added. The reaction was stirred an additional 3 h at room temperature. Purification of the crude reaction mixture by reverse phase HPLC (30-9₅% MeCN/H₂O, linear gradient, with constant 0.1% v/v TFA additive) afforded 39.4 mg (64%, TFA salt) of 104_{HTL} as a green solid. ¹H NMR (CD₃OD, 400 MHz) δ 8.74 (t, J=5.5 Hz, 1H), 8.27 (d, J=8.1 Hz, 1H), 8.12 (dd, J=8.2, 1.7 Hz, 1H), 7.72 (d, J=1.6 Hz, 1H), 7.10 (s, 2H), 6.61 (s, 2H), 5.46 (q, J=1.2 Hz, 2H), 3.65 (t, J=5.5 Hz, 2H), 3.62-3.53 (m, 6H), 3.50 (t, J=6.6 Hz, 2H), 3.41 (t, J=6.5 Hz, 2H), 3.20 (s, 6H), 1.70 (dq, J=8.0, 6.7 Hz, 2H), 1.55 (d, J=1.4 Hz, 6H), 1.52-1.47 (m, 2H), 1.45 (s, 6H), 1.43 (s, 6H), 1.41-1.28 (m, 4H), 0.63 (s, 3H), 0.58 (s, 3H); Analytical HPLC: t_{R}=11.5 min, >99% purity (30-95% MeCN/H₂O, linear gradient, with constant 0.1% v/v TFA additive; 20 min run; 1 mL/min flow; ESI; positive ion mode; detection at 725 nm); HRMS (ESI) calcd for C₄₇H₆₁ClN₃O₅Si [M+H]⁺ 810.4064, found 810.4046.

### Example 163: Synthesis of Other HaloTag® and SNAP-tag® Ligands: JFX₆₁₂⁻SNAP-tag® ligand (88_{STL})

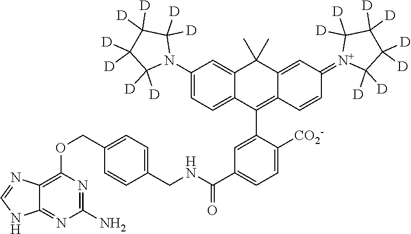

JFX₆₁₂⁻NHS⁴⁵ (25 mg, 40.2 mol) and 6⁻((4⁻(aminomethyl)benzyl)oxy)-9H-purin-2⁻amine (BG-NH₂; 16.3 mg, 60.3 mol, 1.5 eq) were combined in DMF (1.5 mL), and DIEA (21.0 μL, 0.121 mmol, 3 eq) was added. After stirring the reaction at room temperature for 4 h, it was directly purified by reverse phase HPLC (10-50% MeCN/H₂O, linear gradient, with constant 0.1% v/v TFA additive) to yield 33.6 mg (94%, TFA salt) of 88_{STL} as a purple solid. 1H NMR (CD₃OD, 400 MHz) δ 9.29 (t, J=6.1 Hz, 1H), 8.35 (d, J=8.2 Hz, 1H), 8.23 (s, 1H), 8.16 (dd, J=8.3, 1.8 Hz, 1H), 7.78 (d, J=1.7 Hz, 1H), 7.51 (d, J=8.2 Hz, 2H), 7.40 (d, J=8.2 Hz, 2H), 7.09 (d, J=2.4 Hz, 2H), 6.98 (d, J=9.2 Hz, 2H), 6.65 (dd, J=9.3, 2.4 Hz, 2H), 5.60 (s, 2H), 4.60 (d, J=6.0 Hz, 2H), 1.86 (s, 3H), 1.75 (s, 3H); Analytical HPLC: t_{R}=10.3 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₄₅H₂₉D₁₆N₈O₄ [M+H]⁺ 777.4563, found 777.4567.

### Example 164: Synthesis of Other HaloTag® and SNAP-tag® Ligands: JFX₆₅₀⁻SNAP-tag® ligand (89_{STL})

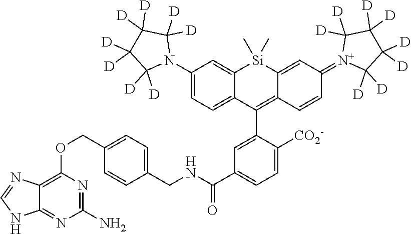

JFX₆₅₀⁻NHS⁴⁵ (75 mg, 0.118 mmol) and 6⁻((4⁻(aminomethyl)benzyl)oxy)-9H-purin-2⁻amine (BG-NH₂; 47.7 mg, 0.176 mmol, 1.5 eq) were combined in DMF (5 mL), and DIEA (61.4 μL, 0.353 mmol, 3 eq) was added. After stirring the reaction at room temperature for 18 h, it was diluted with saturated NaHCO₃ and extracted with 10% MeOH/CH₂Cl₂ (3×). The combined organic extracts were dried over anhydrous MgSO₄, filtered, and evaporated. Flash chromatography on silica gel (0-10% MeOH/EtOAc, linear gradient) provided 89_{STL} as a blue solid (73 mg, 78%). ¹H NMR (CD₃OD, 400 MHz) δ 8.02 (dd, J=8.0, 1.4 Hz, 1H), 7.98 (dd, J=8.0, 0.5 Hz, 1H), 7.81 (s, 1H), 7.69-7.67 (m, 1H), 7.42 (d, J=8.2 Hz, 2H), 7.29 (d, J=8.1 Hz, 2H), 6.84 (d, J=2.8 Hz, 2H), 6.67 (d, J=8.9 Hz, 2H), 6.40 (dd, J=8.9, 2.8 Hz, 2H), 5.47 (s, 2H), 4.50 (s, 2H), 0.60 (s, 3H), 0.53 (s, 3H); Analytical HPLC: t_{R}=10.4 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₄₄H₂₉D₁₆N₈O₄Si [M+H]⁺ 793.4332, found 793.4341.

### Example 165: Synthesis of Other HaloTag® and SNAP-tag® Ligands: tert-Butyl 10,10⁻dimethyl-3,6⁻bis(methyl(phenyl)amino)-3′-oxo-3′H,10H-spiro[anthracene-9,1′-isobenzofuran]-6′-carboxylate (S59)

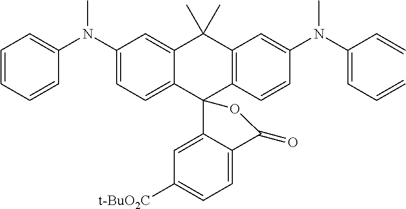

A vial was charged with 6⁻tert-butoxycarbonylcarbofluorescein ditriflate¹⁸ (S58; 200 mg, 0.277 mmol), Pd₂dba₃ (25.3 mg, 27.7 mol, 0.1 eq), XPhos (39.6 mg, 83.0 mol, 0.3 eq), and Cs₂CO₃ (252 mg, 0.775 mmol, 2.8 eq). The vial was sealed and evacuated/backfilled with nitrogen (3×). Dioxane (2 mL) was added, and the reaction was flushed again with nitrogen (3×). Following the addition of N-methylaniline (66.0 μL, 0.609 mmol, 2.2 eq), the reaction was stirred at 100° C. for 4 h. It was then cooled to room temperature, filtered through Celite with CH₂Cl₂, and concentrated to dryness. Purification by silica gel chromatography (0-30% EtOAc/hexanes, linear gradient) afforded S₅₉ (150 mg, 85%) as an off-white solid. 1H NMR (CDCl₃, 400 MHz) δ 8.15 (dd, J=8.0, 1.3 Hz, 1H), 8.04-7.99 (m, 1H), 7.71-7.68 (m, 1H), 7.35-7.28 (m, 4H), 7.16 (d, J=2.5 Hz, 2H), 7.14-7.09 (m, 4H), 7.08-7.01 (m, 2H), 6.70 (dd, J=8.7, 2.4 Hz, 2H), 6.57 (d, J=8.7 Hz, 2H), 3.35 (s, 6H), 1.73 (s, 3H), 1.67 (s, 3H), 1.55 (s, 9H); 13C NMR (CDCl₃, 101 MHz) δ 170.0 (C), 164.6 (C), 155.3 (C), 149.6 (C), 148.4 (C), 146.6 (C), 137.9 (C), 130.2 (CH), 130.1 (C), 129.5 (CH), 128.9 (CH), 125.1 (CH), 125.0 (CH), 123.0 (CH), 122.7 (CH), 122.3 (C), 117.3 (CH), 115.5 (CH), 87.7 (C), 82.5 (C), 40.3 (CH₃), 38.5 (C), 35.0 (CH₃), 33.0 (CH₃), 28.2 (CH₃); Analytical HPLC: t_{R}=12.7 min, >99% purity (50-95% MeCN/H₂O, linear gradient, with constant 0.1% v/v TFA additive; 20 min run; 1 mL/min flow; ESI; positive ion mode; detection at 254 nm); HRMS (ESI) calcd for C₄₂H₄₁N₂O₄ [M+H]⁺ 637.3061, found 637.3061.

### Example 166: Synthesis of Other HaloTag® and SNAP-tag® Ligands: 10,10-Dimethyl-3,6⁻bis(methyl(phenyl)amino)-3′-oxo-3′H,10H-spiro[anthracene-9,1′-isobenzofuran]-6′-carboxylic acid (S₆₀)

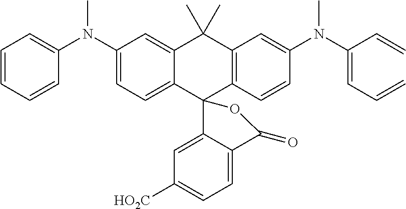 [SMILES: C=C/C=C(\C=C)N(C)c1ccc2c(c1)C(C)(C)c1cc(N(C)c3ccccc3)ccc1C21OC(=O)c2ccc(C(=O)O)cc21]

Ester S59 (120 mg, 0.188 mmol) was taken up in CH₂Cl₂ (5 mL), and trifluoroacetic acid (1 mL) was added. The reaction was stirred at room temperature for 6 h. Toluene (5 mL) was added; the reaction mixture was concentrated to dryness and then azeotroped with MeOH three times to provide S₆₀ as a dark blue solid (128 mg, 98%, TFA salt). Analytical HPLC and NMR indicated that the material was >95% pure and did not require further purification prior to amide coupling. ¹H NMR (CD₃OD, 400 MHz) δ 8.34-8.28 (m, 2H), 7.87-7.82 (m, 1H), 7.56-7.48 (m, 4H), 7.43-7.36 (m, 2H), 7.34-7.29 (m, 4H), 7.15 (d, J=2.5 Hz, 2H), 6.94 (d, J=9.3 Hz, 2H), 6.71 (dd, J=9.3, 2.4 Hz, 2H), 3.57 (s, 6H), 1.67 (s, 3H), 1.59 (s, 3H); 13C NMR (CD₃OD, 101 MHz) δ 168.0 (C), 167.8 (C), 156.9 (C), 146.8 (C), 136.7 (CH), 136.0 (C), 135.2 (C), 131.7 (C), 131.6 (CH), 131.5 (CH), 131.1 (CH), 128.7 (CH), 127.2 (CH), 123.0 (C), 116.0 (CH), 113.9 (CH), 42.5 (C), 41.6 (CH₃), 35.3 (CH₃), 32.1 (CH₃); Analytical HPLC: t_{R}=11.6 min, >99% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₃₈H₃₃N₂O₄ [M+H]⁺ 581.2435, found 581.2428.

### Example 167: Synthesis of Other HaloTag® and SNAP-tag® Ligands: N-(2-(2⁻((6⁻Chlorohexyl)oxy)ethoxy)ethyl)-10,10⁻dimethyl-3,6⁻bis(methyl(phenyl)amino)-3′-oxo-3′H,10H-spiro[anthracene-9,1′-isobenzofuran]-6′-carboxamide (91_{HTL})

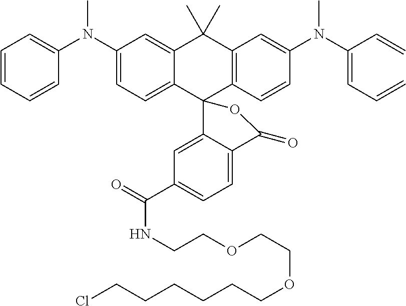 [SMILES: C=C/C=C(\C=C)N(C)c1ccc2c(c1)C(C)(C)c1cc(N(C)c3ccccc3)ccc1C21OC(=O)c2ccc(C(=O)NCCOCCOCCCCCCCl)cc21]

Acid S₆₀ (50 mg, 72.0 mol) was combined with DSC (44.2 mg, 0.173 mmol, 2.4 eq) in DMF (4 mL). After adding Et₃N (60.2 μL, 0.432 mmol, 6 eq) and DMAP (0.9 mg, 7.2 mol, 0.1 eq), the reaction was stirred at room temperature for 30 min. A solution of HaloTag®(O2)amine (HTL-NH₂, 111; TFA salt; 72.9 mg, 0.216 mmol, 3 eq) in DMF (500 L) was added; the reaction was then stirred for an additional 18 h at room temperature. It was subsequently diluted with saturated NaHCO₃ and extracted with EtOAc (2×). The combined organic extracts were washed with water and brine, dried over anhydrous MgSO₄, filtered, and evaporated. Purification of the crude product by silica gel chromatography (5-75% EtOAc/toluene, linear gradient) provided 91H_{TL} as a pale blue solid (44 mg, 78%). 1H NMR (CDCl₃, 400 MHz) δ 8.03 (d, J=7.9 Hz, 1H), 7.92 (dd, J=8.0, 1.4 Hz, 1H), 7.52 (s, 1H), 7.35-7.28 (m, 4H), 7.16 (d, J=2.4 Hz, 2H), 7.14-7.09 (m, 4H), 7.07-7.01 (m, 2H), 6.82 (t, J=5.1 Hz, 1H), 6.70 (dd, J=8.7, 2.4 Hz, 2H), 6.57 (d, J=8.7 Hz, 2H), 3.67-3.58 (m, 6H), 3.57-3.53 (m, 2H), 3.50 (t, J=6.6 Hz, 2H), 3.41 (t, J=6.7 Hz, 2H), 3.35 (s, 6H), 1.78-1.68 (m, 2H), 1.73 (s, 3H), 1.66 (s, 3H), 1.57-1.50 (m, 2H), 1.46-1.37 (m, 2H), 1.36-1.28 (m, 2H); Analytical HPLC: t_{R}=14.9 min, >99% purity (30-95% MeCN/H₂O, linear gradient, with constant 0.1% v/v TFA additive; 20 min run; 1 mL/min flow; ESI; positive ion mode; detection at 254 nm); HRMS (ESI) calcd for C₄₈H₅₃C₁N₃O₅[M+H]⁺ 786.3668, found 786.3658.

### Example 168: Synthesis of Other HaloTag® and SNAP-tag® Ligands: 4,5,7-Trifluoro-Si-fluorescein-SNAP-tag® ligand (55_{STL})

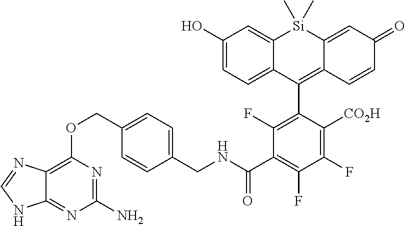 [SMILES: C[Si]1(C)C2=CC(=O)C=CC2=C(c2c(F)c(C(=O)NCc3ccc(COc4nc(N)nc5[nH]cnc45)cc3)c(F)c(F)c2C(=O)O)c2ccc(O)cc21]

4,5,7⁻Trifluoro-6⁻(MOM-MAC)—Si-fluorescein (55_{MAC}; 60 mg, 0.109 mmol) was taken up in CH₂Cl₂ (4 mL); triethylsilane (400 μL) was added, followed by trifluoroacetic acid (800 L). The reaction was stirred at room temperature for 24 h. Toluene (5 mL) was added, and the reaction mixture was concentrated to dryness. The residue was combined with a premixed solution of 6⁻((4⁻(aminomethyl)benzyl)oxy)-9H-purin-2⁻amine (BG-NH₂; 58.7 mg, 0.217 mmol, 2 eq) and DIEA (189 μL, 1.09 mmol, 10 eq) in DMF (4 mL), and the reaction was stirred at room temperature for 4 h. It was then acidified with 1 M HCl, diluted with water, and extracted with 15% i-PrOH/CHCl₃ (2×). The combined organic extracts were dried over anhydrous MgSO₄, filtered, and concentrated in vacuo. Purification by reverse phase HPLC (10-75% MeCN/H₂O, linear gradient, with constant 0.1% v/v TFA additive) afforded 44.4 mg (49%, TFA salt) of 55_{STL} as a pale pink solid. ¹H NMR (CD₃OD, 400 MHz) δ 9.44 (t, J=6.0 Hz, 1H), 8.21 (s, 1H), 7.43 (d, J=8.1 Hz, 2H), 7.29 (d, J=8.1 Hz, 2H), 7.03 (d, J=2.7 Hz, 2H), 6.76 (dd, J=8.7, 1.3 Hz, 2H), 6.63 (dd, J=8.7, 2.7 Hz, 2H), 5.54 (s, 2H), 4.52-4.45 (m, 2H), 0.45 (s, 3H), 0.41 (s, 3H); 19F NMR (CD₃OD, 376 MHz) δ−75.49 (s, 3F), −118.89 (d, J=22.1 Hz, 1F),-134.74 (d, J=20.7 Hz, 1F), −143.01 (t, J=21.6 Hz, 1F); Analytical HPLC: t_{R}=10.1 min, >99% purity (10-95% MeCN/H₂O, linear gradient, with constant 0.1% v/v TFA additive; 20 min run; 1 mL/min flow; ESI; positive ion mode; detection at 254 nm); HRMS (ESI) calcd for C₃₆H₂₈F₃N₆O6Si [M+H]⁺ 725.1786, found 725.1781

### Example 169: Synthesis of Other HaloTag® and SNAP-tag® Ligands: JF₅₇₁-cpSNAP-tag® ligand (85_{CSTL})

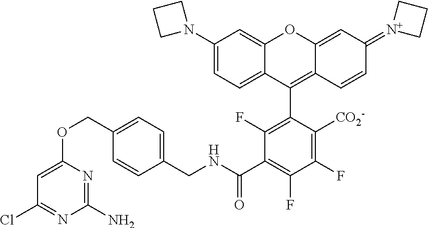

6⁻(MOM-MAC)-JF₅₇₁ (85_{MAC}16; acetate salt; 60 mg, 92.5 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 4⁻((4⁻(aminomethyl)benzyl)oxy)-6⁻chloropyrimidin-2⁻amine (49.0 mg, 0.185 mmol, 2 eq) and DIEA (161 μL, 0.925 mmol, 10 eq) in DMF (4 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive) to afford 85_{CSTL} as a dark red-purple solid (40.6 mg, 50%, TFA salt). ¹H NMR (CD₃OD, 400 MHz) δ 9.42 (t, J=5.8 Hz, 1H), 7.39 (AB quartet, v_{A}=2963.2 Hz, v_{B}=2947.9 Hz, J_{AB}=8.3 Hz, 4H), 7.25 (dd, J=9.2, 0.8 Hz, 2H), 6.65 (dd, J=9.2, 2.2 Hz, 2H), 6.53 (d, J=2.1 Hz, 2H), 6.08 (s, 1H), 5.33 (s, 2H), 4.64-4.56 (m, 2H), 4.33 (t, J=7.7 Hz, 8H), 2.57 (p, J=7.7 Hz, 4H); 19F NMR (CD₃OD, 376 MHz) δ−75.52 (s, 3F), −116.61 (d, J=15.4 Hz, 1F), −132.32 (d, J=22.5 Hz, 1F), −139.34 (dd, J=22.0, 14.8 Hz, 1F); Analytical HPLC: t_{R}=12.2 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₃₉H₃₁C₁F₃N₆O₅ [M+H]⁺ 755.1991, found 755.1986.

### Example 170: Synthesis of Other HaloTag® and SNAP-tag® Ligands: JF₆₃₂-SNAP-tag® ligand (44_{STL})

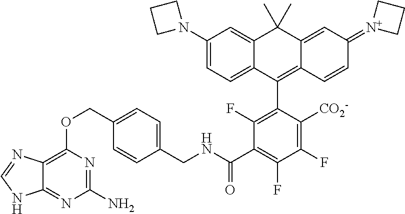

6⁻(MOM-MAC)-JF₆₃₂ (44_{MAC}; 14 mg, 0.0228 mmol) was taken up in CH₂Cl₂ (2 mL); triethylsilane (200 μL) was added, followed by trifluoroacetic acid (400 L). The reaction was stirred at room temperature for 6 h. Toluene (3 mL) was added, and the reaction mixture was concentrated to dryness. The residue was combined with a premixed solution of 6⁻((4-(aminomethyl)benzyl)oxy)-9H-purin-2⁻amine (BG-NH₂; 12.3 mg, 0.0456 mmol, 2 eq) and DIEA (39.7 μL, 0.228 mmol, 10 eq) in DMF (2 mL), and the reaction was stirred at room temperature for 18 h. The solvent was removed by rotary evaporation, and the crude material was purified by reverse phase HPLC (10-75% MeCN/H₂O, linear gradient, with constant 0.1% v/v TFA additive). The pooled product fractions were partially concentrated to remove MeCN, diluted with saturated NaHCO₃, and extracted with EtOAc (2×). The organic extracts were dried over anhydrous MgSO₄, filtered, and evaporated to afford 44_{STL} as a blue solid (7.1 mg, 39%). 1H NMR (CD₃OD, 400 MHz) δ 7.83 (s, 1H), 7.51 (d, J=8.1 Hz, 2H), 7.38 (d, J=8.0 Hz, 2H), 7.18 (d, J=9.1 Hz, 2H), 6.77 (d, J=2.2 Hz, 2H), 6.40 (dd, J=9.1, 2.2 Hz, 2H), 5.53 (s, 2H), 4.59 (s, 2H), 4.33 (t, J=7.6 Hz, 8H), 2.54 (p, J=7.6 Hz, 4H), 1.73 (s, 3H), 1.67 (s, 3H); 19F NMR (CD₃OD, 376 MHz) δ−118.52 (d, J=15.6 Hz, 1F), −135.08 (d, J=23.2 Hz, 1F), −143.94 (dd, J=22.8, 16.4 Hz, 1F); Analytical HPLC: t_{R}=9.5 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₄₃H₃₈F₃N₈O₄[M+H]⁺ 787.2963, found 787.2950.

### Example 171: Synthesis of Other HaloTag® and SNAP-tag® Ligands: JF₆₃₂-cpSNAP-tag® ligand (44_{CSTL})

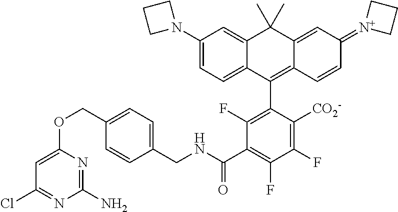

6⁻(MOM-MAC)-JF₆₃₂ (44_{MAC}; 16 mg, 0.0260 mmol) was taken up in CH₂Cl₂ (2 mL); triethylsilane (200 μL) was added, followed by trifluoroacetic acid (400 μL). The reaction was stirred at room temperature for 6 h. Toluene (3 mL) was added, and the reaction mixture was concentrated to dryness. The residue was combined with a premixed solution of 4⁻((4-(aminomethyl)benzyl)oxy)-6⁻chloropyrimidin-2⁻amine (13.8 mg, 0.0521 mmol, 2 eq) and DIEA (45.3 μL, 0.260 mmol, 10 eq) in DMF (3 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive). The pooled product fractions were partially concentrated to remove MeCN, diluted with saturated NaHCO₃, and extracted with EtOAc (2×). The organic extracts were dried over anhydrous MgSO₄, filtered, and evaporated to afford 44_{CSTL} as a blue solid (6.4 mg, 32%). 1H NMR (CDCl₃, 400 MHz) δ 7.34 (d, J=7.9 Hz, 2H), 7.28 (d, J=8.3 Hz, 2H), 6.67 (d, J=8.6 Hz, 2H), 6.52 (d, J=2.3 Hz, 2H), 6.25 (dd, J=8.6, 2.3 Hz, 2H), 6.19 (bs, 1H), 6.14 (s, 1H), 5.29 (s, 2H), 5.02 (s, 2H), 4.56 (d, J=5.8 Hz, 2H), 3.94 (t, J=7.3 Hz, 8H), 2.39 (p, J=7.2 Hz, 4H), 1.72 (s, 3H), 1.69 (s, 3H); 19F NMR (CDCl₃, 376 MHz) δ−120.88 (d, J=20.2 Hz, 1F), −133.00 (d, J=21.6 Hz, 1F), −142.20 (t, J=22.0 Hz, 1F); Analytical HPLC: t_{R}=12.5 min, >99% purity (10-95% MeCN/H₂O, linear gradient, with constant 0.1% v/v TFA additive; 20 min run; 1 mL/min flow; ESI; positive ion mode; detection at 625 nm); HRMS (ESI) calcd for C₄₂H₃₇C₁F₃N₆O₄ [M+H]⁺ 781.2511, found 781.2502.

### Example 172: Synthesis of Other HaloTag® and SNAP-tag® Ligands: JFX₅₇₆⁻cpSNAP-tag® ligand (8_{CSTL})

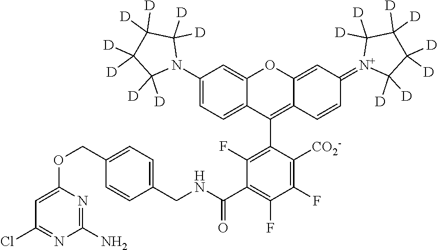

6⁻(MOM-MAC)-JFX₅₇₆ (8_{MAC}; 60 mg, 86.6 mol) was taken up in CH₂Cl₂ (5 mL); triethylsilane (500 μL) was added, followed by trifluoroacetic acid (1 mL). The reaction was stirred at room temperature for 6 h. Toluene (5 mL) was added, and the reaction mixture was concentrated to dryness. The residue was combined with a premixed solution of 4⁻((4-(aminomethyl)benzyl)oxy)-6⁻chloropyrimidin-2⁻amine (45.9 mg, 0.173 mmol, 2 eq) and DIEA (151 μL, 0.866 mmol, 10 eq) in DMF (4 mL), and the reaction was stirred at room temperature for 18 h. The solvent was removed by rotary evaporation, and the crude material was purified by reverse phase HPLC (30-70% MeCN/H₂O, linear gradient, with constant 0.1% v/v TFA additive) to afford 8_{CSTL} as a dark red-purple solid (52.8 mg, 67%, TFA salt). ¹H NMR (CD₃OD, 400 MHz) δ 9.46 (t, J=5.8 Hz, 1H), 7.39 (AB quartet, v_{A}=2963.6 Hz, v_{B}=2949.4 Hz, J_{AB}=8.2 Hz, 4H), 7.30 (d, J=9.3 Hz, 2H), 6.95 (dd, J=9.4, 2.3 Hz, 2H), 6.81 (d, J=2.3 Hz, 2H), 6.08 (s, 1H), 5.33 (s, 2H), 4.63-4.58 (m, 2H); 19F NMR (CD₃OD, 376 MHz) δ−75.41 (s, 3F),-116.58 (d, J=15.5 Hz, 1F), −132.40 (d, J=22.1 Hz, 1F), −139.51-−139.67 (m, 1F); Analytical HPLC: t_{R}=12.8 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₄₁H₁₉D₁₆C₁F₃N₆O₅ [M+H]⁺ 799.3308, found 799.3299.

### Example 173: Synthesis of Other HaloTag® and SNAP-tag® Ligands: JFX₆₃₇⁻SNAP-tag® ligand (40_{STL})

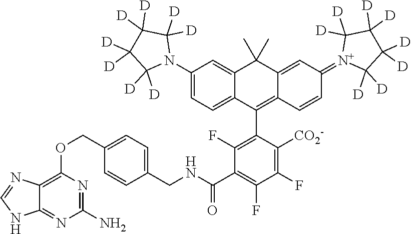

6⁻(MOM-MAC)-JFX₆₃₇ (40_{MAC}; 50 mg, 75.9 mol) was taken up in CH₂Cl₂ (3 mL); triethylsilane (300 μL) was added, followed by trifluoroacetic acid (600 L). The reaction was stirred at room temperature for 6 h. Toluene (4 mL) was added, and the reaction mixture was concentrated to dryness. The residue was combined with a premixed solution of 6⁻((4-(aminomethyl)benzyl)oxy)-9H-purin-2⁻amine (BG-NH₂; 41.0 mg, 0.152 mmol, 2 eq) and DIEA (132 μL, 0.759 mmol, 10 eq) in DMF (4 mL), and the reaction was stirred at room temperature for 18 h. The solvent was removed by rotary evaporation, and the crude material was purified by reverse phase HPLC (10-75% MeCN/H₂O, linear gradient, with constant 0.1% v/v TFA additive) to afford 40_{STL} as a deep blue solid (57.0 mg, 79%, TFA salt). ¹H NMR (CD₃OD, 400 MHz) δ 8.24 (s, 1H), 7.54 (d, J=8.2 Hz, 2H), 7.43 (d, J=8.1 Hz, 2H), 7.19 (dd, J=9.3, 1.0 Hz, 2H), 7.10 (d, J=2.4 Hz, 2H), 6.72 (dd, J=9.3, 2.4 Hz, 2H), 5.63 (s, 2H), 4.62 (s, 2H), 1.83 (s, 3H), 1.70 (s, 3H); 19F NMR (CD₃OD, 376 MHz) δ−75.39 (s, 3F), −117.30 (d, J=15.2 Hz, 1F),-133.85 (dd, J=22.5, 2.3 Hz, 1F), −140.50 (dd, J=22.2, 15.3 Hz, 1F); Analytical HPLC: t_{R}=10.4 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₄₅H₂₆D₁₆F₃N₈O₄[M+H]⁺ 831.4280, found 831.4280.

### Example 174: Synthesis of Other HaloTag® and SNAP-tag® Ligands: JFX₆₃₇⁻cpSNAP-tag® ligand (40_{CSTL})

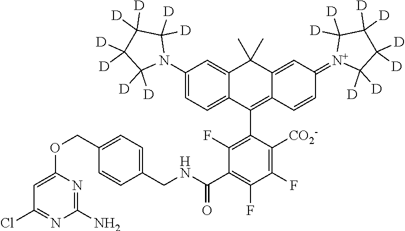

6⁻(MOM-MAC)-JFX₆₃₇ (40_{MAC}; 15 mg, 0.0228 mmol) was taken up in CH₂Cl₂ (2 mL); triethylsilane (200 μL) was added, followed by trifluoroacetic acid (400 μL). The reaction was stirred at room temperature for 6 h. Toluene (3 mL) was added, and the reaction mixture was concentrated to dryness. The residue was combined with a premixed solution of 4⁻((4-(aminomethyl)benzyl)oxy)-6⁻chloropyrimidin-2⁻amine (12.1 mg, 0.0455 mmol, 2 eq) and DIEA (39.7 μL, 0.227 mmol, 10 eq) in DMF (3 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive) to afford 40_{CSTL} as a blue solid (10 mg, 48%, TFA salt). ¹H NMR (CD₃OD, 400 MHz) δ 9.35 (t, J=5.7 Hz, 1H), 7.29 (AB quartet, v_{A}=2926.6 Hz, v_{B}=2911.2 Hz, J_{AB}=8.1 Hz, 4H), 7.08 (d, J=9.3 Hz, 2H), 7.00 (d, J=2.4 Hz, 2H), 6.62 (dd, J=9.3, 2.4 Hz, 2H), 5.99 (s, 1H), 5.24 (s, 2H), 4.53-4.49 (m, 2H), 1.73 (s, 3H), 1.60 (s, 3H); 19F NMR (CD₃OD, 376 MHz) δ-75.38 (s, 3F), −117.16 (d, J=15.2 Hz, 1F), −133.75 (d, J=22.0 Hz, 1F), −140.25 (dd, J=21.4, 15.4 Hz, 1F); Analytical HPLC: t_{R}=13.1 min, 98.2% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₄₄H_{2 5}D₁₆C₁F₃N₆O₄ [M+H]⁺ 825.3829, found 825.3816.

### Example 175: Synthesis of Other HaloTag® and SNAP-tag® Ligands JFX₆₇₃⁻SNAP-tag® ligand (25_{STL})

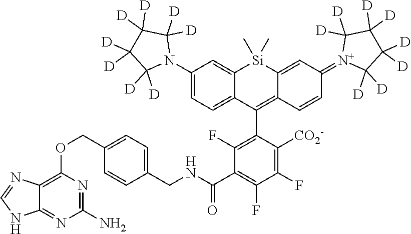

6⁻(MOM-MAC)-JFX₆₇₃ (25_{MAC}; 18 mg, 0.0267 mmol) was taken up in CH₂Cl₂ (2 mL); triethylsilane (200 μL) was added, followed by trifluoroacetic acid (400 L). The reaction was stirred at room temperature for 6 h. Toluene (3 mL) was added, and the reaction mixture was concentrated to dryness. The residue was combined with a premixed solution of 6⁻((4-(aminomethyl)benzyl)oxy)-9H-purin-2⁻amine (BG-NH₂; 14.4 mg, 0.0533 mmol, 2 eq) and DIEA (46.5 μL, 0.267 mmol, 10 eq) in DMF (3 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive) to afford 25_{STL} as a blue solid (8.5 mg, 34%, TFA salt). ¹H NMR (CD₃OD, 400 MHz) δ 9.50 (t, J=5.9 Hz, 1H), 8.22 (s, 1H), 7.54 (d, J=8.1 Hz, 2H), 7.42 (d, J=8.0 Hz, 2H), 7.18 (d, J=2.7 Hz, 2H), 7.14 (d, J=9.5 Hz, 2H), 6.66 (dd, J=9.5, 2.7 Hz, 2H), 5.62 (s, 2H), 4.64-4.60 (m, 2H), 0.61 (s, 3H), 0.53 (s, 3H); 19F NMR (CD₃OD, 376 MHz) δ−75.35 (s, 3F), −117.89 (d, J=15.4 Hz, 1F), −134.59 (d, J=22.1 Hz, 1F), −140.85-−141.01 (m, 1F); Analytical HPLC: t_{R}=10.5 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₄₄H₂₆D₁₆F₃N₈O4Si [M+H]⁺ 847.4049, found 847.4038.

### Example 176: Synthesis of Other HaloTag® and SNAP-tag® Ligands: JFX₆₇₃⁻cpSNAP-tag® ligand (25_{CSTL})

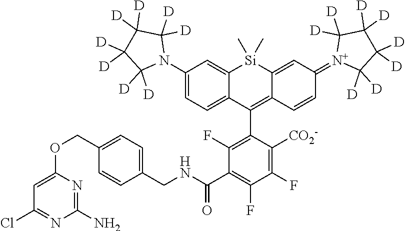

6⁻(MOM-MAC)-JFX₆₇₃ (25_{MAC}; 28 mg, 0.0415 mmol) was taken up in CH₂Cl₂ (3 mL); triethylsilane (300 μL) was added, followed by trifluoroacetic acid (600 μL). The reaction was stirred at room temperature for 6 h. Toluene (3 mL) was added, and the reaction mixture was concentrated to dryness. The residue was combined with a premixed solution of 4⁻((4-(aminomethyl)benzyl)oxy)-6⁻chloropyrimidin-2⁻amine (22.0 mg, 0.0830 mmol, 2 eq) and DIEA (72.3 μL, 0.415 mmol, 10 eq) in DMF (3 mL), and the reaction was stirred at room temperature for 18 h. The solvent was removed by rotary evaporation, and the crude material was purified by reverse phase HPLC (30-60% MeCN/H₂O, linear gradient, with constant 0.1% v/v TFA additive) to afford 25_{CSTL} as a blue solid (14 mg, 35%, TFA salt). ¹H NMR (CD₃OD, 400 MHz) δ 9.47 (t, J=5.7 Hz, 1H), 7.41 (AB quartet, v_{A}=2974.9 Hz, v_{B}=2958.5 Hz, J_{AB}=8.1 Hz, 4H), 7.21 (d, J=2.7 Hz, 2H), 7.16 (d, J=9.5 Hz, 2H), 6.68 (dd, J=9.5, 2.7 Hz, 2H), 6.11 (s, 1H), 5.36 (s, 2H), 4.64-4.60 (m, 2H), 0.63 (s, 3H), 0.54 (s, 3H); 19F NMR (CD₃OD, 376 MHz) δ-75.39 (s, 3F), −117.71 (d, J=15.3 Hz, 1F), −134.44 (d, J=22.1 Hz, 1F), −140.57 (dd, J=21.6, 15.3 Hz, 1F); Analytical HPLC: t_{R}=13.6 min, >99% purity (10-95% MeCN/H₂O, linear gradient, with constant 0.1% v/v TFA additive; 20 min run; 1 mL/min flow; ESI; positive ion mode; detection at 675 nm); HRMS (ESI) calcd for C₄₃H_{2 5}D₁₆C₁F₃N₆O₄Si [M+H]⁺ 841.3598, found 841.3588.

### Example 177: Synthesis of Other HaloTag® and SNAP-tag® Ligands: JF₅₆₃-SNAP-tag® ligand (18_{STL})

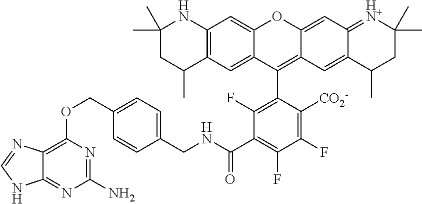

6⁻(MOM-MAC)-JF₅₆₃ (18_{MAC}; acetate salt; 45 mg, 61.4 mol) was taken up in CH₂Cl₂ (4 mL); anisole (400 μL) was added, followed by trifluoroacetic acid (800 L). The reaction was stirred at room temperature for 24 h. Toluene (5 mL) was added, and the reaction mixture was concentrated to dryness. The residue was combined with a premixed solution of 6-((4⁻(aminomethyl)benzyl)oxy)-9H-purin-2⁻amine (BG-NH₂; 33.2 mg, 0.123 mmol, 2 eq) and DIEA (107 μL, 0.614 mmol, 10 eq) in DMF (5 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive) to yield 34.4 mg (58%, TFA salt) of 18_{STL} as a red-purple solid (mixture of diastereomers). ¹H NMR (CD₃OD, 400 MHz) δ 8.28-8.24 (m, 1H), 7.55 (d, J=8.1 Hz, 2H), 7.43 (d, J=7.9 Hz, 2H), 7.11-7.05 (m, 2H), 6.65-6.62 (m, 2H), 5.65-5.61 (m, 2H), 4.65-4.60 (m, 2H), 2.97-2.87 (m, 2H), 1.92-1.85 (m, 2H), 1.46-1.36 (m, 8H), 1.32-1.23 (m, 12H); 19F NMR (CD₃OD, 376 MHz) δ−75.41 (s, 3F), −116.23-−116.40 (m, 1F), −132.06-−132.19 (m, 1F), −139.47-−140.58 (m, 1F); Analytical HPLC: t_{R}=10.3 min, 98.1% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₄₆H₄₄F₃N₈O₅ [M+H]⁺ 845.3381, found 845.3366.

### Example 178: Synthesis of Other HaloTag® and SNAP-tag® Ligands: JF₅₆₃-cpSNAP-tag® ligand (18_{CSTL})

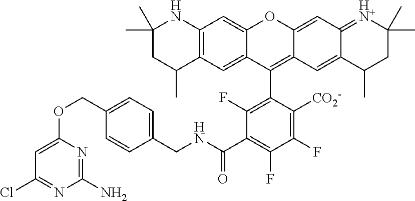

6⁻(MOM-MAC)-JF₅₆₃ (18_{MAC}; acetate salt; 45 mg, 61.4 mol) was taken up in CH₂Cl₂ (4 mL); anisole (400 μL) was added, followed by trifluoroacetic acid (800 L). The reaction was stirred at room temperature for 24 h. Toluene (5 mL) was added, and the reaction mixture was concentrated to dryness. The residue was combined with a premixed solution of 4-((4⁻(aminomethyl)benzyl)oxy)-6⁻chloropyrimidin-2⁻amine (32.5 mg, 0.123 mmol, 2 eq) and DIEA (107 μL, 0.614 mmol, 10 eq) in DMF (5 mL), and the reaction was stirred at room temperature for 2 h. The solvent was removed by rotary evaporation, and the crude material was purified by reverse phase HPLC (30-60% MeCN/H₂O, linear gradient, with constant 0.1% v/v TFA additive) to yield 46.7 mg (80%, TFA salt) of 18_{CSTL} as a red-purple solid (mixture of diastereomers). ¹H NMR (CDCl₃, 400 MHz) δ 7.39 (AB quartet, v_{A}=2965.0 Hz, v_{B}=2950.7 Hz, J_{AB}=8.3 Hz, 4H), 7.10-7.04 (m, 2H), 6.67-6.63 (m, 2H), 6.10-6.07 (m, 1H), 5.36-5.31 (m, 2H), 4.67-4.56 (m, 2H), 2.97-2.86 (m, 2H), 1.93-1.84 (m, 2H), 1.46-1.36 (m, 8H), 1.32-1.22 (m, 12H); 19F NMR (CDCl₃, 376 MHz) δ−75.52 (s, 3F), −115.81-−116.06 (m, 1F), −131.65-−131.94 (m, 1F), −139.10-−139.65 (m, 1F); Analytical HPLC: t_{R}=13.2 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₄₅H₄₃C₁F₃N₆O₅ [M+H]⁺ 839.2930, found 839.2917.

### Example 179: Synthesis of Other HaloTag® and SNAP-tag® Ligands: 4⁻((4-(((2⁻Amino-9H-purin-6⁻yl)oxy)methyl)benzyl)carbamoyl)-2,3,5⁻trifluoro-6-(1,2,2,4,8,10,10,11⁻octamethyl-1,2,10,11⁻tetrahydropyrano[3,2⁻g:5,6⁻g′]diquinolin-13⁻ium-6⁻yl)benzoate (11_{STL})

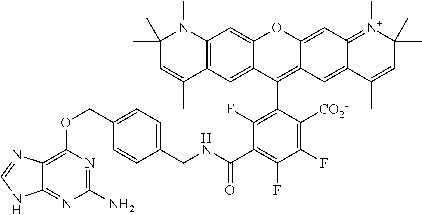

Rhodamine 11_{MAC} (acetate salt; 60 mg, 79.3 μmol) was taken up in CH₂Cl₂ (5 mL), and trifluoroacetic acid (1 mL) was added. The reaction was stirred at room temperature for 18 h. Toluene (5 mL) was added, and the reaction mixture was concentrated to dryness. The residue was combined with a premixed solution of 6⁻((4⁻(aminomethyl)benzyl)oxy)-9H-purin-2-amine (BG-NH₂; 42.9 mg, 0.159 mmol, 2 eq) and DIEA (138 μL, 0.793 mmol, 10 eq) in DMF (5 mL), and the reaction was stirred at room temperature for 18 h. The solvent was removed by rotary evaporation, and the crude material was purified by reverse phase HPLC (20-60% MeCN/H₂O, linear gradient, with constant 0.1% v/v TFA additive) to afford 11S_{TL} as a purple solid (39.6 mg, 51%, TFA salt). ¹H NMR (CD₃OD, 400 MHz) δ 8.30 (s, 1H), 7.55 (d, J=8.0 Hz, 2H), 7.43 (d, J=7.9 Hz, 2H), 6.86 (s, 2H), 6.84 (s, 2H), 5.71 (s, 2H), 5.64 (s, 2H), 4.63 (s, 2H), 3.21 (s, 6H), 1.89 (s, 6H), 1.52 (s, 6H), 1.51 (s, 6H); 19F NMR (CD₃OD, 376 MHz) δ−75.47 (s, 3F), −115.97 (d, J=15.4 Hz, 1F), −131.64 (d, J=23.1 Hz, 1F), −139.44 (dd, J=22.5, 15.3 Hz, 1F); Analytical HPLC: t_{R}=11.1 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₄₈H₄₄F₃N₈O₅[M+H]⁺ 869.3381, found 869.3371.

### Example 180: Synthesis of Other HaloTag® and SNAP-tag® Ligands: 4⁻((4-(((2⁻Amino-6⁻chloropyrimidin-4⁻yl)oxy)methyl)benzyl)carbamoyl)-2,3,5⁻trifluoro-6-(1,2,2,4,8,10,10,11⁻octamethyl-1,2,10,11⁻tetrahydropyrano[3,2⁻g:5,6⁻g′]diquinolin-13⁻ium-6⁻yl)benzoate (11_{CSTL})

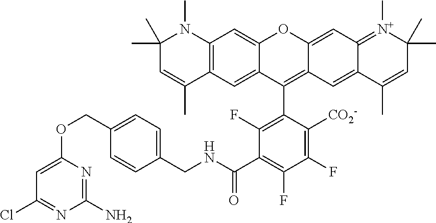

Rhodamine 11_{MAC} (acetate salt; 60 mg, 79.3 μmol) was taken up in CH₂Cl₂ (5 mL), and trifluoroacetic acid (1 mL) was added. The reaction was stirred at room temperature for 18 h. Toluene (5 mL) was added, and the reaction mixture was concentrated to dryness. The residue was combined with a premixed solution of 4⁻((4⁻(aminomethyl)benzyl)oxy)-6-chloropyrimidin-2⁻amine (42.0 mg, 0.159 mmol, 2 eq) and DIEA (138 μL, 0.793 mmol, 10 eq) in DMF (5 mL), and the reaction was stirred at room temperature for 18 h. The solvent was removed by rotary evaporation, and the crude material was purified by reverse phase HPLC (30-70% MeCN/H₂O, linear gradient, with constant 0.1% v/v TFA additive) to afford 11_{CSTL} as a purple solid (40.4 mg, 52%, TFA salt). ¹H NMR (CD₃OD, 400 MHz) δ 7.39 (AB quartet, v_{A}=2962.5 Hz, v_{B}=2949.3 Hz, J_{AB}=8.2 Hz, 4H), 6.85 (s, 2H), 6.84 (s, 2H), 6.08 (s, 1H), 5.71 (s, 2H), 5.33 (s, 2H), 4.61 (s, 2H), 3.21 (s, 6H), 1.88 (s, 6H), 1.52 (s, 6H), 1.52 (s, 6H); 19F NMR (CD₃OD, 376 MHz) δ−75.57 (s, 3F), −115.71 (d, J=15.3 Hz, 1F), −131.42 (dd, J=22.4, 3.0 Hz, 1F), −139.04 (dd, J=22.3, 15.3 Hz, 1F); Analytical HPLC: t_{R}=13.9 min, >99% purity (10-95% MeCN/H₂O, linear gradient, with constant 0.1% v/v TFA additive; 20 min run; 1 mL/min flow; ESI; positive ion mode; detection at 600 nm); HRMS (ESI) calcd for C₄₇H₄₃C₁F₃N₆O₅ [M+H]⁺ 863.2930, found 863.2924.

### Example 181: Synthesis of Other HaloTag® and SNAP-tag® Ligands: JF₆₅₇-SNAP-tag® ligand (59_{STL})

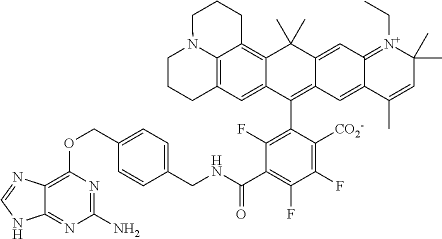

6⁻(MOM-MAC)-JF₆₅₇ (59_{MAC}; 40 mg, 55.2 mol) was taken up in CH₂Cl₂ (4 mL); triethylsilane (400 μL) was added, followed by trifluoroacetic acid (800 L). The reaction was stirred at room temperature for 8 h. Toluene (5 mL) was added, and the reaction mixture was concentrated to dryness. The residue was combined with a premixed solution of 6⁻((4-(aminomethyl)benzyl)oxy)-9H-purin-2⁻amine (BG-NH₂; 29.8 mg, 0.110 mmol, 2 eq) and DIEA (96.1 μL, 0.552 mmol, 10 eq) in DMF (4 mL), and the reaction was stirred at room temperature for 18 h. The solvent was removed by rotary evaporation, and the crude material was purified by reverse phase HPLC (20-50% MeCN/H₂O, linear gradient, with constant 0.1% v/v TFA additive) to afford 37.9 mg (68%, TFA salt) of 59_{STL} as a dark blue solid (mixture of two diastereomers). ¹H NMR (CD₃OD, 400 MHz) δ 8.23 (s, 0.5H), 8.22 (s, 0.5H), 7.54 (d, J=7.9 Hz, 2H), 7.43 (d, J=8.0 Hz, 2H), 6.94 (s, 1H), 6.84-6.78 (m, 2H), 5.63 (s, 1H), 5.62 (s, 1H), 4.62 (s, 2H), 3.83 (dq, J=14.1, 7.0 Hz, 1H), 3.71-3.55 (m, 5H), 3.25-3.14 (m, 2H), 2.85-2.74 (m, 1H), 2.65 (t, J=6.4 Hz, 2H), 2.12-2.01 (m, 2H), 1.97-1.85 (m, 3H), 1.91 (s, 1.5H), 1.90 (s, 1.5H), 1.84 (s, 1.5H), 1.83 (s, 1.5H), 1.58-1.50 (m, 1H), 1.482 (s, 1.5H), 1.477 (s, 1.5H), 1.39-1.32 (m, 6H), 1.16 (d, J=6.5 Hz, 1.5H), 1.14 (d, J=6.5 Hz, 1.5H); 19F NMR (CD₃OD, 376 MHz) δ−75.39 (s, 3F), −116.89 (d, J=15.1 Hz, 0.5F), −116.99 (d, J=15.3 Hz, 0.5F), −133.73 (dd, J=22.4, 2.0 Hz, 0.5F), −133.85 (dd, J=22.0, 2.1 Hz, 0.5F), −140.51 (dd, J=22.1, 15.2 Hz, 0.5F), −140.80 (dd, J=22.3, 15.4 Hz, 0.5F); Analytical HPLC: t_{R} (two isomers)=10.8 min, 10.9 min; >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₅₁H₅₂F₃N₈O₄[M+H]⁺ 897.4058, found 897.4046.

### Example 182: Synthesis of Other HaloTag® and SNAP-tag® Ligands: JF₆₅₇-cpSNAP-tag® ligand (59_{CSTL})

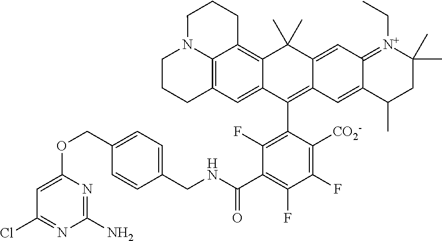

6⁻(MOM-MAC)-JF₆₅₇ (59_{MAC}; 40 mg, 55.2 mol) was taken up in CH₂Cl₂ (4 mL); triethylsilane (400 μL) was added, followed by trifluoroacetic acid (800 L). The reaction was stirred at room temperature for 8 h. Toluene (5 mL) was added, and the reaction mixture was concentrated to dryness. The residue was combined with a premixed solution of 4⁻((4-(aminomethyl)benzyl)oxy)-6⁻chloropyrimidin-2⁻amine (29.2 mg, 0.110 mmol, 2 eq) and DIEA (96.1 μL, 0.552 mmol, 10 eq) in DMF (4 mL), and the reaction was stirred at room temperature for 18 h. The solvent was removed by rotary evaporation, and the crude material was purified by reverse phase HPLC (30-60% MeCN/H₂O, linear gradient, with constant 0.1% v/v TFA additive) to afford 34.0 mg (61%, TFA salt) of 59_{CSTL} as a dark blue solid (mixture of two diastereomers). ¹H NMR (CD₃OD, 400 MHz) δ 7.39 (AB quartet, v_{A}=2965.7 Hz, v_{B}=2951.3 Hz, J_{AB}=8.3 Hz, 4H), 6.944 (s, 0.5H), 6.939 (s, 0.5H), 6.83-6.77 (m, 2H), 6.085 (s, 0.5H), 6.080 (s, 0.5H), 5.340 (s, 1H), 5.336 (s, 1H), 4.61 (s, 2H), 3.83 (dq, J=14.1, 7.0 Hz, 1H), 3.70-3.54 (m, 5H), 3.26-3.14 (m, 2H), 2.84-2.74 (m, 1H), 2.64 (t, J=6.3 Hz, 2H), 2.12-2.02 (m, 2H), 1.98-1.85 (m, 3H), 1.915 (s, 1.5H), 1.906 (s, 1.5H), 1.84 (s, 1.5H), 1.83 (s, 1.5H), 1.58-1.50 (m, 1H), 1.48 (s, 3H), 1.39-1.33 (m, 6H), 1.15 (d, J=6.5 Hz, 1.5H), 1.13 (d, J=6.5 Hz, 1.5H); 19F NMR (CD₃OD, 376 MHz) δ−75.43 (s, 3F), −116.65 (d, J=15.1 Hz, 0.5F), −116.82 (d, J=15.2 Hz, 0.5F), −133.54 (dd, J=22.5, 2.3 Hz, 0.5F), −133.73 (dd, J=22.6, 2.5 Hz, 0.5F),-140.30 (dd, J=22.1, 15.4 Hz, 0.5F), −140.44 (dd, J=22.3, 15.3 Hz, 0.5F); Analytical HPLC: t_{R}(two isomers)=14.0 min, 14.2 min; 97.4% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₅₀H₅₁C₁F₃N₆O₄ [M+H]⁺ 891.3607, found 891.3596.

### Example 183: Synthesis of Other HaloTag® and SNAP-tag® Ligands: JF₆₉₈-SNAP-tag® ligand (31_{STL})

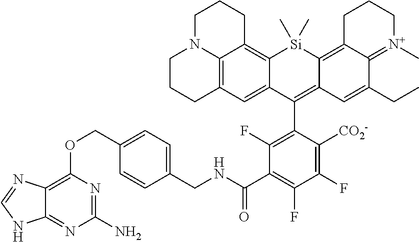

6⁻(MOM-MAC)-JF₆₉₈ (31_{MAC}; 45 mg, 63.3 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 6⁻((4-(aminomethyl)benzyl)oxy)-9H-purin-2⁻amine (BG-NH₂; 34.2 mg, 0.127 mmol, 2 eq) and DIEA (110 μL, 0.633 mmol, 10 eq) in DMF (4 mL), and the reaction was stirred at room temperature for 18 h. The solvent was removed by rotary evaporation, and the crude material was purified by reverse phase HPLC (20-50% MeCN/H₂O, linear gradient, with constant 0.1% v/v TFA additive) to afford 31_{STL} as a blue-green solid (30.0 mg, 48%, TFA salt). ¹H NMR (CD₃OD, 400 MHz) δ 8.22 (s, 1H), 7.54 (d, J=8.1 Hz, 2H), 7.42 (d, J=8.2 Hz, 2H), 6.65 (s, 2H), 5.63 (s, 2H), 4.62 (s, 2H), 3.57 (t, J=6.2 Hz, 4H), 3.54 (t, J=6.2 Hz, 4H), 3.02-2.93 (m, 4H), 2.57 (t, J=6.2 Hz, 4H), 2.12-2.00 (m, 4H), 1.97-1.86 (m, 4H), 0.70 (s, 3H), 0.67 (s, 3H); 19F NMR (CD₃OD, 376 MHz) δ−75.39 (s, 3F), −117.90 (d, J=15.2 Hz, 1F), −134.96 (d, J=22.1 Hz, 1F),-141.26 (dd, J=22.2, 15.2 Hz, 1F); Analytical HPLC: t_{R}=10.2 min, >99% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₄₈H₄₆F₃N₈O₄Si [M+H]⁺ 883.3358, found 883.3346.

### Example 184: Synthesis of Other HaloTag® and SNAP-tag® Ligands: JF₆₉₈-cpSNAP-tag® ligand (31_{CSTL})

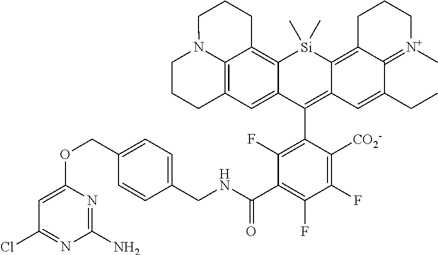

6⁻(MOM-MAC)-JF₆₉₈ (3_{MAC}; 45 mg, 63.3 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 4⁻((4-(aminomethyl)benzyl)oxy)-6⁻chloropyrimidin-2⁻amine (33.5 mg, 0.127 mmol, 2 eq) and DIEA (110 μL, 0.633 mmol, 10 eq) in DMF (4 mL), and the reaction was stirred at room temperature for 18 h. The solvent was removed by rotary evaporation, and the crude material was purified by reverse phase HPLC (30-60% MeCN/H₂O, linear gradient, with constant 0.1% v/v TFA additive) to afford 31_{CSTL} as a blue-green solid (29.0 mg, 46%, TFA salt). ¹H NMR (CD₃OD, 400 MHz) δ 7.40 (AB quartet, v_{A}=2967.5 Hz, v_{B}=2951.1 Hz, J_{A}B=8.2 Hz, 4H), 6.65 (s, 2H), 6.09 (s, 1H), 5.34 (s, 2H), 4.61 (s, 2H), 3.58 (t, J=6.2 Hz, 4H), 3.54 (t, J=6.2 Hz, 4H), 2.97 (t, J=6.1 Hz, 4H), 2.56 (t, J=6.2 Hz, 4H), 2.12-2.01 (m, 4H), 1.97-1.86 (m, 4H), 0.71 (s, 3H), 0.67 (s, 3H); 19F NMR (CD₃OD, 376 MHz) δ−75.44 (s, 3F), −117.74 (d, J=15.1 Hz, 1F),-134.86 (d, J=21.9 Hz, 1F), −141.05 (dd, J=22.1, 15.2 Hz, 1F); Analytical HPLC: t_{R}=13.2 min, >99% purity (10-95% MeCN/H₂O, linear gradient, with constant 0.1% v/v TFA additive; 20 min run; 1 mL/min flow; ESI; positive ion mode; detection at 700 nm); HRMS (ESI) calcd for C₄₇H₄₅C₁F₃N₆O₄Si [M+H]⁺ 877.2907, found 877.2901.

### Example 185: Synthesis of Other HaloTag® and SNAP-tag® Ligands: 4⁻((4-(((2⁻Amino-9H-purin-6⁻yl)oxy)methyl)benzyl)carbamoyl)-2⁻(3,6-bis(methyl(phenyl)amino)xanthylium-9⁻yl)-3,5,6⁻trifluorobenzoate (64_{STL})

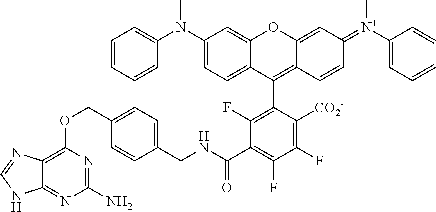

Rhodamine 64_{MAC} (50 mg, 72.6 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 6⁻((4-(aminomethyl)benzyl)oxy)-9H-purin-2⁻amine (BG-NH₂; 39.3 mg, 0.145 mmol, 2 eq) and DIEA (126 μL, 0.726 mmol, 10 eq) in DMF (5 mL), and the reaction was stirred at room temperature for 18 h. The solvent was removed by rotary evaporation, and the crude material was purified by reverse phase HPLC (20-80% MeCN/H₂O, linear gradient, with constant 0.1% v/v TFA additive) to afford 64_{STL} as a purple solid (53.6 mg, 76%, TFA salt). ¹H NMR (CD₃OD, 400 MHz) δ 9.45 (t, J=5.9 Hz, 1H), 8.30 (s, 1H), 7.61-7.45 (m, 8H), 7.44-7.35 (m, 6H), 7.32 (d, J=9.4 Hz, 2H), 6.99 (d, J=2.4 Hz, 2H), 6.92 (dd, J=9.5, 2.4 Hz, 2H), 5.64 (s, 2H), 4.63-4.57 (m, 2H), 3.62 (s, 6H); 19F NMR (CD₃OD, 376 MHz) δ−75.50 (s, 3F), −116.67 (d, J=15.2 Hz, 1F), −132.09 (d, J=22.5 Hz, 1F), −139.29 (dd, J=22.3, 15.2 Hz, 1F); Analytical HPLC: t_{R}=10.9 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₄₈H₃₆F₃N₈O₅[M+H]⁺ 861.2755, found 861.2742.

### Example 186: Synthesis of Other HaloTag® and SNAP-tag® Ligands: 4⁻((4-(((2⁻Amino-6⁻chloropyrimidin-4⁻yl)oxy)methyl)benzyl)carbamoyl)-2⁻(3,6-bis(methyl(phenyl)amino)-xanthylium-9⁻yl)-3,5,6⁻trifluorobenzoate (64_{CSTL})

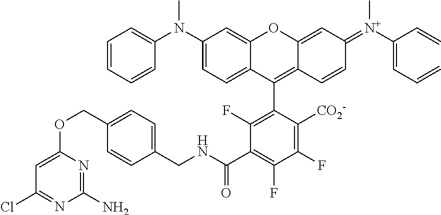

Rhodamine 64_{MAC} (50 mg, 72.6 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 4⁻((4-(aminomethyl)benzyl)oxy)-6⁻chloropyrimidin-2⁻amine (38.4 mg, 0.145 mmol, 2 eq) and DIEA (126 μL, 0.726 mmol, 10 eq) in DMF (5 mL), and the reaction was stirred at room temperature for 18 h. The solvent was removed by rotary evaporation, and the crude material was purified by reverse phase HPLC (30-70% MeCN/H₂O, linear gradient, with constant 0.1% v/v TFA additive) to afford 64_{CSTL} as a purple solid (40.6 mg, 58%, TFA salt). ¹H NMR (CD₃OD, 400 MHz) δ 9.39 (t, J=5.8 Hz, 1H), 7.61-7.54 (m, 4H), 7.51-7.44 (m, 2H), 7.42-7.33 (m, 8H), 7.31 (d, J=9.4 Hz, 2H), 6.99 (d, J=2.4 Hz, 2H), 6.92 (dd, J=9.4, 2.4 Hz, 2H), 6.08 (s, 1H), 5.33 (s, 2H), 4.61-4.56 (m, 2H), 3.62 (s, 6H); 19F NMR (CD₃OD, 376 MHz) δ−75.59 (s, 3F),-116.36 (d, J=14.8 Hz, 1F), −131.85 (d, J=22.4 Hz, 1F), −138.58 (dd, J=22.0, 15.3 Hz, 1F); Analytical HPLC: t_{R}=13.7 min, >99% purity (10-95% MeCN/H₂O, linear gradient, with constant 0.1% v/v TFA additive; 20 min run; 1 mL/min flow; ESI; positive ion mode; detection at 575 nm); HRMS (ESI) calcd for C₄₇H₃₅C₁F₃N₆O₅ [M+H]⁺ 855.2304, found 855.2296.

### Example 187: Synthesis of Other HaloTag® and SNAP-tag® Ligands: JQ₆₄₅—snap-tag® ligand (65_{STL})

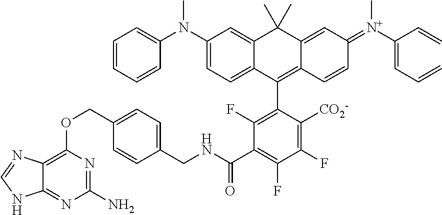

6⁻(MOM-MAC)-JQ₆₄₅ (65_{MAC}; 50 mg, 70.0 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 6⁻((4-(aminomethyl)benzyl)oxy)-9H-purin-2⁻amine (BG-NH₂; 37.8 mg, 0.140 mmol, 2 eq) and DIEA (122 μL, 0.700 mmol, 10 eq) in DMF (5 mL), and the reaction was stirred at room temperature for 18 h. The solvent was removed by rotary evaporation, and the crude material was purified by reverse phase HPLC (20-80% MeCN/H₂O, linear gradient, with constant 0.1% v/v TFA additive) to afford 65_{STL} as a blue solid (37.8 mg, 54%, TFA salt). ¹H NMR (CD₃OD, 400 MHz) δ 9.46 (t, J=6.1 Hz, 1H), 8.30 (s, 1H), 7.53 (d, J=8.1 Hz, 2H), 7.50-7.43 (m, 4H), 7.39 (d, J=8.0 Hz, 2H), 7.33-7.23 (m, 6H), 7.15 (d, J=2.5 Hz, 2H), 7.00 (d, J=9.1 Hz, 2H), 6.75 (dd, J=9.1, 2.5 Hz, 2H), 5.63 (s, 2H), 4.60-4.55 (m, 2H), 3.51 (s, 6H), 1.63 (s, 3H), 1.58 (s, 3H); 19F NMR (CD₃OD, 376 MHz) δ−75.53 (s, 3F), −119.50-−119.77 (m, 1F), −134.08 (d, J=21.3 Hz, 1F), −141.32-−141.60 (m, 1F); Analytical HPLC: t_{R}=12.7 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₅₁H₄₂F₃NsO₄ [M+H]⁺ 887.3276, found 887.3263.

### Example 188: Synthesis of Other HaloTag® and SNAP-tag® Ligands: JQ₆₄₅-cpSNAP-tag® ligand (65_{CSTL})

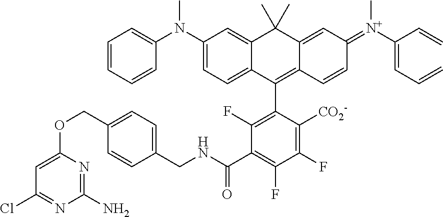

6⁻(MOM-MAC)-JQ₆₄₅ (65_{MAC}; 50 mg, 70.0 mol) was taken up in CH₂Cl₂ (4 mL); triethylsilane (400 μL) was added, followed by trifluoroacetic acid (800 L). The reaction was stirred at room temperature for 6 h. Toluene (5 mL) was added, and the reaction mixture was concentrated to dryness. The residue was combined with a premixed solution of 4⁻((4-(aminomethyl)benzyl)oxy)-6⁻chloropyrimidin-2⁻amine (37.0 mg, 0.140 mmol, 2 eq) and DIEA (122 μL, 0.700 mmol, 10 eq) in DMF (5 mL), and the reaction was stirred at room temperature for 18 h. The solvent was removed by rotary evaporation, and the crude material was purified by reverse phase HPLC (10-95% MeCN/H₂O, linear gradient, with constant 0.1% v/v TFA additive) to afford 65_{CSTL} as a blue solid (24.8 mg, 36%, TFA salt). ¹H NMR (CD₃OD, 400 MHz) δ 9.41 (t, J=5.8 Hz, 1H), 7.56-7.48 (m, 4H), 7.42-7.29 (m, 10H), 7.16 (d, J=2.5 Hz, 2H), 7.10 (d, J=9.2 Hz, 2H), 6.75 (dd, J=9.2, 2.5 Hz, 2H), 6.09 (s, 1H), 5.34 (s, 2H), 4.60-4.55 (m, 2H), 3.58 (s, 6H), 1.64 (s, 3H), 1.56 (s, 3H); 19F NMR (CD₃OD, 376 MHz) δ−75.68 (s, 3F), −118.21 (d, J=16.2 Hz, 1F), −133.50 (d, J=21.8 Hz, 1F), −140.05 (m, 1F); Analytical HPLC: t_{R}=16.0 min, >99% purity (10-95% MeCN/H₂O, linear gradient, with constant 0.1% v/v TFA additive; 20 min run; 1 mL/min flow; ESI; positive ion mode; detection at 650 nm); HRMS (ESI) calcd for C₅₀H₄₁C₁F₃N₆O₄ [M+H]⁺ 881.2824, found 881.2813.

### Example 189: Synthesis of Other HaloTag® and SNAP-tag® Ligands: FMGL-SNAP-tag® ligand (49_{STL})

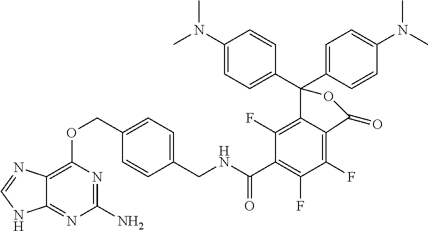 [SMILES: CN(C)c1ccc(C2(c3ccc(N(C)C)cc3)OC(=O)c3c(F)c(F)c(C(=O)NCc4ccc(COc5nc(N)nc6[nH]cnc56)cc4)c(F)c32)cc1]

4,5,7⁻Trifluoro-6⁻(MOM-MAC)-MGL (49_{MAC}; 50 mg, 90.8 mol) was taken up in CH₂Cl₂ (4 mL); triethylsilane (400 μL) was added, followed by trifluoroacetic acid (800 L). The reaction was stirred at room temperature for 18 h. Toluene (5 mL) was added, and the reaction mixture was concentrated to dryness. The residue was combined with a premixed solution of 6⁻((4⁻(aminomethyl)benzyl)oxy)-9H-purin-2⁻amine (BG-NH₂; 49.1 mg, 0.182 mmol, 2 eq) and DIEA (158 μL, 0.908 mmol, 10 eq) in DMF (4 mL), and the reaction was stirred at room temperature for 2 h. The solvent was removed by rotary evaporation, and the crude material was purified by reverse phase HPLC (10-75% MeCN/H₂O, linear gradient, with constant 0.1% v/v TFA additive). The pooled product fractions were partially concentrated to remove MeCN, diluted with saturated NaHCO₃, and extracted with 10% MeOH/CH₂Cl₂ (2×). The organic extracts were dried over anhydrous MgSO₄, filtered, and evaporated to provide 49_{STL} (30.4 mg, 46%) as a light blue-green solid. ¹H NMR (CD₃OD with 1% TFA, 400 MHz) δ 8.39 (s, 1H), 7.56 (d, J=8.1 Hz, 2H), 7.42 (d, J=8.1 Hz, 2H), 7.33 (d, J=9.1 Hz, 4H), 7.18 (d, J=9.0 Hz, 4H), 5.67 (s, 2H), 4.59 (s, 2H), 3.13 (s, 12H); 19F NMR (CD₃OD with 1% TFA, 376 MHz) δ-117.71 (d, J=21.8 Hz, 1F), −133.63 (d, J=20.9 Hz, 1F), −142.06 (t, J=21.4 Hz, 1F); Analytical HPLC: t_{R}=10.0 min, >99% purity (10-95% MeCN/H₂O, linear gradient, with constant 0.1% v/v TFA additive; 20 min run; 1 mL/min flow; ESI; positive ion mode; detection at 280 nm); HRMS (ESI) calcd for C₃₈H₃₄F₃N₈O₄[M+H]⁺ 723.2650, found 723.2640.

### Example 190: Synthesis of Other HaloTag® and SNAP-tag® Ligands: FMGL-cpSNAP-tag® ligand (49_{CSTL})

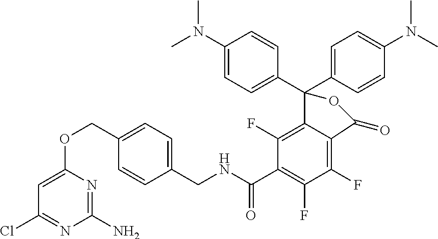 [SMILES: CN(C)c1ccc(C2(c3ccc(N(C)C)cc3)OC(=O)c3c(F)c(F)c(C(=O)NCc4ccc(COc5cc(Cl)nc(N)n5)cc4)c(F)c32)cc1]

4,5,7⁻Trifluoro-6⁻(MOM-MAC)-MGL (49_{MAC}; 50 mg, 90.8 mol) was taken up in CH₂Cl₂ (4 mL); triethylsilane (400 μL) was added, followed by trifluoroacetic acid (800 L). The reaction was stirred at room temperature for 18 h. Toluene (5 mL) was added, and the reaction mixture was concentrated to dryness. The residue was combined with a premixed solution of 4⁻((4⁻(aminomethyl)benzyl)oxy)-6⁻chloropyrimidin-2⁻amine (48.1 mg, 0.182 mmol, 2 eq) and DIEA (158 μL, 0.908 mmol, 10 eq) in DMF (5 mL), and the reaction was stirred at room temperature for 2 h. The solvent was removed by rotary evaporation, and the crude material was purified by reverse phase HPLC (10-75% MeCN/H₂O, linear gradient, with constant 0.1% v/v TFA additive). The pooled product fractions were partially concentrated to remove MeCN, diluted with saturated NaHCO₃, and extracted with 10% MeOH/CH₂Cl₂ (2×). The organic extracts were dried over anhydrous MgSO₄, filtered, and evaporated to provide 49_{CSTL} (31.2 mg, 48%) as a blue-green solid. ¹H NMR (CD₃OD, 400 MHz) δ 7.38 (AB quartet, v_{A}=2966.2 Hz, v_{B}=2941.4 Hz, J_{AB}=8.1 Hz, 4H), 7.10 (d, J=9.0 Hz, 4H), 6.71 (d, J=9.0 Hz, 4H), 6.10 (s, 1H), 5.34 (s, 2H), 4.56 (s, 2H), 2.94 (s, 12H); 19F NMR (CD₃OD, 376 MHz) δ-117.92 (d, J=22.1 Hz, 1F), −135.20 (dd, J=20.9, 2.3 Hz, 1F), −143.30 (t, J=21.7 Hz, 1F); Analytical HPLC: t_{R}=13.3 min, >99% purity (10-95% MeCN/H₂O, linear gradient, with constant 0.1% v/v TFA additive; 20 min run; 1 mL/min flow; ESI; positive ion mode; detection at 280 nm); HRMS (ESI) calcd for C₃₇H₃₃C₁F₃N₆O₄ [M+H]⁺ 717.2198, found 717.2191.

### Example 191: Optical Spectroscopy and Microscopy: Compound sources

Compound 81_{HTL} was purchased from Promega. Compounds 1_{HTL}, 84_{HTL}, 84_{HST}, 85_{HTL}, 86_{HTL}, 89_{HTL}, 105_{HTL}, 106_{HTL}, and 107_{HTL} were available from previous work. Data for dyes 1, 2, 22, 74, 75, 78-91, 93, and 95 were taken from published work of the inventors'; all spectral data was measured under identical conditions.

### Example 192: General UV-vis and fluorescence spectroscopy (Tables 3-4, FIG. 2A, 2B, FIG. 3A-3P, FIG. 4A-4D, FIG. 7A-7L, FIG. 14A-14D)

All dyes for spectroscopy were prepared as stock solutions in DMSO and diluted such that the final DMSO concentration did not exceed 1% v/v. Spectroscopy was performed using 1⁻cm path length, 3.5⁻mL quartz cuvettes or 1⁻cm path length, 1.4⁻mL semi-micro quartz cuvettes from Starna Cells. All measurements were taken at ambient temperature (22±2° C.). Absorption spectra were recorded on a Cary Model 100 spectrometer (Agilent), and fluorescence spectra were recorded on a Cary Eclipse fluorometer (Varian). The spectra, maximum absorption wavelength (labs), extinction coefficient at λ_{abs} (8), and maximum emission wavelength (em) were measured in 10 mM HEPES, pH 7.3 buffer (rhodamines and Malachite Green derivative 49) or 0.1 M NaOH (fluoresceins); the reported values for e are averages (n=3). Normalized spectra are shown for clarity.

### Example 193: Quantum yield determination (Tables 3-5, FIG. 14A-14D)

All reported absolute fluorescence quantum yield values (Φ_{f}) were measured using a Quantaurus-QY spectrometer (model C11374, Hamamatsu). This instrument uses an integrating sphere to determine photons absorbed and emitted by a sample. Measurements were carried out using dilute samples (A<0.1), and self-absorption corrections were performed using the instrument software.⁸¹

### Example 194: HaloTag® Protein Purification

The bacterial expression vector pRSET-A (Invitrogen) was used to recombinantly express HaloTag® (HT7; Promega).⁷ The soluble 6×His-Tagged HaloTag® protein was affinity purified by immobilized metal affinity chromatography (IMAC) on a 5⁻mL Fast Flow HiTrap Sepharose 6 column (Cytiva) with a 0-200 mM imidazole elution gradient using an ÄKTA Avant Protein Purification System (Cytiva). A₂₈₀ peak fractions were pooled, concentrated by a spin concentrator, and dialyzed 3× into tris-buffered saline (TBS; 137 mM NaCl, 20 mM Tris-HCl, pH 7.4).

### Example 195: Absorption increase of ligands on binding HaloTag® protein (FIG. 5B)

HaloTag® protein was used as a 100 M solution in 1× TBS. Absorption measurements were performed in 1.4⁻mL semi-micro quartz cuvettes. A 5 M solution of HaloTag® ligand 65_{HTL} or 91_{HTL} was prepared in 10 mM HEPES, pH 7.3 containing 0.1 mg mL-1 CHAPS. An aliquot of HaloTag® protein (2 equiv, 10 M final [HaloTag®]) was added. To examine chromogenicty (FIG. 5B, left panel), the resulting mixture was incubated until a consistent absorption signal/spectrum was observed (<60 min for 65_{HTL}); an equivalent volume of 1× TBS blank was added to separate “−HT” samples in place of enzyme to record the ligand-only absorption. Spectra are averages (n=2). To compare rates of HaloTag® labeling (FIG. 5B, right panel) absorption at λ_{abs} (645 nm for 65_{HTL}, 621 nm for 91_{HTL}) was recorded at regular intervals (0.1 min, 0-5 min; 1 min, 5-120 min; 5 min, 2-24 h) immediately following addition of HaloTag® protein.

### Example 196: Determination of K_{L-Z} (Tables 4-5)

K_{L-Z} was calculated using the following equation:^{16, 18} K_{L-Z}=(ε_{dw}/εₘₐₓ)/(1−ε_{dw}/εₘₐₓ). ε_{dw} is the extinction coefficient of the dyes in a 1:1 (v/v) dioxane/water solvent mixture; this dioxane/water mixture was chosen to give the maximum spread of K_{L-Z} values across all classes of rhodamines.¹⁸ εₘₐₓ refers to the maximal extinction coefficients measured in different solvent mixtures empirically determined depending on dye type: 0.1% v/v trifluoroacetic acid in ethanol for 18, 20, 29, 34, 36, 70-72, 81-83, 93-95, 99-102, and 104; 0.1% v/v trifluoroacetic acid in 2,2,2⁻trifluoroethanol for all other rhodamine variants. A accurate determination of low K_{L-Z} values is complicated by the relatively poor sensitivity of absorbance measurements. No attempt was made to estimate K_{L-Z} values when no measurable absorbance of the dye was observed in the dioxane/water solution.

### Example 197: pKₐ determination (Table 3, FIG. 2C, 2D)

The pKₐ values for compounds 51, 53, 60, 76, and 77 were determined by measuring the change in absorbance as a function of pH. For pH 2.0-4.0, the McIlvaine citrate/phosphate buffer system was used. For other pH ranges dye absorbance was measured in buffers containing 150 mM NaCl and 10 mM buffer using the following systems: citrate (pH 4.0-6.0), phosphate (pH 5.8-8.0); tris (pH 7.8-9.0); carbonate (pH 9.2-10.0). Buffer solutions containing 5 M of each fluorophore were prepared (36 samples in duplicate from pH 4.0-10.0 for 51, 53, and 60; 48 samples in duplicate from pH 2.0-10.0 for 76 and 77). Absorbance values were recorded on a Cary Model 100 spectrometer and plotted using GraphPad Prism software. The points were fitted to a sigmoidal dose response curve with a variable slope to determine the Hill coefficient (η_{H}).

### Example 198: General Cell Culture Methods

U2OS cells (ATCC) and U2OS cells stably expressing an integrated HaloTag®-histone H₂B fusion protein (U2OS·H2B·HaloTag®) were cultured in Dulbecco's modified Eagle medium (DMEM, phenol red-free; Life Technologies) supplemented with 10% (v/v) fetal bovine serum (FBS, Life Technologies), 1 mM GlutaMAX (Life Technologies) and maintained at 37° C. in a humidified 5% (v/v) CO₂ environment. These cell lines undergo regular mycoplasma testing by the Janelia Cell Culture Facility. For mitochondrial imaging using the HaloTag® system, U2OS cells were transiently transfected using nucleofection (Lonza) with a plasmid constitutively expressing a HaloTag®-TOMM20 fusion protein, which is located on the outer mitochondrial membrane. For 4⁻color imaging, U2OS·H2B·HaloTag® stable cells were transiently transfected by nucleofection with plasmids constitutively expressing a SNAP-tag®-TOMM20 fusion protein, and a GFP-SKL (SKL: serine-lysine-leucine) fusion protein, a prototypical peroxisomal matrix targeting signal. All labeled cells were imaged 18-24 h post-transfection or post-plating.

### Example 199: Labeling and confocal microscopy using ligands 70_{HTL}-72_{HTL} (FIG. 11B-11D)

The U2OS·H2B·HaloTag® stable cells were incubated with 100 nM HaloTag® ligand conjugated dyes (70_{HTL}-70_{HTL}) for 2 h at 37° C., washed 3× with dye-free media then imaged live. Confocal imaging was performed on a Leica SP8 using an HC PL APO 86×/1.20 water objective. Single plane images were processed using FIJI.⁸²

### Example 200: Loading and photostability experiments using ligands 40_{HTL}, 44_{HTL}, 1_{HTL}, and 89_{HTL} (FIG. 12C, 12D)

For loading curve experiments (FIG. 12C), U2OS·H2B·HaloTag® stable cells were incubated with 200 nM HaloTag® ligand conjugated dyes (40_{HTL}, 44_{HTL}, 1_{HTL}, or 89_{HTL}) for 0-4 h at 37° C., washed 3× with dye-free media then fixed with 4% paraformaldehyde in 0.1 M phosphate buffer, pH 7.4 for 15 min at 37° C. Confocal imaging was performed on a Leica Stellaris with an HC PL APO 40×/1.25 glycerol objective using the tunable white light laser (WLL) to excite dyes at their λ_{abs} in constant power mode. Fluorescence was quantified as the integrated density of nuclear signals in single-plane images analyzed in FIJI;⁸² n=100 nuclear signals per compound.

For photostability experiments (FIG. 12D), U2OS·H2B·HaloTag® stable cells were incubated with 200 nM HaloTag® ligands (40_{HTL}, 44_{HTL}, 1_{HTL}, or 89_{HTL}) for 2 h at 37° C., washed 3× with dye-free media then fixed with 4% paraformaldehyde in 0.1 M phosphate buffer, pH 7.4 for 15 min at 37° C. Confocal imaging was performed on a Leica SP8 with an HC PL APO 86×/1.20 water objective using adaptive focus control (AFC). To measure photobleaching, the tunable white light laser (WLL) was used to excite dyes at their λ_{abs} in constant power mode over 30 cycles; n=3 where n indicates the number of separate cellular experiments where the intensity of the entire field of view was measured at the indicated time points. Single plane images were processed using FIJI.⁸²

### Example 201: Labeling and multicolor confocal microscopy using 31_{HTL} (FIG. 15D)

To achieve a 4⁻color image, live U2OS·H2B·HaloTag® stable cells that were transiently transfected with plasmids constitutively expressing a SNAP-tag®-TOMM20 fusion protein and an GFP-SKL fusion protein were incubated with 200 nM JF₆₉₈-HaloTag® ligand (31_{HTL}) and JFX₆₅₀⁻SNAP-tag® ligand (89_{HTL}) for 1 h at 37° C., washed 3× with dye-free media, and then fixed with 4% paraformaldehyde in 0.1 M phosphate buffer, pH 7.4 for 15 min at 37° C. Fixed cells were then washed 3× in 1×phosphate buffered saline, pH 7.4 (PBS) and incubated with JF₅₄₉⁻Hoechst (84_{HST}; 3 μM) for 15 min at ambient temperature (22±2° C.) as a nuclear counterstain. Confocal imaging was performed on a Leica Stellaris with an HC PL APO CS2 86×/1.20 water objective using the tunable white light laser (WLL) to excite each dye or fluorescent protein at their λ_{abs}. Confocal image stacks were processed using FIJI and displayed as maximum intensity image projections.

### Example 202: Loading experiments using ligands 65_{HTL} and 91_{HTL} (FIG. 18)

For loading curve experiments, U2OS·H2B·HaloTag® stable cells were incubated with 200 nM 65_{HTL} and 91_{HTL} for 0-4 h at 37° C., washed 3× with dye-free media then chased with 200 nM JF₅₅₂-HaloTag® ligand 15 for 30 min at 37° C., washed 3× with dye-free media and fixed with 4% paraformaldehyde in 0.1 M phosphate buffer, pH 7.4 for 15 min at 37° C. Confocal imaging was performed on a Zeiss LSM 980 using a Plan-Apochromat 20×/0.8 M27 objective, and excited by a 561 nm laser. Fluorescence was quantified as the integrated density of nuclear signals from confocal image stack projections analyzed in FIJI;⁸² n=100 nuclear signals per compound.

### Example 203: Labeling and Airyscan microscopy using ligand 40_{HTL} (FIG. 112E)

U2OS cells transiently transfected with a plasmid constitutively expressing a HaloTag®-TOMM20 fusion protein were incubated with 200 nM JFX₆₃₇-HaloTag® ligand (40_{HTL}) for 1 h at 37° C., washed 3× with dye-free media then fixed with 4% paraformaldehyde in 0.1 M phosphate buffer, pH 7.4 for 15 min at 37° C. Fixed cells were then washed 3× in 1×PBS and incubated with Hoechst 33342 (5 g/mL; nuclear co-stain) for 15 min at ambient temperature (22±2° C.) as a nuclear counterstain. High-resolution Airyscan imaging was performed on a Zeiss LSM 980 with Airyscan 2 confocal microscope using a Plan APO 63×/1.4 oil DIC M27 objective. Confocal image stacks were bulk processed in ZEN Blue (Zeiss) with automatic Airyscan settings; the final images were processed in FIJI.⁸²

### Example 204: Labeling and confocal microscopy using ligands 59_{HTL} and 105_{HTL} (FIG. 13B, 13C)

U2OS·H2B·HaloTag® stable cells were incubated with 200 nM of either JF₆₅₇-HaloTag® ligand (59_{HTL}) or ATTO 647N-HaloTag® ligand (105_{HTL}) for 1 h at 37° C., washed 3× with dye-free media, stained with Hoechst 33342 (5 g/mL; nuclear co-stain) and MitoTracker Green FM (100 nM; mitochondrial stain) for 15 min at 37° C. then imaged live. Standard confocal imaging was performed on a Zeiss LSM 980 using a Plan APO 63×/1.4 oil DIC M27 objective. Confocal image stacks were processed using FIJI⁸² and displayed as maximum intensity image projections.

### Example 205: Loading and photostability experiments using ligands 31_{HTL}, 36_{HTL}, 38_{HTL}, 104_{HTL}, 106_{HTL}, 107_{HTL}, and 108_{HTL} (FIG. 15C, FIG. 1611)

For loading curve experiments (FIG. 15C), U2OS·H2B·HaloTag® stable cells were incubated with 200 nM HaloTag® ligand conjugated dyes (31_{HTL}, 36_{HTL}, 38_{HTL}, 104_{HTL}, 106_{HTL}, 107_{HTL}, and 108_{HTL}) for 0-4 h at 37° C., washed 3× with dye-free media then fixed with 4% paraformaldehyde in 0.1 M phosphate buffer, pH 7.4 for 15 min at 37° C. Confocal imaging was performed on a Leica Stellaris with an HC PL APO 40×/1.25 glycerol objective using the tunable white light laser (WLL) to excite dyes at their λ_{abs} in constant power mode. Fluorescence was quantified as the integrated density of nuclear signals in single-plane images analyzed in FIJI;⁸² n=100 nuclear signals per compound.

For photostability experiments (FIG. 1611), U2OS·H2B·HaloTag® stable cells were incubated with 200 nM HaloTag® ligands (31_{HTL}, 106_{HTL}, and 107_{HTL}) for 3 h at 37° C., washed 3× with dye-free media then fixed with 4% paraformaldehyde in 0.1 M phosphate buffer, pH 7.4 for 15 min at 37° C. Fixed cells were then washed 3× in 1×PBS and incubated with Hoechst 33342 (5 g/mL; nuclear costain) for 15 min at ambient temperature (22±2° C.) as a nuclear counterstain. Confocal imaging was performed on a Leica Stellaris with an HC PL APO 86×/1.20 water objective using adaptive focus control (AFC). Based on Leica recommendations the Hoechst 33342 “blue” channel was used to engage the AFC for imaging using the far-red dyes with emission collection windows >750 nm. To measure photobleaching, the tunable white light laser (WLL) was used to excite dyes at their λ_{abs} in constant power mode over 30 cycles; n=3 where n indicates the number of separate cellular experiments where the intensity of the entire field of view was measured at the indicated time points. Single plane images were processed using FIJI.⁸²

### Example 206: Measurement of HaloTag® ligand labeling rates (FIG. 9A-9O, Table 6)

To measure labeling rates a stopped-flow protocol was used where equimolar amounts of purified HaloTag® protein and HaloTag® ligand were mixed; the labeling was monitored by fluorescence polarization.⁸³ HaloTag® protein and HaloTag® ligands were serially diluted in 50 mM HEPES buffer, pH 7.2 containing 50 mM NaCl, and 0.5 mg/mL bovine serum albumin (BSA) to yield final concentrations of 2 μM, 1 μM, 500 nM, 250 nM, and 125 nM. Sample concentrations were evaluated using a Cary Model 100 spectrometer (Agilent) using ε₂₈₀ (64,430 M⁻¹·cm⁻¹) for the HaloTag® protein or the e values for the different fluorophores. Stopped-flow experiments were performed at 37° C. using an Applied Photophysics Stopped Flow Spectrometer, model SX20, in fluorescence polarization mode. Appropriate long-pass filters were used for each dye type. Five replicates of each concentration pair were collected (n=5). The plots of fluorescence polarization vs. time were fit using DynaFit (Biokin) to calculate k₁, k₋₁, and k₂; timepoints <3 ms were omitted from the analysis. The capture rate (kₐₚₚ) was calculated using the following equation: kₐₚₚ=k₁(k₂/k₋₁×k₂). Reported values are averages of the kₐₚₚ values measured for the different concentrations.

### Example 207: Conjugate stability experiments (FIG. 10A-10D)

Purified HaloTag® protein was incubated with a 10-fold molar excess of “pulse” HaloTag® ligands in 1×PBS in LoBind microcentrifuge tubes (Eppendorf) and incubated for 2-4 h at 4° C. while protected from light. For displacement experiments, unbound pulse dye was removed by passing the reaction mixture through two successive Zeba Spin Desalting Column (ThermoFisher) using 1×PBS. These samples were then incubated with a 10-fold molar excess of JF₆₄₆-HaloTag® ligand (1_{HTL}) or a DMSO control for 1 h at 4° C. Reactions were stopped by adding 4× NuPAGE LDS buffer (ThermoFisher) with NuPAGE Sample Reducing Agent (ThermoFisher) and heated for 10 min at 90° C. Samples were run on NuPAGE 4-12% Bis-Tris 1.0-1.5 mm Mini Protein Gels (ThermoFisher) in NuPAGE MES SDS Running Buffer (ThermoFisher) for 60 min at 200 V. Gels were washed in Milli-Q water for 3 min then imaged on a black tray using a ChemiDoc MP Imaging system (Biorad) and 602/50 or 700/50 emission filter. Exposure times during imaging were set to avoid signal saturation. After fluorescence imaging, the gels were stained with Simply Blue Safe Stain (ThermoFisher) and reimaged using the white tray for calorimetric detection. ImageJ was used for signal quantification with background subtraction.

### Example 208: Single-particle tracking (SPT) experiments (FIG. 13E, 13F, FIG. 12F, 12G)

SPT experiments were carried out as previously described.⁸⁴ JM8·N4 mouse embryonic stem cells (ESCs) stably expressing histone H2B-HaloTag® fusion proteins were generated by co-expressing the PiggyBac EF1a-H2B-HaloTag®-IRES-Neo vector with the super piggyBac transposase as described previously,85 followed by G418 selection (500 μg/mL) for 2 weeks and verified by FACS sorting/confocal imaging staining with JF₅₄₉-HaloTag® ligand (84_{HTL}; 100 nM, 30 min). Cells were seeded on 25 mm #1.5 coverglass pre-cleaned with KOH and ethanol and coated with iMatrix-511 (TaKaRa #T304) or rhLaminin-521 (Gibco™ #A²⁹²⁴⁸) according to the manufacturers' instruction. All live-cell imaging experiments were conducted using an ESC imaging medium composed of FluoroBrite DMEM (ThermoFisher) plus 15% v/v ESC-qualified FBS, 1× GlutaMax, 1×NEAA, 0.1 mM 2⁻mercaptoethanol, and LIF. For comparison of 59_{HTL} and 89_{HTL} (FIG. 13E, 13F) the histone H2B-HaloTag® ESCs were stained with 5 nM 59_{HTL} for 1 h or 0.1 nM 89_{HTL} for 15 min at 37° C. with 5% (v/v) CO₂ to achieve sparse labeling with approximately equal localizations per region of interest (ROI), and then washed with dye-free media 3× for 30 min each wash For comparison of 40_{HTL}, 44_{HTL}, 1_{HTL}, or 89_{HTL} (FIG. 12A-12G) the histone H2B-HaloTag® ESCs were stained with 1 nM of HaloTag® ligands for 15 min at 37° C. with 5% (v/v) CO₂ to achieve sparse labeling, and then washed with dye-free media 3× for 30 min each wash.

To quantify the brightness and photostability of the HaloTag® ligands, labeled cells were mounted onto a high speed motorized Nikon Eclipse Ti-E inverted microscope equipped with the following: 100× Apo TIRF 1.49 NA objective with a correction collar; four excitation laser lines (405/488/561/642 nm) and matching TIRF quad cube (405/488/561/640 nm reflection bands); automatic TIRF illuminator with motorized X axis and manual Y axis for beam positioning and focus: perfect focus 3 system; Triple DU-897 iXon Ultra EMCCD cameras on a Cairn Tri-cam emission splitter with filters 525/50 (GFP) 600/50 (RFP) and 705/72 (Cy5); humidified incubation chamber maintained at 37° C. with 5% v/v CO₂ (Tokai Hit). SPT was performed at 100 Hz for the comparison of 59_{HTL} and 89_{HTL} (FIG. 13E, 13F) and 50 Hz for the comparison of 40_{HTL}, 44_{HTL}, 1_{HTL}, or 89_{HTL} (FIG. 12A-12G); the imaging session was performed for ˜5000 frames using 100% of maximal excitation power (˜120 mW at the fiber end before the TIRF unit). The excitation laser was controlled by an acousto-optic tunable filter (AOTF) and reflected into the objective by a multi-band dichroic (405/488/561/633 BrightLine quad-band band pass filter; Semrock). For the comparison of HaloTag® ligands 59_{HTL} and 89_{HTL} (FIG. 13E, 13F) and the comparison of compounds 40_{HTL}, 44_{HTL}, 1_{HTL}, and 90_{HTL} (FIG. 12A-12G), the fluorophores were excited with a 647 nm laser. To minimize drift during imaging, the incubation chamber was fully thermally equilibrated, and all imaging was conducted in an isolated ultra-clean room with minimal mechanical vibrations. The microscope, laser lines, and camera integration were controlled using the Nikon NIS-Elements software. Cells with obvious movement during acquisition were removed from further analysis. SMT analysis was based on multiple target tracing 86 (MTT) with tracking parameters similar to previous work.⁸⁴ For SPT brightness (photon counts/molecule/s), intensity values from sparsely-labeled molecules were analyzed. For the comparison of HaloTag® ligands 59_{HTL} and 89_{HTL} (FIG. 13E, 13F): n=147,298 single-molecule events using 59_{HTL}; n=100,392 single-molecule events using 89_{HTL}. For the comparison of dyes 40_{HTL}, 44_{HTL}, 1_{HTL}, and 90_{HTL} (FIG. 12A-12G): n=655,357 single-molecule events using 40_{HTL}; n=1,048,575 single-molecule events using 44_{HTL}; n=424,136 single-molecule events using 1_{HTL}; n=564,263 single-molecule events using 89_{HTL}. For the track length analysis, single-molecule trajectories of each cell with at least >5 constitutive frames were combined for each condition. For the comparison of dyes 40_{HTL}, 44_{HTL}, 1_{HTL}, and 90_{HTL} (FIG. 12A-12G): n=53 cells using 40_{HTL}; n=62 cells using 44_{HTL}; n=54 cells using 1_{HTL}; n=62 cells using 89_{HTL}.

### Example 209: cAMP FLIM-FRET sensor ScAMPI (FIG. 5)

The Epac-based cAMP sensor “Epac-S^{H189}”, was obtained from Addgene (Plasmid #170348). This sensor consists of a mTurquoise fluorescent protein and a tdDark Venus protein linked via the Epac1 protein.

To construct the SNAP-tag®-Epac1-HaloTag® sensor, the sequence encoding the tdDark Venus domain in Epac-S^{H189} was first replaced with the HaloTag® sequence using Nhe1 and Asc1 fragment ligation (NEB), creating a construct with mTurquoise and HaloTag® flanking the Epac1 domain (i.e, mTurquoise-Epac1-HaloTag®).

A gene block encoding the SNAP-tag® self-labeling protein (IDT) was then used to replace the portion encoding the mTurquoise domain in the mTurquoise-Epac1-HaloTag® using HindIII and AgeI fragment ligation. The sequence of this final plasmid encoding ScAMPI (i.e, SNAP-tag®-Epac1-HaloTag®) was confirmed through sequence analysis (Genewiz).

To evaluate this semisynthetic system in cells, U2OS cells (ATCC) were maintained in DMEM (Corning) containing 10% v/v FBS (ATCC), 1% v/v L-glutamine (Gibco) and 1% v/v Pen/Strep (Gibco). Cells were transfected with plasmid DNA using nucleofection (Lonza) and labeled 48 h post-transfection with 100 nM of JFX₆12⁻SNAP-tag® (88_{STL}) and 200 nM of the JQ₆₄₅-HaloTag® ligand (65_{HTL}) for 1 h. Cells were washed with 1×PBS (3×) and recovered in fresh medium for 30 min before imaging. Fluorescence lifetime imaging was performed with a Leica SP8 Falcon using LASX software for image acquisition and analysis. Excitation was achieved with a pulsed white light laser at 40 and 20 MHz using a 40× oil immersion objective at 1.3 NA. Fluorescence emission was collected by the HyD detector. The SNAP-tag® donor dye was excited at 610 nm and emission was collected at 620-720 nm. FLIM images were acquired before and 10 min after stimulation with 25 M forskolin. FLIM data were fitted with two-component exponential fitting function yielding quenched lifetime (τ₁) and unquenched lifetime (τ₂). Mean amplitude weighted lifetime image (τₘₑₐₙ) was calculated by fixing the two lifetimes and performing an image fit for their respective amplitudes (a₁ and a₂). FRET efficiency was calculated using the mean lifetime obtained in absence (τ_{mean,-forskolin}) and (τ_{mean,+forskolin}) presence of forskolin as 1⁻(τ_{mean,-forskolin}/τ_{mean,+forskolin}).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

(1) Coons, A. H.; Creech, H.; Jones, R.; Berliner, E. The demonstration of pneumococcal antigen in tissues by the use of fluorescent antibody. J. Immunol. 1942, 45, 159-170.
(2) Riggs, J. L.; Seiwald, R. J.; Burckhalter, J. H.; Downs, C. M.; Metcalf, T. G. Isothiocyanate compounds as fluorescent labeling agents for immune serum. Am. J. Pathol. 1958, 34, 1081-1097.
(3) Grimm, J. B.; Lavis, L. D. Caveat fluorophore: An insiders' guide to small-molecule fluorescent labels. Nat. Methods 2022, 19, 149-158.
(4) Keppler, A.; Gendreizig, S.; Gronemeyer, T.; Pick, H.; Vogel, H.; Johnsson, K. A general method for the covalent labeling of fusion proteins with small molecules in vivo. Nat. Biotechnol. 2002, 21, 86-89.
(5) Kolberg, K.; Puettmann, C.; Pardo, A.; Fitting, J.; Barth, S. SNAP-tag® technology: A general introduction. Curr. Pharm. Des. 2013, 19, 5406-5413.
(6) Los, G. V; Encell, L. P.; McDougall, M. G.; Hartzell, D. D.; Karassina, N.; Zimprich, C.; Wood, M. G.; Learish, R.; Ohana, R. F.; Urh, M. HaloTag®: A novel protein labeling technology for cell imaging and protein analysis. ACS Chem. Biol. 2008, 3, 373-382.
(7) Encell, L. P.; Friedman Ohana, R.; Zimmerman, K.; Otto, P.; Vidugiris, G.; Wood, M. G.; Los, G. V.; McDougall, M. G.; Zimprich, C.; Karassina, N.; et al. Development of a dehalogenase-based protein fusion tag capable of rapid, selective and covalent attachment to customizable ligands. Curr. Chem. Genomics 2012, 6, 55-71.
(8) Xue, L.; Karpenko, I. A.; Hiblot, J.; Johnsson, K. Imaging and manipulating proteins in live cells through covalent labeling. Nat. Chem. Biol. 2015, 11, 917-923.
(9) Lavis, L. D. Chemistry is dead. Long live chemistry! Biochemistry 2017, 56, 5165-5170.
(10) Rodriguez, E. A.; Campbell, R. E.; Lin, J. Y; Lin, M. Z.; Miyawaki, A.; Palmer, A. E.; Shu, X.; Zhang, J.; Tsien, R. Y The growing and glowing toolbox of fluorescent and photoactive proteins. Trends Biochem. Sci. 2017, 42, 111-129.
(11) Lavis, L. D.; Raines, R. T. Bright ideas for chemical biology. ACS Chem. Biol. 2008, 3, 142-155.
(12) Beija, M.; Afonso, C. A.; Martinho, J. M. Synthesis and applications of rhodamine derivatives as fluorescent probes. Chem. Soc. Rev. 2009, 38, 2410-2433.
(13) Lavis, L. D.; Raines, R. T. Bright building blocks for chemical biology. ACS Chem. Biol. 2014, 9, 855-866.
(14) Grimm, J. B.; Brown, T. A.; Tkachuk, A. N.; Lavis, L. D. General synthetic method for Si-fluoresceins and Si-rhodamines. ACS Cent. Sci. 2017, 3, 975-985.
(15) Zheng, Q.; Ayala, A. X.; Chung, I.; Weigel, A. V.; Ranjan, A.; Falco, N.; Grimm, J. B.; Tkachuk, A. N.; Wu, C.; Lippincott-Schwartz, J.; et al. Rational design of fluorogenic and spontaneously blinking labels for super-resolution imaging. ACS Cent. Sci. 2019, 5, 1602-1613.
(16) Grimm, J. B.; Tkachuk, A. N.; Xie, L.; Choi, H.; Mohar, B.; Falco, N.; Schaefer, K.; Patel, R.; Zheng, Q.; Liu, Z.; et al. A general method to optimize and functionalize red-shifted rhodamine dyes. Nat. Methods 2020, 17, 815-821.
(17) Grimm, J. B.; English, B. P.; Chen, J.; Slaughter, J. P.; Zhang, Z.; Revyakin, A.; Patel, R.; Macklin, J. J.; Normanno, D.; Singer, R. H.; et al. A general method to improve fluorophores for live-cell and single-molecule microscopy. Nat. Methods 2015, 12, 244-250.
(18) Grimm, J. B.; Muthusamy, A. K.; Liang, Y; Brown, T. A.; Lemon, W. C.; Patel, R.; Lu, R.; Macklin, J. J.; Keller, P. J.; Ji, N.; et al. A general method to fine-tune fluorophores for live-cell and in vivo imaging. Nat. Methods 2017, 14, 987-994.
(19) Sun, W.-C.; Gee, K. R.; Klaubert, D. H.; Haugland, R. P. Synthesis of fluorinated fluoresceins. J. Org. Chem. 1997, 62, 6469-6475.
(20) Liu, J.; Diwu, Z.; Leung, W.-Y; Lu, Y; Patch, B.; Haugland, R. P. Rational design and synthesis of a novel class of highly fluorescent rhodamine dyes that have strong absorption at long wavelengths. Tetrahedron Lett. 2003, 44, 4355-4359.
(21) Kolmakov, K.; Hebisch, E.; Wolfram, T.; Nordwig, L. A.; Wurm, C. A.; Ta, H.; Westphal, V; Belov, V. N.; Hell, S. W. Far-red emitting fluorescent dyes for optical nanoscopy: Fluorinated silicon-rhodamines (SiRF dyes) and phosphorylated oxazines. Chem.—Eur. J. 2015, 21, 13344-13356.
(22) Nemoto, H.; Kubota, Y; Yamamoto, Y Development of a new acyl anion equivalent for the preparation of masked activated esters, and their use to prepare a dipeptide. J. Org. Chem. 1990, 55, 4515-4516.
(23) Stagge, F.; Mitronova, G. Y; Belov, V. N.; Wurm, C. A.; Jakobs, S. SNAP-, CLIP- and Halo-tag labelling of budding yeast cells. PloS One 2013, 8, e78745.
(24) Corrêa, I. R.; Baker, B.; Zhang, A.; Sun, L.; Provost, C. R.; Lukinavičius, G.; Reymond, L.; Johnsson, K.; Xu, M.-Q. Substrates for improved live-cell fluorescence labeling of SNAP-tag®. Curr. Pharm. Des. 2013, 19, 5414-5420.
(25) Khanna, P. L.; Ullman, E. F. 4′,5′-Dimethoxy-6⁻carboxyfluorescein: A novel dipole-dipole coupled fluorescence energy transfer acceptor useful for fluorescence immunoassays. Anal. Biochem. 1980, 108, 156-161.
(26) Haugland, R. P.; Spence, M. T. Z.; Johnson, I. D.; Basey, A. The Handbook: A Guide to Fluorescent Probes and Labeling Technologies, 10th ed.; Molecular Probes, 2005.
(27) Uddin, M. J.; Marnett, L. J. Synthesis of 5⁻and 6⁻carboxy-X-rhodamines. Org. Lett. 2008, 10, 4799-4801.
(28) Fu, M.; Zhang, X. W.; Wang, J.; Chen, H. W.; Gao, Y Progress of synthesis and separation of regioisomerically pure 5(6)-substituted rhodamine. Curr. Org. Chem. 2016, 20, 1584-1590.
(29) Lukinavičius, G.; Umezawa, K.; Olivier, N.; Honigmann, A.; Yang, G.; Plass, T.; Mueller, V.; Reymond, L.; Corrêa Jr, I. R.; Luo, Z. G.; et al. A near-infrared fluorophore for live-cell super-resolution microscopy of cellular proteins. Nat. Chem. 2013, 5, 132-139.
(30) Butkevich, A. N.; Mitronova, G. Y; Sidenstein, S. C.; Klocke, J. L.; Kamin, D.; Meineke, D. N.; D′Este, E.; Kraemer, P. T.; Danzl, J. G.; Belov, V. N.; et al. Fluorescent rhodamines and fluorogenic carbopyronines for super-resolution STED microscopy in living cells. Angew. Chem. Int. Ed. 2016, 55, 3290-3294.
(31) Lukinavicius, G.; Reymond, L.; Umezawa, K.; Sallin, O.; D′Este, E.; Gottfert, F.; Ta, H.; Hell, S. W.; Urano, Y; Johnsson, K. Fluorogenic probes for multicolor imaging in living cells. J. Am. Chem. Soc. 2016, 138, 9365-9368.
(32) Grimm, J. B.; Klein, T.; Kopek, B. G.; Shtengel, G.; Hess, H. F.; Sauer, M.; Lavis, L. D. Synthesis of a far-red photoactivatable silicon-containing rhodamine for super-resolution microscopy. Angew. Chem. Int. Ed. 2016, 55, 1723-1727.
(33) Butkevich, A. N.; Belov, V. N.; Kolmakov, K.; Sokolov, V. V; Shojaei, H.; Sidenstein, S. C.; Kamin, D.; Matthias, J.; Vlijm, R.; Engelhardt, J.; et al. Hydroxylated fluorescent dyes for live-cell labeling: Synthesis, spectra and super-resolution STED. Chem.—Eur. J. 2017, 23, 12114-12119.
(34) Butkevich, A. N.; Ta, H.; Ratz, M.; Stoldt, S.; Jakobs, S.; Belov, V N.; Hell, S. W. Two-color 810 nm STED nanoscopy of living cells with endogenous SNAP-tag®ged fusion proteins. ACS. Chem. Biol. 2018, 13, 475-480.
(35) Fischer, C.; Sparr, C. Direct transformation of esters into heterocyclic fluorophores. Angew. Chem. Int. Ed. 2018, 57, 2436-2440.
(36) Butkevich, A. N. Modular synthetic approach to silicon-rhodamine homologues and analogues via bis-aryllanthanum reagents. Org. Lett. 2021, 23, 2604-2609.
(37) Schnermann, M. J.; Lavis, L. D. Rejuvenating old fluorophores with new chemistry. Curr. Opin. Chem. Biol. 2023, 75, 102335.
(38) Lee, L. G.; Graham, R. J.; Werner, W. E.; Swartzman, E.; Lu, L. Water-soluble rhodamine dyes and conjugates thereof. U.S. Pat. No. 6,191,278 B1, 2001.
(39) Kolmakov, K.; Wurm, C. A.; Meineke, D. N.; Gottfert, F.; Boyarskiy, V. P.; Belov, V. N.; Hell, S. W. Polar red-emitting rhodamine dyes with reactive groups: Synthesis, photophysical properties, and two-color STED nanoscopy applications. Chem.—Eur. J. 2014, 20, 146-157.
(40) Grimm, J. B.; Lavis, L. D. Synthesis of rhodamines from fluoresceins using Pd-catalyzed C-N cross-coupling. Org. Lett. 2011, 13, 6354-6357.
(41) Grimm, J. B.; Sung, A. J.; Legant, W. R.; Hulamm, P.; Matlosz, S. M.; Betzig, E.; Lavis, L. D. Carbofluoresceins and carborhodamines as scaffolds for high-contrast fluorogenic probes. ACS Chem. Biol. 2013, 8, 1303-1310.
(42) Dwight, S. J.; Levin, S. Scalable regioselective synthesis of rhodamine dyes. Org. Lett. 2016, 18, 5316-5319.
(43) Richardson, R. D.; Zayed, J. M.; Altermann, S.; Smith, D.; Wirth, T. Tetrafluoro-IBA and -IBX: hypervalent iodine reagents. Angew. Chem. Int. Ed. 2007, 46, 6529-6532.
(44) Wheeler, D. D.; Young, D. C.; Erley, D. S. Reactions of phthalaldehydic acid. J. Org. Chem. 1957, 22, 547-556.
(45) Grimm, J. B.; Xie, L.; Casler, J. C.; Patel, R.; Tkachuk, A. N.; Falco, N.; Choi, H.; Lippincott-Schwartz, J.; Brown, T. A.; Glick, B. S.; et al. A general method to improve fluorophores using deuterated auxochromes. JACS Au 2021, 1, 690-696.
(46) Belov, V. N.; Bossi, M. L.; Folling, J.; Boyarskiy, V. P.; Hell, S. W. Rhodamine spiroamides for multicolor single-molecule switching fluorescent nanoscopy. Chemistry 2009, 15, 10762-10776.
(47) Kuks, E. L.; Schwarz, R.; Sella, E.; Armon, M. S.; Szwarcman, D. Rhodamine derivatives dyes and uses thereof. U.S. Pat. No. 9,711,480 B2, 2017.
(48) Koide, Y; Urano, Y; Hanaoka, K.; Terai, T.; Nagano, T. Evolution of Group 14 rhodamines as platforms for near-infrared fluorescence probes utilizing photoinduced electron transfer. ACS Chem. Biol. 2011, 6, 600-608.
(49) Fu, M.; Xiao, Y; Qian, X.; Zhao, D.; Xu, Y A design concept of long-wavelength fluorescent analogs of rhodamine dyes: Replacement of oxygen with silicon atom. Chem. Commun. 2008, 1780-1782.
(50)Kushida, Y; Nagano, T.; Hanaoka, K. Silicon-substituted xanthene dyes and their applications in bioimaging. Analyst 2015, 140, 685-695.
(51) Arden-Jacob, J.; Frantzeskos, J.; Kemnitzer, N. U.; Zilles, A.; Drexhage, K. H. New fluorescent markers for the red region. Spectrochim. Acta, Part A 2001, 57, 2271-2283.
(52) Kolmakov, K.; Belov, V. N.; Wurm, C. A.; Harke, B.; Leutenegger, M.; Eggeling, C.; Hell, S. W. Aversatile route to red-emitting carbopyronine dyes for optical microscopy and nanoscopy. Eur. J. Org. Chem. 2010, 2010, 3593-3610.
(53) Bucevicius, J.; Gerasimaite, R.; Kiszka, K. A.; Pradhan, S.; Kostiuk, G.; Koenen, T.; Lukinavicius, G. A general highly efficient synthesis of biocompatible rhodamine dyes and probes for live-cell multicolor nanoscopy. Nat. Commun. 2023, 14, 1306.
(54) Grimm, J. B.; Gruber, T. D.; Ortiz, G.; Brown, T. A.; Lavis, L. D. Virginia Orange: Aversatile, red-shifted fluorescein scaffold for single- and dual-input fluorogenic probes. Bioconjugate Chem. 2016, 27, 474-480.
(55) Kolmakov, K.; Wurm, C.; Sednev, M. V; Bossi, M. L.; Belov, V. N.; Hell, S. W. Masked red-emitting carbopyronine dyes with photosensitive 2⁻diazo-1⁻indanone caging group. Photochem. Photobiol. Sci. 2012, 11, 522-532.
(56) Lukhtanov, E. A. Carborhodamine compounds and methods of preparation thereof. U.S. Pat. No. 11,155,713 B2, 2021.
(57) Bachman, J. L.; Pavlich, C. I.; Boley, A. J.; Marcotte, E. M.; Anslyn, E. V. Synthesis of carboxy ATTO 647N using redox cycling for xanthone access. Org. Lett. 2020, 22, 381-385.
(58) Bunnett, J. F.; Zahler, R. E. Aromatic nucleophilic substitution reactions. Chem. Rev. 1951, 49, 273-412.
(59) Wall, L. A.; Fearn, J. E.; Pummer, W. J.; Antonucci, J. M. Reactions of polyfluorobenzenes with nucleophilic reagents. J. Res. Natl. Bur. Stand. (U. S.) 1963, 67A, 481-504.
(60) Martineau, M.; Somasundaram, A.; Grimm, J. B.; Gruber, T. D.; Choquet, D.; Taraska, J. W.; Lavis, L. D.; Perrais, D. Semisynthetic fluorescent pH sensors for imaging exocytosis and endocytosis. Nat. Commun. 2017, 8, 1412.
(61) Englman, R.; Jortner, J. The energy gap law for radiationless transitions in large molecules. Mol. Phys. 1970, 18, 145-164.
(62) Zhang, X. F. The effect of phenyl substitution on the fluorescence characteristics of fluorescein derivatives via intramolecular photoinduced electron transfer. Photochem. Photobiol. Sci. 2010, 9, 1261-1268.
(63) Chi, W.; Qi, Q.; Lee, R.; Xu, Z.; Liu, X. A unified push-pull model for understanding the ring-opening mechanism of rhodamine dyes. J. Phys. Chem. C 2020, 124, 3793-3801.
(64) Hansch, C.; Leo, A.; Taft, R. W. A survey of Hammett substituent constants and resonance and field parameters. Chem. Rev. 1991, 91, 165-195.
(65) Castelino, R. W.; Hallas, G. Electronic absorption spectra of some julolidine (2,3,6,7-tetrahydro-1H, 5H-benzo[ij]quinolizine) analogues of 4⁻dimethylaminoazobenzenes. J. Chem. Soc. B 1971, 793-795.
(66) Hallas, G.; Jalil, M. A. The effects of cyclic terminal groups in 4⁻aminoazobenzene and related azo dyes: Part 4—Electronic absorption spectra of some Monoazo dyes derived from N-phenylaziridine and N-Phenylazetidine11Part 3 was published in J. Chem. Soc. Perkin Trans., 2 (1986) 123.1. Dyes Pigm. 1992, 20, 13-23.
(67) Kim, D.; Stoldt, S.; Weber, M.; Jakobs, S.; Belov, V. N.; Hell, S. W. A bright surprise: Live-cell labeling with negatively charged fluorescent probes based on disulfonated rhodamines and HaloTag®. Chem. Methods 2023, e202200076.
(68) Wolff, J. O.; Scheiderer, L.; Engelhardt, T.; Engelhardt, J.; Matthias, J.; Hell, S. W. MINFLUX dissects the unimpeded walking of kinesin-1. Science 2023, 379, 1004-1010.
(69) Meissner, G. W.; Grimm, J. B.; Johnston, R. M.; Sutcliffe, B.; Ng, J.; Jefferis, G.; Cachero, S.; Lavis, L. D.; Malkesman, O. Optimization of fluorophores for chemical tagging and immunohistochemistry of Drosophila neurons. PLoS One 2018, 13, e0200759.
(70) Berlier, J. E.; Rothe, A.; Buller, G.; Bradford, J.; Gray, D. R.; Filanoski, B. J.; Telford, W. G.; Yue, S.; Liu, J.; Cheung, C. Y; et al. Quantitative comparison of long-wavelength Alexa Fluor dyes to Cy dyes: Fluorescence of the dyes and their bioconjugates. J. Histochem. Cytochem. 2003, 51, 1699-1712.
(71) Koide, Y; Urano, Y; Hanaoka, K.; Piao, W.; Kusakabe, M.; Saito, N.; Terai, T.; Okabe, T.; Nagano, T. Development of NIR fluorescent dyes based on Si-rhodamine for in vivo imaging. J. Am. Chem. Soc. 2012, 134, 5029-5031.
(72) Jeon, J.; Lee, K. H.; Rao, J. A strategy to enhance the binding affinity of fluorophore-aptamer pairs for RNA tagging with neomycin conjugation. Chem. Commun. (Camb.) 2012, 48, 10034-10036.
(73) Hanaoka, K.; Iwaki, S.; Yagi, K.; Myochin, T.; Ikeno, T.; Ohno, H.; Sasaki, E.; Komatsu, T.; Ueno, T.; Uchigashima, M.; et al. General design strategy to precisely control the emission of fluorophores via a twisted intramolecular charge transfer (TICT) process. J. Am. Chem. Soc. 2022, 144, 19778-19790.
(74) Myochin, T.; Hanaoka, K.; Iwaki, S.; Ueno, T.; Komatsu, T.; Terai, T.; Nagano, T.; Urano, Y Development of a series of near-infrared dark quenchers based on Si-rhodamines and their application to fluorescent probes. J. Am. Chem. Soc. 2015, 137, 4759-4765.
(75) Wu, L.; Huang, C.; Emery, B. P.; Sedgwick, A. C.; Bull, S. D.; He, X. P.; Tian, H.; Yoon, J.; Sessler, J. L.; James, T. D. Forster resonance energy transfer (FRET)-based small-molecule sensors and imaging agents. Chem. Soc. Rev. 2020, 49, 5110-5139.
(76) Bos, J. L. Epac proteins: Multi-purpose cAMP targets. Trends Biochem. Sci. 2006, 31, 680-686.
(77) Klarenbeek, J.; Goedhart, J.; van Batenburg, A.; Groenewald, D.; Jalink, K. Fourth-generation epac-based FRET sensors for cAMP feature exceptional brightness, photostability and dynamic range: Characterization of dedicated sensors for FLIM, for ratiometry and with high affinity. PLoS One 2015, 10, e0122513.
(78) Drexhage, K. H. Fluorescence efficiency of laser dyes. J. Res. Natl. Bur. Stand. A Phys. Chem. 1976, 80A, 421-428.Coons, A. H.; Creech, H.; Jones, R.; Berliner, E. The demonstration of pneumococcal antigen in tissues by the use of fluorescent antibody. J Immunol. 1942, 45, 159-170.
(79) Wang, D. X.; Wang, L. L.; Xue, L.; Zhou, D. B.; Feng, S. Y; Zhao, X. Tetrahedral silicon-based luminescent molecules: Synthesis and comparison of thermal and photophysical properties by various effect factors. J. Organomet. Chem. 2013, 735, 58-64.
(80) Egawa, T.; Koide, Y; Hanaoka, K.; Komatsu, T.; Terai, T.; Nagano, T. Development of a fluorescein analogue, TokyoMagenta, as a novel scaffold for fluorescence probes in red region. Chem. Commun. 2011, 47, 4162-4164.
(81) Suzuki, K.; Kobayashi, A.; Kaneko, S.; Takehira, K.; Yoshihara, T.; Ishida, H.; Shiina, Y; Oishi, S.; Tobita, S. Reevaluation of absolute luminescence quantum yields of standard solutions using a spectrometer with an integrating sphere and a back-thinned CCD detector. Phys. Chem. Chem. Phys. 2009, 11, 9850-9860.
(82) Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J. Y; White, D. J.; Hartenstein, V; Eliceiri, K.; Tomancak, P.; Cardona, A. Fiji: An open-source platform for biological-image analysis. Nat. Methods 2012, 9, 676-682.
(83) Wilhelm, J.; Kuhn, S.; Tarnawski, M.; Gotthard, G.; Tunnermann, J.; Tanzer, T.; Karpenko, J.; Mertes, N.; Xue, L.; Uhrig, U.; Reinstein, J.; Hiblot, J.; Johnsson, K. Kinetic and structural characterization of the self-labeling protein tags HaloTag@7, SNAP-tag®, and CLIP-tag. Biochemistry 2021, 60, 2560-2575.
(84) Xie, L.; Torigoe, S. E.; Xiao, J.; Mai, D. H.; Li, L.; Davis, F. P.; Dong, P.; Marie-Nelly, H.; Grimm, J.; Lavis, L.; Darzacq, X.; Cattoglio, C.; Liu, Z.; Tjian, R. A dynamic interplay of enhancer elements regulates Klf4 expression in naïve pluripotency. Genes Dev. 2017, 31, 1795-1808.
(85) Chen, J.; Zhang, Z.; Li, L.; Chen, B.-C.; Revyakin, A.; Hajj, B.; Legant, W.; Dahan, M.; Lionnet, T.; Betzig, E.; Tjian, R.; Liu, Z. Single-molecule dynamics of enhanceosome assembly in embryonic stem cells. Cell 2014, 156, 1274-1285.
(86) Sergé, A.; Bertaux, N.; Rigneault, H.; Marguet, D. Dynamic multiple-target tracing to probe spatiotemporal cartography of cell membranes. Nat. Methods 2008, 5, 687-694.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound of the following structure: 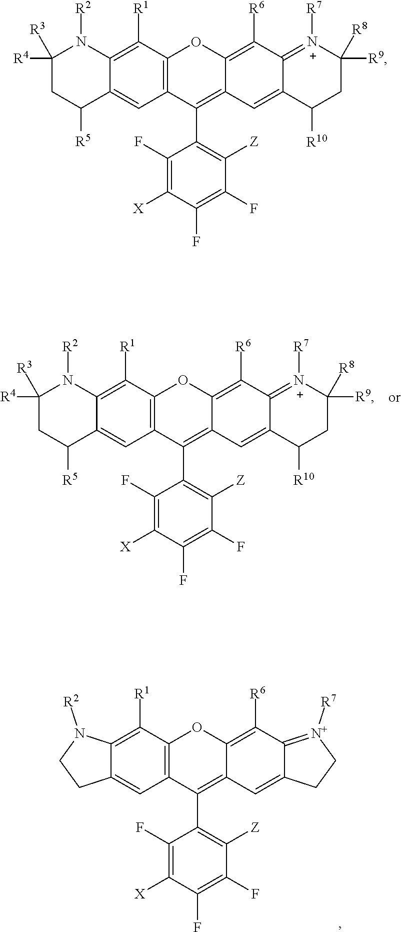
and salts thereof, wherein
Q is selected from the group consisting of C(alkyl)₂, NH, N(alkyl), O, S, SO₂, Si(alkyl)₂, P(O)(aryl), P(O)(alkyl), PO ₂H, PO ₂(alkyl), Se, and replaced with two H atoms;
R¹ and R⁶ are independently selected from the group consisting of H, D, halogen, CN, OH, O(alkyl), O(aryl), SH, S(alkyl), S(aryl), N₃, NH₂, NH(alkyl), N(alkyl)₂, NH(aryl), N(aryl)₂, NO₂, CHO, C(O)(alkyl), C(O)(aryl), COOH, COO(alkyl), COO(aryl), C(O)NH(alkyl), C(O)NH(aryl), PO ₃H2, SO₃H, alkyl and substituted alkyl, aryl and substituted aryl, alkenyl and substituted alkenyl, alkynyl or substituted alkynyl, or wherein the R¹ substituent and R² substituent and/or the R⁶ substituent and R⁷ substituent, taken together with the carbon atoms to which they are bonded, independently form a substituted or unsubstituted ring containing 3, 4, 5, 6, 7, 8, or 9 atoms;
R² and R⁷ are independently selected from the group consisting of H, alkyl, deuterated alkyl, and aryl so long as when Q=C(CH₃)₂, R¹ and R² form a 6⁻membered ring, and R¹, R⁹, and R¹⁰ are methyl, then R⁷ cannot be methyl;
each R³, R⁴, R⁵, R¹, R⁹, R¹⁰ is independently selected from the group consisting of H and alkyl, so long as when R¹ and R⁶ are H, and R³, R⁴, R⁵, R¹, R⁹, and R¹⁰ are methyl, then Q cannot be 0;
Z is selected from the group consisting of C(O), COOH, COO⁻, SO₃⁻, PO ₃²⁻, or CR₃ where each R is independently selected from the group consisting of H, OH, alkyl, and substituted alkyl; and
X is selected from the group consisting of H, alkyl, substituted alkyl, aryl, substituted aryl, alkenyl, substituted alkenyl, alkynyl, substituted alkynyl, CN, COOH, COO⁻, COO(alkyl), COO(aryl), C(O)NH(alkyl), C(O)N(alkyl)₂, C(O)NH(aryl), C(O)N(aryl)₂, PO ₃H2, and SO₃H.

2. The compound of claim 1, wherein X is selected from the group consisting of alkyl, substituted alkyl, aryl, substituted aryl, alkenyl, substituted alkenyl, alkynyl, substituted alkynyl, CN, COOH, COO(alkyl), COO(aryl), C(O)NH(alkyl), C(O)N(alkyl)₂, C(O)NH(aryl), C(O)N(aryl)₂.

3. The compound of claim 1, wherein X is selected from the group consisting of

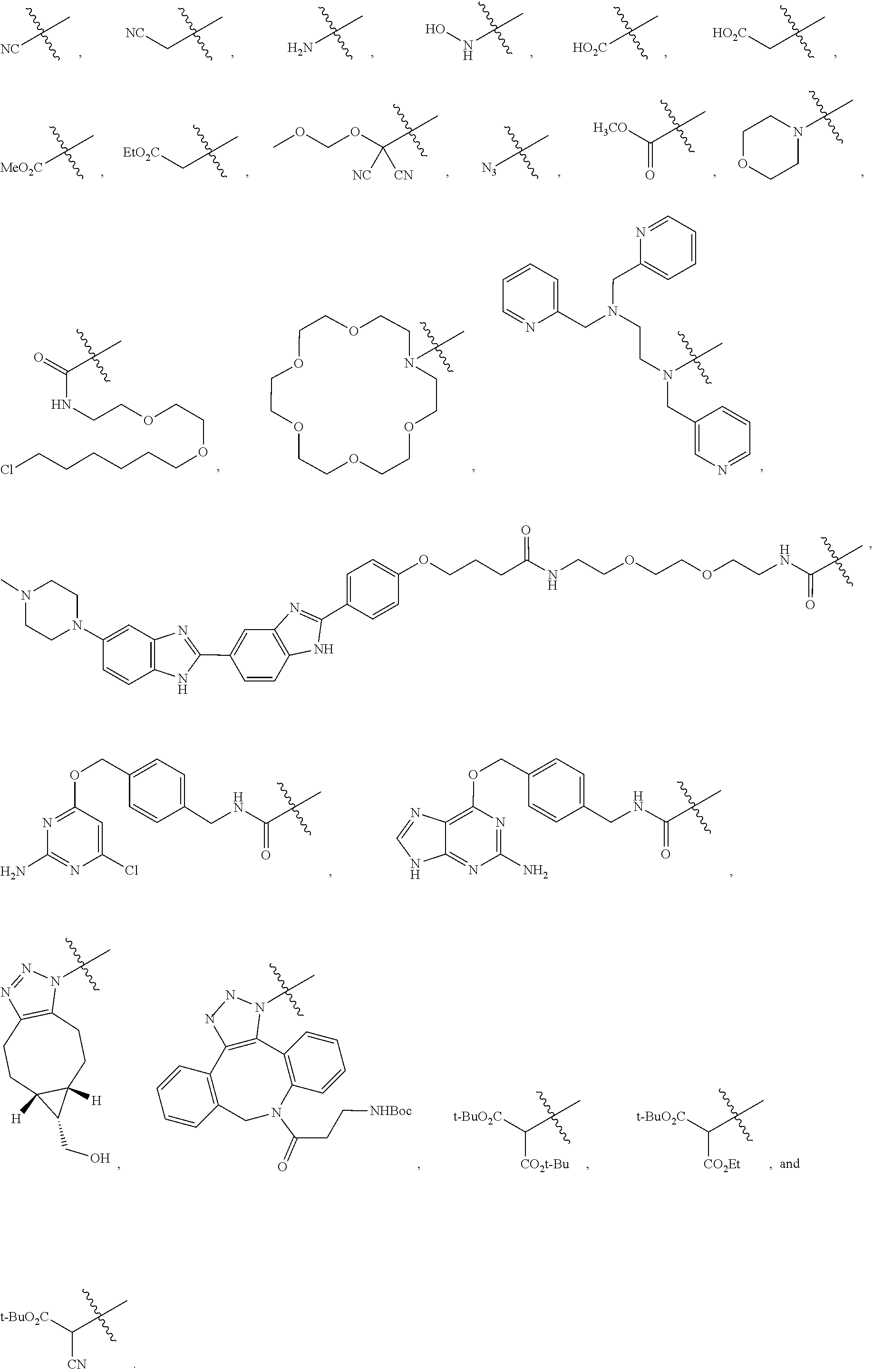

4. The compound of claim 1, wherein Q is selected from selected from the group consisting of 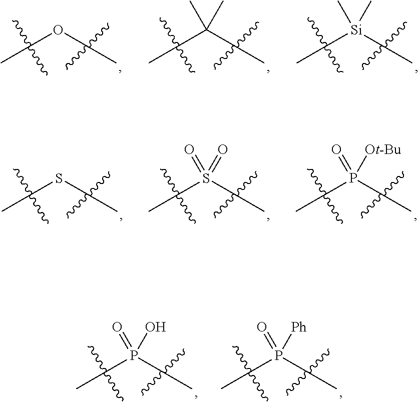
and replaced with two H atoms.

5. The compound of claim 1, wherein Q is selected from selected from the group consisting of

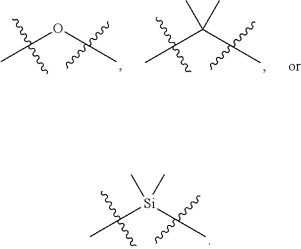

6. The compound of claim 1, wherein R² and R⁷ are independently selected from the group consisting of H and alkyl.

7. The compound of claim 1, wherein the R¹ substituent and R² substituent and/or the R⁶ substituent and R⁷ substituent, taken together with the carbon atoms to which they are bonded, independently form a substituted or unsubstituted ring containing 3, 4, 5, 6, 7, 8, or 9 atoms.

8. The compound of claim 1, wherein the R¹ substituent and R² substituent and/or the R⁶ substituent and R⁷ substituent, taken together with the carbon atoms to which they are bonded, independently form a substituted or unsubstituted ring containing 6 atoms.

9. The compound of claim 1, wherein each R³, R⁴, R⁵, R¹, R⁹, and R¹⁰ is independently selected from the group consisting of H and methyl.

10. The compound of claim 1, having the following structure: 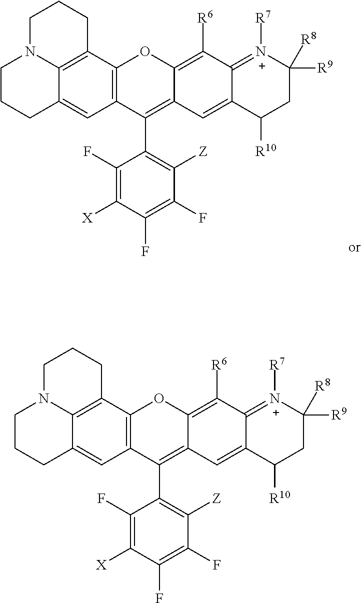
wherein
Q is selected from the group consisting of C(alkyl)₂, NH, N(alkyl), O, S, SO₂, Si(alkyl)₂, P(O)(aryl), P(O)(alkyl), PO ₂H, PO ₂(alkyl), Se, and replaced with two H atoms;
R⁶ is selected from the group consisting of H, D, halogen, CN, OH, O(alkyl), O(aryl), SH, S(alkyl), S(aryl), N₃, NH₂, NH(alkyl), N(alkyl)₂, NH(aryl), N(aryl)₂, NO₂, CHO, C(O)(alkyl), C(O)(aryl), COOH, COO(alkyl), COO(aryl), C(O)NH(alkyl), C(O)NH(aryl), PO ₃H2, SO₃H, alkyl and substituted alkyl, aryl and substituted aryl, alkenyl and substituted alkenyl, alkynyl or substituted alkynyl, or where the R⁶ substituent and R⁷ substituent, taken together with the carbon atoms to which they are bonded, independently form a substituted or unsubstituted ring containing 3, 4, 5, 6, 7, 8, or 9 atoms;
R⁷ is selected from the group consisting of H, alkyl, deuterated alkyl, and aryl, so long as when Q=C(CH₃)₂ and R⁸, R⁹, and R¹⁰ are methyl, then R⁷ cannot be methyl.
each R⁸, R⁹, and R¹⁰ is independently selected from the group consisting of H and alkyl;
Z is selected from the group consisting of C(O), COOH, COO⁻, SO₃⁻, PO ₃²⁻, or CR₃ where each R is independently selected from the group consisting of H, OH, alkyl, and substituted alkyl; and
X is selected from the group consisting of H, alkyl, substituted alkyl, aryl, substituted aryl, alkenyl, substituted alkenyl, alkynyl, substituted alkynyl, CN, COOH, COO⁻, COO(alkyl), COO(aryl), C(O)NH(alkyl), C(O)N(alkyl)₂, C(O)NH(aryl), C(O)N(aryl)₂, PO ₃H2, and SO₃H.

11. The compound of claim 1, having a structure chosen from:

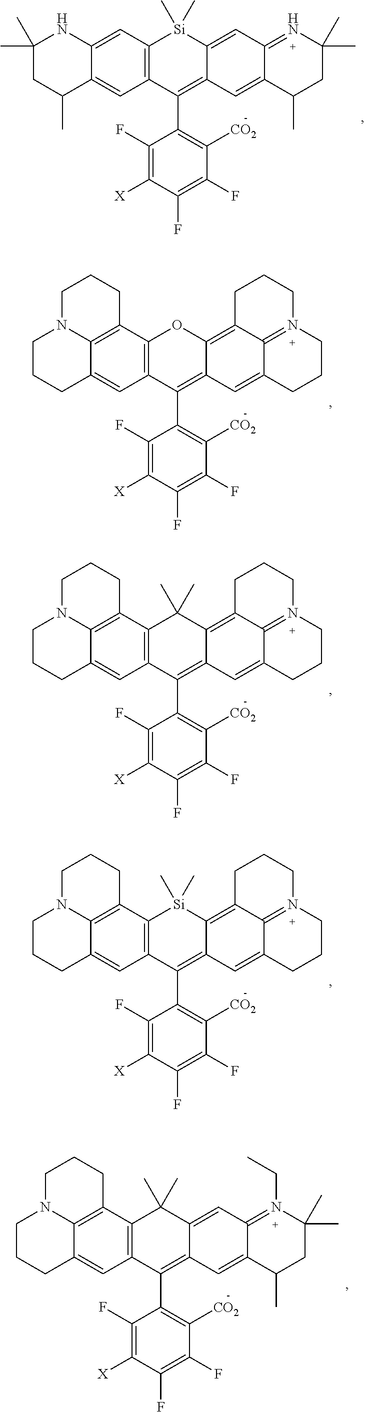

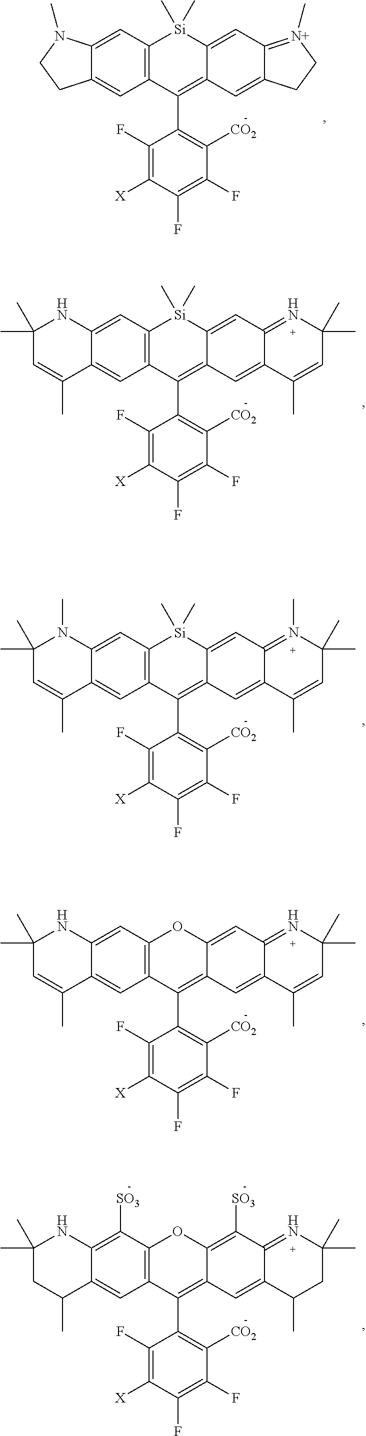

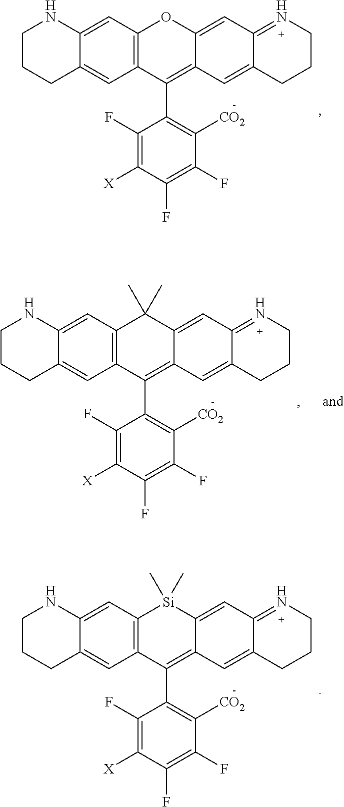

12. The compound of claim 11, wherein X is selected from the group consisting of alkyl, substituted alkyl, aryl, substituted aryl, alkenyl, substituted alkenyl, alkynyl, substituted alkynyl, CN, COOH, COO(alkyl), COO(aryl), C(O)NH(alkyl), C(O)N(alkyl)₂, C(O)NH(aryl), C(O)N(aryl)₂.

13. The compound of claim 11, wherein X is selected from the group consisting of

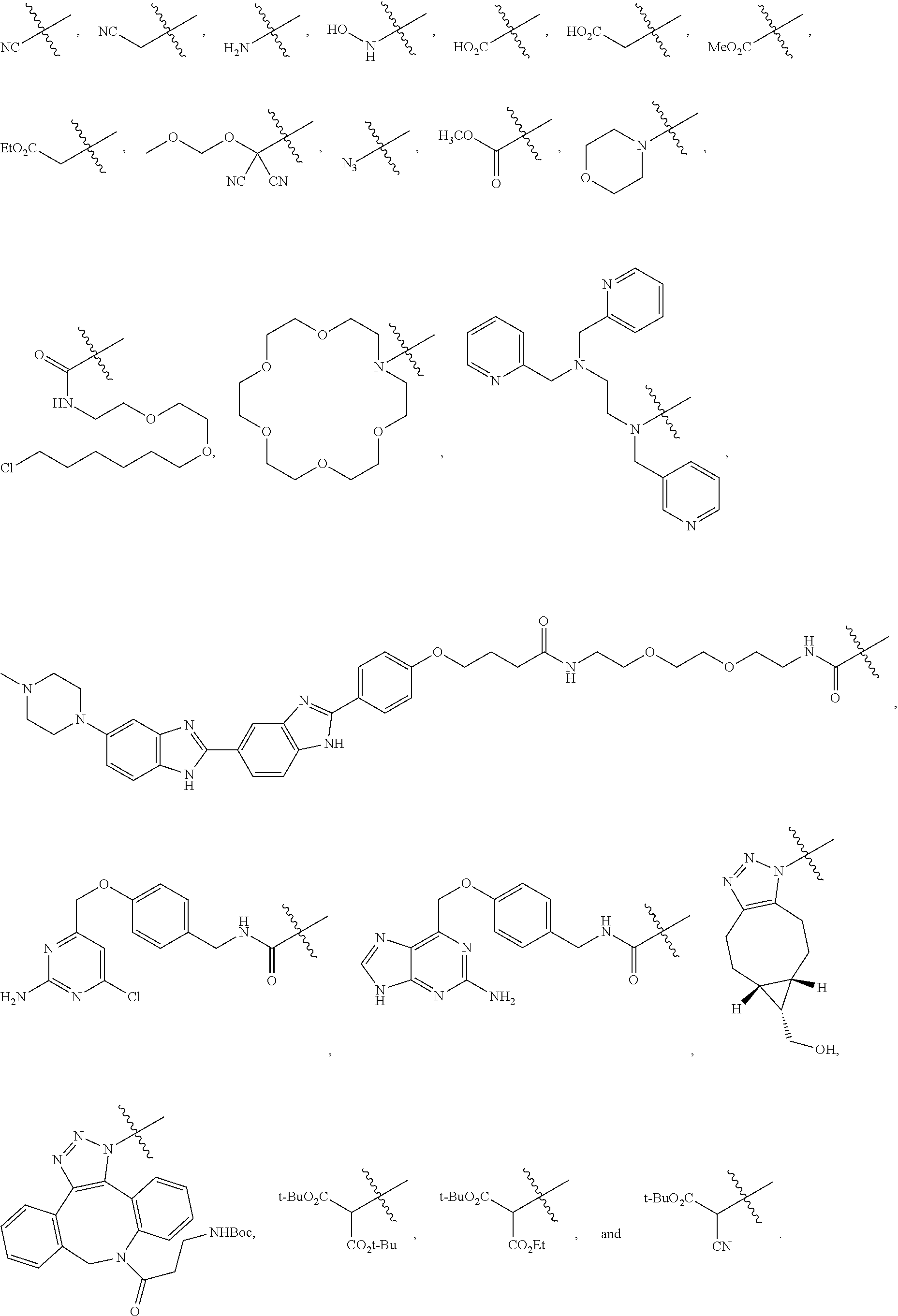

14. A method for detecting a target substance, comprising contacting a sample with the compound of claim 1.

15. The method of claim 14, wherein the target substance is selected from a protein, a carbohydrate, a polysaccharide, a glycoprotein, a hormone, a receptor, an antigen, an antibody, a virus, a substrate, a metabolite, an inhibitor, a drug, a nutrient, a growth factor, a liprotein, and a combination thereof.

16. The method of claim 14, wherein the detecting step is performed with a microscope.

17. The method of claim 14, wherein the contact step and the detecting step are performed in a live cell.

18. The method of claim 14, comprising contacting the sample with the first compound of claim 1 and a second compound of claim 1, wherein the first compound is selective for a first target substance and capable of emitting a first emission light, the second compound is selective for a second target substance and capable of emitting a second emission light, and the detecting step includes detecting the first emission light that indicates the presence of the first target substance and the second emission light that indicates the presence of the second target substance.
